(19) United States
(12) Patent Application Publication
(10) Pub. No.: US 2026/0091026 A1
(43) Pub. Date: Apr. 2, 2026

(54) METHODS OF TREATING INFLAMMATORY DISEASES

(71) Applicant: INTERLINE THERAPEUTICS, INC., Brisbane, CA (US)

(72) Inventors: Robert Joseph MOREAU, Walnut Creek, CA (US); Naomi S. RAJAPAKSA, Belmont, CA (US); David C. TULLY, Danville, CA (US); Mario G. CARDOZO, San Francisco, CA (US); Zuojun GUO, Pleasanton, CA (US); Meera RAO, San Francisco, CA (US); Steffen GREßIES, Berlin (DE); Kai THEDE, Berlin (DE); Zachary K. SWEENEY, Redwood City, CA (US)

(21) Appl. No.: 19/411,055

(22) Filed: Dec. 5, 2025

Related U.S. Application Data
(63) Continuation of application No. PCT/US24/33114, filed on Jun. 7, 2024.
(60) Provisional application No. 63/472,233, filed on Jun. 9, 2023.

(51) Int. Cl. A61K 31/437; A61K 31/497; A61K 31/501; A61K 31/506; A61P 29/00; C07D 471/04

(52) U.S. Cl. CPC A61K 31/437; A61K 31/497; A61K 31/501; A61K 31/506; A61P 29/00; C07D 471/04

(57) ABSTRACT

The present application relates to compounds of Formula (I) and Formula (II), as defined herein, and pharmaceutically acceptable salts thereof, and compositions comprising same. Also described are methods of treating the diseases and disorders disclosed herein, with the compounds of Formula (I) or Formula (II), and pharmaceutically acceptable salts of the foregoing, and the compositions comprising same.

This application is a continuation application of International Application No. PCT/US2024/033114, filed on Jun. 7, 2024, which claims priority to U.S. Provisional Application No. 63/472,233 filed on Jun. 9, 2023; the entire contents of which are hereby incorporated by reference.

### FIELD

The present application relates to the fields of chemistry and biology, in particular to compounds of Formula (I) and Formula (II), as defined herein, and pharmaceutically acceptable salts thereof, and compositions comprising same. Also described are methods of treating the diseases and disorders disclosed herein, with the compounds of Formula (I) and/or Formula (II), and pharmaceutically acceptable salts thereof, and the compositions comprising same.

### BACKGROUND

Receptor interacting protein kinase 2 (RIPK2) is a serine-threonine protein kinase, and is a signaling molecule downstream of nucleotide-binding oligomerization domain 1 (NOD1), NOD2, and Toll-like receptors (TLRs). The RIPK2 protein includes a kinase domain (KD), an intermediate domain (INTD), and a caspase activation and recruitment domain (CARD). The CARD domain of RIPK2 mediates interaction with NOD1 and NOD2. RIPK2 is expressed in the cytoplasm of antigen-presenting cells including dendritic cells and macrophages, and is also expressed in T cells and epithelial cells.

NOD receptors function in the innate immune system, detecting bacterial pathogens by binding to diaminopimelic acid or muramyl dipeptide residues present in bacterial peptidoglycans. Interactions between RIPK2 and NOD1, NOD2 and TLRs trigger the release of pro-inflammatory cytokines including TNF-α, IL-6, and IL-12/23p40, and RIPK2-mediated induction of NF-kappa-B-dependent inflammatory responses. Activation of RIPK2 and dysregulation of the RIPK2-NOD signaling pathways may also have a role in the pathogenesis of various inflammatory diseases. RIPK2 has been reported to be a prognostic indicator and candidate therapeutic target for various cancers.

### SUMMARY

Some embodiments provide a compound of Formula (I) or Formula (II):

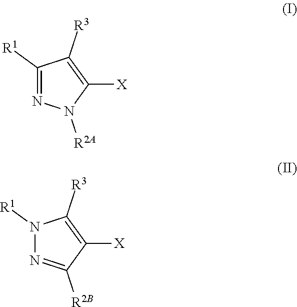

or a pharmaceutically acceptable salt of either of the foregoing, wherein:
R¹ is a 5-10 membered heteroaryl optionally substituted with 1-3 substituents independently selected from C1-C6 alkyl, C1-C6 thioalkyl, C1-C6 alkoxy optionally substituted with hydroxyl, —S(O₂) C1-C6 alkyl, NR^{1A}R^{1B}, 4-10 membered heterocyclyl optionally substituted with 1-3 substituents independently selected from halogen and C1-C6 alkyl, —(NH)—C1-C6 alkyl, and —(NH)-4-10 membered heterocyclyl optionally substituted with 1-3 substituents independently selected from halogen and C1-C6 alkyl;
R^{1A} and R^{1B} are independently selected from hydrogen, C1-C6 alkyl, and C1-C6 haloalkyl;
R^{2A} is selected from hydrogen, halogen, C1-C6 alkyl, C1-C6 haloalkyl, C1-C6 alkoxyalkyl, C1-C6 hydroxyalkyl optionally substituted with 1 or 2 halogens, C3-C6 cycloalkyl, 4-10 membered heterocyclyl optionally substituted with 1 or 2 substituents independently selected from halogen, C1-C6 alkyl, C(O)O C1-C6 alkyl;
R^{2B} is hydrogen, C1-C6 alkyl, C1-C6 haloalkyl, or C1-C6 hydroxyalkyl;
R³ is hydrogen or halogen;
X is selected from

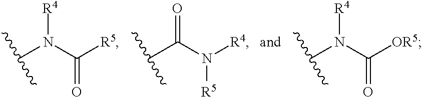

R⁴ is hydrogen or C1-C6 alkyl;
R⁵ is selected from:
(i) C1-C6 alkyl optionally substituted with 1-4 substituents independently selected from:
   (a) C3-C6 cycloalkyl optionally substituted with 1-3 substituents independently from halogen or phenyl,
   (b) phenyl optionally substituted with 1-3 substituents independently selected from halogen, C1-C6 alkoxy, C1-C6 hydroxyalkyl, and cyano,
   (c) —NR^{A}R^{B},
   (d) hydroxyl,
   (e) halogen,
   (f) 4-10 membered heterocyclyl optionally substituted with 1-3 substituents independently selected from benzyl, C1-C6 alkyl, and phenyl optionally substituted with C1-C6 alkyl, and
   (g) 5-10 membered heteroaryl optionally substituted with 1-3 substituents independently selected from halogen, cyano, C1-C6 alkyl, and C1-C6 alkoxy;
(ii) C3-C10 cycloalkyl optionally substituted with 1-3 substituents independently selected from halogen, hydroxyl, C1-C6 alkyl, phenyl optionally substituted with 1-2 independently selected halogen and 5-10 membered heteroaryl;
(iii) 4-10 membered heterocyclyl optionally substituted with 1-3 substituents independently selected from benzyl, C1-C6 alkyl, C3-C10 cycloalkyl, and phenyl optionally substituted with C1-C6 alkyl;
(iv) phenyl optionally substituted with 1-3 substituents independently selected from halogen, nitro, and C1-C6 haloalkyl;
(v) 5-10 membered heteroaryl optionally substituted with 1-3 substituents independently selected from halogen and C1-C6 alkyl;
(vi) C1-C6 alkoxy; or
(vii) C3-C6 cycloalkoxy;
or R⁴ and R⁵ with the atom to which they are attached together form a 5-12 membered heterocyclyl or 9-10 membered heteroaryl each optionally substituted with phenyl; and
R^{A} and R^{B} are independently selected from hydrogen and C1-C6 alkyl.

Also provided herein is a method of treating a RIPK2-associated disease or disorder in a subject in need thereof, comprising administering to the subject an effective amount of a compound of Formula (I), or a pharmaceutically acceptable salt thereof, or a pharmaceutical composition comprising subject an effective amount of a compound of Formula (I), or a pharmaceutically acceptable salt thereof.

Also provided herein is a method of treating a RIPK2-associated disease or disorder in a subject in need thereof, comprising administering to the subject an effective amount of a compound of Formula (II), or a pharmaceutically acceptable salt thereof, or a pharmaceutical composition comprising subject an effective amount of a compound of Formula (II), or a pharmaceutically acceptable salt thereof.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety, unless expressly indicated otherwise. In case of conflict, the present specification, including definitions, will control.

Other features and advantages of the disclosure will be apparent from the following detailed description and FIGURES, and from the claims.

### DETAILED DESCRIPTION

Biologics and small molecules targeting pro-inflammatory signaling pathways have been used to successfully treat inflammatory and other diseases in patients, however, a significant fraction of patients are refractory to existing therapies. Therefore, there exists a need for identification of novel therapeutic molecules that modulate or inhibit these pathways, such as the compounds of Formula (I) and Formula (II), and pharmaceutically acceptable salts thereof described herein.

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties. In case of conflict, the present specification, including definitions, will control.

The term “about” when referring to a number or a numerical range means that the number or numerical range referred to is an approximation, for example, within experimental variability and/or statistical experimental error, and thus the number or numerical range may vary up to +10% of the stated number or numerical range.

The phrase “therapeutically effective amount” means an amount of compound that, when administered to a subject in need of such treatment, is sufficient to (i) treat a disease or disorder as described herein (e.g., a RIPK2-associated disease or disorder), (ii) attenuate, ameliorate, or eliminate one or more symptoms of the particular disease or disorder, or (iii) delay the onset of one or more symptoms of the particular disease or disorder described herein.

As used herein, terms “treat” or “treatment” refer to therapeutic or palliative measures. Beneficial or desired clinical results include, but are not limited to, alleviation, in whole or in part, of symptoms associated with a disease or disorder, diminishment of the extent of a neurological disorder, stabilized (i.e., not worsening) state of a disease or disorder, delay or slowing of disease progression, amelioration or palliation of the disease state (e.g., one or more symptoms of the disease or disorder), and remission (whether partial or total), whether detectable or undetectable and can be determined by various clinical assessments including clinical evaluation and self-reporting. “Treatment” can also mean prolonging survival as compared to expected survival if not receiving treatment.

The term “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described hereinform with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid, and organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “pharmaceutically acceptable excipients”), such as stabilizers, diluents, dispersing agents, suspending agents, thickening agents, and/or other excipients. The pharmaceutical composition facilitates administration of the compound to an organism.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The term “halo” or “halogen” refers to one of the halogens, group 17 of the periodic table. In particular the term refers to fluorine, chlorine, bromine and iodine. Preferably, the term refers to fluorine or chlorine.

The term “oxo” refers to a divalent doubly bonded oxygen atom (i.e., “═O”). As used herein, oxo groups are attached to carbon atoms to form carbonyls.

The term “alkyl” refers to a saturated acyclic hydrocarbon radical that may be a straight chain or branched, containing the indicated number of carbon atoms. For example, C1-10 indicates that the group may have from 1 to 10 (inclusive) carbon atoms in it. Non-limiting examples include methyl, ethyl, iso-propyl, tert-butyl, n-hexyl.

The term “alkenyl” refers to an acyclic hydrocarbon radical that may be a straight chain or branched, containing the indicated number of carbon atoms and one or more carbon-carbon double bonds. Non-limiting examples include ethylenyl and allyl.

The term “haloalkyl” refers to an alkyl, in which one or more hydrogen atoms is/are replaced with an independently selected halogen.

The term “hydroxyalkyl” refers to an alkyl group as described herein, in which one or more hydrogen atoms is/are replaced with one or more hydroxyl groups, as described herein.

The term “alkoxy” refers to an —O-alkyl radical (e.g., —OCH₃).

The term “thioalkyl” refers to an alkyl group as described herein, which is attached to a molecule via a sulfur atom (e.g., —SCH₃).

The term “haloalkoxy” refers to a haloalkyl group which is attached to a molecule via an oxygen atom (e.g., —OCF₃).

The term “alkoxyalkyl” refers to an alkyl group as described herein, in which one or more hydrogen atoms is/are replaced with one or more alkoxy groups as described herein.

As used herein, the term “cyano” refers to a —CN radical.

As used herein, the term “hydroxyl” refers to an —OH radical.

As used herein, the term “amino” refers to a —NH₂ radical.

As used herein, the term “phosphate” refers to a —P(═O)₂ (OH)₂ radical.

As used herein, the term “heteroaryl” refers to a 5-14 membered mono-, bi-, or tricyclic group wherein at least one ring in the system is aromatic; and wherein one or more carbon atoms in at least one ring in the system is/are replaced with an heteroatom independently selected from the group consisting of N, O, S, B, Si, and P. For example, there may be 1, 2 or 3 heteroatoms, optionally 1 or 2. A heteroaryl may further contain one or more oxo, N-oxide, S-oxide, and/or S,S-dioxide groups, valence permitting. Non-limiting examples of heteroaryl groups include furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, 2-pyridone, pyridazine, pyrimidine, pyrazine, purine, pteridine, quinoline, isoquinoline, quinazoline, quinoxaline, cinnoline, triazine, 2,3-dihydro-1H-pyrrolo[2,3-b]pyridine, 6,7-dihydro-5H-cyclopenta[b]pyridine, 3,4-dihydro-2H-pyrido[3,2-b][1,4]oxazine, 1′,2′-dihydrospiro[cyclopropane-1,3′-pyrrolo[2,3-b]pyridine], and 3′,4′-dihydrospiro[cyclopropane-1,2′-pyrido[3,2-b][1,4]oxazine].

For purposes of clarification, heteroaryl also includes aromatic lactams, aromatic cyclic ureas, or vinylogous analogs thereof, in which each ring nitrogen adjacent to a carbonyl is tertiary (i.e., all three valences are occupied by non-hydrogen substituents), such as one or more of pyridone

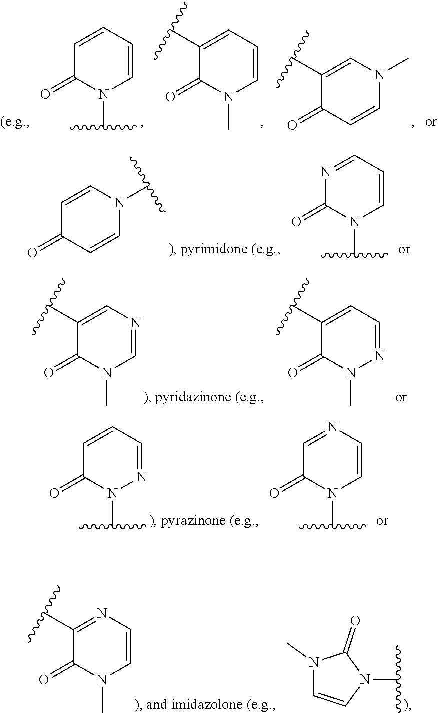

wherein each ring nitrogen adjacent to a carbonyl is tertiary (i.e., the oxo group (i.e., “═O”) herein is a constituent part of the heteroaryl ring).

As used herein, the term “cycloalkyl” refers to a saturated or partially unsaturated mono-, bi-, or tricyclic carbon group having 3 to 20 carbon atoms. Bicyclic and tricyclic cycloalkyl groups include fused, spiro, and bridged ring systems. Non-limiting examples of cycloalkyl groups include cyclopropyl, cyclohexyl, spiro[2.3]hexyl, and bicyclo[1.1.1]pentyl.

The term “cycloalkoxy” refers to an —O-cycloalkyl radical (e.g., —O-cyclopropyl).

The term “aryl” refers to a 6-20 carbon mono-, bi-, tri- or polycyclic group wherein at least one ring in the system is aromatic (e.g., 6-carbon monocyclic, 10-carbon bicyclic, or 14-carbon tricyclic aromatic ring system. Examples of aryl groups include phenyl, naphthyl, tetrahydronaphthyl, and the like.

The term “heterocyclyl” refers to a saturated or partially unsaturated hydrocarbon monocyclic, bicyclic, or tricyclic ring system having from 3 to 20 ring atoms, that is not aromatic, and having at least one heteroatom within the ring system selected from the group consisting of N, O, S, B, Si, and P. Bicyclic and tricyclic heterocyclyl groups include fused, spiro, and bridged ring systems. A heterocyclyl group may be denoted as a “5 to 10 membered heterocyclyl group,” which is a ring system containing 5, 6, 7, 8, 9 or 10 atoms at least one being a heteroatom. A heterocycle may further contain one or more oxo, thiocarbonyl, N-oxide, S-oxide, and/or S,S-dioxide groups, valence permitting, so as to make the definition include oxo-systems and thio-systems such as lactams, lactones, cyclic imides, cyclic thioimides and cyclic carbamates. A heterocyclyl group may be bonded to the rest of the molecule through any carbon atom or through a heteroatom such as nitrogen. Exemplary heterocyclyl groups include, but are not limited to 1,3-dioxolane, 1,4-dioxolane, maleimide, succinimide, dioxopiperazine, hydantoin, imidazoline, imidazolidine, isoxazoline, isoxazolidine, oxazoline, oxazolidine, oxazolidinone, thiazoline, thiazolidine, morpholine, oxirane, piperidine N-oxide, piperidine, piperazine, pyrrolidine, pyrrolidone, pyrrolidione, 4-piperidone, pyrazoline, pyrazolidine, 2-oxopyrrolidine, pyrrolidinyl, tetrahydrofuryl, thiolanyl, pyrazolinyl, oxathiolanyl, isoxazolidinyl, isothiazolidinyl, pyrrolinyl, pyrrolidinonyl, pyrazolidinyl, imidazolinyl, dioxolanyl, sulfolanyl, thiazolidedionyl, succinimidyl, dihydrofuranonyl, pyrazolidinonyl, oxazolidinyl, isoxazolidinonyl, hydantionyl, thiohydantionyl, imidazolidinonyl, oxazolidinonyl, thiazolidinonyl, oxathiolanonyl, dioxolanonyl, dioxazolidinonyl, oxadiazolidinonyl, triazolidinonyl, triazolidinethionyl, oxadiazolidinethionyl, dioxazolidinethionyl, dioxolanethionyl, oxazolidinethionyl, imidazolidinethionyl, isothiazolidinonyl, piperidinyl, tetrahydropyranyl, thianyl, morpholinyl, thiomorpholinyl, dioxanyl, piperazinyl, dithianyl, oxazinyl, tetrahydropyranonyl, piperidinonyl, dioxanonyl, oxazinanonyl, morpholinonyl, thiomorpholinonyl, piperazinonyl, tetrahydropyrimidinonyl, piperidinedionyl, oxazinanedionyl, dihydropyrimidindione, tetrahydropyridazinonyl, triazinanonyl, oxadiazinanonyl, dioxazinanonyl, morpholinedionyl, piperazinedionyl, piperazinetrionyl, triazinanedionyl and 2-azaspiro[3.3]heptanyl.

The term “saturated” as used in this context means only single bonds present between constituent atoms.

As used herein, when a ring is described as being “partially unsaturated”, it means said ring has one or more additional degrees of unsaturation (in addition to the degree of unsaturation attributed to the ring itself; e.g., one or more double or triple bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like.

As used herein, the symbol [symbol] depicts the point of attachment of an atom or moiety to the indicated atom or group in the remainder of the molecule.

Whenever a group is described as being “optionally substituted” that group may be unsubstituted or substituted with one or more of the indicated substituents. When a group is substituted, that substitution can include the sharing of a carbon atom between the parent group and the substitution to form a spiro ring. For example, an n-butyl group substituted with cyclopropyl includes both

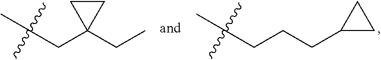

amongst others.

For the avoidance of doubt, and unless otherwise specified, for rings and cyclic groups (e.g., carbocycle, aryl, cycloalkyl, heterocyclyl, heteroaryl, and the like described herein) containing a sufficient number of ring atoms to form bicyclic or higher order ring systems (e.g., tricyclic, polycyclic ring systems), it is understood that such rings and cyclic groups encompass those having fused rings, including those in which the points of fusion are located (i) on adjacent ring atoms (e.g., [x.x.0] ring systems, in which 0 represents a zero atom bridge

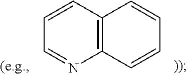

(ii) a single ring atom (spiro-fused ring systems)

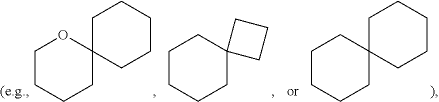

or (iii) a contiguous array of ring atoms (bridged ring systems having all bridge lengths >0)

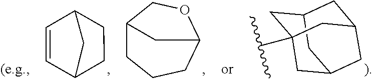

In addition, any compound or structure given herein, is also intended to represent unlabeled forms as well as isotopically labeled forms of the compounds. These forms of compounds are referred to as “isotopically enriched.” Isotopically enriched compounds have structures depicted herein, except that one or more atoms are replaced by an atom having a selected atomic mass or mass number.

Examples of isotopes that can be incorporated into the disclosed compounds include isotopes of hydrogen, carbon, nitrogen, oxygen, phosphorous, fluorine, chlorine and iodine, such as ²H, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Various isotopically enriched compounds of the present disclosure, for example those into which radioactive isotopes such as ¹³C and ¹⁴C are incorporated. Such isotopically enriched compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The term “isotopically enriched” compounds includes “deuterated” compounds described herein in which one or more hydrogens is/are replaced by deuterium, such as a hydrogen on a carbon atom. Such compounds exhibit increased resistance to metabolism and are thus useful for increasing the half-life of any compound when administered to a mammal, particularly a human. Such compounds are synthesized by means known in the art, for example by employing starting materials in which one or more hydrogens have been replaced by deuterium. Indeed, isotopically enriched compounds of this disclosure can generally be prepared by carrying out the procedures disclosed in the schemes or in the examples and preparations described below by substituting a readily available isotopically enriched reagent for a non-isotopically enriched reagent.

Deuterium enriched compounds of the present disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index relative to the corresponding non-enriched compound.

The concentration of a heavier isotope, such as deuterium, may be defined by an isotopic enrichment factor. In some embodiments, the positions noted as “H” or “hydrogen” in the compounds described herein have hydrogen at its natural abundance isotopic composition. In some embodiments, the positions noted as “H” or “hydrogen” in the compounds described herein have hydrogen enriched in deuterium above its natural abundance isotopic composition, i.e., the compound is a deuterium enriched compound. Examples of deurated groups in the compounds described herein include, but are not limited to deuteromethine

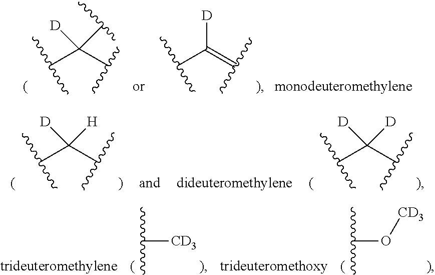

and the like.

In addition, the compounds generically or specifically disclosed herein are intended to include all tautomeric forms. Thus, by way of example, a compound containing the moiety:

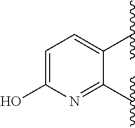 [SMILES: *c1ccc(O)nc1*]

encompasses the tautomeric form containing the moiety:

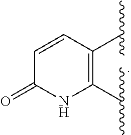 [SMILES: *c1ccc(=O)[nH]c1*]

Similarly, a pyridinyl or pyrimidinyl moiety that is described to be optionally substituted with hydroxyl encompasses pyridone or pyrimidone tautomeric forms.

The compounds provided herein may encompass various stereochemical forms. The compounds also encompass enantiomers (e.g., R and S isomers), diastereomers, as well as mixtures of enantiomers (e.g., R and S isomers) including racemic mixtures and mixtures of diastereomers, as well as individual enantiomers and diastereomers, which arise as a consequence of structural asymmetry in certain compounds. Unless otherwise indicated, when a disclosed compound is named or depicted by a structure without specifying the stereochemistry (e.g., a “flat” structure) and has one or more chiral centers, it is understood to represent all possible stereoisomers of the compound. Likewise, unless otherwise indicated, when a disclosed compound is named or depicted by a structure that specifies the stereochemistry (e.g., a structure with “wedge” and/or “dashed” bonds) and has one or more chiral centers, it is understood to represent the indicated stereoisomer of the compound.

The details of one or more embodiments of this disclosure are set forth in the accompanying drawings and the description below. Other features and advantages of the present disclosure will be apparent from the description and from the claims.

A “RIPK2 inhibitor” as defined herein includes any compound exhibiting RIPK2 inhibition activity. In some embodiments, a RIPK2 inhibitor is selective for RIPK2. Exemplary RIPK2 inhibitors can exhibit inhibition activity (IC₅₀) against a RIPK2 of less than about 1000 nM, less than about 500 nM, less than about 200 nM, less than about 100 nM, less than about 50 nM, less than about 25 nM, less than about 10 nM, less than about 5 nM, or less than about 1 nM as measured in an assay as described herein. In some embodiments, a RIPK2 inhibitor can exhibit inhibition activity (IC₅₀) against RIPK2 of less than about 25 nM, less than about 10 nM, less than about 5 nM, or less than about 1 nM as measured in an assay as provided herein.

### Compounds of Formula (I) and Formula (II)

RIPK2 mediates inflammatory signaling via NOD1, NOD2, and TLRs. However, in vitro inhibition of RIPK2 kinase activity alone is not fully predictive of suppression of downstream cellular RIPK2 signaling. Rather, interfering with the interaction of RIPK2 and XIAP is necessary to robustly reduce inflammatory signaling. In some cases, potent inhibitors of RIPK2 kinase activity lack the ability to disrupt RIPK2/XIAP binding and thus lack the ability to completely block inflammatory signaling in cells or in human subjects. The present disclosure is based, in part, on the discovery that selected compounds described herein exert RIPK2 inhibition via simultaneous inhibition of RIPK2 kinase activity and disruption of the RIPK2/XIAP interaction and thus reduce cellular inflammatory signaling.

Some embodiments provide a compound of Formula (I) or Formula (II):

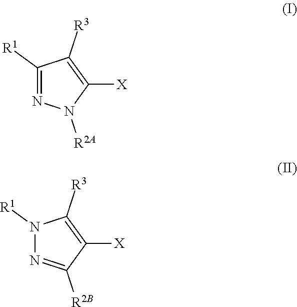

or a pharmaceutically acceptable salt of either of the foregoing, wherein:
R¹ is a 5-10 membered heteroaryl optionally substituted with 1-3 substituents independently selected from C1-C6 alkyl, C1-C6 thioalkyl, C1-C6 alkoxy optionally substituted with hydroxyl, —S(O₂) C1-C6 alkyl, NR^{1A}R^{1B}, 4-10 membered heterocyclyl optionally substituted with 1-3 substituents independently selected from halogen and C1-C6 alkyl, —(NH)—C1-C6 alkyl, and —(NH)-4-10 membered heterocyclyl optionally substituted with 1-3 substituents independently selected from halogen and C1-C6 alkyl;
R^{1A} and R^{1B} are independently selected from hydrogen, C1-C6 alkyl, and C1-C6 haloalkyl;
R^{2A} is selected from hydrogen, halogen, C1-C6 alkyl, C1-C6 haloalkyl, C1-C6 alkoxyalkyl, C1-C6 hydroxyalkyl optionally substituted with 1 or 2 halogens, C3-C6 cycloalkyl, 4-10 membered heterocyclyl optionally substituted with 1 or 2 substituents independently selected from halogen, C1-C6 alkyl, C(O)O C1-C6 alkyl;
R^{2B} is hydrogen, C1-C6 alkyl, C1-C6 haloalkyl, or C1-C6 hydroxyalkyl;
R³ is hydrogen or halogen;
X is selected from

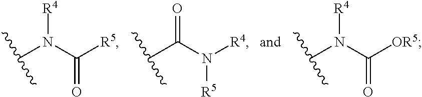

R⁴ is hydrogen or C1-C6 alkyl;
R⁵ is selected from:
(i) C1-C6 alkyl optionally substituted with 1-4 substituents independently selected from:
   (a) C3-C6 cycloalkyl optionally substituted with 1-3 substituents independently from halogen or phenyl,
   (b) phenyl optionally substituted with 1-3 substituents independently selected from halogen, C1-C6 alkoxy, C1-C6 hydroxyalkyl, and cyano,
   (c) —NR^{A}R^{B},
   (d) hydroxyl,
   (e) halogen,
   (f) 4-10 membered heterocyclyl optionally substituted with 1-3 substituents independently selected from benzyl, C1-C6 alkyl, and phenyl optionally substituted with C1-C6 alkyl, and
   (g) 5-10 membered heteroaryl optionally substituted with 1-3 substituents independently selected from halogen, cyano, C1-C6 alkyl, and C1-C6 alkoxy;
(ii) C3-C10 cycloalkyl optionally substituted with 1-3 substituents independently selected from halogen, hydroxyl, C1-C6 alkyl, phenyl optionally substituted with 1-2 independently selected halogen and 5-10 membered heteroaryl;
(iii) 4-10 membered heterocyclyl optionally substituted with 1-3 substituents independently selected from benzyl, C1-C6 alkyl, C3-C10 cycloalkyl, and phenyl optionally substituted with C1-C6 alkyl;
(iv) phenyl optionally substituted with 1-3 substituents independently selected from halogen, nitro, and C1-C6 haloalkyl;
(v) 5-10 membered heteroaryl optionally substituted with 1-3 substituents independently selected from halogen and C1-C6 alkyl;
(vi) C1-C6 alkoxy; or
(vii) C3-C6 cycloalkoxy;
or R⁴ and R⁵ with the atom to which they are attached together form a 5-12 membered heterocyclyl or 9-10 membered heteroaryl each optionally substituted with phenyl; and
R^{A} and R^{B} are independently selected from hydrogen and C1-C6 alkyl.

Some embodiments provide a compound of Formula (I)

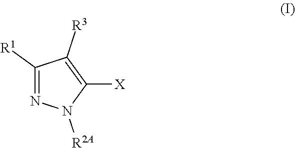

or a pharmaceutically acceptable salt thereof, wherein:
R¹ is a 5-10 membered heteroaryl optionally substituted with 1-3 substituents independently selected from C1-C6 alkyl, C1-C6 thioalkyl, C1-C6 alkoxy optionally substituted with hydroxyl, —S(O₂) C1-C6 alkyl, NR^{1A}R^{1B}, 4-10 membered heterocyclyl optionally substituted with 1-3 substituents independently selected from halogen and C1-C6 alkyl, —(NH)—C1-C6 alkyl, and —(NH)-4-10 membered heterocyclyl optionally substituted with 1-3 substituents independently selected from halogen and C1-C6 alkyl;
R^{1A} and R^{1B} are independently selected from hydrogen, C1-C6 alkyl, and C1-C6 haloalkyl;
R^{2A} is selected from hydrogen, halogen, C1-C6 alkyl, C1-C6 haloalkyl, C1-C6 alkoxyalkyl, C1-C6 hydroxyalkyl optionally substituted with 1 or 2 halogens, C3-C6 cycloalkyl, 4-10 membered heterocyclyl optionally substituted with 1 or 2 substituents independently selected from halogen, C1-C6 alkyl, C(O)O C1-C6 alkyl;
R³ is hydrogen or halogen;
X is selected from

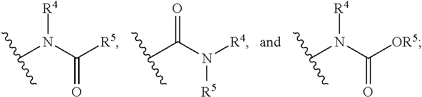

R⁴ is hydrogen or C1-C6 alkyl;
R⁵ is selected from:
(i) C1-C6 alkyl optionally substituted with 1-4 substituents independently selected from:
   (a) C3-C6 cycloalkyl optionally substituted with 1-3 substituents independently from halogen or phenyl,
   (b) phenyl optionally substituted with 1-3 substituents independently selected from halogen, C1-C6 alkoxy, C1-C6 hydroxyalkyl, and cyano,
   (c) —NR^{A}R^{B},
   (d) hydroxyl,
   (e) halogen,
   (f) 4-10 membered heterocyclyl optionally substituted with 1-3 substituents independently selected from benzyl, C1-C6 alkyl, and phenyl optionally substituted with C1-C6 alkyl, and
   (g) 5-10 membered heteroaryl optionally substituted with 1-3 substituents independently selected from halogen, cyano, C1-C6 alkyl, and C1-C6 alkoxy;
(ii) C3-C10 cycloalkyl optionally substituted with 1-3 substituents independently selected from halogen, hydroxyl, C1-C6 alkyl, phenyl optionally substituted with 1-2 independently selected halogen and 5-10 membered heteroaryl;
(iii) 4-10 membered heterocyclyl optionally substituted with 1-3 substituents independently selected from benzyl, C1-C6 alkyl, C3-C10 cycloalkyl, and phenyl optionally substituted with C1-C6 alkyl;
(iv) phenyl optionally substituted with 1-3 substituents independently selected from halogen, nitro, and C1-C6 haloalkyl;
(v) 5-10 membered heteroaryl optionally substituted with 1-3 substituents independently selected from halogen and C1-C6 alkyl;
(vi) C1-C6 alkoxy; or
(vii) C3-C6 cycloalkoxy;
or R⁴ and R⁵ with the atom to which they are attached together form a 5-12 membered heterocyclyl or 9-10 membered heteroaryl each optionally substituted with phenyl; and
R^{A} and R^{B} are independently selected from hydrogen and C1-C6 alkyl.

Some embodiments provide a compound of Formula (II)

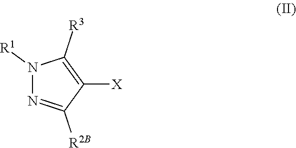

or a pharmaceutically acceptable salt thereof, wherein:
R¹ is a 5-10 membered heteroaryl optionally substituted with 1-3 substituents independently selected from C1-C6 alkyl, C1-C6 thioalkyl, C1-C6 alkoxy optionally substituted with hydroxyl, —S(O₂) C1-C6 alkyl, NR^{1A}R^{1B}, 4-10 membered heterocyclyl optionally substituted with 1-3 substituents independently selected from halogen and C1-C6 alkyl, —(NH)—C1-C6 alkyl, and —(NH)-4-10 membered heterocyclyl optionally substituted with 1-3 substituents independently selected from halogen and C1-C6 alkyl;
R^{1A} and R^{1B} are independently selected from hydrogen, C1-C6 alkyl, and C1-C6 haloalkyl;
R^{2B} is hydrogen, C1-C6 alkyl, C1-C6 haloalkyl, or C1-C6 hydroxyalkyl;
R³ is hydrogen or halogen;
X is selected from

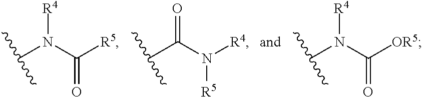

R⁴ is hydrogen or C1-C6 alkyl;
R⁵ is selected from:
(i) C1-C6 alkyl optionally substituted with 1-4 substituents independently selected from:
   (a) C3-C6 cycloalkyl optionally substituted with 1-3 substituents independently from halogen or phenyl,
   (b) phenyl optionally substituted with 1-3 substituents independently selected from halogen, C1-C6 alkoxy, C1-C6 hydroxyalkyl, and cyano,
   (c) —NR^{A}R^{B},
   (d) hydroxyl,
   (e) halogen,
   (f) 4-10 membered heterocyclyl optionally substituted with 1-3 substituents independently selected from benzyl, C1-C6 alkyl, and phenyl optionally substituted with C1-C6 alkyl, and
   (g) 5-10 membered heteroaryl optionally substituted with 1-3 substituents independently selected from halogen, cyano, C1-C6 alkyl, and C1-C6 alkoxy;
(ii) C3-C10 cycloalkyl optionally substituted with 1-3 substituents independently selected from halogen, hydroxyl, C1-C6 alkyl, phenyl optionally substituted with 1-2 independently selected halogen and 5-10 membered heteroaryl;
(iii) 4-10 membered heterocyclyl optionally substituted with 1-3 substituents independently selected from benzyl, C1-C6 alkyl, C3-C10 cycloalkyl, and phenyl optionally substituted with C1-C6 alkyl;
(iv) phenyl optionally substituted with 1-3 substituents independently selected from halogen, nitro, and C1-C6 haloalkyl;
(v) 5-10 membered heteroaryl optionally substituted with 1-3 substituents independently selected from halogen and C1-C6 alkyl;
(vi) C1-C6 alkoxy; or
(vii) C3-C6 cycloalkoxy;
or R⁴ and R⁵ with the atom to which they are attached together form a 5-12 membered heterocyclyl or 9-10 membered heteroaryl each optionally substituted with phenyl; and
R^{A} and R^{B} are independently selected from hydrogen and C1-C6 alkyl.

Unless stated otherwise, the selected embodiments below describe substituents of Formula (I) and Formula (II).

In some embodiments, R¹ is a 5-10 membered heteroaryl optionally substituted with 1-3 substituents independently selected from C1-C6 alkyl, C1-C6 thioalkyl, C1-C6 alkoxy optionally substituted with hydroxyl, —S(O₂) C1-C6 alkyl, NR^{1A}R^{1B}, 4-10 membered heterocyclyl optionally substituted with 1-3 substituents independently selected from halogen and C1-C6 alkyl, —(NH)—C1-C6 alkyl, and —(NH)-4-10 membered heterocyclyl optionally substituted with 1-3 substituents independently selected from halogen and C1-C6 alkyl. In some embodiments, R¹ is a 5-10 membered heteroaryl substituted with 1-3 substituents independently selected from C1-C6 alkyl, C1-C6 thioalkyl, C1-C6 alkoxy optionally substituted with hydroxyl, —S(O₂) C1-C6 alkyl, NR^{1A}R^{1B}, 4-10 membered heterocyclyl optionally substituted with 1-3 substituents independently selected from halogen and C1-C6 alkyl, —(NH)—C1-C6 alkyl, and —(NH)-4-10 membered heterocyclyl optionally substituted with 1-3 substituents independently selected from halogen and C1-C6 alkyl. In some embodiments, R¹ is a 5-10 membered heteroaryl optionally substituted with 1-3 substituents independently selected from C1-C6 alkyl, C1-C6 thioalkyl, C1-C6 alkoxy optionally substituted with hydroxyl, —S(O₂) C1-C6 alkyl, 4-10 membered heterocyclyl optionally substituted with 1-3 substituents independently selected from halogen and C1-C6 alkyl, —(NH)—C1-C6 alkyl, and —(NH)-4-10 membered heterocyclyl optionally substituted with 1-3 substituents independently selected from halogen and C1-C6 alkyl. In some embodiments, R¹ is a 5-10 membered heteroaryl optionally substituted with 1-3 substituents independently selected from C1-C6 alkyl, C1-C6 thioalkyl, C1-C6 alkoxy optionally substituted with hydroxyl, —S(O₂) C1-C6 alkyl, 4-10 membered heterocyclyl optionally substituted with 1-3 substituents independently selected from halogen and C1-C6 alkyl, —(NH)—C1-C6 alkyl, and —(NH)-4-10 membered heterocyclyl optionally substituted with 1-3 substituents independently selected from halogen and C1-C6 alkyl. In some embodiments, R¹ is a 5-6 membered heteroaryl substituted with 2 substituents independently selected from C1-C6 alkyl, C1-C6 thioalkyl, C1-C6 alkoxy optionally substituted with hydroxyl, —S(O₂) C1-C6 alkyl, 4-10 membered heterocyclyl optionally substituted with 1-3 substituents independently selected from halogen and C1-C6 alkyl, —(NH)—C1-C6 alkyl, and —(NH)-4-10 membered heterocyclyl optionally substituted with 1-3 substituents independently selected from halogen and C1-C6 alkyl. In some embodiments, R¹ is a 9-10 membered heteroaryl substituted with 2 substituents independently selected from C1-C6 alkyl, C1-C6 thioalkyl, C1-C6 alkoxy optionally substituted with hydroxyl, —S(O₂) C1-C6 alkyl, 4-10 membered heterocyclyl optionally substituted with 1-3 substituents independently selected from halogen and C1-C6 alkyl, —(NH)—C1-C6 alkyl, and —(NH)-4-10 membered heterocyclyl optionally substituted with 1-3 substituents independently selected from halogen and C1-C6 alkyl. In some embodiments, R¹ is a 5-10 membered heteroaryl substituted with 1-3 substituents independently selected from C1-C6 alkyl, C1-C6 thioalkyl, C1-C6 alkoxy, —S(O₂) C1-C6 alkyl, and —(NH)-4-10 membered heterocyclyl optionally substituted with 1-3 substituents independently selected from halogen and C1-C6 alkyl. In some embodiments, R¹ is a 9-10 membered heteroaryl substituted with 2 substituents independently selected from C1-C6 alkoxy, —S(O₂) C1-C6 alkyl, and —(NH)-4-10 membered heterocyclyl optionally substituted with C1-C6 alkyl. In some embodiments, R¹ is a 5-6 membered heteroaryl substituted with 2 substituents independently selected from C1-C6 alkyl, C1-C6 thioalkyl, C1-C6 alkoxy, —S(O₂) C1-C6 alkyl, and —(NH)-4-10 membered heterocyclyl optionally substituted with 1-3 substituents independently selected from halogen and C1-C6 alkyl.

In some embodiments, R¹⁴ and R^{1B} are independently selected from hydrogen, C1-C6 alkyl, and C1-C6 haloalkyl. In some embodiments, R¹⁴ and R^{1B} are independently selected from hydrogen and C1-C6 alkyl. In some embodiments, R^{1A} is H and R^{1B} is C1-C6 alkyl. In some embodiments, R¹⁴ and R^{1B} are independently selected from C1-C6 alkyl.

In some embodiments, R¹ is a 9 membered heteroaryl. In some embodiments, R¹ is a 9 membered heteroaryl linked to the remaining portion of Formulae (I) or (II) via a 5-membered ring. In some embodiments, R¹ is a 9 membered heteroaryl linked to the remaining portion of Formulae (I) or (II) via a 6-membered ring. In some embodiments, R¹ is

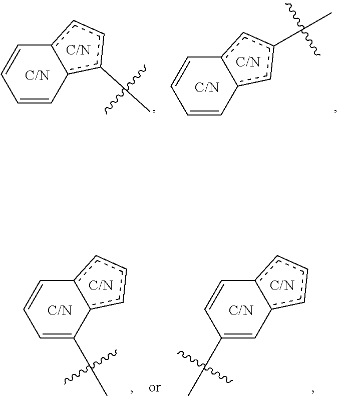

wherein the “C/N” indicates one or more atoms of the 5-membered membered ring and/or the 6-membered ring is carbon or nitrogen; the “ [symbol] ” indicates the point of attachment to the remaining portion of Formulae (I) or (II); and R¹ is optionally substituted with 1-3 substituents independently selected from C1-C6 alkoxy, —S(O₂) C1-C6 alkyl, and —(NH)-4-10 membered heterocyclyl optionally substituted with 1-3 substituents independently selected from halogen and C1-C6 alkyl. In some embodiments, R¹ is

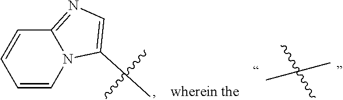

indicates the point of attachment to the remaining portion of Formulae (I) or (II); and R¹ is optionally substituted with 1-3 substituents independently selected from C1-C6 alkoxy, —S(O₂) C1-C6 alkyl, and —(NH)-4-10 membered heterocyclyl optionally substituted with 1-3 substituents independently selected from halogen and C1-C6 alkyl.

In some embodiments, R¹ is a 9 membered heteroaryl substituted with 2 substituents independently selected from C1-C6 thioalkyl, C1-C6 alkoxy, —S(O₂) C1-C6 alkyl, and —(NH)-4-10 membered heterocyclyl optionally substituted with 1-3 substituents independently selected from halogen and C1-C6 alkyl.

In some embodiments, R¹ is a 5-6 membered heteroaryl substituted with 2 substituents independently selected from C1-C6 thioalkyl, C1-C6 alkoxy, —S(O₂) C1-C6 alkyl, and —(NH)-4-10 membered heterocyclyl optionally substituted with 1-3 substituents independently selected from halogen and C1-C6 alkyl.

In some embodiments, when R¹ is substituted with a and —(NH)-4-10 membered heterocyclyl optionally substituted with 1-3 substituents independently selected from halogen and C1-C6 alkyl, the —(NH)-4-10 membered heterocyclyl is substituted with 1 or 2 substituents independently selected from halogen and C1-C6 alkyl. In some embodiments, when R¹ is substituted with a and —(NH)-4-10 membered heterocyclyl optionally substituted with 1-3 substituents independently selected from halogen and C1-C6 alkyl, the —(NH)-4-10 membered heterocyclyl is unsubstituted.

In some embodiments, R¹ is a 9 membered heteroaryl substituted with 2 substituents independently selected from C1-C6 thioalkyl, C1-C6 alkoxy, and —S(O₂) C1-C6 alkyl.

In some embodiments, R¹ is a 5-6 membered heteroaryl substituted with 2 substituents independently selected from C1-C6 thioalkyl, C1-C6 alkoxy, —S(O₂) C1-C6 alkyl.

In some embodiments, R^{2A} is hydrogen.

In some embodiments, R^{2A} is halogen. In some embodiments, R^{2A} is fluoro or chloro. In some embodiments, R^{2A} is bromo.

In some embodiments, R^{2A} is C1-C6 alkyl. In some embodiments, R^{2A} is C1-C3 alkyl. In some embodiments, R^{2A} is methyl.

In some embodiments, R^{2A} is C1-C6 haloalkyl. In some embodiments, R^{2A} is C1-C3 haloalkyl. In some embodiments, R^{2A} is trifluoromethyl.

In some embodiments, R^{2A} is C1-C6 hydroxyalkyl. In some embodiments, R²⁴ is C1-C3 hydroxyalkyl. In some embodiments, R^{2A} is hydroxyethyl.

In some embodiments, R^{2B} is hydrogen.

In some embodiments, R^{2B} is C1-C6 alkyl. In some embodiments, R^{2B} is C1-C3 alkyl. In some embodiments, R^{2B} is methyl.

In some embodiments, R²Bis C1-C6 haloalkyl. In some embodiments, R^{2B} is C1-C3 haloalkyl. In some embodiments, R^{2B} is trifluoromethyl.

In some embodiments, R^{2B} is C1-C6 hydroxyalkyl. In some embodiments, R^{2B} is C1-C3 hydroxyalkyl. In some embodiments, R^{2B} is hydroxyethyl.

In some embodiments, R³ is hydrogen.

In some embodiments, R³ is halogen. In some embodiments, R³ is fluoro or chloro. In some embodiments, R³ is bromo.

In some embodiments, X is

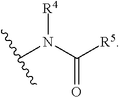

In some embodiments, X is

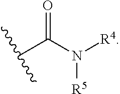

In some embodiments, X is

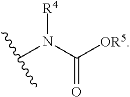

In some embodiments, R⁴ is C1-C6 alkyl. In some embodiments, R⁴ is methyl.

In some embodiments, R⁴ is hydrogen.

In some embodiments, R⁵ is C1-C6 alkyl optionally substituted with 1-3 substituents independently selected from: C3-C6 cycloalkyl optionally substituted with phenyl, phenyl optionally substituted with 1-3 independently selected halogen, —NR^{A}R^{B}, hydroxyl, 4-10 membered heterocyclyl optionally substituted with 1-3 substituents independently selected from benzyl, C1-C6 alkyl, and phenyl optionally substituted with C1-C6 alkyl; and 5-10 membered heteroaryl optionally substituted with 1-3 substituents independently selected from halogen, cyano, C1-C6 alkyl, and C1-C6 alkoxy.

In some embodiments, R⁵ is C1-C6 alkyl substituted with 1-3 substituents independently selected from: C3-C6 cycloalkyl optionally substituted with phenyl, phenyl optionally substituted with 1-3 independently selected halogen, —NR^{A}R^{B}, hydroxyl, 4-10 membered heterocyclyl optionally substituted with 1-3 substituents independently selected from benzyl, C1-C6 alkyl, phenyl optionally substituted with C1-C6 alkyl; and 5-10 membered heteroaryl optionally substituted with 1-3 substituents independently selected from halogen and C1-C6 alkyl and 5-9 membered heteroaryl optionally substituted with 1-3 substituents independently selected from halogen, cyano, C1-C6 alkyl, and C1-C6 alkoxy.

In some embodiments, R⁵ is C1-C6 alkyl substituted with 1 or 2 substituents independently selected from: C3-C6 cycloalkyl optionally substituted with phenyl, phenyl optionally substituted with 1-3 independently selected halogen, —NR^{A}R^{B}, hydroxyl, 4-10 membered heterocyclyl optionally substituted with 1-3 substituents independently selected from benzyl, C1-C6 alkyl, and phenyl optionally substituted with C1-C6 alkyl; and 5-9 membered heteroaryl optionally substituted with 1-3 substituents independently selected from halogen, cyano, C1-C6 alkyl, and C1-C6 alkoxy.

In some embodiments, R⁵ is C1-C6 alkyl substituted with 1 or 2 substituents independently selected from: C3-C6 cycloalkyl optionally substituted with phenyl, phenyl optionally substituted with 1-3 independently selected halogen, —NR^{A}R^{B}, hydroxyl, 4-8 membered heterocyclyl optionally substituted with 1 or 2 substituents independently selected from benzyl, C1-C6 alkyl, and phenyl optionally substituted with C1-C6 alkyl; and 5-9 membered heteroaryl optionally substituted with 1 or 2 substituents independently selected from halogen, cyano, C1-C6 alkyl, and C1-C6 alkoxy.

In some embodiments, the C1-C6 alkyl of R⁵ is methyl.

In some embodiments, R⁵ is C3-C10 cycloalkyl optionally substituted with 1-3 substituents independently selected from halogen, hydroxyl, C1-C6 alkyl, and phenyl optionally substituted with 1-2 independently selected halogen.

In some embodiments, R⁵ is C3-C10 cycloalkyl substituted with 1-3 substituents independently selected from halogen, hydroxyl, C1-C6 alkyl, and phenyl optionally substituted with 1-2 independently selected halogen.

In some embodiments, R⁵ is C1-C6 alkyl optionally substituted with 1-3 substituents independently selected from: C3-C6 cycloalkyl optionally substituted with phenyl, phenyl optionally substituted with 1-3 independently selected halogen,—NR^{A}R^{B}, 4-10 membered heterocyclyl optionally substituted with 1-3 substituents independently selected from benzyl, C1-C6 alkyl, and phenyl optionally substituted with C1-C6 alkyl; and 5-10 membered heteroaryl optionally substituted with 1-3 substituents independently selected from halogen and C1-C6 alkyl.

In some embodiments, R⁵ is C1-C6 alkyl substituted with 1-3 substituents independently selected from: C3-C6 cycloalkyl optionally substituted with phenyl, phenyl optionally substituted with 1-3 independently selected halogen,—NR^{A}R^{B}, 4-10 membered heterocyclyl optionally substituted with 1-3 substituents independently selected from benzyl, C1-C6 alkyl, and phenyl optionally substituted with C1-C6 alkyl; and 5-10 membered heteroaryl optionally substituted with 1-3 substituents independently selected from halogen and C1-C6 alkyl.

In some embodiments, R⁵ is C1-C6 alkyl substituted with 1 or 2 substituents independently selected from: C3-C6 cycloalkyl optionally substituted with phenyl, phenyl optionally substituted with 1-3 independently selected halogen,-NR^{A}R^{B}, 4-10 membered heterocyclyl optionally substituted with 1-3 substituents independently selected from benzyl, C1-C6 alkyl, and phenyl optionally substituted with C1-C6 alkyl; and 5-10 membered heteroaryl optionally substituted with 1-3 substituents independently selected from halogen and C1-C6 alkyl.

In some embodiments, R⁵ is C1-C6 alkyl substituted with 1 or 2 substituents independently selected from: C3-C6 cycloalkyl optionally substituted with phenyl, phenyl optionally substituted with 1-3 independently selected halogen,-NR^{A}R^{B}, 4-8 membered heterocyclyl optionally substituted with 1 or 2 substituents independently selected from benzyl, C1-C6 alkyl, and phenyl optionally substituted with C1-C6 alkyl; and 5-10 membered heteroaryl optionally substituted with 1 or 2 substituents independently selected from halogen and C1-C6 alkyl.

In some embodiments, R⁵ is C1-C6 alkyl substituted with C3-C6 cycloalkyl optionally substituted with phenyl, phenyl optionally substituted with 1-3 independently selected halogen,-NR^{A}R^{B}, 4-8 membered heterocyclyl optionally substituted with 1 or 2 substituents independently selected from benzyl, C1-C6 alkyl, and phenyl optionally substituted with C1-C6 alkyl; and 5-10 membered heteroaryl optionally substituted with 1 or 2 substituents independently selected from halogen and C1-C6 alkyl.

In some embodiments, R⁵ is C1-C6 alkyl substituted with 2 substituents independently selected from C3-C6 cycloalkyl optionally substituted with phenyl, phenyl optionally substituted with 1-3 independently selected halogen,-NR^{A}R^{B}, 4-8 membered heterocyclyl optionally substituted with 1 or 2 substituents independently selected from benzyl, C1-C6 alkyl, and phenyl optionally substituted with C1-C6 alkyl; and 5-10 membered heteroaryl optionally substituted with 1 or 2 substituents independently selected from halogen and C1-C6 alkyl.

In some embodiments, R⁵ is benzyl, where the phenyl is optionally substituted with 1-3 independently selected halogen. In some embodiments, R⁵ is benzyl, where the phenyl is substituted with 1-3 independently selected halogen. In some embodiments, R⁵ is benzyl.

In some embodiments, the C1-C6 alkyl of R⁵ is methyl.

In some embodiments, R⁵ is C3-C10 cycloalkyl optionally substituted with 1-3 substituents independently selected from hydroxyl, C1-C6 alkyl, and phenyl optionally substituted with 1-2 independently selected halogen.

In some embodiments, R⁵ is C3-C10 cycloalkyl substituted with 1-3 substituents independently selected from halogen, hydroxyl, C1-C6 alkyl, and phenyl optionally substituted with 1-2 independently selected halogen.

In some embodiments, R⁵ is C3-C6 cycloalkyl substituted with 1-3 substituents independently selected from halogen, hydroxyl, C1-C6 alkyl, and phenyl optionally substituted with 1-2 independently selected halogen.

In some embodiments, R⁵ is C3-C6 cycloalkyl substituted with 1 or 2 substituents independently selected from halogen, hydroxyl, C1-C6 alkyl, and phenyl optionally substituted with 1-2 independently selected halogen.

In some embodiments, R⁵ is C3-C6 cycloalkyl substituted with two independently selected halogen.

In some embodiments, R⁵ is C3-C10 cycloalkyl. In some embodiments, R⁵ is C3-C6 cycloalkyl.

In some embodiments, R⁵ is 4-10 membered heterocyclyl optionally substituted with 1-3 substituents independently selected from benzyl, C1-C6 alkyl, and phenyl optionally substituted with C1-C6 alkyl.

In some embodiments, R⁵ is 4-10 membered heterocyclyl substituted with 1-3 substituents independently selected from benzyl, C1-C6 alkyl, and phenyl optionally substituted with C1-C6 alkyl.

In some embodiments, R⁵ is 4-10 membered heterocyclyl substituted with 1 or 2 substituents independently selected from benzyl, C1-C6 alkyl, and phenyl optionally substituted with C1-C6 alkyl.

In some embodiments, R⁵ is 4-8 membered heterocyclyl substituted with 1 or 2 substituents independently selected from benzyl, C1-C6 alkyl, and phenyl optionally substituted with C1-C6 alkyl.

In some embodiments, R⁵ is 4-8 membered heterocyclyl substituted with phenyl. In some embodiments, R⁵ is 4-8 membered heterocyclyl.

In some embodiments, R⁵ is phenyl optionally substituted with 1-3 substituents independently selected from halogen, nitro, and C1-C6 haloalkyl.

In some embodiments, R⁵ is phenyl substituted with 1-3 substituents independently selected from halogen, nitro, and C1-C6 haloalkyl.

In some embodiments, R⁵ is phenyl substituted with 1 or 2 substituents independently selected from halogen, nitro, and C1-C6 haloalkyl.

In some embodiments, R⁵ is 5-10 membered heteroaryl optionally substituted with 1-3 substituents independently selected from halogen and C1-C6 alkyl.

In some embodiments, R⁵ is 5-10 membered heteroaryl substituted with 1-3 substituents independently selected from halogen and C1-C6 alkyl. In some embodiments, R⁵ is 5-10 membered heteroaryl substituted with 1-3 substituents independently selected from halogen and methyl.

In some embodiments, R⁵ is 5-10 membered heteroaryl substituted with 1 or 2 substituents independently selected from halogen and C1-C6 alkyl. In some embodiments, R⁵ is 5-10 membered heteroaryl substituted with 1 or 2 substituents independently selected from halogen and methyl.

In some embodiments, R⁵ is 5-6 membered heteroaryl substituted with 1 or 2 substituents independently selected from halogen and C1-C6 alkyl. In some embodiments, R⁵ is 5-6 membered heteroaryl substituted with 1 or 2 substituents independently selected from halogen and methyl.

In some embodiments, R⁵ is C1-C6 alkoxy. In some embodiments, R⁵ is C1-C3 alkoxy. In some embodiments, R⁵ is methoxy.

In some embodiments, R⁵ is C3-C6 cycloalkoxy.

In some embodiments, R⁴ and R⁵ with the atom to which they are attached together form a 5-12 membered heterocyclyl optionally substituted with phenyl. In some embodiments, R⁴ and R⁵ with the atom to which they are attached together form a 5-12 membered heterocyclyl substituted with phenyl. In some embodiments, R⁴ and R⁵ with the atom to which they are attached together form a 5-12 membered heterocyclyl.

In some embodiments, R⁴ and R⁵ with the atom to which they are attached together form a 9-10 membered heteroaryl optionally substituted with phenyl. In some embodiments, R⁴ and R⁵ with the atom to which they are attached together form a 9-10 membered heteroaryl substituted with phenyl. In some embodiments, R⁴ and R⁵ with the atom to which they are attached together form a 9-10 membered heteroaryl.

In some embodiments, R^{A} is hydrogen.

In some embodiments, R^{A} is C1-C6 alkyl. In some embodiments, R^{A} is C1-C3 alkyl. In some embodiments, R^{A} is methyl.

In some embodiments, R^{B} is hydrogen.

In some embodiments, R^{B} is C1-C6 alkyl. In some embodiments, R^{B} is C1-C3 alkyl. In some embodiments, R^{B} is methyl.

In some embodiments, Formula (I) is (I-a):

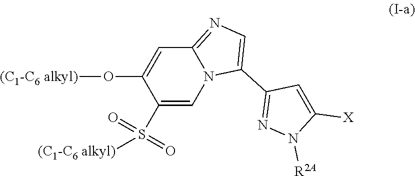

or a pharmaceutically acceptable salt thereof.

In some embodiments, Formula (I) is (I-b):

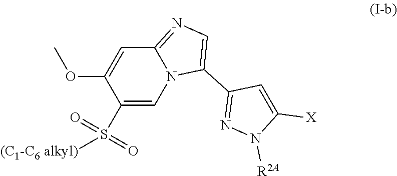

or a pharmaceutically acceptable salt thereof.

In some embodiments, Formula (I) is (I-c):

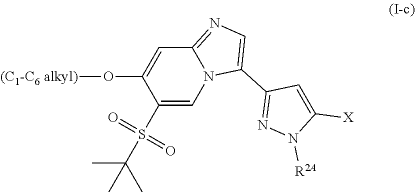

or a pharmaceutically acceptable salt thereof.

In some embodiments, Formula (I) is (I-d):

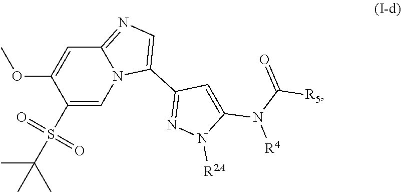

or a pharmaceutically acceptable salt thereof.

In some embodiments, Formula (I) is (I-e):

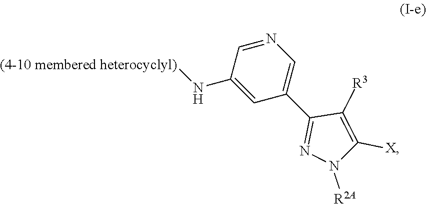

or a pharmaceutically acceptable salt thereof.

In some embodiments, Formula (I) is (I-f):

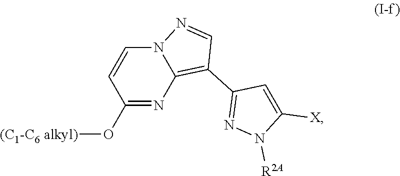

or a pharmaceutically acceptable salt thereof.

In some embodiments, Formula (I) is (I-g):

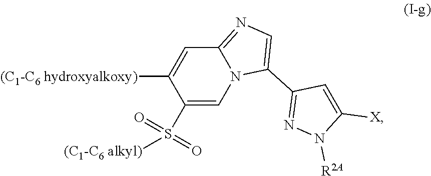

or a pharmaceutically acceptable salt thereof.

In some embodiments, Formula (I) is (I-h):

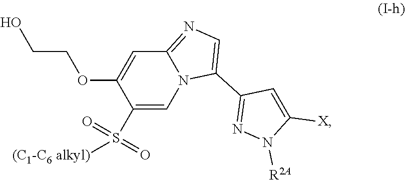

or a pharmaceutically acceptable salt thereof.

In some embodiments, Formula (I) is (I-i):

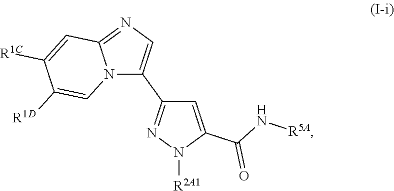

or a pharmaceutically acceptable salt thereof, wherein:
R^{1C} is C1-C6 alkoxy optionally substituted with hydroxyl;
R^{1D} is —S(O₂)—C1-C6 alkyl;
R^{2A1} is C1-C3 alkyl or C3-C4 cycloalkyl; and
R^{5A} is phenyl optionally substituted with halogen or C1-C6 alkyl; or 5-6 membered heteroaryl optionally substituted with halogen or C1-C6 alkyl.

In some embodiments, Formula (I) is (I-j):

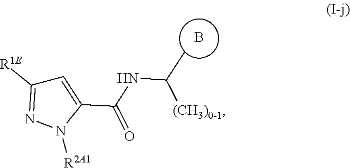

or a pharmaceutically acceptable salt thereof, wherein:
R^{1E} is a 5-10 membered heteroaryl substituted with 2 substituents independently selected from C1-C6 alkyl, C1-C6 thioalkyl, C1-C6 alkoxy optionally substituted with hydroxyl, and —S(O₂) C1-C6 alkyl;
R^{2A1} is C1-C3 alkyl or C3-C4 cycloalkyl; and
Ring B is (i) phenyl optionally substituted with 1-3 substituents independently selected from halogen, C1-C6 alkoxy, C1-C6 hydroxyalkyl, and cyano; or (ii)₅-6 membered heteroaryl optionally substituted with 1-3 substituents independently selected from halogen, cyano, C1-C6 alkyl, and C1-C6 alkoxy;
wherein the —(CH₃)₀₋₁ is intended indicate the presence or absence of the methyl group; when the methyl group is absent a hydrogen is in its place.

In some embodiments, Formula (II) is (II-a):

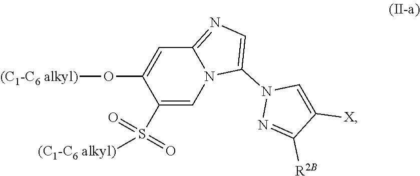

or a pharmaceutically acceptable salt thereof.

In some embodiments, Formula (II) is (II-b):

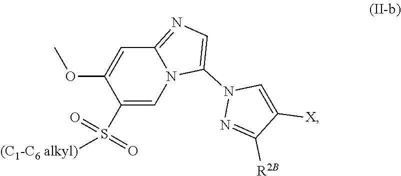

or a pharmaceutically acceptable salt thereof.

In some embodiments, Formula (II) is (II-c):

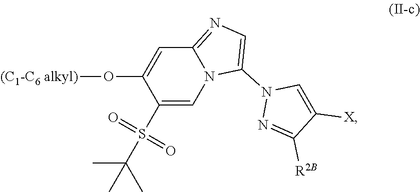

or a pharmaceutically acceptable salt thereof.

In some embodiments, Formula (II) is (II-d):

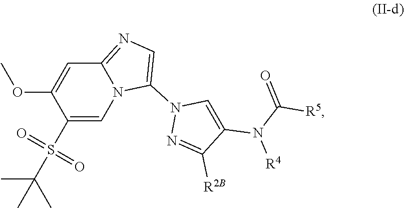

or a pharmaceutically acceptable salt thereof.

In some embodiments, Formula (II) is (II-e):

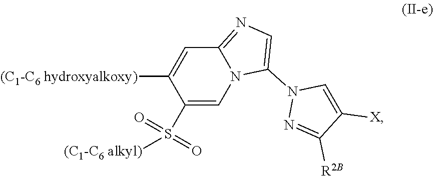

or a pharmaceutically acceptable salt thereof.

In some embodiments, Formula (II) is (II-f):

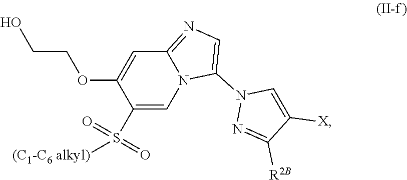

or a pharmaceutically acceptable salt thereof.

In some embodiments, Formula (I) is (I-g):

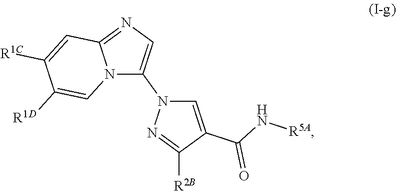

or a pharmaceutically acceptable salt thereof, wherein:
R^{1C} is C1-C6 alkoxy optionally substituted with hydroxyl;
R^{1D} is —S(O₂)—C1-C6 alkyl; and
R^{5A} is phenyl optionally substituted with halogen or C1-C6 alkyl; or 5-6 membered heteroaryl optionally substituted with halogen or C1-C6 alkyl.

In some embodiments, Formula (I) is (I-h):

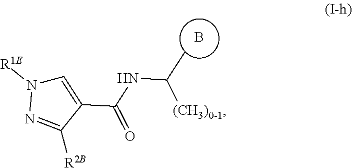

or a pharmaceutically acceptable salt thereof, wherein:
R^{1E} is a 5-10 membered heteroaryl substituted with 2 substituents independently selected from C1-C6 alkyl, C1-C6 thioalkyl, C1-C6 alkoxy optionally substituted with hydroxyl, and —S(O₂) C1-C6 alkyl; and
Ring B is (i) phenyl optionally substituted with 1-3 substituents independently selected from halogen, C1-C6 alkoxy, C1-C6 hydroxyalkyl, and cyano; or (ii) 5-6 membered heteroaryl optionally substituted with 1-3 substituents independently selected from halogen, cyano, C1-C6 alkyl, and C1-C6 alkoxy;
wherein the —(CH₃)₀₋₁ is intended indicate the presence or absence of the methyl group; when the methyl group is absent a hydrogen is in its place.

In some embodiments, Formula (I) is selected from the group consisting of:

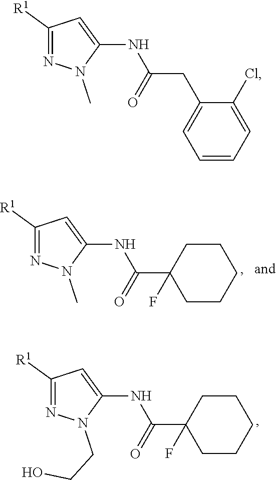

or a pharmaceutically acceptable salt of any of the foregoing.

In some embodiments, Formula (I) is selected from the group consisting of:

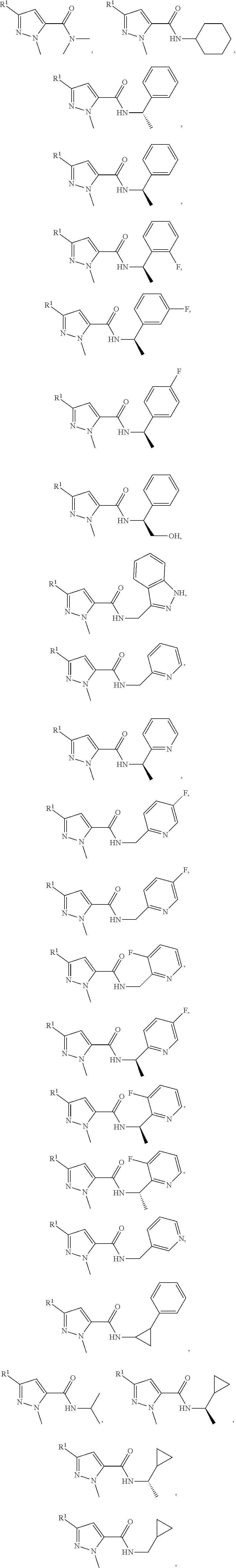

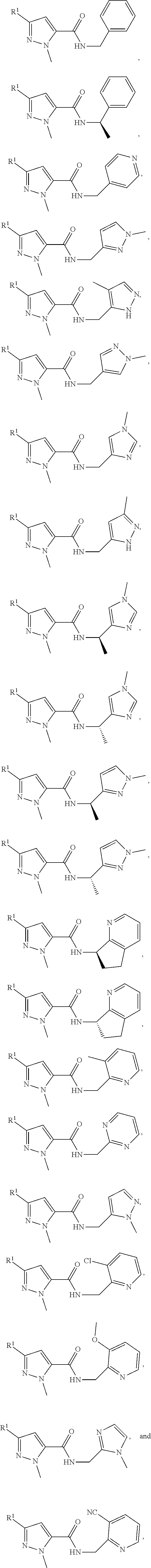

or a pharmaceutically acceptable salt thereof.

In some embodiments, Formula (I) is selected from the group consisting of:

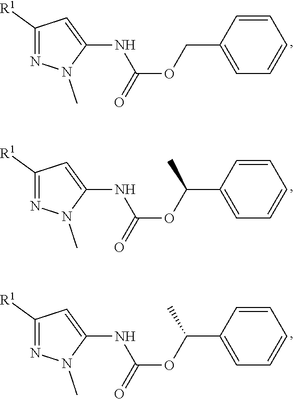

or a pharmaceutically acceptable salt thereof.

In some embodiments, Formula (II) is selected from the group consisting of:

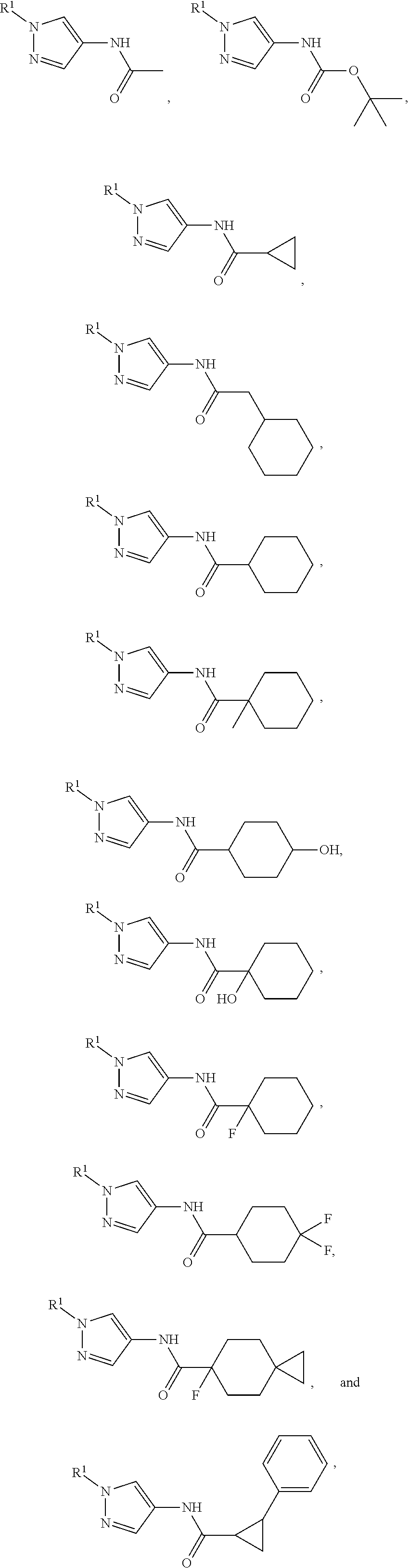

or a pharmaceutically acceptable salt of any of the foregoing.

In some embodiments, Formula (II) is selected from the group consisting of:

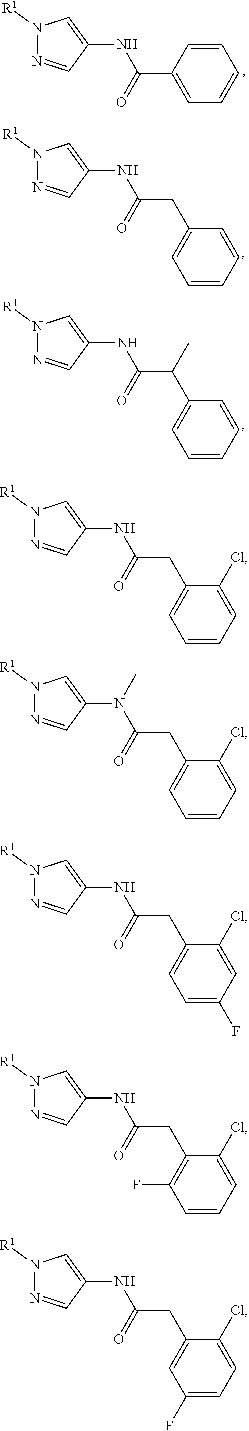

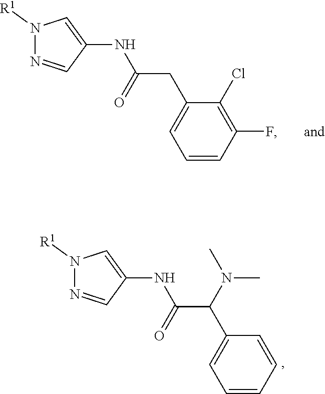

or a pharmaceutically acceptable salt of any of the foregoing.

In some embodiments, Formula (II) is selected from the group consisting of:

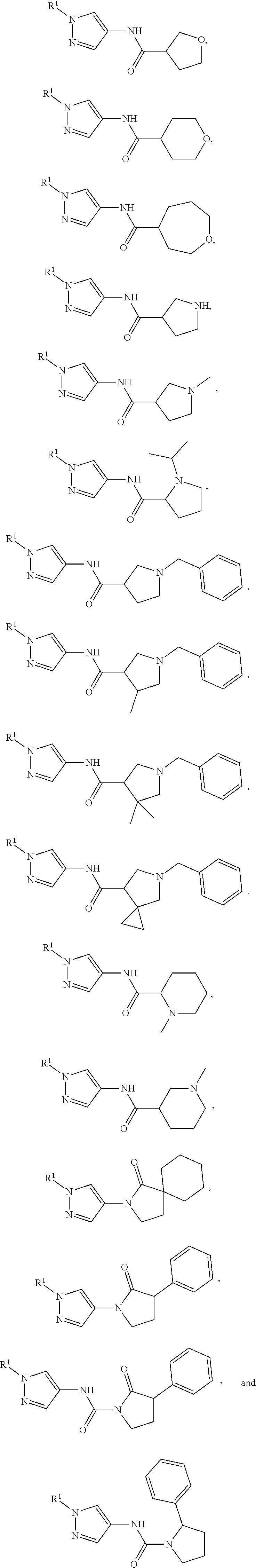

or a pharmaceutically acceptable salt thereof.

The compounds of Formula (I), and pharmaceutically acceptable salts thereof, do not include the compounds exemplified in PCT Appl. No. PCT/US2023/024988, or pharmaceutically acceptable salts thereof.

In some embodiments, the compounds of Formula (I) and Formula (II), include the compounds of Examples 1-194 and pharmaceutically acceptable salts thereof. In some embodiments, the compounds of Examples 1-194 are in the free base form. In some embodiments, the compounds of Examples 1-194 are in salt form, e.g., pharmaceutically acceptable salt form.

The ability of test compounds to act as RIPK2 inhibitors may be demonstrated by the biological assays described herein. See, e.g., Table A1 and Table A.

### Methods of Treatment

The compounds and compositions disclosed herein are effective for modulating the activity of RIPK2. In some embodiments, the compounds and compositions disclosed herein are RIPK2 inhibitors.

The term “RIPK2-associated disease or disorder” as used herein refers to diseases or disorders associated with or having a dysregulation of a RIPK2 gene, a RIPK2 protein, or the expression or activity or level of any (e.g., one or more) of the same (e.g., any of the types of dysregulation of a RIPK2 gene, a RIPK2 protein, a RIPK2 protein domain, or the expression or activity or level of any of the same described herein).

An exemplary sequence of human RIPK2 is shown below (UniParc Accession No. UPI00001338F2):

MNGEAICSALPTIPYHKLADLRYLSRGASGTVSSARHADWRVQVAVKHLH
IHTPLLDSERKDVLREAEILHKARFSYILPILGICNEPEFLGIVTEYMPN
GSLNELLHRKTEYPDVAWPLRFRILHEIALGVNYLHNMTPPLLHHDLKTQ
NILLDNEFHVKIADFGLSKWRMMSLSQSRSSKSAPEGGTIIYMPPENYEP
GQKSRASIKHDIYSYAVITWEVLSRKQPFEDVTNPLQIMYSVSQGHRPVI
NEESLPYDIPHRARMISLIESGWAQNPDERPSFLKCLIELEPVLRTFEEI
TFLEAVIQLKKTKLQSVSSAIHLCDKKKMELSLNIPVNHGPQEESCGSSQ
LHENSGSPETSRSLPAPQDNDFLSRKAQDCYFMKLHHCPGNHSWDSTISG
SQRAAFCDHKTTPCSSAIINPLSTAGNSERLQPGIAQQWIQSKREDIVNQ
MTEACLNQSLDALLSRDLIMKEDYELVSTKPTRTSKVRQLLDTTDIQGEE
FAKVIVQKLKDNKQMGLQPYPEILVVSRSPSLNLLQNKSM

Some embodiments provide a method of treating a RIPK2-associated disease or disorder in a subject in need thereof, comprising administering to the subject an effective amount of a compound of Formula (I), or a pharmaceutically acceptable salt thereof, or a pharmaceutical composition comprising an effective amount of a compound of Formula (I), or a pharmaceutically acceptable salt thereof.

Some embodiments provide a method of treating a RIPK2-associated disease or disorder in a subject in need thereof, comprising administering to the subject an effective amount of a compound of Formula (II), or a pharmaceutically acceptable salt thereof, or a pharmaceutical composition comprising an effective amount of a compound of Formula (II), or a pharmaceutically acceptable salt thereof.

Some embodiments provide a method of treating a RIPK2-associated disease or disorder in a subject in need thereof, comprising (a) determining that the subject is suffering from a RIPK2-associated disease or disorder; and (b) administering to the subject an effective amount of a compound of Formula (I), or a pharmaceutically acceptable salt thereof, or a pharmaceutical composition comprising an effective amount of a compound of Formula (I), or a pharmaceutically acceptable salt thereof.

Some embodiments provide a method of treating a RIPK2-associated disease or disorder in a subject in need thereof, comprising (a) determining that the subject is suffering from a RIPK2-associated disease or disorder; and (b) administering to the subject an effective amount of a compound of Formula (II), or a pharmaceutically acceptable salt thereof, or a pharmaceutical composition comprising an effective amount of a compound of Formula (II), or a pharmaceutically acceptable salt thereof.

Some embodiments provide a method of treating a RIPK2-associated disease or disorder in a subject previously identified or diagnosed as having a RIPK2-associated disease or disorder, the method comprising administering to the subject an effective amount of a compound of Formula (I), or a pharmaceutically acceptable salt thereof, or a pharmaceutical composition comprising an effective amount of a compound of Formula (I), or a pharmaceutically acceptable salt thereof.

Some embodiments provide a method of treating a RIPK2-associated disease or disorder in a subject previously identified or diagnosed as having a RIPK2-associated disease or disorder, the method comprising administering to the subject an effective amount of a compound of Formula (II), or a pharmaceutically acceptable salt thereof, or a pharmaceutical composition comprising an effective amount of a compound of Formula (II), or a pharmaceutically acceptable salt thereof.

In some embodiments, the RIPK2-associated disease or disorder is a cardiovascular disease, an allergic disorder, an autoimmune disease, an inflammatory disease, a cardiovascular disease, a fibrotic disease, or a disease associated with abnormal cell growth.

In some embodiments, the RIPK2-associated disease or disorder is a Type I hypersensitivity (allergic) reaction. In some embodiments, the Type I hypersensitivity (allergic) reaction is allergic inflammation. In some embodiments, the allergic inflammation is allergic rhinitis, allergic asthma, allergic conjunctivitis, atopic- and vernal keratoconjunctivitis, or atopic dermatitis.

In some embodiments, the RIPK2-associated disease or disorder is an autoimmune disease. In some embodiments, the autoimmune disease is Crohn's disease, ulcerative colitis, rheumatoid arthritis, multiple sclerosis, encephalomyelitis, systemic lupus erythematosus, psoriasis, lupus nephritis, immune thrombocytopenia purpura, Sjogren's syndrome, ankylosing spondylitis, psoriatic arthritis, juvenile dermatomyositis, juvenile rheumatoid arthritis, juvenile spondyloarthopathy, non-radiographic spondyloarthopathy, Behcet's disease, dermatomyositis, diabetes mellitus type 1, Goodpasture's syndrome, Graves' disease, Guillain-Barre syndrome, Hashimoto's disease, mixed connective tissue damage, myasthenia gravis, narcolepsy, pemphigus vulgaris, pernicious anemia, polymyositis, primary biliary cirrhosis, temporal arteritis, or vasculitis.

In some embodiments, the autoimmune disease is Crohn's disease, ulcerative colitis, inflammatory bowel disease, or multiple sclerosis. In some embodiments, the autoimmune disease is Crohn's disease. In some embodiments, the autoimmune disease is ulcerative colitis. In some embodiments, the autoimmune disease is inflammatory bowel disease. In some embodiments, the autoimmune disease is multiple sclerosis.

In some embodiments, the RIPK2-associated disease or disorder is a metabolic disease. In some embodiments, the metabolic disease is dysglycemia, type 2 diabetes, non-alcoholic fatty liver disease (including non-alcoholic steatohepatitis), or obesity.

In some embodiments, the RIPK2-associated disease or disorder is an inflammatory disease. In some embodiments, the inflammatory disease is chronic lung inflammatory disease, osteoarthritis, inflammatory arthritis, asthma, early onset sarcoidosis, sarcoidosis, eczema, allergic eczema, uveitis, reactive arthritis, chronic inflammation, chronic prostatitis, inflammatory bowel disease, glomerulonephritis, bursitis, carpal tunnel syndrome, tendinitis, inflammation of the lung (e.g., chronic obstructive pulmonary disease), pelvic inflammatory disease, transplant rejection, vasculitis, regional enteritis, distal ileitis, regional ileitis, and terminal ileitis, central areolar choroidal dystrophy, macular degeneration, retinosis pigmentosa, adult vitelliform disease, pattern dystrophy, diabetic retinopathy, BEST disease, myopic degeneration, central serous retinopathy, Stargardt's disease, Cone-Rod dystrophy, North Carolina dystrophy, infectious retinitis, inflammatory retinitis, uveitis, toxic retinitis, or systemic inflammatory response syndrome. In some embodiments, the inflammatory disease is inflammatory bowel disease.

In some embodiments, the RIPK2-associated disease or disorder isgranulomatous inflammatory disease. In some embodiments, the granulomatous inflammatory disease is Wegener's granulomatosis, Churg-Strauss syndrome, relapsing polychondritis, polyarteritis nodosa, giant cell arteritis, primary biliary cirrhosis, hepatic granulomatous disease, Langerhan's granulomatosis, granulomatous enteritis, orofacial granulomatosis, or Peyronie's disease.

In some embodiments, the RIPK2-associated disease or disorder is a cardiovascular disease. In some embodiments, the cardiovascular disease is atherosclerosis, thrombosis, myocardial infarction, stroke, aortic aneurysm, arterial hypertension, sickle cell crisis, or ischemia-reperfusion injury.

In some embodiments, the RIPK2-associated disease is lethal systemic inflammatory response syndrome, chronic gut and skin inflammation, or acute pancreatitis.

In some embodiments, the RIPK2-associated disease or disorder is a fibrotic disease. In some embodiments, the fibrotic disease is scleroderma, asbestosis, or idiopathic pulmonary fibrosis.

In some embodiments, the RIPK2-associated disease or disorder comprises neuroinflammation. In some embodiments, the RIPK2-associated disease or disorder is Alzheimer's disease, amyotrophic lateral sclerosis (ALS), Parkinson's disease, Huntington's disease, Lewy body disease, Niemann-Pick disease, type C1 (NPC1), Friedreich's ataxia, spinal muscular atrophy, corticobasal degeneration, progress supranuclear palsy (PSP), or multiple system atrophy (MSA).

In some embodiments, the RIPK2-associated disease or disorder is a disease related to abnormal cell growth. In some embodiments, the disease related to abnormal cell growth is cancer, including hematological malignancies and solid tumors.

Hematological malignancies include, but are not limited to leukemias, such as acute myeloid leukemia, chronic myelogenous leukemia, chronic lymphocytic leukemia, B-cell chronic lymphocytic leukemia, and lymphomas and myelomas, such as B-cell lymphoma (e.g., mantle cell lymphoma), T-cell lymphoma (e.g., peripheral T-cell lymphoma), non-Hodgkin's lymphoma, and multiple myeloma.

Solid tumors include lung cancer (small cell lung cancer and non-small cell lung cancer), pancreatic cancer, colon cancer, breast cancer, genitourinary cancer, skin cancer, bone cancer, prostate cancer, liver cancer, brain cancer, laryngeal cancer, gall bladder cancer, rectal cancer, parathyroid cancer, thyroid cancer, adrenal cancer, neural tissue cancer, bladder cancer, head and neck cancer, stomach cancer, gastric cancer, bronchial cancer, and kidney cancer (e.g., renal clear cell carcinoma), colorectal cancer, clear cell carcinoma, basal cell carcinoma, squamous cell carcinoma, esophageal cancer, metastatic skin carcinoma, osteosarcoma, Ewing's sarcoma, reticulum cell sarcoma, Kaposi's sarcoma, giant cell tumor, islet cell tumor, acute and chronic lymphocytic and granulocytic tumors, hairy-cell tumor, adenoma, medullary carcinoma, pheochromocytoma, mucosal neuromas, intestinal ganglioneuromas, hyperplastic corneal nerve tumor, marfanoid habitus tumor, Wilms' tumor, seminoma, ovarian tumor, leiomyomata tumor, cervical dysplasia, neuroblastoma, retinoblastoma, myelodysplastic syndrome, rhabdomyosarcoma, astrocytoma, malignant hypercalcemia, polycythemia vera, adenocarcinoma, glioblastoma multiforma, glioma, and malignant melanoma.

In some embodiments, the RIPK2-associated disease or disorder is a disease related to abnormal cell growth that is a non-malignant proliferative disease. In some embodiments, the non-malignant proliferative disease is benign prostatic hypertrophy, restenosis, hyperplasia, synovial proliferation disorder, idiopathic plasmacytic lymphadenopathy, or retinopathy.

In some embodiments, the RIPK2-associated disease or disorder is selected from the group consisting of: avascular necrosis, calcium pyrophosphate dihydrate crystal deposition disease (pseudo gout), Blau syndrome, Ehlers-Danlos syndrome, fibromyalgia, Fifth disease, giant cell arteritis, gout, Lyme disease, Marfan syndrome, myositis, osteoarthritis, osteogenesis imperfecta, osteoporosis, Paget's disease, Raynaud's phenomenon, reactive arthritis, reflex sympathetic dystrophy syndrome, spinal stenosis, and Still's disease.

In some embodiments, the RIPK2-associated disease or disorder is a cancer associated with chronic inflammation. In some embodiments, the cancer associated with chronic inflammation that can be treated (including reduction in the likelihood of recurrence) include colitis-associated colorectal cancer, gastric cancer, gastric mucosal lymphoma, lung cancer, hepatocellular carcinoma, thyroid cancer, breast cancer, oral cancer, head and neck cancer, nasopharyngeal carcinoma, endometrial cancer, uterine cancer, ovarian cancer, prostate cancer, bladder cancer, pancreatic cancer, esophageal cancer, skin cancer, and non-Hodgkin lymphoma.

Some embodiments provide a method of treating inflammatory bowel disease in a subject in need thereof, comprising administering to the subject an effective amount of a compound of Formula (I), or a pharmaceutically acceptable salt thereof, or a pharmaceutical composition comprising an effective amount of a compound of Formula (I), or a pharmaceutically acceptable salt thereof.

Some embodiments provide a method of treating inflammatory bowel disease in a subject in need thereof, comprising administering to the subject an effective amount of a compound of Formula (II), or a pharmaceutically acceptable salt thereof, or a pharmaceutical composition comprising an effective amount of a compound of Formula (II), or a pharmaceutically acceptable salt thereof.

Some embodiments provide a method of treating inflammatory bowel disease in a subject in need thereof, comprising (a) determining that the subject is suffering from inflammatory bowel disease; and (b) administering to the subject an effective amount of a compound of Formula (I), or a pharmaceutically acceptable salt thereof, or a pharmaceutical composition comprising an effective amount of a compound of Formula (I), or a pharmaceutically acceptable salt thereof.

Some embodiments provide a method of treating inflammatory bowel disease in a subject in need thereof, comprising (a) determining that the subject is suffering from inflammatory bowel disease; and (b) administering to the subject an effective amount of a compound of Formula (II), or a pharmaceutically acceptable salt thereof, or a pharmaceutical composition comprising an effective amount of a compound of Formula (II), or a pharmaceutically acceptable salt thereof.

Some embodiments provide a method of treating inflammatory bowel disease in a subject previously identified or diagnosed as having inflammatory bowel disease, the method comprising administering to the subject an effective amount of a compound of Formula (I), or a pharmaceutically acceptable salt thereof, or a pharmaceutical composition comprising an effective amount of a compound of Formula (I), or a pharmaceutically acceptable salt thereof.

Some embodiments provide a method of treating inflammatory bowel disease in a subject previously identified or diagnosed as having inflammatory bowel disease, the method comprising administering to the subject an effective amount of a compound of Formula (II), or a pharmaceutically acceptable salt thereof, or a pharmaceutical composition comprising an effective amount of a compound of Formula (II), or a pharmaceutically acceptable salt thereof.

Some embodiments provide a method of treating Crohn's disease in a subject in need thereof, comprising administering to the subject an effective amount of a compound of Formula (I), or a pharmaceutically acceptable salt thereof, or a pharmaceutical composition comprising an effective amount of a compound of Formula (I), or a pharmaceutically acceptable salt thereof.

Some embodiments provide a method of treating Crohn's disease in a subject in need thereof, comprising administering to the subject an effective amount of a compound of Formula (II), or a pharmaceutically acceptable salt thereof, or a pharmaceutical composition comprising an effective amount of a compound of Formula (II), or a pharmaceutically acceptable salt thereof.

Some embodiments provide a method of treating Crohn's disease in a subject in need thereof, comprising (a) determining that the subject is suffering from Crohn's disease; and (b) administering to the subject an effective amount of a compound of Formula (I), or a pharmaceutically acceptable salt thereof, or a pharmaceutical composition comprising an effective amount of a compound of Formula (I), or a pharmaceutically acceptable salt thereof.

Some embodiments provide a method of treating Crohn's disease in a subject in need thereof, comprising (a) determining that the subject is suffering from Crohn's disease; and (b) administering to the subject an effective amount of a compound of Formula (II), or a pharmaceutically acceptable salt thereof, or a pharmaceutical composition comprising an effective amount of a compound of Formula (II), or a pharmaceutically acceptable salt thereof.

Some embodiments provide a method of treating Crohn's disease in a subject previously identified or diagnosed as having Crohn's disease, the method comprising administering to the subject an effective amount of a compound of Formula (I), or a pharmaceutically acceptable salt thereof, or a pharmaceutical composition comprising an effective amount of a compound of Formula (I), or a pharmaceutically acceptable salt thereof.

Some embodiments provide a method of treating Crohn's disease in a subject previously identified or diagnosed as having Crohn's disease, the method comprising administering to the subject an effective amount of a compound of Formula (II), or a pharmaceutically acceptable salt thereof, or a pharmaceutical composition comprising an effective amount of a compound of Formula (II), or a pharmaceutically acceptable salt thereof.

In some embodiments, the subject is a human.

### Inhibiting RIPK2 Activity and XIAP Binding

Some embodiments provide a method for inhibiting RIPK2 activity in a mammalian cell, comprising contacting the mammalian cell with a compound of Formula (I), or a pharmaceutically acceptable salt thereof. In some embodiments, the mammalian cell comprises a RIPK2 protein.

Also provided is a method for inhibiting RIPK2 activity in a mammalian cell comprising a RIPK2 protein, the method comprising contacting the mammalian cell with a compound of Formula (I), or a pharmaceutically acceptable salt thereof.

Also provided is a method for inhibiting the binding of a RIPK2 protein to a XIAP protein in a mammalian cell comprising a RIPK2 protein, the method comprising contacting the mammalian cell with a compound of Formula (I), or a pharmaceutically acceptable salt thereof.

In some embodiments, the contacting is in vitro. In some embodiments, the contacting is in vivo. In some embodiments, the amount of a compound of Formula (I), or a pharmaceutically acceptable salt thereof, is sufficient to inhibit RIPK2 activity in the cell. In some embodiments, the contacting is in vivo, wherein the method comprises administering a therapeutically effective amount of a compound of Formula (I), or a pharmaceutically acceptable salt thereof, to a subject having a mammalian cell having RIPK2 activity. In some embodiments, the mammalian cell is a mammalian immune cell. In some embodiments, the mammalian cell is an cancer cell.

In some embodiments, the RIPK2 activity is inhibited by about 10% to about 99%, for example, about 10% to about 50%, about 25% to about 75%, about 50% to about 99%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or any value in between.

As used herein, the term “contacting” refers to the bringing together of indicated moieties in an in vitro system or an in vivo system. For example, “contacting” RIPK2 (e.g., a RIPK2 protein) with a compound provided herein includes the administration of a compound provided herein to a subject, such as a human, having a RIPK2 protein, as well as, for example, introducing a compound provided herein into a sample containing a mammalian cellular or purified preparation containing a RIPK2 protein.

### Pharmaceutical Compositions

Some embodiments provide a pharmaceutical composition comprising a compound of Formula (I), or a pharmaceutically acceptable salt thereof, and at least one pharmaceutically acceptable excipient.

Also provided herein is a pharmaceutical composition comprising a compound of Formula (II), or a pharmaceutically acceptable salt thereof, and at least one pharmaceutically acceptable excipient.

### NUMBERED EMBODIMENTS

Embodiment 1: A compound of Formula (I) or Formula (II):

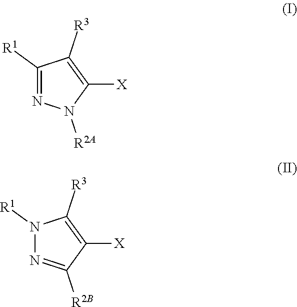

or a pharmaceutically acceptable salt of either of the foregoing, wherein:
R¹ is a 5-10 membered heteroaryl optionally substituted with 1-3 substituents independently selected from C1-C6 alkyl, C1-C6 thioalkyl, C1-C6 alkoxy optionally substituted with hydroxyl, —S(O₂) C1-C6 alkyl, NR^{1A}R^{1B}, 4-10 membered heterocyclyl optionally substituted with 1-3 substituents independently selected from halogen and C1-C6 alkyl, —(NH)—C1-C6 alkyl, and —(NH)-4-10 membered heterocyclyl optionally substituted with 1-3 substituents independently selected from halogen and C1-C6 alkyl;
R^{1A} and R^{1B} are independently selected from hydrogen, C1-C6 alkyl, and C1-C6 haloalkyl;
R^{2A} is selected from hydrogen, halogen, C1-C6 alkyl, C1-C6 haloalkyl, C1-C6 alkoxyalkyl, C1-C6 hydroxyalkyl optionally substituted with 1 or 2 halogens, C3-C6 cycloalkyl, 4-10 membered heterocyclyl optionally substituted with 1 or 2 substituents independently selected from halogen, C1-C6 alkyl, and C(O)O C1-C6 alkyl;
R^{2B} is hydrogen, C1-C6 alkyl, C1-C6 haloalkyl, or C1-C6 hydroxyalkyl;
R³ is hydrogen or halogen;
X is selected from

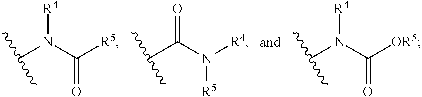

R⁴ is hydrogen or C1-C6 alkyl;
R⁵ is selected from:
(i) C1-C6 alkyl optionally substituted with 1-4 substituents independently selected from:
   (a) C3-C6 cycloalkyl optionally substituted with 1-3 substituents independently from halogen or phenyl,
   (b) phenyl optionally substituted with 1-3 substituents independently selected from halogen, C1-C6 alkoxy, C1-C6 hydroxyalkyl, and cyano,
   (c) —NR^{A}R^{B},
   (d) hydroxyl,
   (e) halogen,
   (f) 4-10 membered heterocyclyl optionally substituted with 1-3 substituents independently selected from benzyl, C1-C6 alkyl, and phenyl optionally substituted with C1-C6 alkyl, and
   (g) 5-10 membered heteroaryl optionally substituted with 1-3 substituents independently selected from halogen, cyano, C1-C6 alkyl, and C1-C6 alkoxy;
(ii) C3-C10 cycloalkyl optionally substituted with 1-3 substituents independently selected from halogen, hydroxyl, C1-C6 alkyl, phenyl optionally substituted with 1-2 independently selected halogen and 5-10 membered heteroaryl;
(iii) 4-10 membered heterocyclyl optionally substituted with 1-3 substituents independently selected from benzyl, C1-C6 alkyl, C3-C10 cycloalkyl, and phenyl optionally substituted with C1-C6 alkyl;
(iv) phenyl optionally substituted with 1-3 substituents independently selected from halogen, nitro, and C1-C6 haloalkyl;
(v) 5-10 membered heteroaryl optionally substituted with 1-3 substituents independently selected from halogen and C1-C6 alkyl;
(vi) C1-C6 alkoxy; or
(vii) C3-C6 cycloalkoxy;
or R⁴ and R⁵ with the atom to which they are attached together form a 5-12 membered heterocyclyl or 9-10 membered heteroaryl each optionally substituted with phenyl; and
R^{A} and R^{B} are independently selected from hydrogen and C1-C6 alkyl.

Embodiment 2: The compound of Embodiment 1, wherein R¹ is a 5-10 membered heteroaryl optionally substituted with 1-3 substituents independently selected from C1-C6 alkyl, C1-C6 thioalkyl, C1-C6 alkoxy optionally substituted with hydroxyl, —S(O₂) C1-C6 alkyl, NR^{1A}R^{1B}, 4-10 membered heterocyclyl optionally substituted with 1-3 substituents independently selected from halogen and C1-C6 alkyl, —(NH)—C1-C6 alkyl, and —(NH)-4-10 membered heterocyclyl optionally substituted with 1-3 substituents independently selected from halogen and C1-C6 alkyl.

Embodiment 3: The compound of Embodiment 1 or 2, wherein R¹ is a 5-10 membered heteroaryl substituted with 1-3 substituents independently selected from C1-C6 alkyl, C1-C6 thioalkyl, C1-C6 alkoxy optionally substituted with hydroxyl, —S(O₂) C1-C6 alkyl, NR^{1A}R^{1B}, 4-10 membered heterocyclyl optionally substituted with 1-3 substituents independently selected from halogen and C1-C6 alkyl, —(NH)—C1-C6 alkyl, and —(NH)-4-10 membered heterocyclyl optionally substituted with 1-3 substituents independently selected from halogen and C1-C6 alkyl.

Embodiment 4: The compound of Embodiment 1 or 2, wherein R¹ is a 9-10 membered heteroaryl substituted with 2 substituents independently selected from C1-C6 alkyl, C1-C6 thioalkyl, C1-C6 alkoxy optionally substituted with hydroxyl, —S(O₂) C1-C6 alkyl, NR^{1A}R^{1B}, 4-10 membered heterocyclyl optionally substituted with 1-3 substituents independently selected from halogen and C1-C6 alkyl, —(NH)—C1-C6 alkyl, and —(NH)-4-10 membered heterocyclyl optionally substituted with 1-3 substituents independently selected from halogen and C1-C6 alkyl.

Embodiment 5: The compound of any one of Embodiments 1-4, wherein R^{2A} is hydrogen.

Embodiment 6: The compound of any one of Embodiments 1-4, wherein R^{2A} is halogen.

Embodiment 7: The compound of any one of Embodiments 1-4, wherein R^{2A} is C1-C6 alkyl.

Embodiment 8: The compound of any one of Embodiments 1-4, wherein R^{2A} is C1-C6 haloalkyl.

Embodiment 9: The compound of any one of Embodiments 1-4, wherein R^{2A} is C1-C6 hydroxyalkyl optionally substituted with 1 or 2 halogens.

Embodiment 10: The compound of any one of Embodiments 1-4, wherein R^{2A} is C1-C6 hydroxyalkyl substituted with 1 or 2 halogens.

Embodiment 11: The compound of any one of Embodiments 1-4, wherein R^{2A} is C1-C6 alkoxyalkyl.

Embodiment 12: The compound of any one of Embodiments 1-4, wherein R^{2A} is C3-C6 cycloalkyl.

Embodiment 13: The compound of any one of Embodiments 1-4, wherein R^{2A} is 4-10 membered heterocyclyl optionally substituted with 1 or 2 substituents independently selected from halogen, C1-C6 alkyl, and C(O)O C1-C6 alkyl.

Embodiment 14: The compound of any one of Embodiments 1-4, wherein R^{2A} is 4-10 membered heterocyclyl substituted with 1 or 2 substituents independently selected from halogen, C1-C6 alkyl, and C(O)O C1-C6 alkyl.

Embodiment 15: The compound of any one of Embodiments 1-14, wherein R^{2B} is hydrogen.

Embodiment 16: The compound of any one of Embodiments 1-14, wherein R^{2B} is C1-C6 alkyl.

Embodiment 17: The compound of any one of Embodiments 1-14, wherein R^{2B} is C1-C6 haloalkyl.

Embodiment 18: The compound of any one of Embodiments 1-14, wherein R^{2B} is C1-C6 hydroxyalkyl.

Embodiment 19: The compound of any one of Embodiments 1-18, wherein R³ is hydrogen.

Embodiment 20: The compound of any one of Embodiments 1-18, wherein R³ is halogen.

Embodiment 21: The compound of any one of Embodiments 1-120, wherein X is

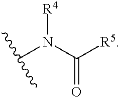

Embodiment 22: The compound of any one of Embodiments 1-20, wherein X is

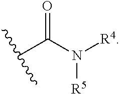

Embodiment 23: The compound of any one of Embodiments 1-20, wherein X is

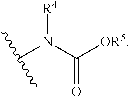

Embodiment 24: The compound of any one of Embodiments 1-23, wherein R⁴ is C1-C6 alkyl.

Embodiment 25: The compound of any one of Embodiments 1-24, wherein R⁴ is methyl.

Embodiment 26: The compound of any one of Embodiments 1-23, wherein R⁴ is hydrogen.

Embodiment 27: The compound of any one of Embodiments 1-26, wherein R⁵ is C1-C6 alkyl optionally substituted with 1-4 substituents independently selected from C3-C6 cycloalkyl optionally substituted with 1-3 substituents independently from halogen or phenyl, phenyl optionally substituted with 1-3 substituents independently selected from halogen, C1-C6 alkoxy, C1-C6 hydroxyalkyl, and cyano, —NR^{A}R^{B}, hydroxyl, halogen, 4-10 membered heterocyclyl optionally substituted with 1-3 substituents independently selected from benzyl, C1-C6 alkyl, and phenyl optionally substituted with C1-C6 alkyl, and 5-10 membered heteroaryl optionally substituted with 1-3 substituents independently selected from halogen, cyano, C1-C6 alkyl, and C1-C6 alkoxy.

Embodiment 28: The compound of any one of Embodiments 1-27, wherein R⁵ is C1-C6 alkyl substituted with 1-4 substituents independently selected from C3-C6 cycloalkyl optionally substituted with 1-3 substituents independently from halogen or phenyl, phenyl optionally substituted with 1-3 substituents independently selected from halogen, C1-C6 alkoxy, C1-C6 hydroxyalkyl, and cyano, —NR^{A}R^{B}, hydroxyl, halogen, 4-10 membered heterocyclyl optionally substituted with 1-3 substituents independently selected from benzyl, C1-C6 alkyl, and phenyl optionally substituted with C1-C6 alkyl, and 5-10 membered heteroaryl optionally substituted with 1-3 substituents independently selected from halogen, cyano, C1-C6 alkyl, and C1-C6 alkoxy.

Embodiment 28: The compound of any one of Embodiments 1-28, wherein R⁵ is C1-C6 alkyl substituted with 1 or 2 substituents independently selected from C3-C6 cycloalkyl optionally substituted with 1-3 substituents independently from halogen or phenyl, phenyl optionally substituted with 1-3 substituents independently selected from halogen, C1-C6 alkoxy, C1-C6 hydroxyalkyl, and cyano, —NR^{A}R^{B}, hydroxyl, halogen, 4-10 membered heterocyclyl optionally substituted with 1-3 substituents independently selected from benzyl, C1-C6 alkyl, and phenyl optionally substituted with C1-C6 alkyl, and 5-10 membered heteroaryl optionally substituted with 1-3 substituents independently selected from halogen, cyano, C1-C6 alkyl, and C1-C6 alkoxy.

Embodiment 30: The compound of any one of Embodiments 1-29, wherein R⁵ is C1-C6 alkyl substituted with 1 or 2 substituents independently selected from: C3-C6 cycloalkyl optionally substituted with phenyl, phenyl optionally substituted with 1-3 independently selected halogen, —NR^{A}R^{B}, hydroxyl, 4-8 membered heterocyclyl optionally substituted with 1 or 2 substituents independently selected from benzyl, C1-C6 alkyl, and phenyl optionally substituted with C1-C6 alkyl; and 5-9 membered heteroaryl optionally substituted with 1 or 2 substituents independently selected from halogen, cyano, C1-C6 alkyl, and C1-C6 alkoxy.

Embodiment 31: The compound of any one of Embodiments 27-30 wherein the C1-C6 alkyl of R⁵ is methyl.

Embodiment 32: The compound of any one of Embodiments 1-26, wherein R⁵ is C3-C10 cycloalkyl optionally substituted with 1-3 substituents independently selected from halogen, hydroxyl, C1-C6 alkyl, phenyl optionally substituted with 1-2 independently selected halogen and 5-10 membered heteroaryl.

Embodiment 33: The compound of any one of Embodiments 1-26 or 32, wherein R⁵ is C3-C10 cycloalkyl substituted with 1-3 substituents independently selected from halogen, hydroxyl, C1-C6 alkyl, phenyl optionally substituted with 1-2 independently selected halogen and 5-10 membered heteroaryl.

Embodiment 34: The compound of any one of Embodiments 1-26 or 32-33, wherein R⁵ is C3-C6 cycloalkyl substituted with 1-3 substituents independently selected from halogen, hydroxyl, C1-C6 alkyl, and phenyl optionally substituted with 1-2 independently selected halogen.

Embodiment 35: The compound of any one of Embodiments 1-26 or 32-34, wherein R⁵ is C3-C6 cycloalkyl substituted with 1 or 2 substituents independently selected from halogen, hydroxyl, C1-C6 alkyl, phenyl optionally substituted with 1-2 independently selected halogen and 5-10 membered heteroaryl.

Embodiment 36: The compound of any one of Embodiments 1-26, wherein R⁵ is 4-10 membered heterocyclyl optionally substituted with 1-3 substituents independently selected from benzyl, C1-C6 alkyl, C3-C10 cycloalkyl, and phenyl optionally substituted with C1-C6 alkyl.

Embodiment 37: The compound of any one of Embodiments 1-26 or 36, wherein R⁵ is 4-10 membered heterocyclyl substituted with 1-3 substituents independently selected from benzyl, C1-C6 alkyl, C3-C10 cycloalkyl, and phenyl optionally substituted with C1-C6 alkyl.

Embodiment 38: The compound of any one of Embodiments 1-26 or 37-38, wherein R⁵ is 4-8 membered heterocyclyl substituted with 1-3 substituents independently selected from benzyl, C1-C6 alkyl, C3-C10 cycloalkyl, and phenyl optionally substituted with C1-C6 alkyl.

Embodiment 39: The compound of any one of Embodiments 1-26, wherein R⁵ is phenyl optionally substituted with 1-3 substituents independently selected from halogen, nitro, and C1-C6 haloalkyl.

Embodiment 40: The compound of any one of Embodiments 1-26 or 39, wherein R⁵ is phenyl substituted with 1-3 substituents independently selected from halogen, nitro, and C1-C6 haloalkyl.

Embodiment 41: The compound of any one of Embodiments 1-26 or 39-40, wherein R⁵ is phenyl substituted with 1 or 2 substituents independently selected from halogen, nitro, and C1-C6 haloalkyl.

Embodiment 42: The compound of any one of Embodiments 1-26, wherein R⁵ is 5-10 membered heteroaryl optionally substituted with 1-3 substituents independently selected from halogen and C1-C6 alkyl.

Embodiment 43: The compound of any one of Embodiments 1-26 or 42, wherein R⁵ is 5-10 membered heteroaryl substituted with 1-3 substituents independently selected from halogen and C1-C6 alkyl.

Embodiment 44: The compound of any one of Embodiments 1-26 or 43, wherein R⁵ is 5-10 membered heteroaryl substituted with 1 or 2 substituents independently selected from halogen and C1-C6 alkyl.

Embodiment 45: The compound of any one of Embodiments 1-26 or 43-44, wherein R⁵ is 5-6 membered heteroaryl substituted with 1 or 2 substituents independently selected from halogen and C1-C6 alkyl.

Embodiment 46: The compound of any one of Embodiments 1-26, wherein R⁵ is C1-C6 alkoxy.

Embodiment 47: The compound of any one of Embodiments 1-26, wherein R⁵ is C3-C6 cycloalkoxy.

Embodiment 48: The compound of any one of Embodiments 1-23, wherein R⁴ and R⁵ with the atom to which they are attached together form a 5-12 membered heterocyclyl optionally substituted with phenyl.

Embodiment 49: The compound of any one of Embodiments 1-23 or 48, wherein R⁴ and R⁵ with the atom to which they are attached together form a 5-12 membered heterocyclyl.

Embodiment 50: The compound of any one of Embodiments 1-23, wherein R⁴ and R⁵ with the atom to which they are attached together form a 9-10 membered heteroaryl optionally substituted with phenyl.

Embodiment 51: The compound of any one of Embodiments 1-23 or 50, wherein R⁴ and R⁵ with the atom to which they are attached together form a 9-10 membered heteroaryl.

Embodiment 52: The compound of any one of Embodiments 1-51, wherein R^{A} is hydrogen.

Embodiment 53: The compound of any one of Embodiments 1-51, wherein R^{A} is C1-C6 alkyl.

Embodiment 54: The compound of any one of Embodiments 1-53, wherein R^{B} is hydrogen.

Embodiment 55: The compound of any one of Embodiments 1-53, wherein R^{B} is C1-C6 alkyl.

Embodiment 56: The compound of Embodiment 1, wherein Formula (I) is (I-a):

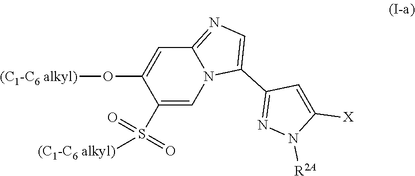

or a pharmaceutically acceptable salt thereof.

Embodiment 57: The compound of Embodiment 1, wherein Formula (I) is (I-b):

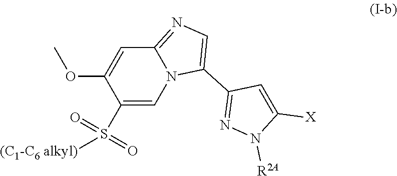

or a pharmaceutically acceptable salt thereof.

Embodiment 58: The compound of Embodiment 1, wherein Formula (I) is (I-c):

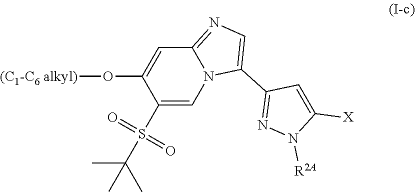

or a pharmaceutically acceptable salt thereof.

Embodiment 59: The compound of Embodiment 1, wherein Formula (I) is (I-d):

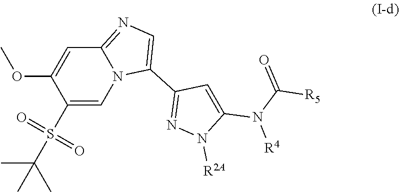

or a pharmaceutically acceptable salt thereof.

Embodiment 60: The compound of Embodiment 1, wherein Formula (I) is (I-e):

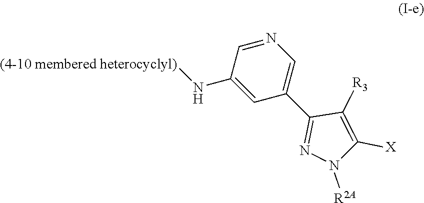

or a pharmaceutically acceptable salt thereof.

Embodiment 61: The compound of Embodiment 1, wherein Formula (I) is (I-f):

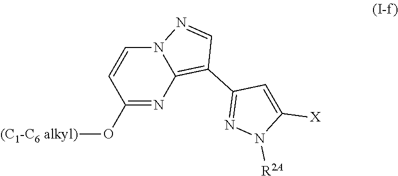

or a pharmaceutically acceptable salt thereof.

Embodiment 62: The compound of Embodiment 1, wherein Formula (II) is (II-a):

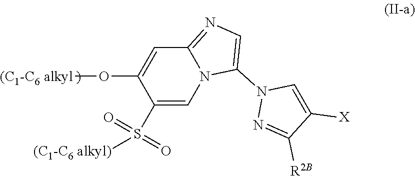

or a pharmaceutically acceptable salt thereof.

Embodiment 63: The compound of Embodiment 1, wherein Formula (II) is (II-b):

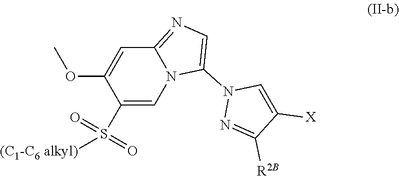

or a pharmaceutically acceptable salt thereof.

Embodiment 64: The compound of Embodiment 1, wherein Formula (II) is (II-c):

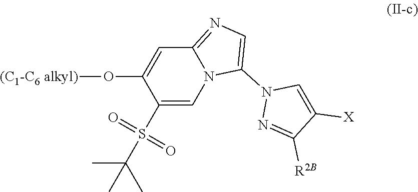

or a pharmaceutically acceptable salt thereof.

Embodiment 65: The compound of Embodiment 1, wherein Formula (II) is (II-d):

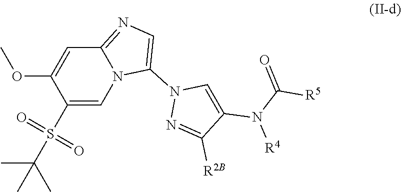

or a pharmaceutically acceptable salt thereof.

Embodiment 66: The compound of embodiment 1, wherein Formula (I) is selected from the group consisting of:

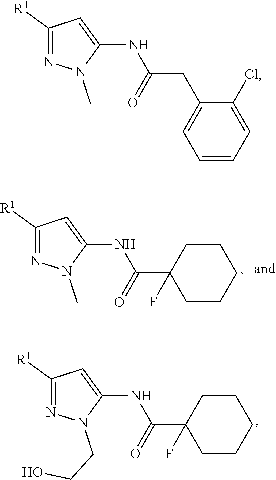

or a pharmaceutically acceptable salt thereof.

Embodiment 67: The compound of embodiment 1, wherein Formula (I) is selected from the group consisting of:

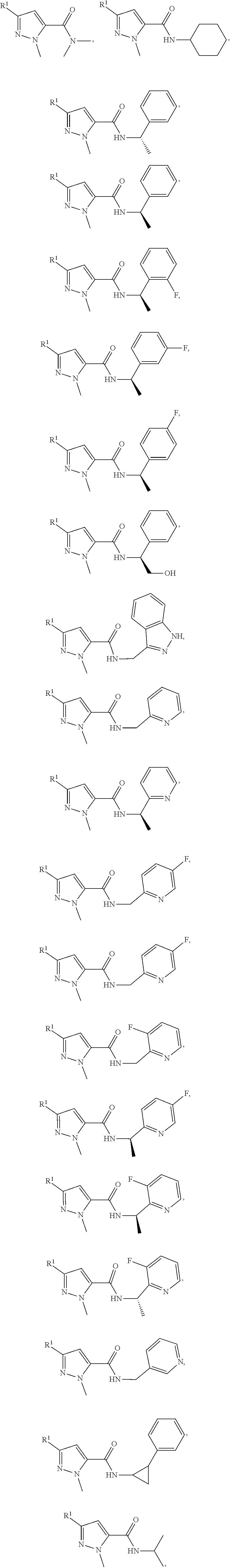

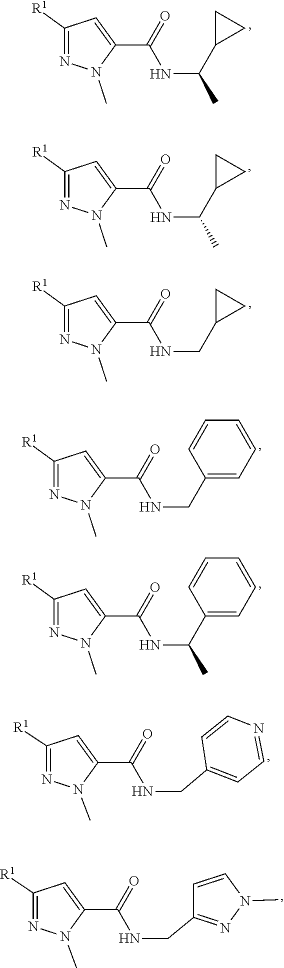

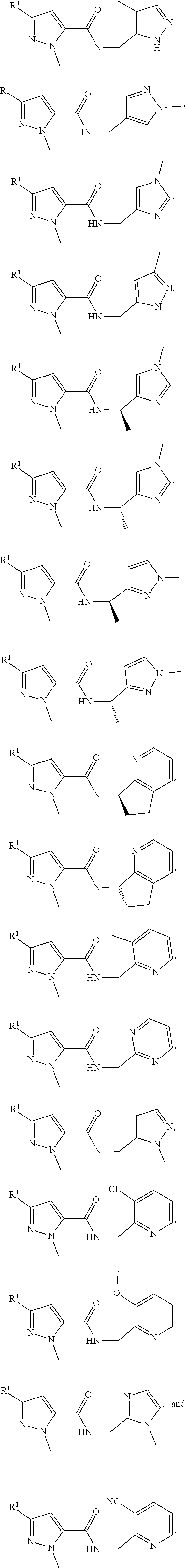

or a pharmaceutically acceptable salt thereof.

Embodiment 68: The compound of embodiment 1, wherein Formula (I) is selected from the group consisting of:

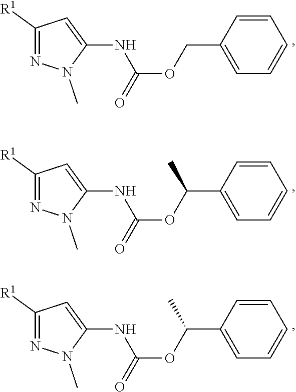

or a pharmaceutically acceptable salt thereof.

Embodiment 69: The compound of embodiment 1, wherein Formula (II) is selected from the group consisting of:

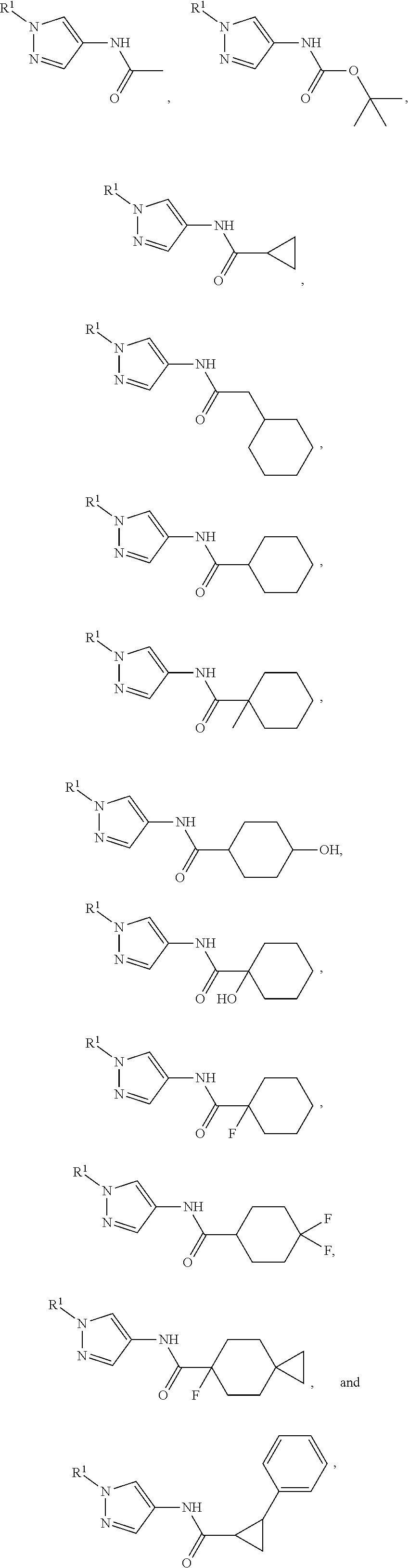

or a pharmaceutically acceptable salt thereof.

Embodiment 70: The compound of embodiment 1, wherein Formula (II) is selected from the group consisting of:

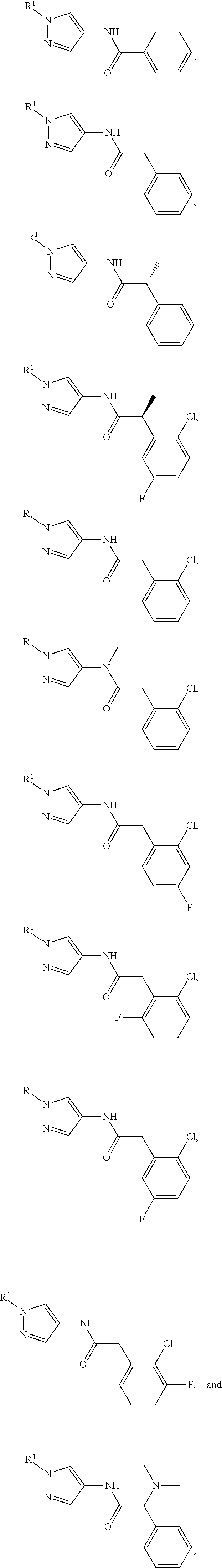

or a pharmaceutically acceptable salt thereof.

Embodiment 71: The compound of embodiment 1, wherein Formula (II) is selected from the group consisting of:

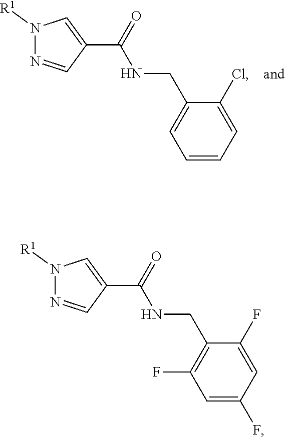

or a pharmaceutically acceptable salt thereof.

Embodiment 72: The compound of embodiment 1, wherein Formula (II) is selected from the group consisting of:

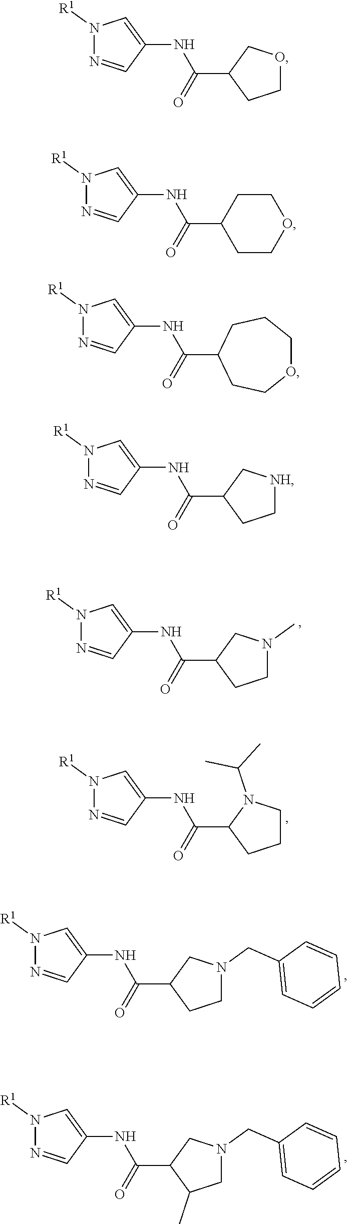

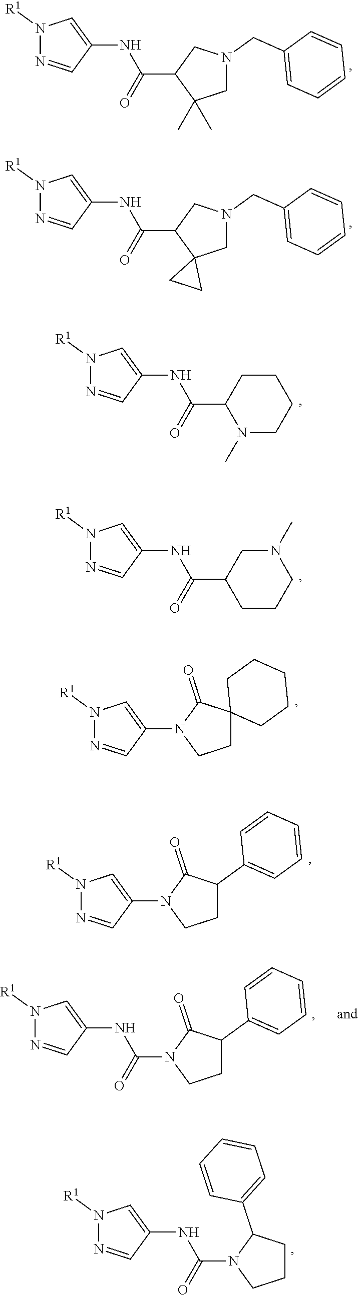

or a pharmaceutically acceptable salt thereof.

Embodiment 73: A compound selected from the group consisting of the compounds in Examples 1-194, or a pharmaceutically acceptable salt of any of the foregoing.

Embodiment 74: A pharmaceutical composition comprising a compound of any one of Embodiments 1-73, or a pharmaceutically acceptable salt thereof, and one or more pharmaceutically acceptable excipients.

Embodiment 75: A method of treating a RIPK2-associated disease or disorder in a subject in need thereof, comprising administering to the subject an effective amount of a compound of any one of Embodiments 1-73, or a pharmaceutically acceptable salt thereof, or the pharmaceutical composition of Embodiment 74.

Embodiment 76: The method of Embodiment 75, wherein the RIPK2-associated disease or disorder is a cardiovascular disease, an allergic disorder, an autoimmune disease, an inflammatory disease, a cardiovascular disease, a fibrotic disease, or a disease associated with abnormal cell growth.

Embodiment 77: The method of Embodiment 75 or 76, wherein the RIPK2-associated disease or disorder is an inflammatory disease.

Embodiment 78: The method of Embodiment 77, wherein the inflammatory disease is chronic lung inflammatory disease, osteoarthritis, inflammatory arthritis, asthma, early onset sarcoidosis, sarcoidosis, eczema, allergic eczema, uveitis, reactive arthritis, chronic inflammation, chronic prostatitis, inflammatory bowel disease, glomerulonephritis, bursitis, carpal tunnel syndrome, tendinitis, inflammation of the lung (e.g., chronic obstructive pulmonary disease), pelvic inflammatory disease, transplant rejection, vasculitis, regional enteritis, distal ileitis, regional ileitis, and terminal ileitis, central areolar choroidal dystrophy, macular degeneration, retinosis pigmentosa, adult vitelliform disease, pattern dystrophy, diabetic retinopathy, BEST disease, myopic degeneration, central serous retinopathy, Stargardt's disease, Cone-Rod dystrophy, North Carolina dystrophy, infectious retinitis, inflammatory retinitis, uveitis, toxic retinitis, or systemic inflammatory response syndrome.

Embodiment 79: A method of treating inflammatory bowel disease in a subject in need thereof, comprising administering to the subject an effective amount of the compound of any one of Embodiments 1-73, or a pharmaceutically acceptable salt thereof, or the pharmaceutical composition of Embodiment 74.

Embodiment 80: A method of treating inflammatory bowel disease in a subject in need thereof, comprising (a) determining that the subject is suffering from inflammatory bowel disease; and (b) administering to the subject an effective amount of the compound of the compound of any one of Embodiments 1-73, or a pharmaceutically acceptable salt thereof, or the pharmaceutical composition of Embodiment 74.

Embodiment 81: A method of treating inflammatory bowel disease in a subject previously identified or diagnosed as having inflammatory bowel disease, the method comprising administering to the subject an effective amount of the compound of the compound of any one of Embodiments 1-73, or a pharmaceutically acceptable salt thereof, or the pharmaceutical composition of Embodiment 74.

Embodiment 82: A method of treating Crohn's disease in a subject in need thereof, comprising administering to the subject an effective amount of the compound of the compound of any one of Embodiments 1-73, or a pharmaceutically acceptable salt thereof, or the pharmaceutical composition of Embodiment 74.

Embodiment 83: A method of treating Crohn's disease in a subject in need thereof, comprising (a) determining that the subject is suffering from Crohn's disease; and (b) administering to the subject an effective amount of the compound of the compound of any one of Embodiments 1-73, or a pharmaceutically acceptable salt thereof, or the pharmaceutical composition of Embodiment 74.

Embodiment 84: A method of treating Crohn's disease in a subject previously identified or diagnosed as having Crohn's disease, the method comprising administering to the subject an effective amount of the compound of the compound of any one of Embodiments 1-73, or a pharmaceutically acceptable salt thereof, or the pharmaceutical composition of Embodiment 74.

### EXAMPLES

### Materials and Methods

The compounds provided herein, including salts thereof, can be prepared using known organic synthesis techniques and can be synthesized according to any of numerous possible synthetic routes.

The reactions for preparing the compounds provided herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of the compounds provided herein can involve the protection and deprotection of various chemical groups. The need for protection and deprotection, and the selection of appropriate protecting groups, can be readily determined by one skilled in the art. The chemistry of protecting groups can be found, for example, in Protecting Group Chemistry, 1^{st} Ed., Oxford University Press, 2000; March's Advanced Organic Chemistry: Reactions, Mechanisms, and Structure, 5th Ed., Wiley-Interscience Publication, 2001; and Peturssion, S. et al., “Protecting Groups in Carbohydrate Chemistry,” J. Chem. Educ., 74 (11), 1297 (1997).

### Intermediates

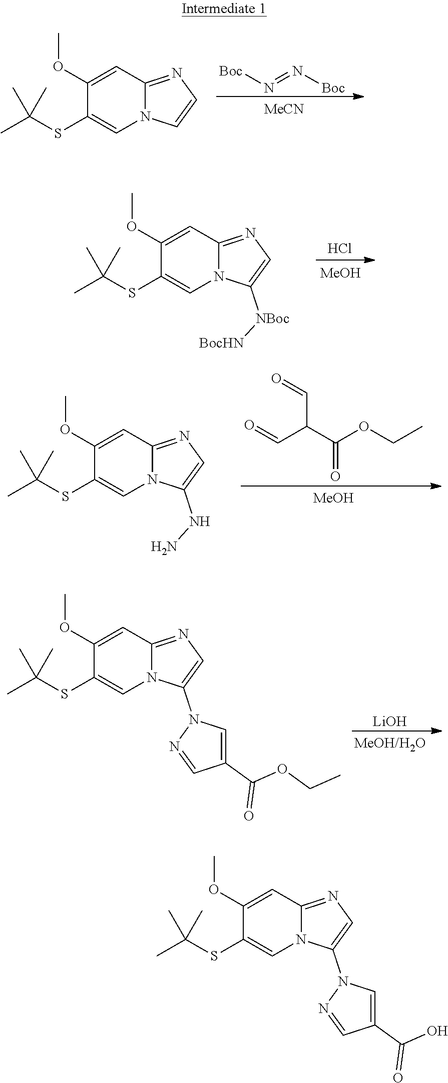

di-tert-butyl 1-(6-(tert-butylthio)-7-methoxyimidazo[1,2-a]pyridin-3-yl)hydrazine-1,2-dicarboxylate: To a solution of 6-(tert-butylthio)-7-methoxyimidazo[1,2-a]pyridine (1.80 g, 7.62 mmol, 1 eq) in MeCN (80 mL) was added di-tert-butyl azodicarboxylate (10.52 g, 45.70 mmol, 6 eq). The mixture was stirred at 80° C. for 12 hours before it was diluted with H₂O (50 mL) and extracted with EtOAc (3×50 mL). The combined organic extracts were dried over Na₂SO₄, filtered and concentrated under reduced pressure to give a residue, which was purified by column chromatography (SiO₂, 10-50% EtOAc/petroleum ether) to give the title compound (2.65 g, 67% yield) as a light yellow solid. [M+H]+=467.4. ¹H NMR (400 MHZ, CDCl₃) δ ppm 8.62 (s, 1H), 7.42 (s, 1H), 7.00-6.90 (m, 1H), 6.86 (s, 1H), 3.91 (s, 3H), 1.45 (s, 18H), 1.30 (s, 9H).

6-(tert-butylthio)-3-hydrazineyl-7-methoxyimidazo[1,2-a]pyridine dihydrochloride: A mixture of di-tert-butyl 1-(6-(tert-butylthio)-7-methoxyimidazo[1,2-a]pyridin-3-yl) hydrazine-1,2-dicarboxylate (1 g, 1.93 mmol, 1 eq) in 4 M HCl in MeOH (20 mL) was degassed and purged with N₂ three times, and then the mixture was stirred at 25° C. for 3 hours under a N₂ atmosphere. The mixture was concentrated under reduced pressure to give the crude title compound (500 mg) as a yellow solid.

ethyl 1-(6-(tert-butylthio)-7-methoxyimidazo[1,2-a]pyridin-3-yl)-1H-pyrazole-4-carboxylate: To a solution of 6-(tert-butylthio)-3-hydrazineyl-7-methoxyimidazo[1,2-a]pyridine dihydrochloride (500 mg, 1.88 mmol, 1 eq) in MeOH (10 mL) was added ethyl 2-formyl-3-oxopropanoate (1.35 g, 9.39 mmol, 5 eq). The mixture was stirred at 25° C. for 12 hours before it was concentrated under reduced pressure. The residue was purified by column chromatography (SiO₂, 0-4% MeOH/DCM) to give the title compound (518 mg, 66% yield) as a yellow solid. [M+H]⁺=375.2.

1-(6-(tert-butylthio)-7-methoxyimidazo[1,2-a]pyridin-3-yl)-1H-pyrazole-4-carboxylic acid: To a solution of ethyl 1-(6-(tert-butylthio)-7-methoxyimidazo[1,2-a]pyridin-3-yl)-1H-pyrazole-4-carboxylate (518 mg, 1.25 mmol, 90% purity, 1 eq) in MeOH (24 mL) and H₂O (4 mL) was added LiOH·H₂O (522.40 mg, 12.45 mmol, 10 eq). The mixture was stirred at 25° C. for 12 hours before it was concentrated under reduced pressure. The residue was dissolved in water (2 mL) and neutralized with 1 N HCl. The precipitate was isolated by filtration, and then the filtered cake was washed with water (3 mL) and dried to give the title compound (300 mg, 69% yield) as a light yellow solid. [M+H]⁺=347.0. ¹H NMR (400 MHz, DMSO-d₆) δ ppm 13.09-12.36 (m, 1H), 8.73 (s, 1H), 8.27 (d, J=8.4 Hz, 2H), 7.80 (br s, 1H), 7.14 (br s, 1H), 3.91 (s, 3H), 1.22 (s, 9H).

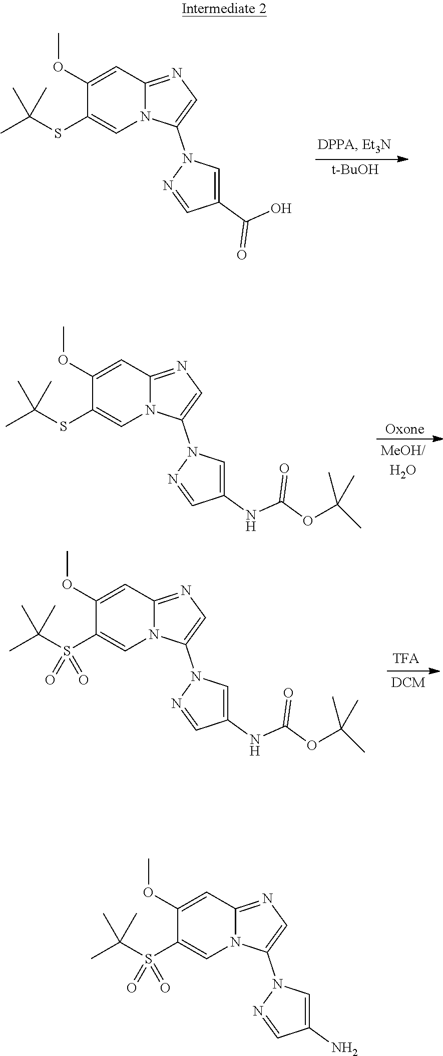

tert-butyl (1-(6-(tert-butylthio)-7-methoxyimidazo[1,2-a]pyridin-3-yl)-1H-pyrazol-4-yl) carbamate: A mixture of 1-(6-(tert-butylthio)-7-methoxyimidazo[1,2-a]pyridin-3-yl)-1H-pyrazole-4-carboxylic acid (50 mg, 142.41 μmol, 98.66% purity, 1 eq) and Et₃N (15.85 mg, 156.65 μmol, 21.80 μL, 1.1 eq) in t-BuOH (5 mL) was degassed and purged with N₂ three times, and then DPPA (43.11 mg, 156.65 μmol, 33.94 μL, 1.1 eq) was added. The mixture was stirred under a N₂ atmosphere at 20° C. for 30 min, and then at 90° C. for another 4 hours before it was concentrated under reduced pressure. The residue was purified by column chromatography (SiO₂, 1-10% MeOH/DCM) to give the title compound (80 mg, 40% yield) as a white solid. [M+H]⁺=418.0.

¹H NMR (400 MHz, DMSO-d₆) δ ppm 9.47 (s, 1H), 8.25 (s, 1H), 8.03 (s, 1H), 7.77 (s, 1H), 7.68 (s, 1H), 7.10 (s, 1H), 3.89 (s, 3H), 1.47 (s, 9H), 1.22 (s, 9H).

tert-butyl (1-(6-(tert-butylsulfonyl)-7-methoxyimidazo[1,2-a]pyridin-3-yl)-1H-pyrazol-4-yl) carbamate: To a solution of tert-butyl (1-(6-(tert-butylthio)-7-methoxyimidazo[1,2-a]pyridin-3-yl)-1H-pyrazol-4-yl) carbamate (110 mg, 237.11 μmol, 90% purity, 1 eq) in MeOH (12 mL) and H₂O (3 mL) was added Oxone® (874.61 mg, 1.42 mmol, 6 eq). The mixture was stirred at 25° C. for 12 hours before it was quenched with saturated Na₂SO₃ (5 mL) and extracted with EtOAc (2×10 mL). The combined organic extracts were dried over Na₂SO₄, filtered and concentrated under reduced pressure to give a residue, which was purified by RP-HPLC to give the title compound (105 mg, 98% yield) as a white solid. [M+H]⁺=450.1. ¹H NMR (400 MHZ, DMSO-d₆) δ ppm 9.51 (s, 1H), 8.73 (s, 1H), 8.10 (s, 1H), 7.84 (s, 1H), 7.78 (s, 1H), 7.27 (s, 1H), 3.93 (s, 3H), 1.47 (s, 9H), 1.29 (s, 9H).

1-(6-(tert-butylsulfonyl)-7-methoxyimidazo[1,2-a]pyridin-3-yl)-1H-pyrazol-4-amine: A solution of tert-butyl (1-(6-(tert-butylsulfonyl)-7-methoxyimidazo[1,2-a]pyridin-3-yl)-1H-pyrazol-4-yl) carbamate (60 mg, 132.46 μmol, 99.24% purity, 1 eq) in DCM (2 mL) and TFA (0.4 mL) was stirred at 25° C. for 1 hour before it was concentrated under reduced pressure. The solid was dissolved in water (5 mL) and extracted with EtOAc (2×5 mL), and then saturated NaHCO₃ was added to the aqueous phase and it was extracted again with EtOAc (2×5 mL). The combined organic extracts were washed with brine (10 mL), dried over Na₂SO₄, filtered and concentrated to give the title compound (40 mg, 84% yield) as a pink solid. [M+H]⁺=349.9. ¹H NMR (400 MHZ, DMSO-d₆) δ ppm 8.80 (s, 1H), 7.71 (s, 1H), 7.48-7.40 (m, 2H), 7.24 (s, 1H), 4.31 (br s, 2H), 3.92 (s, 3H), 1.29 (s, 9H).

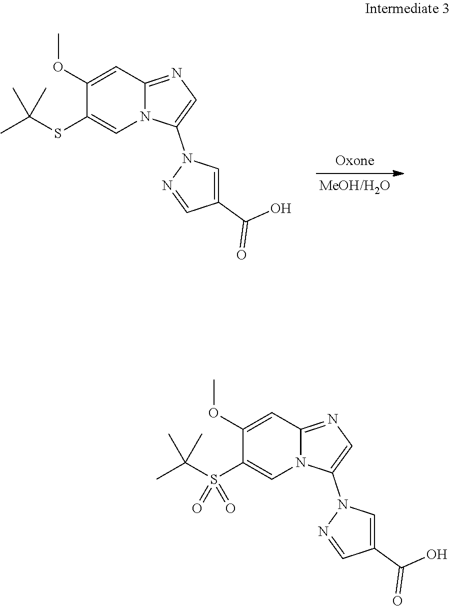 [SMILES: COc1cc2ncc(-n3cc(C(=O)O)cn3)n2cc1SC(C)(C)C ; COc1cc2ncc(-n3cc(C(=O)O)cn3)n2cc1S(=O)(=O)C(C)(C)C]

1-(6-(tert-butylsulfonyl)-7-methoxyimidazo[1,2-a]pyridin-3-yl)-1H-pyrazole-4-carboxylic acid: To a solution of 1-(6-(tert-butylthio)-7-methoxyimidazo[1,2-a]pyridin-3-yl)-1H-pyrazole-4-carboxylic acid (100 mg, 259.81 μmol, 90% purity, 1 eq) in MeOH (10 mL) and H₂O (2.5 mL) was added Oxone® (798.63 mg, 1.30 mmol, 5 eq). The mixture was stirred at 25° C. for 3 hours before it was quenched with saturated Na₂SO₃ (20 mL), and extracted with EtOAc (2×20 mL). The combined organic layers were washed with brine (10 mL), dried over Na₂SO₄, filtered and concentrated under reduced pressure to give a residue. The residue was purified by RP-HPLC to give the title compound (5.8 mg, 6% yield) as a white solid. [M+H]⁺=378.9. ¹H NMR (400 MHz, DMSO-d₆) δ ppm 8.85 (s, 1H), 8.81 (s, 1H), 8.34 (s, 1H), 8.17 (s, 1H), 7.41 (s, 1H), 4.00 (s, 3H), 1.30 (s, 9H).

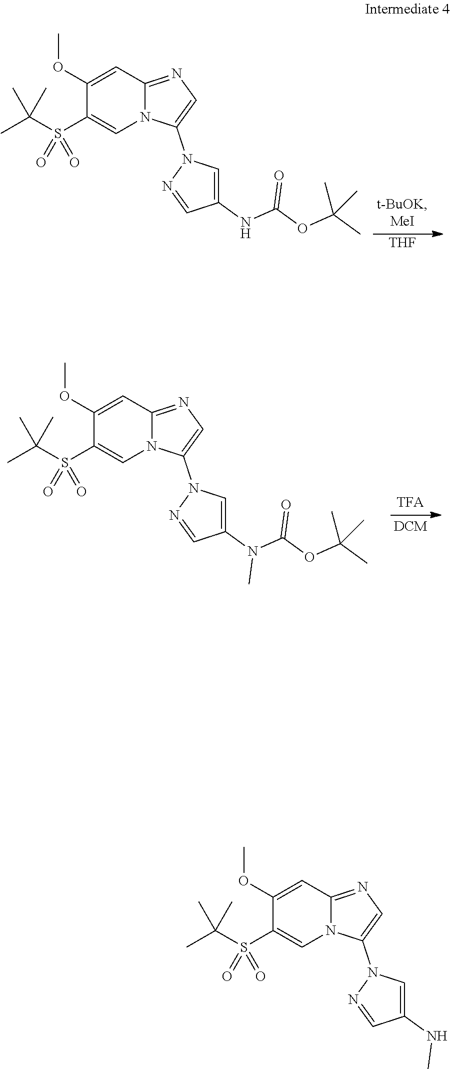

tert-butyl (1-(6-(tert-butylsulfonyl)-7-methoxyimidazo[1,2-a]pyridin-3-yl)-1H-pyrazol-4-yl)(methyl) carbamate: To a mixture of tert-butyl (1-(6-(tert-butylsulfonyl)-7-methoxyimidazo[1,2-a]pyridin-3-yl)-1H-pyrazol-4-yl) carbamate (100 mg, 218.59 μmol, 1 eq) and t-BuOK (36.79 mg, 327.88 μmol, 1.5 eq) in THF (10 mL) was added CH₃I (37.23 mg, 262.30 μmol, 16.33 μL, 1.2 eq). The mixture was stirred at 25° C. for 1 hour under a N₂ atmosphere before it was diluted with water (10 mL) and extracted with DCM (3×10 mL). The combined organic extracts were dried over Na₂SO₄, filtered and concentrated under reduced pressure. The residue was purified by column chromatography (SiO₂, 0-10% MeOH/DCM) to give the title compound (60 mg, 53% yield) as yellow oil. [M+H]⁺=464.1. ¹H NMR (400 MHz, DMSO-d₆) δ ppm 8.77 (s, 1H), 8.36-7.96 (m, 2H), 7.84 (s, 1H), 7.29 (s, 1H), 3.94 (s, 3H), 3.23 (s, 3H), 1.49 (s, 9H), 1.29 (s, 9H).

1-(6-(tert-butylsulfonyl)-7-methoxyimidazo[1,2-a]pyridin-3-yl)-N-methyl-1H-pyrazol-4-amine: To a mixture of tert-butyl (1-(6-(tert-butylsulfonyl)-7-methoxyimidazo[1,2-a]pyridin-3-yl)-1H-pyrazol-4-yl)(methyl) carbamate (60 mg, 116.49 μmol, 1 eq) in DCM (4 mL) was added TFA (3.07 g, 26.93 mmol, 2 mL, 231.13 eq). The mixture was stirred at 25° C. for 1 hour under a N₂ atmosphere before it was diluted with saturated NaHCO₃ (20 mL) and extracted with DCM (3×10 mL). The combined organic extracts were dried over Na₂SO₄, filtered and concentrated under reduced pressure to give the title compound (45 mg, 96% yield) as a yellow solid. [M+H]⁺=363.9.

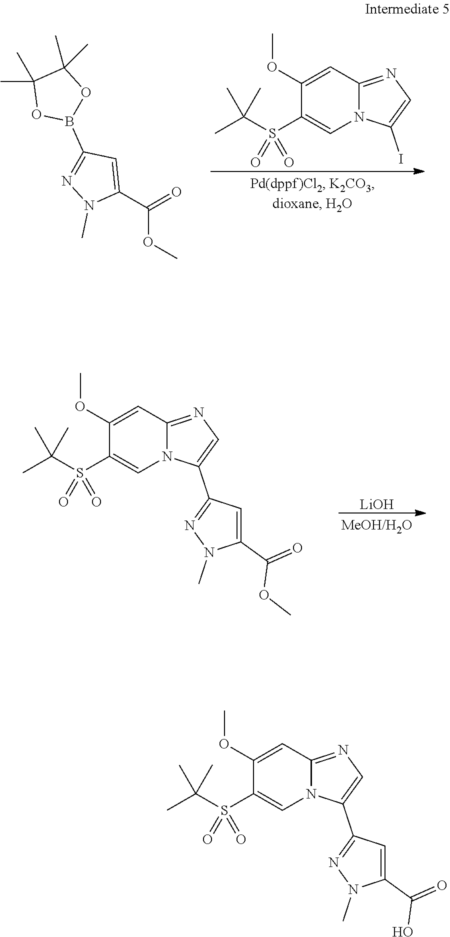

methyl 3-(6-(tert-butylsulfonyl)-7-methoxyimidazo[1,2-a]pyridin-3-yl)-1-methyl-1H-pyrazole-5-carboxylate: A mixture of methyl 1-methyl-3-(4,4,5,5-tetramethyl-1,3,2-dioxaborolan-2-yl)-1H-pyrazole-5-carboxylate (850 mg, 2.87 mmol, 1 eq), 6-(tert-butylsulfonyl)-3-iodo-7-methoxyimidazo[1,2-a]pyridine (1.13 g, 2.87 mmol, 1 eq), K₂CO₃ (1.19 g, 8.62 mmol, 3 eq), Pd (dppf) Cl₂ (210.36 mg, 287.49 μmol, 0.1 eq) in 1,4-dioxane (56 mL) and H₂O (14 mL) was degassed and purged with N₂ three times, and then the mixture was stirred at 80° C. for 3 hours under a N₂ atmosphere. The mixture was diluted with water (100 mL) and extracted with EtOAc (3×100 mL). The combined organic extracts were dried over Na₂SO₄, filtered and concentrated under reduced pressure. The residue was purified by column chromatography (SiO₂, 1-10% MeOH/DCM) to give the title compound (340 mg, 26% yield) as a brown solid. 1H NMR (400 MHz, DMSO-d₆) δ ppm 9.84 (s, 1H), 8.17 (s, 1H), 7.45 (s, 1H), 7.30 (s, 1H), 4.18 (s, 3H), 3.94 (s, 3H), 3.89 (s, 3H), 1.33 (s, 9H).

3-(6-(tert-butylsulfonyl)-7-methoxyimidazo[1,2-a]pyridin-3-yl)-1-methyl-1H-pyrazole-5-carboxylic acid: To a solution of methyl 3-(6-(tert-butylsulfonyl)-7-methoxyimidazo[1,2-a]pyridin-3-yl)-1-methyl-1H-pyrazole-5-carboxylate (340 mg, 752.85 μmol, 1 eq) in MeOH (6 mL) and H₂O (1 mL) was added LiOH (315.92 mg, 7.53 mmol, 10 eq). The mixture was stirred at 25° C. for 2 hours before it was diluted with water (2 mL) and acidified with 2 N HCl (pH=5) at 0° C. The resulting mixture was diluted with water (100 mL) and extracted with EtOAc (3×100 mL). The combined organic extracts were dried over Na₂SO₄, filtered and concentrated under reduced pressure. The residue was purified by column chromatography (SiO₂, 1-10% MeOH/DCM) to give the title compound (200 mg, 61% yield) as a yellow solid. [M+H]⁺=393.0. ¹H NMR (400 MHZ, DMSO-d₆) δ ppm 10.00 (s, 1H), 8.57 (s, 1H), 7.50 (s, 2H), 4.19 (s, 3H), 4.06 (s, 3H), 1.35 (s, 9H).

The following intermediate was prepared following procedures analogous to that described for Intermediate 5.

| Int. | | | |
|---|---|---|---|
| No. | Structure | Name | LCMS, ¹H NMR |
| 6 | 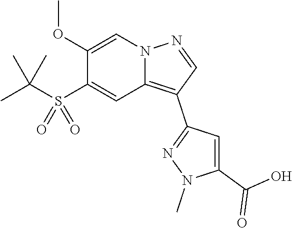 [SMILES: COc1cn2ncc(-c3cc(C(=O)O)n(C)n3)c2cc1S(=O)(=O)C(C)(C)C] | 3-(5-(tert-butylsulfonyl)-6- methoxypyrazolo[1,5- a]pyridin-3-yl)-1-methyl- 1H-pyrazole-5-carboxylic acid | [M + H]⁺ = 393.1 |

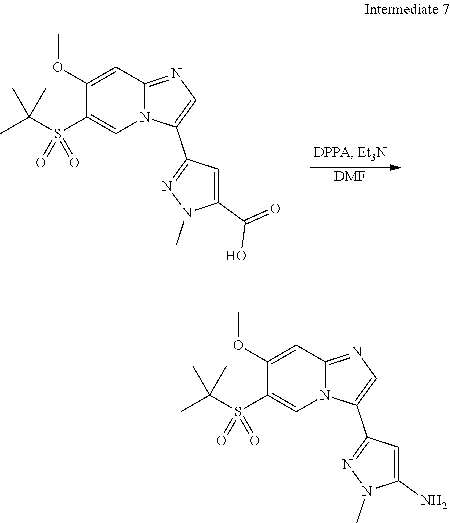

3-(6-(tert-butylsulfonyl)-7-methoxyimidazo[1,2-a]pyridin-3-yl)-1-methyl-1H-pyrazol-5-amine: A mixture of 3-(6-(tert-butylsulfonyl)-7-methoxyimidazo[1,2-a]pyridin-3-yl)-1-methyl-1H-pyrazole-5-carboxylic acid (200 mg, 458.68 μmol, 1 eq), DPPA (138.85 mg, 504.55 μmol, 108.90 μL, 1.1 eq) and Et₃N (139.24 mg, 1.38 mmol, 191.53 μL, 3 eq) in DMF (25 mL) was degassed and purged with N₂ three times, and then the mixture was stirred at 90° C. for 12 hours under a N₂ atmosphere. The reaction mixture was concentrated under reduced pressure to give a residue, which was purified by column chromatography (SiO₂, 1-5% MeOH/DCM) to give the title compound (85 mg, 46% yield) as a white solid. 1H NMR (400 MHZ, DMSO-d₆) δ ppm 9.94 (s, 1H), 7.86 (s, 1H), 7.21 (s, 1H), 5.72 (s, 1H), 5.47 (s, 2H), 3.92 (s, 3H), 3.60 (s, 3H), 1.31 (s, 9H).

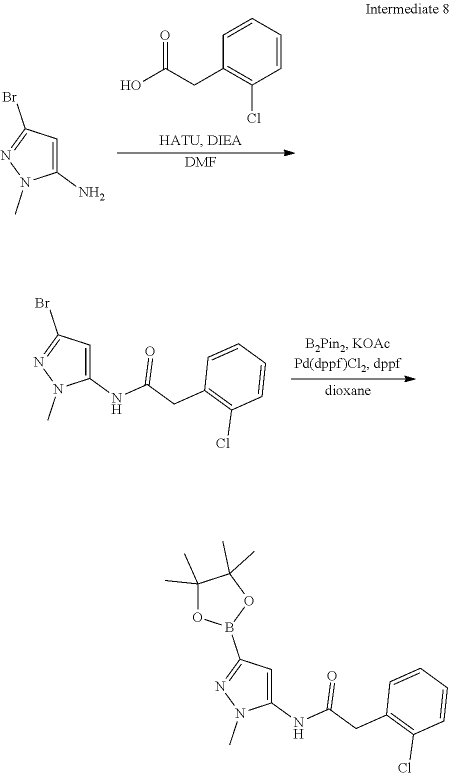

N-(3-bromo-1-methyl-1H-pyrazol-5-yl)-2-(2-chlorophenyl) acetamide: To a solution of 3-bromo-1-methyl-1H-pyrazol-5-amine (10 mg, 56.81 μmol, 1 eq) in DMF (1 mL) at 20° C. was added DIEA (14.69 mg, 113.63 μmol, 19.79 μL, 2 eq) dropwise, followed by HATU (43.20 mg, 113.63 μmol, 2 eq). The mixture was stirred at 20° C. for 30 min, and then 2-(2-chlorophenyl) acetic acid (9.69 mg, 56.81 μmol, 1 eq) was added. The resulting mixture was stirred at 20° C. for 16 hours before it was diluted with water (5 mL) and extracted with EtOAc (3×5 mL). The combined organic layers were washed with brine (3×5 mL), dried over Na₂SO₄, filtered and concentrated under reduced pressure. The residue was purified by column chromatography (SiO₂, 0-5% MeOH/DCM) to give the title compound (150 mg, 72% yield) as a yellow solid. 1H NMR (400 MHz, DMSO-d₆) δ ppm 10.40 (s, 1H), 7.46-7.39 (m, 2H), 7.35-7.27 (m, 2H), 6.31 (s, 1H), 3.88 (s, 2H), 3.67 (s, 3H).

2-(2-chlorophenyl)-N-(1-methyl-3-(4,4,5,5-tetramethyl-1,3,2-dioxaborolan-2-yl)-1H-pyrazol-5-yl) acetamide: A mixture of N-(3-bromo-1-methyl-1H-pyrazol-5-yl)-2-(2-chlorophenyl) acetamide (100 mg, 273.90 μmol, 1 eq), B₂Pin₂ (83.46 mg, 328.68 μmol, 1.2 eq), Pd (dppf) Cl₂ (20.04 mg, 27.39 μmol, 0.1 eq), dppf (15.18 mg, 27.39 μmol, 0.1 eq) and KOAc (80.64 mg, 821.69 μmol, 3 eq) in dioxane (1 mL) was degassed and purged with N₂ three times, and then the mixture was stirred at 100° C. for 2 hours under a N₂ atmosphere. The reaction mixture was filtered and concentrated under reduced pressure to give the title compound (150 mg) as a black-brown solid, which was used in the next step without further purification.

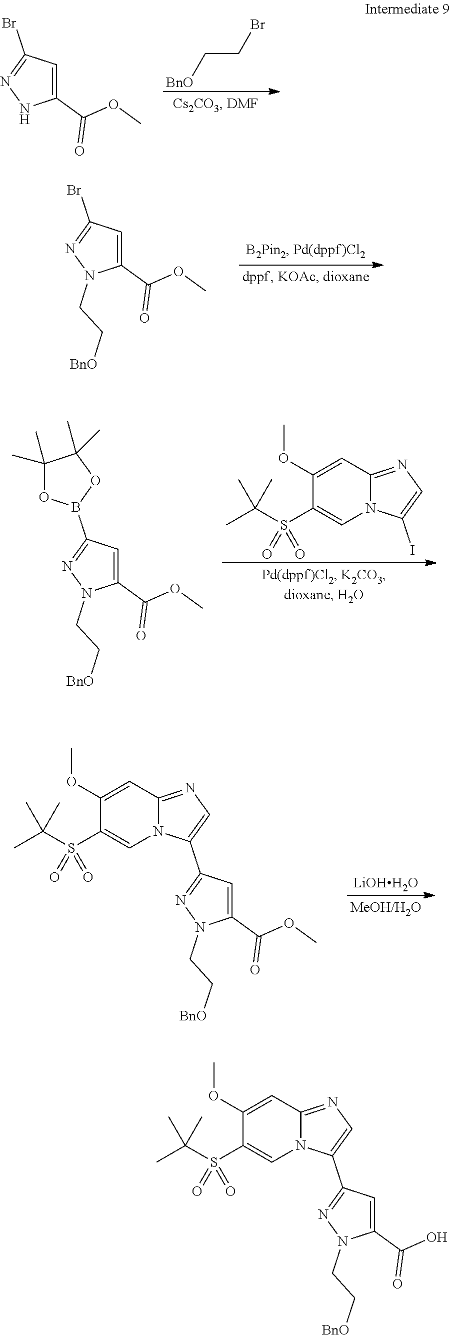

methyl 1-(2-(benzyloxy)ethyl)-3-bromo-1H-pyrazole-5-carboxylate: To a solution of methyl 3-bromo-1H-pyrazole-5-carboxylate (1 g, 4.88 mmol, 1 eq) in DMF (2 mL) was added Cs₂CO₃ (4.77 g, 14.63 mmol, 3 eq) and ((2-bromoethoxy)methyl)benzene (2.62 g, 12.19 mmol, 1.93 mL, 2.5 eq). The mixture was stirred at 25° C. for 1 hour before it was diluted with water (30 mL) at 0° C., and then extracted with EtOAc (3×30 mL). The combined organic extracts were washed with brine (30 mL), dried over Na₂SO₄, filtered and concentrated under reduced pressure to give a residue, which was purified by column chromatography (SiO₂, 0-50% EtOAc/petroleum ether) to give the title compound (1.1 g, 60% yield) as a colorless oil. [M+H]⁺=338.8. ¹H NMR (400 MHZ, CDCl₃) δ ppm 7.34-7.26 (m, 3H), 7.25-7.17 (m, 2H), 6.80 (s, 1H), 4.78 (t, J=5.6 Hz, 2H), 4.48 (s, 2H), 3.85-3.78 (m, 5H).

methyl 1-(2-(benzyloxy)ethyl)-3-(4,4,5,5-tetramethyl-1,3,2-dioxaborolan-2-yl)-1H-pyrazole-5-carboxylate: A mixture of methyl 1-(2-(benzyloxy)ethyl)-3-bromo-1H-pyrazole-5-carboxylate (450 mg, 1.19 mmol, 1 eq), B₂Pin₂ (909.64 mg, 3.58 mmol, 3 eq), KOAc (351.56 mg, 3.58 mmol, 3 eq), Pd (dppf) Cl₂ (87.37 mg, 119.40 μmol, 0.1 eq) and dppf (132.39 mg, 238.81 μmol, 0.2 eq) in dioxane (8 mL) was degassed and purged with N₂ three times, and then the mixture was stirred at 100° C. for 3 hours under a N₂ atmosphere. The mixture was filtered, the filter cake was washed with dioxane (3×5 mL) and the filtrate was concentrated under reduced pressure to give the title compound (900 mg) as brown oil, which was used in the next step without further purification.

methyl 1-(2-(benzyloxy)ethyl)-3-(6-(tert-butylsulfonyl)-7-methoxyimidazo[1,2-a]pyridin-3-yl)-1H-pyrazole-5-carboxylate: A mixture of methyl 1-(2-(benzyloxy)ethyl)-3-(4,4,5,5-tetramethyl-1,3,2-dioxaborolan-2-yl)-1H-pyrazole-5-carboxylate (900 mg, 2.10 mmol, 1 eq), 6-(tert-butylsulfonyl)-3-iodo-7-methoxyimidazo[1,2-a]pyridine (918.59 mg, 2.10 mmol, 1 eq), K₂CO₃ (869.49 mg, 6.29 mmol, 3 eq), and Pd (dppf) Cl₂ (153.45 mg, 209.71 μmol, 0.1 eq) in dioxane (8 mL) and H₂O (2 mL) was degassed and purged with N₂ three times. The mixture was stirred at 80° C. for 2 hours under a N₂ atmosphere before it was diluted with water (100 mL), extracted with EtOAc (3×100 mL). The combined organic extracts were dried over Na₂SO₄, filtered and concentrated under reduced pressure to give a residue, which was purified by column chromatography (SiO₂, 0-100% EtOAc/petroleum ether) to give the title compound (680 mg, 55% yield) as a yellow solid. [M+H]⁺=527.0. ¹H NMR (400 MHZ, DMSO-d₆) δ ppm 9.89 (s, 1H), 8.20 (s, 1H), 7.47 (s, 1H), 7.30 (s, 1H), 7.25-7.11 (m, 5H), 4.81 (t, J=5.2 Hz, 2H), 4.46 (s, 2H), 3.96-3.88 (m, 5H), 3.86 (s, 3H), 1.29 (s, 9H).

1-(2-(benzyloxy)ethyl)-3-(6-(tert-butylsulfonyl)-7-methoxyimidazo[1,2-a]pyridin-3-yl)-1H-pyrazole-5-carboxylic acid: To a solution of methyl 1-(2-(benzyloxy)ethyl)-3-(6-(tert-butylsulfonyl)-7-methoxyimidazo[1,2-a]pyridin-3-yl)-1H-pyrazole-5-carboxylate (680 mg, 1.16 mmol, 1 eq) in MeOH (6 mL) and H₂O (1 mL) was added LiOH—H₂O (487.69 mg, 11.62 mmol, 10 eq). The mixture was stirred at 25° C. for 4 hours before it was diluted with water (2 mL) and adjusted to pH=5 with 2 N HCl at 0° C. The precipitate was isolated by filtration, washed with water (3×10 mL) and dried under vacuum to give the title compound (500 mg, 76% yield) as a yellow solid. [M+H]⁺=513.0. ¹H NMR (400 MHZ, DMSO-d₆) δ ppm 9.91 (s, 1H), 8.15 (s, 1H), 7.35 (s, 1H), 7.29 (s, 1H), 7.24-7.13 (m, 5H), 4.82 (t, J=5.2 Hz, 2H), 4.47 (s, 2H), 3.95-3.90 (m, 5H), 1.29 (s, 9H).

The following intermediates were prepared following procedures analogous to that described for Intermediate 9.

| Int. | | | |
|---|---|---|---|
| No. | Structure | Name | LCMS, ¹H NMR |
| 10 | 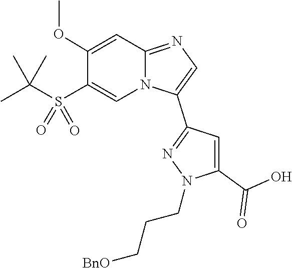 [SMILES: COc1cc2ncc(-c3cc(C(=O)O)n(CCCOCc4ccccc4)n3)n2cc1S(=O)(=O)C(C)(C)C] | 1-(3- (benzyloxy)propyl)- 3-(6-(tert- butylsulfonyl)-7- methoxyimidazo[1,2- a]pyridin-3-yl)-1H- pyrazole-5- carboxylic acid | ¹H NMR (400 MHz, DMSO-d₆) δ ppm 10.00 (s, 1H), 7.95 (s, 1H), 7.36-7.19 (m, 6H), 6.78 (s, 1H), 4.74 (t, J = 6.8 Hz, 2H), 4.45 (s, 2H), 3.95-3.88 (m, 3H), 3.49 (t, J = 6.4 Hz, 2H), 2.16-2.07 (m, 2H), 1.30 (s, 9H) |
| 11 | 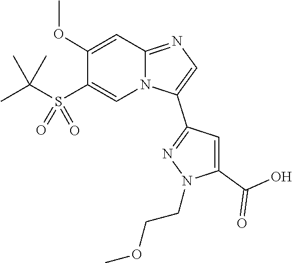 [SMILES: COCCn1nc(-c2cnc3cc(OC)c(S(=O)(=O)C(C)(C)C)cn23)cc1C(=O)O] | 3-(6-(tert- butylsulfonyl)-7- methoxyimidazo[1,2- a]pyridin-3-yl)-1-(2- methoxyethyl)-1H- pyrazole-5- carboxylic acid | [M + H]⁺ = 437.0 ¹H NMR (400 MHz, DMSO-d₆) δ ppm 9.91 (s, 1H), 8.15 (s, 1H), 7.35 (s, 1H), 7.29 (s, 1H), 4.76 (t, J = 5.2 Hz, 2H), 3.94 (s, 3H), 3.83 (t, J = 5.6 Hz, 2H), 3.25 (s, 3H), 1.33 (s, 9H) |
| 12 | 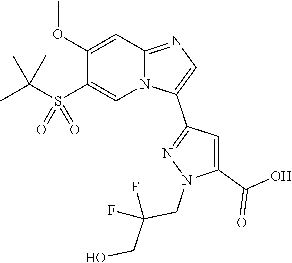 [SMILES: COc1cc2ncc(-c3cc(C(=O)O)n(CC(F)(F)CO)n3)n2cc1S(=O)(=O)C(C)(C)C] | 3-(6-(tert- butylsulfonyl)-7- methoxyimidazo[1,2- a]pyridin-3-yl)-1- (2,2-difluoro-3- hydroxypropyl)-1H- pyrazole-5- carboxylic acid | [M + H]⁺ = 473.1 ¹H NMR (400 MHz, DMSO-d₆) δ ppm 9.90 (s, 1H), 8.39 (s, 1H), 7.52 (s, 1H), 7.39 (s, 1H), 5.19 (t, J = 14.4 Hz, 2H), 3.99 (s, 3H), 3.75-3.67 (m, 2H), 1.32 (s, 9H) |
| 13 | 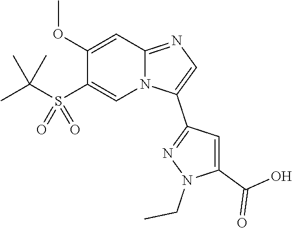 [SMILES: CCn1nc(-c2cnc3cc(OC)c(S(=O)(=O)C(C)(C)C)cn23)cc1C(=O)O] | 3-(6-(tert- butylsulfonyl)-7- methoxyimidazo[1,2- a]pyridin-3-yl)-1- ethyl-1H-pyrazole-5- carboxylic acid | [M + H]⁺ = 407.0 |
| 14 | 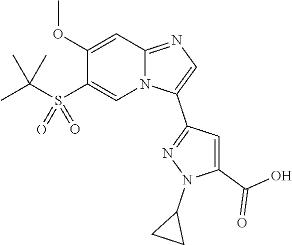 [SMILES: COc1cc2ncc(-c3cc(C(=O)O)n(C4CC4)n3)n2cc1S(=O)(=O)C(C)(C)C] | 3-(6-(tert- butylsulfonyl)-7- methoxyimidazo[1,2- a]pyridin-3-yl)-1- cyclopropyl-1H- pyrazole-5- carboxylic acid | ¹H NMR (400 MHz, CDCl₃) δ ppm 9.98 (s, 1H), 7.94 (s, 1H), 7.22 (s, 1H), 6.77 (s, 1H), 5.46-5.33 (m, 1H), 3.92 (s, 3H), 1.33 (s, 9H), 1.18 (br s, 2H), 0.93-0.87 (m, 2H) |
| 15 | 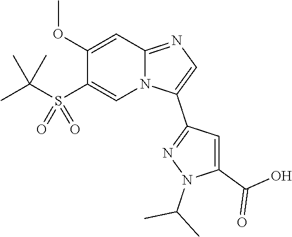 [SMILES: COc1cc2ncc(-c3cc(C(=O)O)n(C(C)C)n3)n2cc1S(=O)(=O)C(C)(C)C] | 3-(6-(tert- butylsulfonyl)-7- methoxyimidazo[1,2- a]pyridin-3-yl)-1- isopropyl-1H- pyrazole-5- carboxylic acid | ¹H NMR (400 MHz, DMSO-d₆) δ ppm 10.18 (s, 1H), 7.93 (s, 1H), 7.22 (s, 1H), 6.72 (s, 1H), 6.09-6.00 (m, 1H), 3.92 (s, 3H), 1.39 (d, J = 6.4 Hz, 6H), 1.32 (s, 9H) |
| 16 | 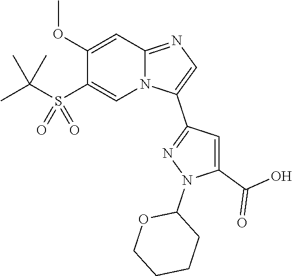 [SMILES: COc1cc2ncc(-c3cc(C(=O)O)n(C4CCCCO4)n3)n2cc1S(=O)(=O)C(C)(C)C] | 3-(6-(tert- butylsulfonyl)-7- methoxyimidazo[1,2- a]pyridin-3-yl)-1- (tetrahydro-2H- pyran-2-yl)-1H- pyrazole-5- carboxylic acid | ¹H NMR (400 MHz, DMSO-d₆) δ ppm 8.49 (s, 1H), 7.73 (s, 1H), 7.30 (s, 1H), 6.73 (s, 1H), 5.17-5.12 (m, 1H), 3.96-3.86 (m, 5H), 2.41-2.26 (m, 2H), 1.96-1.90 (m, 1H), 1.85- 1.81 (m, 1H), 1.48 (s, 2H), 1.30 (s, 9H) |
| 17 | 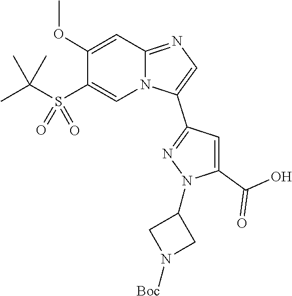 [SMILES: COc1cc2ncc(-c3cc(C(=O)O)n(C4CN(C(=O)OC(C)(C)C)C4)n3)n2cc1S(=O)(=O)C(C)(C)C] | 1-(1-(tert- butoxycarbonyl)azeti- din-3-yl)-3-(6-(tert- butylsulfonyl)-7- methoxyimidazo[1,2- a]pyridin-3-yl)-1H- pyrazole-5- carboxylic acid | [M + H]⁺ = 534.1 ¹H NMR (400 MHz, DMSO-d₆) δ ppm 9.99 (s, 1H), 8.23 (br s, 1H), 7.47 (s, 1H), 7.32 (s, 1H), 5.96-5.83 (m, 1H), 4.40-4.22 (m, 4H), 3.95 (s, 3H), 1.41 (s, 9H), 1.34 (s, 9H) |
| 18 | 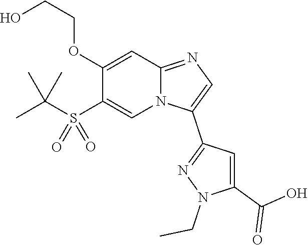 [SMILES: CCn1nc(-c2cnc3cc(OCCO)c(S(=O)(=O)C(C)(C)C)cn23)cc1C(=O)O] | 3-(6-(tert- butylsulfonyl)-7-(2- hydroxyethoxy)imidazo [1,2-a]pyridin-3- yl)-1-ethyl-1H- pyrazole-5- carboxylic acid | [M + H]⁺ = 436.9 ¹H NMR (400 MHz, DMSO-d₆) δ ppm 9.93 (s, 1H), 8.14 (s, 1H), 7.35 (s, 1H), 7.30 (s, 1H), 4.88-4.77 (m, 1H), 4.59 (q, J = 6.4 Hz, 2H), 4.23- 4.16 (m, 2H), 3.81-3.74 (m, 2H), 1.46 (t, J = 6.8 Hz, 3H), 1.34 (s, 9H) |
| 19 | 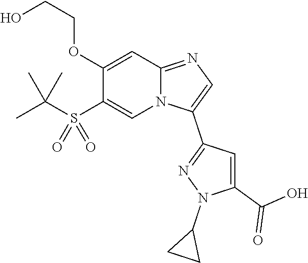 [SMILES: CC(C)(C)S(=O)(=O)c1cn2c(-c3cc(C(=O)O)n(C4CC4)n3)cnc2cc1OCCO] | 3-(6-(tert- butylsulfonyl)-7-(2- hydroxyethoxy)imidazo [1,2-a]pyridin-3- yl)-1-cyclopropyl- 1H-pyrazole-5- carboxylic acid | ¹H NMR (400 MHz, DMSO-d₆) δ ppm 9.98 (s, 1H), 7.94 (s, 1H), 7.67- 7.40 (m, 1H), 7.23 (s, 1H), 6.84 (s, 1H), 5.33 (d, J = 0.8 Hz, 1H), 4.88 (s, 1H), 4.17 (s, 2H), 3.77 (s, 2H), 1.34 (s, 9H), 1.06 (s, 2H), 0.92 (d, J = 1.6 Hz, 2H) |
| 20 | 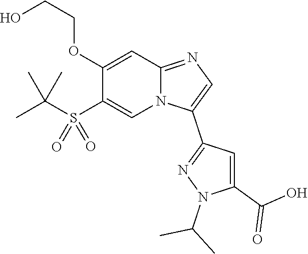 [SMILES: CC(C)n1nc(-c2cnc3cc(OCCO)c(S(=O)(=O)C(C)(C)C)cn23)cc1C(=O)O] | 3-(6-(tert- butylsulfonyl)-7-(2- hydroxyethoxy)imidazo [1,2-a]pyridin-3- yl)-1-isopropyl-1H- pyrazole-5- carboxylic acid | ¹H NMR (400 MHz, DMSO-d₆) δ ppm 10.18 (s, 1H), 7.93 (s, 1H), 7.23 (s, 1H), 6.74 (s, 1H), 6.09-5.99 (m, 1H), 4.98-4.80 (m, 1H), 4.17 (t, J = 4.8 Hz, 2H), 4.07-3.95 (m, 1H), 3.77 (t, J = 4.8 Hz, 2H), 1.40 (d, J = 6.4 Hz, 6H), 1.34 (s, 9H) |
| 21 | 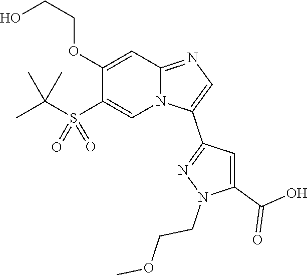 [SMILES: COCCn1nc(-c2cnc3cc(OCCO)c(S(=O)(=O)C(C)(C)C)cn23)cc1C(=O)O] | 3-(6-(tert- butylsulfonyl)-7-(2- hydroxyethoxy)imidazo [1,2-a]pyridin-3- yl)-1-(2- methoxyethyl)-1H- pyrazole-5- carboxylic acid | ¹H NMR (400 MHz, DMSO-d₆) δ ppm 9.91 (s, 1H), 8.14 (s, 1H), 7.36 (s, 1H), 7.30 (s, 1H), 4.82 (br s, 1H), 4.76 (t, J = 5.2 Hz, 2H), 4.19 (t, J = 4.8 Hz, 2H), 3.83 (t, J = 5.2 Hz, 2H), 3.78 (br s, 2H), 3.25 (s, 3H), 1.34 (s, 9H) |
| 22 | 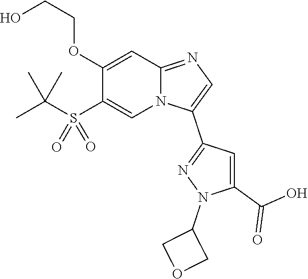 [SMILES: CC(C)(C)S(=O)(=O)c1cn2c(-c3cc(C(=O)O)n(C4COC4)n3)cnc2cc1OCCO] | 3-(6-(tert- butylsulfonyl)-7-(2- hydroxyethoxy)imidazo [1,2-a]pyridin-3- yl)-1-(oxetan-3-yl)- 1H-pyrazole-5- carboxylic acid | ¹H NMR (400 MHz, DMSO-d₆) δ ppm 10.13 (s, 1H), 8.01 (s, 1H), 7.27 (s, 1H), 6.88 (s, 1H), 6.56 (s, 1H), 5.02 (s, 2H), 4.89 (s, 2H), 4.18 (s, 2H), 3.78 (s, 2H), 1.35 (s, 9H) |

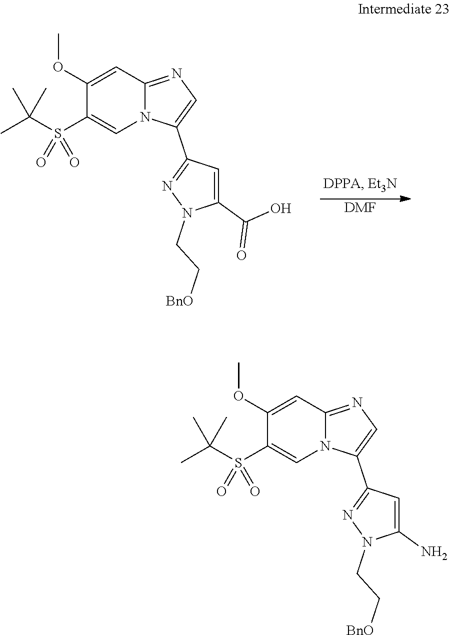

1-(2-(benzyloxy)ethyl)-3-(6-(tert-butylsulfonyl)-7-methoxyimidazo[1,2-a]pyridin-3-yl)-1H-pyrazol-5-amine: To a solution of 1-(2-(benzyloxy)ethyl)-3-(6-(tert-butylsulfonyl)-7-methoxyimidazo[1,2-a]pyridin-3-yl)-1H-pyrazole-5-carboxylic acid (500 mg, 877.92 μmol, 1 eq) in DMF (15 mL) was added Et₃N (106.60 mg, 1.05 mmol, 146.63 μL, 1.2 eq) and DPPA (314.08 mg, 1.14 mmol, 246.34 μL, 1.3 eq). The mixture was stirred at 90° C. for 12 hours under a N₂ atmosphere before it was concentrated under reduced pressure to give a residue, which was purified by RP-HPLC to give the title compound (180 mg, 38% yield) as a yellow solid. [M+H]⁺=484.0.

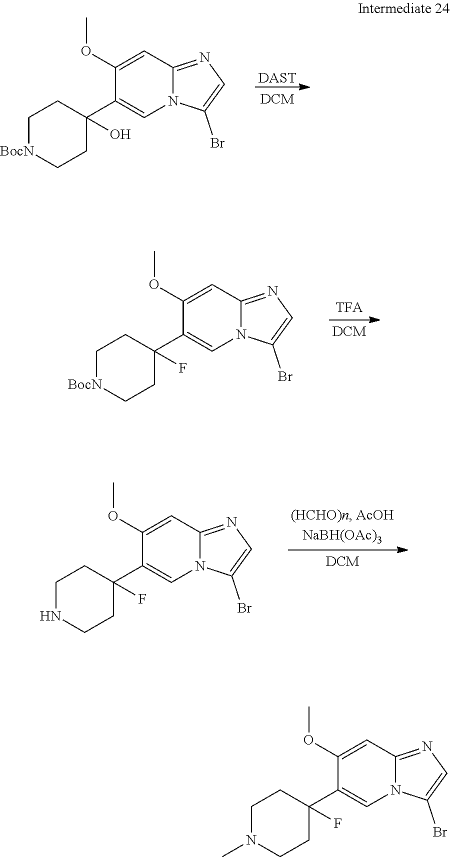

tert-butyl 4-(3-bromo-7-methoxyimidazo[1,2-a]pyridin-6-yl)-4-fluoropiperidine-1-carboxylate: To a mixture of tert-butyl 4-(3-bromo-7-methoxyimidazo[1,2-a]pyridin-6-yl)-4-hydroxypiperidine-1-carboxylate (1.1 g, 2.32 mmol, 1 eq) in DCM (10 mL) was added a solution of DAST (3.58 g, 22.20 mmol, 2.93 mL, 9.56 eq) in THF (2.5 mL) at −65° C. The mixture was stirred at 25° C. for 1 hour under a N₂ atmosphere before it was quenched with saturated NaHCO₃ (20 mL) and extracted with DCM (3×20 mL). The combined organic extracts were dried over Na₂SO₄, filtered and concentrated under reduced pressure to give a residue, which was purified by column chromatography (SiO₂, 0-10% MeOH/DCM) to give the title compound (70 mg, 6% yield) as a yellow solid. [M+H]⁺=430.2. ¹H NMR (400 MHZ, DMSO-d₆) δ ppm 8.05 (s, 1H), 7.58 (s, 1H), 7.17 (s, 1H), 3.99-3.90 (m, 2H), 3.88 (s, 3H), 3.18-3.00 (m, 2H), 2.48-2.30 (m, 2H), 1.89-1.79 (m, 2H), 1.45 (s, 9H).

3-bromo-6-(4-fluoropiperidin-4-yl)-7-methoxyimidazo[1,2-a]pyridine: To a mixture of tert-butyl 4-(3-bromo-7-methoxyimidazo[1,2-a]pyridin-6-yl)-4-fluoropiperidine-1-carboxylate (70 mg, 147.09 μmol, 1 eq) in DCM (2 mL) was added TFA (1.54 g, 13.46 mmol, 1 mL, 91.52 eq) at 25° C. The mixture was stirred at 25° C. for 1 hour under a N₂ atmosphere before it was concentrated under reduced pressure to give the title compound (45 mg, 84% yield) as a yellow solid. 1H NMR (400 MHZ, DMSO-d₆) δ ppm 8.03 (s, 1H), 7.58 (s, 1H), 7.17 (s, 1H), 3.92 (s, 3H), 3.09-2.99 (m, 2H), 2.98-2.88 (m, 2H), 2.60-2.53 (m, 1H), 2.49-2.35 (m, 1H), 1.88-1.76 (m, 2H).

3-bromo-6-(4-fluoro-1-methylpiperidin-4-yl)-7-methoxyimidazo[1,2-a]pyridine: A mixture of 3-bromo-6-(4-fluoropiperidin-4-yl)-7-methoxyimidazo[1,2-a]pyridine (45 mg, 123.41 μmol, 1 eq), paraformaldehyde (90 mg, 493.63 μmol, 4 eq), and AcOH (2.10 g, 34.94 mmol, 2 mL, 283.10 eq) in DCM (2 mL) was stirred at 25° C. for 1 hour, and then NaBH (OAc)₃ (104.62 mg, 493.63 μmol, 4 eq) was added at 0° C. The mixture was stirred at 25° C. for 1 hour under a N₂ atmosphere before it was quenched with saturated NaHCO₃ (10 mL) and extracted with DCM (3×10 mL). The combined organic extracts were dried over Na₂SO₄, filtered and concentrated under reduced pressure to give the title compound (20 mg, 43% yield) as yellow oil. [M+H]⁺=344.0.

¹H NMR (400 MHZ, DMSO-d₆) δ ppm 8.04 (s, 1H), 7.57 (s, 1H), 7.16 (s, 1H), 3.91 (s, 3H), 2.76-2.50 (m, 4H), 2.30-2.15 (m, 5H), 1.83-1.71 (m, 2H).

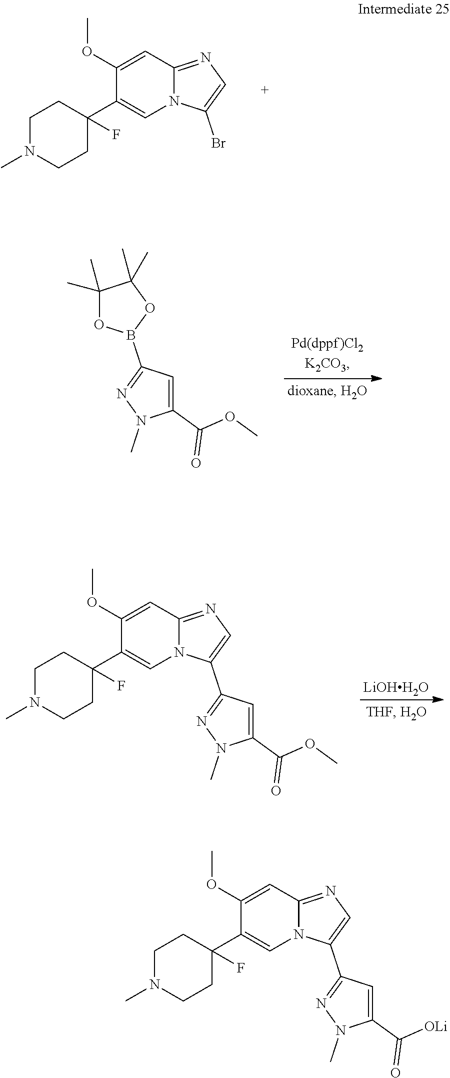

methyl 3-(6-(4-fluoro-1-methylpiperidin-4-yl)-7-methoxyimidazo[1,2-a]pyridin-3-yl)-1-methyl-1H-pyrazole-5-carboxylate: A mixture of 3-bromo-6-(4-fluoro-1-methylpiperidin-4-yl)-7-methoxyimidazo[1,2-a]pyridine (0.139 g, 365.57 μmol, 1 eq), methyl 1-methyl-3-(4,4,5,5-tetramethyl-1,3,2-dioxaborolan-2-yl)-1H-pyrazole-5-carboxylate (129.70 mg, 438.68 μmol, 1.2 eq), K₂CO₃ (151.57 mg, 1.10 mmol, 3 eq) and Pd (dppf) Cl₂ (26.75 mg, 36.56 μmol, 0.1 eq) in dioxane (1 mL) and water (0.2 mL) was degassed and purged with N₂ three times. The resulting mixture was stirred at 80° C. for 2 hours under a N₂ atmosphere before it was filtered and concentrated under reduced pressure. The residue was purified by column chromatography (SiO₂, 1-10% MeOH/DCM) to give the title compound (50 mg, 31% yield) as a black-brown solid. 1H NMR (400 MHZ, DMSO-d₆) δ ppm 9.33 (s, 1H), 8.01 (s, 1H), 7.37 (s, 1H), 7.16 (s, 1H), 4.18 (s, 3H), 3.92 (s, 3H), 3.88 (s, 3H), 2.80-2.70 (m, 2H), 2.54 (s, 3H), 2.27 (s, 4H), 1.80 (t, J=12.4 Hz, 2H).

lithium 3-(6-(4-fluoro-1-methylpiperidin-4-yl)-7-methoxyimidazo[1,2-a]pyridin-3-yl)-1-methyl-1H-pyrazole-5-carboxylate: To a solution of methyl 3-(6-(4-fluoro-1-methylpiperidin-4-yl)-7-methoxyimidazo[1,2-a]pyridin-3-yl)-1-methyl-1H-pyrazole-5-carboxylate (0.04 g, 89.68 μmol, 1 eq) in THF (1 mL) and water (0.2 mL) was added LiOH·H₂O (5.27 mg, 125.55 μmol, 1.4 eq). The mixture was stirred at 25° C. for 1 hour before it was concentrated under reduced pressure to give the title compound (30 mg, 78% yield) as a white solid. 1H NMR (400 MHZ, DMSO-d₆) δ ppm 9.39 (s, 1H), 7.78 (s, 1H), 7.09 (s, 1H), 6.69 (s, 1H), 4.13 (s, 3H), 3.91 (s, 3H), 2.69 (d, J=6.8 Hz, 2H), 2.54 (s, 3H), 2.33-2.23 (m, 4H), 1.78 (t, J=12.4 Hz, 2H).

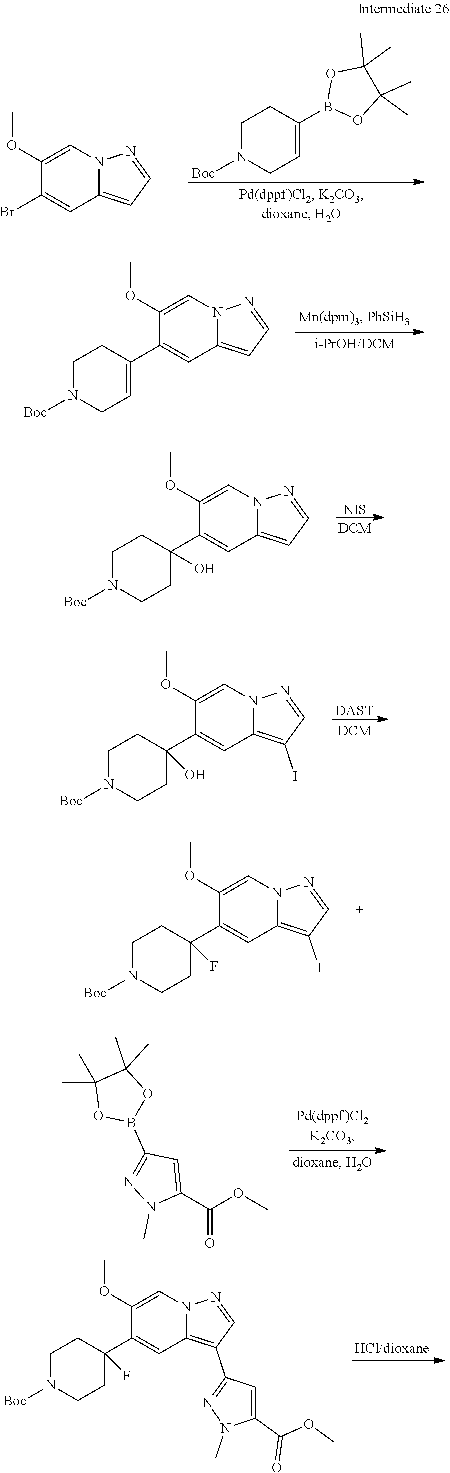

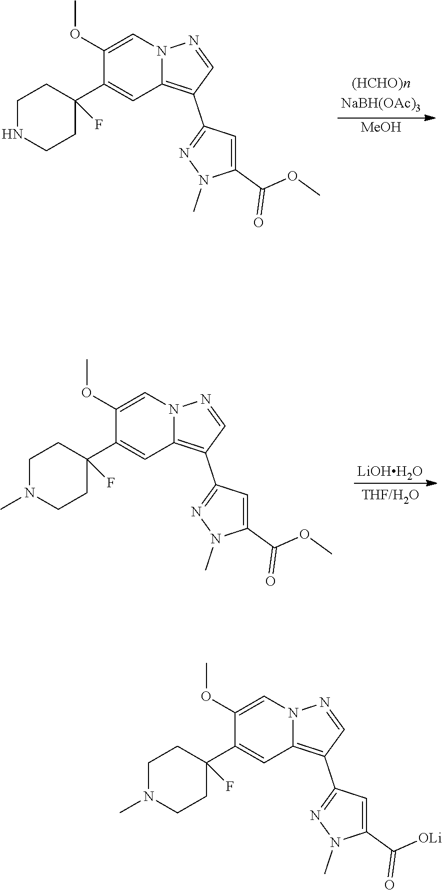

tert-butyl 4-(6-methoxypyrazolo[1,5-a]pyridin-5-yl)-3,6-dihydropyridine-1 (2H)-carboxylate: To a solution of 5-bromo-6-methoxypyrazolo[1,5-a]pyridine (462.01 mg, 1.63 mmol, 1 eq) and tert-butyl 4-(4,4,5,5-tetramethyl-1,3,2-dioxaborolan-2-yl)-3,6-dihydropyridine-1 (2H)-carboxylate (755 mg, 2.44 mmol, 1.5 eq) in dioxane (5 mL) and water (1 mL) was added Pd (dppf) Cl₂ (119.11 mg, 162.78 μmol, 0.1 eq) and K₂CO₃ (674.92 mg, 4.88 mmol, 3 eq). The mixture was stirred at 80° C. for 12 h under a N₂ atmosphere before it was filtered and concentrated in vacuo. The resulting crude material was purified by column chromatography (SiO₂, 0-30% EtOAc/petroleum ether) to yield the title compound as a yellow solid. 1H NMR (400 MHZ, DMSO-d₆) δ ppm 8.35 (s, 1H), 7.84 (d, J=2.0 Hz, 1H), 7.44 (s, 1H), 6.49 (d, J=2.4 Hz, 1H), 5.95 (s, 1H), 3.98 (s, 2H), 3.82 (s, 3H), 3.50 (t, J=5.6 Hz, 2H), 2.41 (s, 2H), 1.43 (s, 9H).

tert-butyl 4-hydroxy-4-(6-methoxypyrazolo[1,5-a]pyridin-5-yl) piperidine-1-carboxylate: A mixture of tert-butyl 4-(6-methoxypyrazolo[1,5-a]pyridin-5-yl)-3,6-dihydropyridine-1 (2H)-carboxylate (400 mg, 1.09 mmol, 1 eq), Mn (dpm)₃ (66.09 mg, 109.29 μmol, 0.1 eq) and PhSiH3 (236.54 mg, 2.19 mmol, 269.71 μL, 2 eq) in iPrOH (10 mL) and DCM (2 mL) was degassed and purged with O₂ three times. The reaction mixture was stirred at 25° C. for 1 h under a O₂ atmosphere (15 psi) before it was diluted with H₂O (10 mL) and extracted with EtOAc (3×10 mL). The combined organic layers were washed with brine (10 mL), dried over Na₂SO₄, filtered and concentrated in vacuo. The resulting crude material was purified by column chromatography (SiO₂, 0-100% EtOAc/petroleum ether) to yield the title compound as a brown oil. 1H NMR (400 MHz, CDCl₃) δ ppm 8.34 (s, 1H). 7.88 (d. J=2.4 Hz, 1H). 7.48 (s, 1H). 6.51 (d. J=2.0 Hz, 1H), 4.07-4.01 (m, 2H). 3.96 (s, 3H). 3.38-3.24 (m, 2H). 2.02-1.92 (m, 4H). 1.48 (s, 9H).

tert-butyl 4-hydroxy-4-(3-iodo-6-methoxypyrazolo[1,5-a]pyridin-5-yl) piperidine-1-carboxylate: To a solution of tert-butyl 4-hydroxy-4-(6-methoxypyrazolo[1,5-a]pyridin-5-yl) piperidine-1-carboxylate (500 mg, 1.15 mmol, 1 eq) in MeCN (10 mL) was added NIS (310.85 mg. 1.38 mmol, 1.2 eq) at 0° C. The mixture was stirred at 0° C. for 0.5 hour before it was quenched with saturated Na₂SO₃ (10 mL) at 0° C. and then diluted with EtOAc (50 mL). The organic layer was separated and washed with saturated NaHCO₃ (2×30 mL), dried over Na₂SO₄, filtered and concentrated under reduced pressure. The residue was purified by column chromatography (SiO₂, 0-50% EtOAc/petroleum ether) to give the title compound (400 mg. 66% yield) as a white solid. [M+H]=474.0. ¹H NMR (400 MHZ. CDCl₃) δ ppm 8.11 (s, 1H). 7.85 (s, 1H). 7.32 (s, 1H), 4.13-3.98 (m, 2H). 3.94 (s, 3H). 3.87-3.80 (m, 1H). 3.36-3.23 (m, 2H). 2.13-1.96 (m, 4H). 1.49 (s, 9H).

tert-butyl 4-fluoro-4-(3-iodo-6-methoxypyrazolo[1,5-a]pyridin-5-yl) piperidine-1-carboxylate: To a solution of tert-butyl 4-hydroxy-4-(3-iodo-6-methoxypyrazolo[1,5-a]pyridin-5-yl) piperidine-1-carboxylate (400 mg. 760.61 μmol. 1 eq) in DCM (10 mL) was added DAST (367.81 mg. 2.28 mmol. 301.48 μL. 3 eq) dropwise at −78° C. under a N₂ atmosphere. The mixture was stirred at −40° C. for 2 hours under a N₂ atmosphere before it was quenched with saturated NaHCO₃ (20 mL), and then diluted with water (40 mL) and extracted with DCM (3×40 mL). The combined organic layers were dried over Na₂SO₄, filtered and concentrated under reduced pressure. The residue was purified by column chromatography (SiO₂, 0-5% EtOAc/petroleum ether) to give the title compound (250 mg. 62% yield) as a light yellow solid. 1H NMR (400 MHZ. CDCl₃)₈ ppm 8.06 (s. 1H). 7.85 (s, 1H). 7.58 (s. 1H). 4.22-3.98 (m, 2H). 3.85 (s, 3H). 3.18 (br s, 2H). 2.68-2.39 (m, 2H). 1.79-1.72 (m, 2H). 1.51 (s, 9H).

tert-butyl 4-fluoro-4-(6-methoxy-3-(5-(methoxycarbonyl)-1-methyl-1H-pyrazol-3-yl)pyrazolo[1,5-a]pyridin-5-yl) piperidine-1-carboxylate: A mixture of tert-butyl 4-fluoro-4-(3-iodo-6-methoxypyrazolo[1,5-a]pyridin-5-yl) piperidine-1-carboxylate (250 mg. 473.39 μmol. 1 eq), methyl 1-methyl-3-(4,4,5,5-tetramethyl-1,3,2-dioxaborolan-2-yl)-1H-pyrazole-5-carboxylate (209.95 mg. 710.09 μmol. 1.5 eq). Pd(dppf)Cl₂ (34.64 mg. 47.34 μmol. 0.1 eq) and K₂CO₃ (196.28 mg. 1.42 mmol, 3 eq) in dioxane (10 mL) and H₂O (2.5 mL) was degassed and purged with N₂ three times. The mixture was stirred at 80° C. for 4 hours under a N₂ atmosphere before it was concentrated under reduced pressure. The residue was purified by column chromatography (SiO₂, 0-40% EtOAc/petroleum ether) to give the title compound (160 mg, 62% yield) as a white solid. 1H NMR (400 MHZ, CDCl₃) δ ppm 8.23 (s, 1H), 8.12 (s, 1H), 8.07 (s, 1H), 7.04 (s, 1H), 4.25 (s, 3H), 4.19-4.01 (m, 2H), 3.92 (s, 3H), 3.86 (s, 3H), 3.29-3.09 (m, 2H), 2.69-2.44 (m, 2H), 1.80 (t, J=12.4 Hz, 2H), 1.51 (s, 9H).

methyl 3-(5-(4-fluoropiperidin-4-yl)-6-methoxypyrazolo[1,5-a]pyridin-3-yl)-1-methyl-1H-pyrazole-5-carboxylate: tert-butyl 4-fluoro-4-(6-methoxy-3-(5-(methoxycarbonyl)-1-methyl-1H-pyrazol-3-yl)pyrazolo[1,5-a]pyridin-5-yl) piperidine-1-carboxylate (160 mg, 295.37 μmol, 1 eq) was added to HCl in dioxane (4 M, 5 mL). The mixture was stirred at 20° C. for 1 hour before it was concentrated under reduced pressure to provide the title compound (120 mg, 86% yield) as a white solid, which was used in the next step without further purification. 1H NMR (400 MHz, MeOH-d₄) δ ppm 8.33 (s, 1H), 8.31 (s, 1H), 8.24 (s, 1H), 7.15 (s, 1H), 4.21 (s, 3H), 3.99 (s, 3H), 3.92 (s, 3H), 3.51-3.41 (m, 4H), 3.06-2.85 (m, 2H). 2.15-2.11 (m, 2H).

methyl 3-(5-(4-fluoro-1-methylpiperidin-4-yl)-6-methoxypyrazolo[1,5-a]pyridin-3-yl)-1-methyl-1H-pyrazole-5-carboxylate: To a solution of methyl 3-(5-(4-fluoropiperidin-4-yl)-6-methoxypyrazolo[1,5-a]pyridin-3-yl)-1-methyl-1H-pyrazole-5-carboxylate (120 mg, 278.78 μmol, 1 eq) in MeOH (5 mL) was added paraformaldehyde (84.47 mg. 2.79 mmol, 10 eq) and HOAc (100.45 mg, 1.67 mmol, 95.75 μL. 6 eq). The mixture was stirred at 20° C. for 1 hour, and then NaBH₃CN (52.56 mg. 836.33 μmol, 3 eq) was added. The resulting mixture was stirred at 20° C. for 12 hours before it was diluted with H₂O (20 mL) and extracted with DCM (3×20 mL). The combined organic extracts were concentrated under reduced pressure and the residue was purified by column chromatography (SiO₂, 0-12% MeOH/DCM) to give the title compound (120 mg. 97% yield) as a white solid. [M+H]=402.1. ¹H NMR (400 MHZ, DMSO-d₆) δ ppm 8.53 (s, 1H), 8.40 (s. 1H). 8.21 (s, 1H), 7.27 (s, 1H), 4.15 (s, 3H), 3.92 (s, 3H), 3.87 (s, 3H), 3.00-2.65 (m, 6H), 2.54 (s. 3H), 2.08-1.92 (m, 2H).

lithium 3-(5-(4-fluoro-1-methylpiperidin-4-yl)-6-methoxypyrazolo[1,5-a]pyridin-3-yl)-1-methyl-1H-pyrazole-5-carboxylate: To a solution of methyl 3-(5-(4-fluoro-1-methylpiperidin-4-v1)-6-methoxypyrazolo[1,5-a]pyridin-3-yl)-1-methyl-1H-pyrazole-5-carboxylate (120 mg, 269.04 μmol, 1 eq) in THF (5 mL) and H₂O (1 mL) was added LiOH·H₂O (33.87 mg. 807.11 μmol, 3 eq). The mixture was stirred at 20° C. for 2 hours before it was concentrated under reduced pressure to give the title compound (100 mg. 85% yield) as a white solid, which was used for the next step without further purification. 1H NMR (400 MHZ, DMSO-d₆) δ ppm 8.23 (s, 1H), 8.16 (s, 1H), 8.14 (d. J=1.2 Hz, 1H), 6.90 (d. J=1.2 Hz, 1H), 4.19 (d, J=0.8 Hz, 3H), 3.94 (s, 3H), 2.87-2.68 (m, 4H), 2.53-2.44 (m, 2H), 2.36 (s, 3H), 1.83 (t, J=12.2 Hz, 2H).

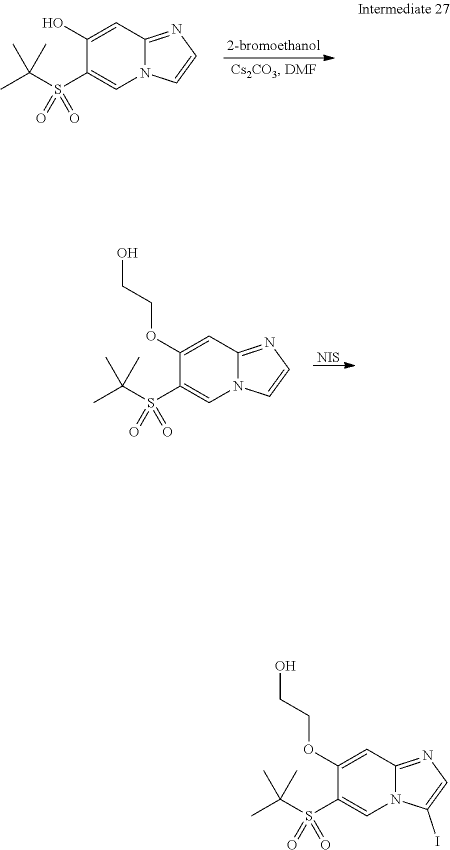

2-((6-(tert-butylsulfonyl) imidazo[1,2-a]pyridin-7-yl)oxy) ethan-1-ol: A mixture of 6-(tert-butylsulfonyl) imidazo[1,2-a]pyridin-7-ol (69.86 mg, 559.01 μmol, 39.62 μL, 1.5 eq), 2-bromoethanol (69.86 mg, 559.01 μmol, 39.62 μL, 1.5 eq), and Cs₂CO₃ (364.27 mg, 1.12 mmol, 3 eq) in DMF (2 mL) was degassed and purged with N₂ three times, and then the mixture was stirred at 80° C. for 2 hours under a N₂ atmosphere. The reaction mixture was diluted with H₂O (20 mL) and the resulting mixture was purified by RP-HPLC to yield the title compound (40 mg, 32% yield) as a brown solid. 1H NMR (400 MHZ, DMSO-d₆) δ ppm 9.12 (s, 1H), 7.99 (s, 1H), 7.54 (d, J=1.2 Hz, 1H), 7.15 (s, 1H), 4.78 (t, J=5.2 Hz, 1H), 4.13 (t, J=4.8 Hz, 2H), 3.80-3.70 (m, 2H), 1.32 (s, 9H).

2-((6-(tert-butylsulfonyl)-3-iodoimidazo[1,2-a]pyridin-7-yl)oxy) ethan-1-ol: To a solution of 2-((6-(tert-butylsulfonyl) imidazo[1,2-a]pyridin-7-yl)oxy) ethan-1-ol (35 mg, 105.58 μmol, 1 eq) in MeOH (1.5 mL) and H₂O (1.5 mL) was added NIS (28.50 mg, 126.69 μmol, 1.2 eq). The mixture was stirred at 25° C. for 12 hours before it was concentrated under reduced pressure to remove the MeOH. The residue was purified by RP-HPLC to yield the title compound (33 mg, 66% yield) as a yellow solid. 1H NMR (400 MHZ, CDCl₃) δ ppm 8.67 (s, 1H), 7.68 (s, 1H), 7.05 (s, 1H), 4.27 (t, J=4.4 Hz, 2H), 3.97 (d, J=2.8 Hz, 2H), 3.24-3.18 (m, 1H), 1.46 (s, 9H).

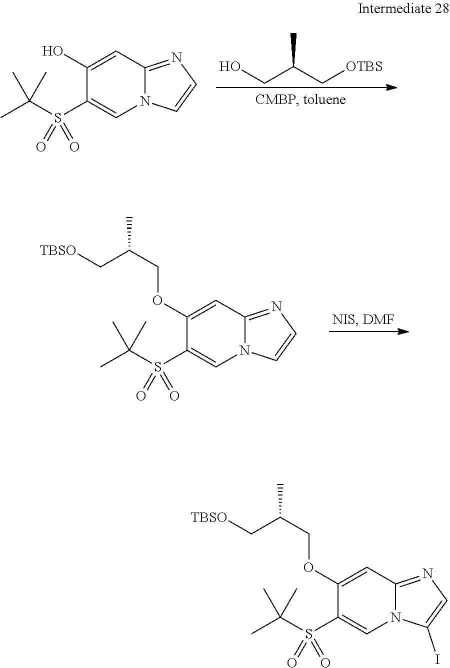

(R)-7-(3-((tert-butyldimethylsilyl)oxy)-2-methylpropoxy)-6-(tert-butylsulfonyl) imidazo[1,2-a]pyridine: To a solution of (R)-3-((tert-butyldimethylsilyl)oxy)-2-methylpropan-1-ol (289.33 mg, 1.42 mmol, 2 eq) and 6-(tert-butylsulfonyl) imidazo[1,2-a]pyridin-7-ol (200 mg, 707.81 μmol, 1 eq) in toluene (5 mL) was added CMBP (1.02 g, 4.25 mmol, 6 eq). The mixture was stirred at 100° C. for 12 hours before it was filtered and concentrated under reduced pressure. The residue was purified by column chromatography (SiO₂, 0-25% EtOAc/petroleum ether) to give the title compound (240 mg, 69% yield) as a brown solid. [M+H]⁺=441.1. ¹H NMR (400 MHZ, DMSO-d₆) δ ppm 9.12 (s, 1H), 7.98 (d, J=0.8 Hz, 1H), 7.53 (d, J=1.6 Hz, 1H), 7.09 (s, 1H), 4.07-3.91 (m, 2H), 3.75-3.65 (m, 1H), 3.68-3.55 (m, 1H), 2.11-2.01 (m, 1H), 1.31 (s, 9H), 1.01 (d, J=6.8 Hz, 3H), 0.85 (s, 9H), 0.02 (d, J=1.0 Hz, 6H).

(R)-7-(3-((tert-butyldimethylsilyl)oxy)-2-methylpropoxy)-6-(tert-butylsulfonyl)-3-iodoimidazo[1,2-a]pyridine: To a solution of (R)-7-(3-((tert-butyldimethylsilyl)oxy)-2-methylpropoxy)-6-(tert-butylsulfonyl) imidazo[1,2-a]pyridine (235 mg, 479.95 μmol, 1 eq) in DMF (5 mL) was added NIS (215.96 mg, 959.90 μmol, 2 eq). The mixture was stirred at 25° C. for 3 hours before it was concentrated under reduced pressure. The residue was diluted with saturated aqueous Na₂SO₃ (50 mL) and then extracted with EtOAc (3×50 mL). The combined organic extracts were washed with saturated aqueous Na₂CO₃ (100 mL), dried over Na₂SO₄, filtered and concentrated to give the title compound (220 mg, 349.47 μmol, 73% yield) as a white solid. [M+H]⁺=567.1. ¹H NMR (400 MHz, DMSO-d₆) δ ppm 8.48 (s, 1H), 7.71 (s, 1H), 7.24 (s, 1H), 4.11-3.94 (m, 2H), 3.79-3.53 (m, 2H), 2.13-2.01 (m, 1H), 1.33 (s, 9H), 1.01 (d, J=6.8 Hz, 3H), 0.85 (s, 9H), 0.02 (d, J=1.2 Hz, 6H).

The following intermediate was prepared following procedures analogous to that described for Intermediate 28.

| Int. | | | |
|---|---|---|---|
| No. | Structure | Name | LCMS, ¹H NMR |
| 29 | 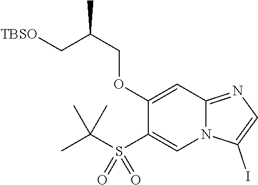 [SMILES: C[C@@H](COc1cc2ncc(I)n2cc1S(=O)(=O)C(C)(C)C)CO[Si](C)(C)C(C)(C)C] | (S)-7-(3-((tert- butyldimethylsilyl)oxy)- 2-methylpropoxy)-6- (tert-butylsulfonyl)-3- iodoimidazo[1,2- a]pyridine | [M + H]⁺ = 567.1 ¹H NMR (400 MHz, CDCl₃) δ ppm 8.68 (s, 1H), 7.66 (s, 1H), 7.12 (s, 1H), 4.16-4.10 (m, 1H), 4.02-3.93 (m, 1H), 3.80- 3.59 (m, 2H), 2.25-2.17 (m, 1H), 1.43 (s, 9H), 1.10 (d, J = 6.8 Hz, 3H), 0.88 (s, 9H), 0.04 (d, J = 2.8 Hz, 6H) |

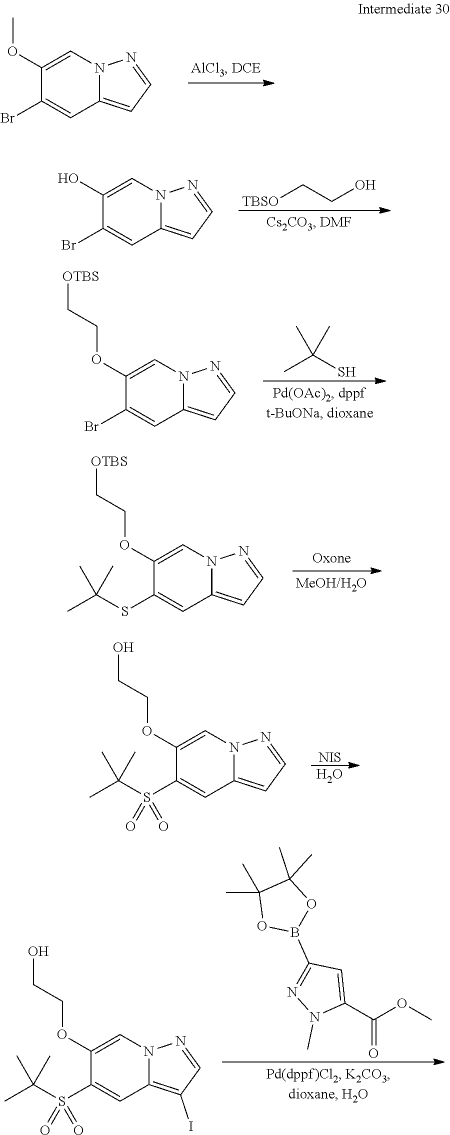

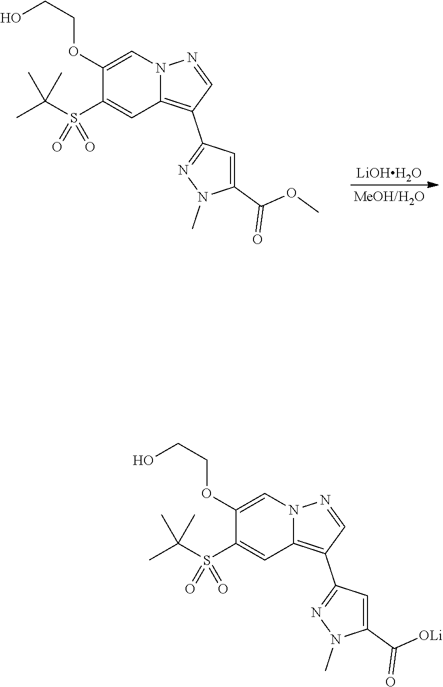

5-bromopyrazolo[1,5-a]pyridin-6-ol: To a solution of 5-bromo-6-methoxypyrazolo[1,5-a]pyridine (500 mg, 1.98 mmol, 1 eq) in DCE (8 mL) was added AlCl3 (1.32 g, 9.91 mmol, 541.53 μL, 5 eq). The mixture was stirred at 80° C. for 1 hour before it was quenched with saturated Na₂SO₄ (5 mL), neutralized with saturated NaHCO₃, and extracted with DCM (120 mL). The organic phase was dried over Na₂SO₄, filtered and concentrated under reduced pressure to give the title compound (800 mg) as a black-brown solid. [M+H]⁺=214.8. ¹H NMR (400 MHZ, DMSO-d₆) δ ppm 10.33 (s, 1H), 8.21 (s, 1H), 8.02 (s, 1H), 7.83 (d, J=1.6 Hz, 1H), 6.47 (d, J=2.0 Hz, 1H) 5-bromo-6-(2-((tert-butyldimethylsilyl)oxy) ethoxy)pyrazolo[1,5-a]pyridine: To a solution of 5-bromopyrazolo[1,5-a]pyridin-6-ol (400 mg, 1.88 mmol, 1 eq) in DMF (5 mL) was added Cs₂CO₃ (1.84 g, 5.63 mmol, 3 eq) and 2-((tert-butyldimethylsilyl)oxy) ethan-1-ol (583.94 mg, 2.44 mmol, 1.3 eq). The mixture was stirred at 80° C. for 12 hours befoer it was diluted with brine (20 mL), and extracted with EtOAc (140 mL). The combined organic layers were dried over Na₂SO₄, filtered and concentrated under reduced pressure to give a residue, which was purified by column chromatography (SiO₂, 10-20% EtOAc/petroleum ether) to give the title compound (80 mg, 10% yield) as a light yellow solid. [M+H]⁺=371.0. ¹H NMR (400 MHZ, DMSO-d₆) δ ppm 8.55 (s, 1H), 8.09 (s, 1H), 7.91 (d, J=2.8 Hz, 1H), 6.52 (d, J=2.4 Hz, 1H), 4.16 (t, J=4.8 Hz, 2H), 3.97 (t, J=4.4 Hz, 2H), 0.87 (s, 9H), 0.09 (s, 6H).

6-(2-((tert-butyldimethylsilyl)oxy) ethoxy)-5-(tert-butylthio)pyrazolo[1,5-a]pyridine: A mixture of 5-bromo-6-(2-((tert-butyldimethylsilyl)oxy) ethoxy)pyrazolo[1,5-a]pyridine (80 mg, 193.89 μmol, 1 eq), Pd(OAc): (870.60 μg, 3.88 μmol, 0.02 eq), dppf (4.30 mg, 7.76 μmol, 0.04 eq) and t-BuONa (55.90 mg, 581.67 μmol, 3 eq) in dioxane (4 mL) was degassed and purged with N₂ three times, and then 2-methylpropane-2-thiol (52.46 mg, 581.67 μmol, 65.49 μL, 3 eq) was added. The mixture was stirred at 90° C. for 5 hours under a N₂ atmosphere before it was filtered and concentrated under reduced pressure to give a residue, which was purified by column chromatography (SiO₂, 5-15% EtOAc/petroleum ether) to give the title compound (80 mg. 98% yield) as a yellow solid. [M+H]=381.2. ¹H NMR (400 MHZ. DMSO-d₆) δ ppm 8.43 (s, 1H). 7.89 (d. J=2.4 Hz, 1H). 7.86 (s, 1H). 6.59 (d. J=2.4 Hz, 1H). 4.10-4.04 (m, 2H). 3.99-3.93 (m. 2H). 1.29 (s, 9H). 0.88 (s, 9H). 0.11 (s. 6H).

2-((5-(tert-butylsulfonyl)pyrazolo[1,5-a]pyridin-6-yl)oxy) ethan-1-ol: To a solution of 6-(2-((tert-butyldimethylsilyl)oxy) ethoxy)-5-(tert-butylthio)pyrazolo[1,5-a]pyridine (80 mg. 189.17 μmol. 1 eq) in MeOH (3 mL) and H₂O (1 mL) was added Oxone® (174.44 mg. 283.75 μmol. 1.5 eq). The mixture was stirred at 25° C. for 12 hours before it was filtered and concentrated to provide the title compound (57 mg) as a yellow liquid that was used in the next step directly. [M+H]=299.0.

2-((5-(tert-butylsulfonyl)-3-iodopyrazolo[1,5-a]pyridin-6-yl)oxy) ethan-1-ol: To a solution of 2-((5-(tert-butylsulfonyl)pyrazolo[1,5-a]pyridin-6-yl)oxy) cthan-1-ol (57 mg, 191.05 μmol. 1 eq) in H₂O (1 mL) was added NIS (64.47 mg. 286.57 μmol. 1.5 eq). The mixture was stirred at 25° C. for 1 hour before it was quenched with saturated Na₂SO; (10 mL) and extracted with EtOAc (30 mL). The combined organic phase was dried over anhydrous sodium sulfate, filtered and concentrated to give a residue, which was purified by column chromatography (SiO₂, 15-100% EtOAc/petroleum ether) to give the title compound (90 mg. 99% yield) as a white solid. [M+H]=424.9. ¹H NMR (400 MHZ. DMSO-d₆) δ ppm 8.82 (s, 1H). 8.22 (s, 1H). 7.88 (s, 1H). 4.82 (t. J=5.6 Hz, 1H). 4.14 (t. J=5.2 Hz, 2H). 3.75 (q. J=5.2 Hz, 2H). 1.33 (s, 9H). methyl 3-(5-(tert-butylsulfonyl)-6-(2-hydroxyethoxy)pyrazolo[1,5-a]pyridin-3-yl)-1-methyl-1H-pyrazole-5-carboxylate: A mixture of 2-((5-(tert-butylsulfonyl)-3-iodopyrazolo[1.5-a]pyridin-6-yl)oxy)ethan-1-ol (0.1 g. 212.14 μmol. 1 eq), methyl 1-methyl-3-(4,4,5,5-tetramethyl-1,3,2-dioxaborolan-2-yl)-1H-pyrazole-5-carboxylate (62.72 mg. 212.14 μmol. 1 eq). Pd (dppf) Cl₂ (155.22 mg. 212.14 μmol. 1 eq). K₂CO₃ (29.32 mg. 212.14 μmol. 1 eq) in dioxane (1 mL) and H₂O (0.2 mL) was degassed and purged with N₂ three times before it was stirred at 80° C. for 2 hours under a N₂ atmosphere. The reaction mixture was diluted with water (20 mL) and extracted with EtOAc (3×10 mL), and then the combined organic extracts were washed with brine (10 mL), dried over Na₂SO₄, filtered and concentrated under reduced pressure. The residue was purified by column chromatography (SiO₂, 10-100% EtOAc/petroleum ether) to give the title compound (85 mg. 83% yield) as a yellow solid. 1H NMR (400 MHz, CDCl₃) δ ppm 8.77 (s, 1H). 8.23 (s, 2H). 7.06 (s, 1H). 4.27-4.24 (m. 5H). 3.97-3.95 (m, 2H). 3.94 (s, 1H). 1.46 (s, 9H).

lithium 3-(5-(tert-butylsulfonyl)-6-(2-hydroxyethoxy)pyrazolo[1,5-a]pyridin-3-yl)-1-methyl-1H-pyrazole-5-carboxylate: To a solution of methyl 3-(5-(tert-butylsulfonyl)-6-(2-hydroxyethoxy)pyrazolo[1,5-a]pyridin-3-yl)-1-methyl-1H-pyrazole-5-carboxylate (85 mg, 175.27 μmol, 1 eq) in THF (2 mL) and H₂O (0.2 mL) was added LiOH—H₂O (11.03 mg, 262.90 μmol, 1.5 eq). The mixture was stirred at 25° C. for 1 hour before it was filtered and concentrated under reduced pressure to give the title compound (65 mg, 79% yield) as a yellow solid. 1H NMR (400 MHz, DMSO-d₆) δ ppm 8.69 (s, 1H), 8.64 (s, 1H), 8.38 (s, 1H), 6.67 (s, 1H), 4.16-4.13 (m, 2H), 4.11 (s, 3H), 3.76 (t, J=4.4 Hz, 2H), 1.33 (s, 9H).

The following intermediate was prepared following procedures analogous to that described for Intermediate 30.

| Int. | | | |
|---|---|---|---|
| No. | Structure | Name | LCMS, ¹H NMR |
| 31 | 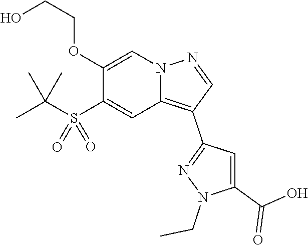 [SMILES: CCn1nc(-c2cnn3cc(OCCO)c(S(=O)(=O)C(C)(C)C)cc23)cc1C(=O)O] | 3-(5-(tert- butylsulfonyl)-6-(2- hydroxyethoxy)pyrazolo [1,5-a]pyridin-3-yl)-1- ethyl-1H-pyrazole-5- carboxylic acid | ¹H NMR (400 MHz, DMSO-d₆) δ ppm 8.74 (s, 1H), 8.70 (s, 1H), 8.54 (s, 1H), 7.26 (s, 1H), 4.82 (t, J = 5.2 Hz, 1H), 4.57 (q, J = 7.2 Hz, 2H), 4.16 (t, J = 4.8 Hz, 2H), 3.77 (q, J = 4.8 Hz, 2H), 1.42 (t, J = 7.2 Hz, 3H), 1.34 (s, 9H) |

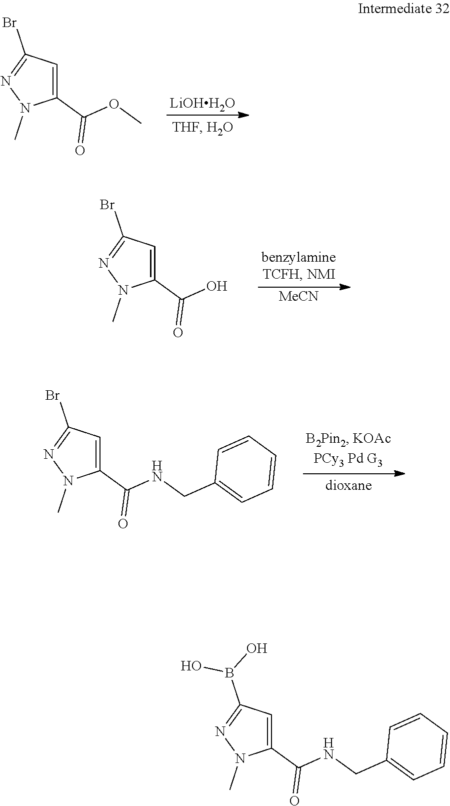

3-bromo-1-methyl-1H-pyrazole-5-carboxylic acid: To a solution of methyl 3-bromo-1-methyl-1H-pyrazole-5-carboxylate (270 mg, 1.23 mmol, 1 eq) in THF (4.5 mL) was added a solution of LiOH·H₂O (98.28 mg, 2.34 mmol, 1.9 eq) in water (4.5 mL) at 25° C. The resulting mixture was stirred at 50° C. for 30 minutes under a N₂ atmosphere before it was diluted with water (10 mL) and washed with EtOAc (3×10 mL) (organic extracts discarded). The aqueous phase was acidified to pH=5 with 1 N HCl, and then the product was extracted with EtOAc (3×10 mL). The product-containing organic extracts were dried over Na₂SO₄, filtered and concentrated under reduced pressure to give the title compound (280 mg, 99% yield) as a white solid. 1H NMR (400 MHz, DMSO-d₆) δ ppm 6.88 (s, 1H), 4.04 (s, 3H).

N-benzyl-3-bromo-1-methyl-1H-pyrazole-5-carboxamide: A solution of 3-bromo-1-methyl-1H-pyrazole-5-carboxylic acid (200 mg, 878.01 μmol, 1 eq), benzylamine (282.24 mg, 2.63 mmol, 287.12 μL, 3 eq), TCFH (492.70 mg, 1.76 mmol, 2 eq) and 1-methylimidazole (216.26 mg, 2.63 mmol, 209.96 μL, 3 eq) in MeCN (7 mL) was stirred at 25° C. for 2 hours under a N₂ atmosphere. The reaction mixture was concentrated under reduced pressure, and then the residue was purified by column chromatography (SiO₂, 0-20% EtOAc/petroleum ether) to give the title compound (170 mg, 59% yield) as a white solid. [M+H]⁺=295.7. ¹H NMR (400 MHZ, DMSO-d₆) δ ppm 9.08 (s, 1H), 7.42-7.18 (m, 5H), 7.00 (s, 1H), 4.43 (d, J=6.0 Hz, 2H), 4.04 (s, 3H).

(5-(benzylcarbamoyl)-1-methyl-1H-pyrazol-3-yl) boronic acid: A mixture of N-benzyl-3-bromo-1-methyl-1H-pyrazole-5-carboxamide (90 mg, 275.37 μmol, 1 eq), B₂Pin₂ (139.86 mg, 550.75 μmol, 2 eq), KOAc (81.08 mg, 826.12 μmol, 3 eq) and PCy₃ Pd G3 (17.90 mg, 27.54 μmol, 0.1 eq) in dioxane (1 mL) was stirred at 100° C. for 12 hours under N₂ atmosphere. The reaction mixture was concentrated under reduced pressure, and then the residue was purified by column chromatography (SiO₂, 0-100% EtOAc/petroleum ether and 0-80% MeOH/DCM) to give the title compound (20 mg, 25% yield) as a white solid. 1H NMR (400 MHZ, DMSO-d₆) δ ppm 9.00 (t, J=6.0 Hz, 1H), 8.14 (s, 2H), 7.36-7.24 (m, 5H), 7.15 (s, 1H), 4.42 (d, J=6.0 Hz, 2H), 4.09 (s, 3H).

The following intermediate was prepared following procedures analogous to that described for Intermediate 32.

| Int. | | | |
|---|---|---|---|
| No. | Structure | Name | LCMS, ¹H NMR |
| 33 | 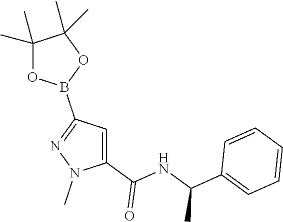 [SMILES: C[C@@H](NC(=O)c1cc(B2OC(C)(C)C(C)(C)O2)nn1C)c1ccccc1] | (R)-1-methyl-N-(1- phenylethyl)-3-(4,4,5,5- tetramethyl-1,3,2- dioxaborolan-2-yl)-1H- pyrazole-5-carboxamide | Used directly |

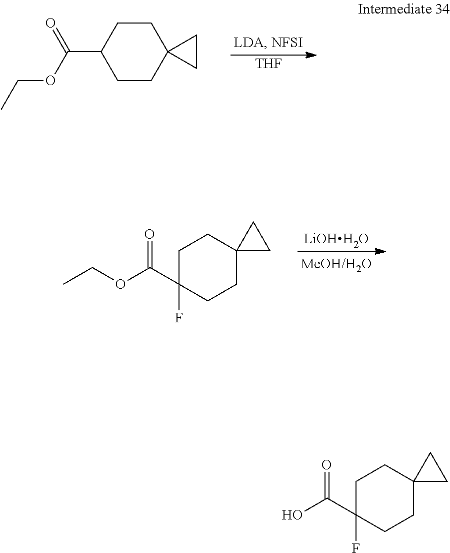

ethyl 6-fluorospiro[2.5]octane-6-carboxylate: To a solution of ethyl spiro[2.5]octane-6-carboxylate (200 mg, 1.10 mmol, 1 eq) in THF (4 mL) was added dropwise LDA (2 M, 1.37 mL, 2.5 eq) at −60° C. After addition, the mixture was stirred at −60° C. for 1 h, and then N-fluorobenzenesulfonimide (415.24 mg, 1.32 mmol, 1.2 eq) in THF (2 mL) was added dropwise at −60° C. The resulting mixture was stirred at 0° C. for 1 hour before it was quenched with saturated NH₄Cl (5 mL) at 0° C., and then extracted with EtOAc (3×10 mL). The combined organic layers were washed with brine (10 mL), dried over Na₂SO₄, filtered and concentrated under reduced pressure to give a residue, which was purified by column chromatography (SiO₂, 0-50% EtOAc/petroleum ether) to give the title compound (45 mg, 14% yield) as yellow oil. 1H NMR (400 MHZ, CDCl₃) δ ppm 4.28-4.20 (m, 2H), 2.10-1.97 (m, 4H), 1.71-1.53 (m, 2H), 1.36-1.25 (m, 3H), 0.89-0.84 (m, 2H), 0.30-0.21 (m, 4H).

6-fluorospiro[2.5]octane-6-carboxylic acid: To a solution of ethyl 6-fluorospiro[2.5]octane-6-carboxylate (45 mg, 157.30 μmol, 1 eq) in MeOH (2 mL) and H₂O (0.5 mL) was added LiOH·H₂O (66.01 mg, 1.57 mmol, 10 eq). The mixture was stirred at 50° C. for 12 hours before it was concentrated under reduced pressure to remove the MeOH. The resulting aqueous mixture was adjusted to pH=2 with 2 M HCl at 0° C., and then the mixture was diluted with H₂O (10 mL) and extracted with EtOAc (3×10 mL). The combined organic layers were washed with brine (10 mL), dried over Na₂SO₄, filtered and concentrated under reduced pressure to give the title compound (35 mg) as yellow oil. 1H NMR (400 MHZ, CDCl₃) δ ppm 2.01-1.93 (m, 4H), 1.69-1.62 (m, 2H), 0.92-0.83 (m, 2H), 0.32-0.16 (m, 4H).

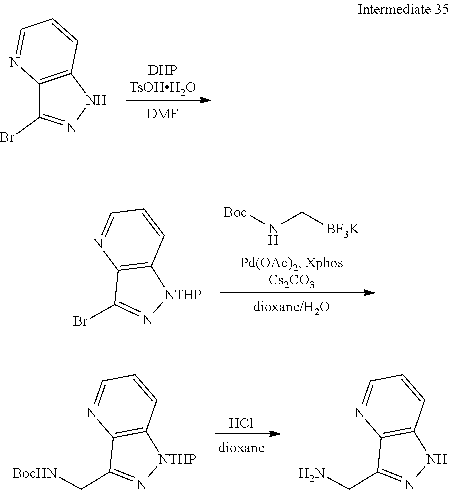

3-bromo-1-(tetrahydro-2H-pyran-2-yl)-1H-pyrazolo[4,3-b]pyridine: To a solution of 3-bromo-1H-pyrazolo[4,3-b]pyridine (100 mg, 505.00 μmol, 1 eq) in DMF (3 mL) was added DHP (212.39 mg, 2.52 mmol, 230.86 μL, 5 eq) and TsOH·H₂O (48.03 mg, 252.50 μmol, 0.5 eq). The mixture was stirred at 100° C. for 12 hours before it was diluted with water (20 mL) and extracted with EtOAc (3×20 mL). The combined organic extracts were washed with sat. NaHCO₃ (3×10 mL), dried over Na₂SO₄, filtered and concentrated under reduced pressure. The residue was purified by column chromatography (SiO₂, 1-25% EtOAc/petroleum ether) to give the title compound (100 mg, 63% yield) as a yellow oil. 1H NMR (400 MHZ, DMSO-d₆) δ ppm 8.64 (d, J=4.4 Hz, 1H), 8.30 (d, J=8.8 Hz, 1H), 7.56 (dd, J=8.4, 4.4 Hz, 1H), 5.92 (dd, J=9.6, 2.0 Hz, 1H), 3.88 (d, J=11.2 Hz, 1H), 3.78-3.70 (m, 1H), 2.37-2.31 (m, 1H), 2.04-1.98 (m, 2H), 1.77-1.70 (m, 1H), 1.61-1.56 (m, 2H).

tert-butyl ((1-(tetrahydro-2H-pyran-2-yl)-1H-pyrazolo[4,3-b]pyridin-3-yl)methyl) carbamate: A mixture of 3-bromo-1-(tetrahydro-2H-pyran-2-yl)-1H-pyrazolo[4,3-b]pyridine (90 mg, 287.10 μmol, 1 eq), tert-butyl ((trifluoro-λ⁴-boraneyl)methyl) carbamate, potassium salt (102.09 mg, 430.64 μmol, 1.5 eq), Pd(OAc): (6.45 mg, 28.71 μmol, 0.1 eq), XPhos (27.37 mg, 57.42 μmol, 0.2 eq) and Cs₂CO₃ (280.62 mg, 861.29 μmol, 3 eq) in 1,4-dioxane (2 mL) and H₂O (0.2 mL) was degassed and purged with N₂ three times. The resulting mixture was stirred at 100° C. for 12 hours under a N₂ atmosphere before it was concentrated under reduced pressure. The residue was purified by column chromatography (SiO₂, 1-40% EtOAc/petroleum ether) to give the title compound (77 mg, 73% yield) as a yellow oil. [M+H]⁺=333.0. ¹H NMR (400 MHz, DMSO-d₆) δ ppm 8.54-8.53 (m, 1H), 8.18-8.15 (m, 1H), 7.44-7.40 (m, 1H), 7.28-7.20 (m, 1H), 5.84 (dd, J=9.6, 2.4 Hz, 1H), 4.54 (d, J=5.6 Hz, 2H), 3.91-3.83 (m, 1H), 3.75-3.69 (m, 1H), 2.42-2.34 (m, 1H), 2.05-2.01 (m, 8.9 Hz, 1H), 1.98-1.91 (m, 1H), 1.77-1.69 (m, 1H), 1.58 (d, J=3.6 Hz, 2H), 1.39 (s, 9H).

(1H-pyrazolo[4,3-b]pyridin-3-yl) methanamine hydrochloride: A solution of tert-butyl ((1-(tetrahydro-2H-pyran-2-yl)-1H-pyrazolo[4,3-b]pyridin-3-yl)methyl) carbamate (77 mg, 208.49 μmol, 1 eq) in HCl/dioxane (2 mL) was stirred at 25° C. for 1 hour before it was concentrated under reduced pressure to give the title compound (34 mg, 99% yield) as a white solid. The material was used in the next step without purification. 1H NMR (400 MHZ, DMSO-d₆) δ ppm 13.75-13.37 (m, 1H), 8.60 (dd, J=4.4, 1.2 Hz, 1H), 8.48 (d, J=2.4 Hz, 2H), 8.11 (dd, J=8.4, 1.2 Hz, 1H), 7.48 (dd, J=8.8, 4.4 Hz, 1H), 4.46 (q, J=5.6 Hz, 2H).

The following intermediate was prepared following procedures analogous to that described for Intermediate 35.

| Int. | | | |
|---|---|---|---|
| No. | Structure | Name | LCMS, ¹H NMR |
| 36 | 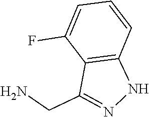 [SMILES: NCc1n[nH]c2cccc(F)c12] | (4-fluoro-1H- indazol-3- yl) methanamine | [M + H]⁺ = 166.2 ¹H NMR (400 MHz, DMSO-d₆) δ ppm 13.65 (s, 1H), 8.53 (s, 2H), 7.44-7.35 (m, 2H), 6.98-6.88 (m, 1H), 4.46-4.37 (m, 2H) |

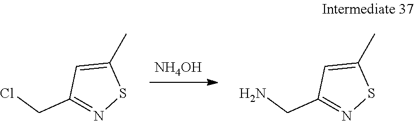

(5-methylisothiazol-3-yl) methanamine: A mixture of 3-(chloromethyl)-5-methylisothiazole (80 mg, 541.91 μmol, 1 eq) in ammonium hydroxide (2 mL) was stirred at 100° C. for 1 hour. The mixture was filtered and concentrated under reduced pressure to give the title compound (50 mg, crude) as a yellow solid, which was used in the next step directly without purification.

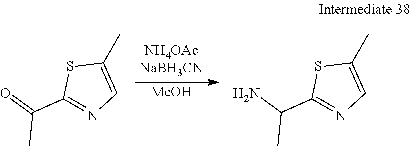

1-(5-methylthiazol-2-yl) ethan-1-amine: To a solution of 1-(5-methylthiazol-2-yl) ethan-1-one (100 mg, 708.26 μmol, 1 eq) in MeOH (10 mL) was added NH₄OAc (666.05 mg, 8.64 mmol, 12.2 eq) and NaBH₃CN (182.49 mg, 2.90 mmol, 4.1 eq). The mixture was stirred at 60° C. for 16 hours before it was concentrated under reduced pressure. The residue was purified by RP-HPLC to yield the title compound (50 mg, 37% yield) as colorless oil. 1H NMR (400 MHZ, DMSO-d₆)₈ ppm 7.40 (d, J=1.0 Hz, 1H), 4.36 (q, J=6.4 Hz, 1H), 2.41 (d, J=0.4 Hz, 3H), 1.41 (d, J=6.4 Hz, 3H).

The following intermediate was prepared following procedures analogous to that described for Intermediate 38.

| Int. | | | |
|---|---|---|---|
| No. | Structure | Name | LCMS, ¹H NMR |
| 39 | 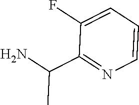 | 1-(3-fluoropyridin-2- yl)ethan-1-amine | ¹H NMR (400 MHz, CDCl₃) δ ppm 8.41 (d, J = 4.8 Hz, 1H), 7.54-7.47 (m, 1H), 7.41-7.35 (m, 1H), 4.93 (q, J = 6.8 Hz, 1H), 1.67 (d, J = 6.8 Hz, 3H) |

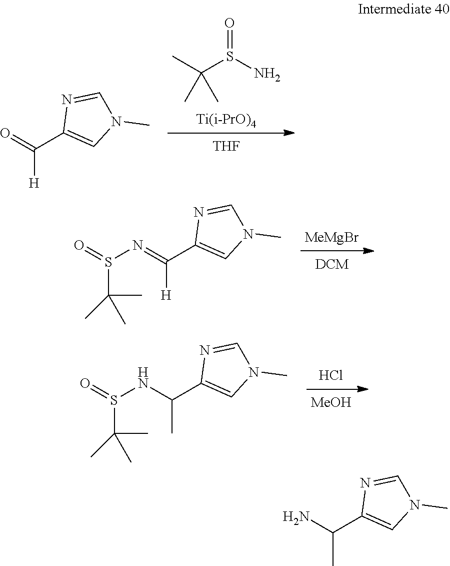

(E)-2-methyl-N-((1-methyl-1H-imidazol-4-yl)methylene) propane-2-sulfinamide: To a solution of 1-methyl-1H-imidazole-4-carbaldehyde (500 mg, 4.54 mmol, 1 eq) and 2-methylpropane-2-sulfinamide (660.41 mg, 5.45 mmol, 1.2 eq) in THF (10 mL) was added Ti (i-PrO)+ (2.58 g, 9.08 mmol, 2.68 mL, 2 eq) at 0° C., and then the mixture was stirred at 25° C. for 12 hours under a N₂ atmosphere. The reaction mixture was diluted with water (20 mL), and then the solids were removed by filtration. The filtrate was extracted with EtOAc (3×20 mL), dried over Na₂SO₄, filtered and concentrated under reduced pressure. The residue was purified by column chromatography (SiO₂, 0-10% MeOH/EtOAc) to give the title compound (560 mg, 52% yield) as a colorless oil. 1H NMR (400 MHZ, DMSO-d₆) δ ppm 8.31 (s, 1H), 7.92 (s, 1H), 7.78 (s, 1H), 3.72 (s, 3H), 1.13 (s, 9H).

2-methyl-N-(1-(1-methyl-1H-imidazol-4-yl)ethyl) propane-2-sulfinamide: To a solution of (E)-2-methyl-N-((1-methyl-1H-imidazol-4-yl)methylene) propane-2-sulfinamide (560 mg, 2.36 mmol, 1 eq) in DCM (13 mL) was added MeMgBr (3 M, 1.58 mL, 2 eq) at −65° C. under a N₂ atmosphere, and then the mixture was stirred at 25° C. for 2 hours under a N₂ atmosphere. The reaction mixture was quenched with MeOH (20 mL) and concentrated under reduced pressure before it was diluted with EtOAc (20 ml), filtered and concentrated under reduced pressure. The residue was purified by column chromatography (SiO₂, 0-7% MeOH/EtOAc) to give the title compound (300 mg, crude) as a white solid. 1H NMR (400 MHZ, DMSO-d₆) δ ppm 7.56 (s, 1H), 6.94 (s, 1H), 5.09 (d, J=6.0 Hz, 1H), 4.29-4.24 (m, 1H), 3.61 (s, 3H), 1.38 (d, J=6.4 Hz, 3H), 1.11 (s, 9H).

1-(1-methyl-1H-imidazol-4-yl) ethan-1-amine dihydrochloride: A solution of 2-methyl-N-(1-(1-methyl-1H-imidazol-4-yl)ethyl) propane-2-sulfinamide (300 mg, 1.31 mmol, 1 eq) in HCl/MeOH (4 mL) was stirred at 25° C. for 0.5 hour under a N₂ atmosphere before it was concentrated under reduced pressure to give the title compound (340 mg, crude) as yellow oil that was taken on without purification.

The following intermediates were prepared following procedures analogous to that described for Intermediate 40.

| Int. | | | |
|---|---|---|---|
| No. | Structure | Name | LCMS, ¹H NMR |
| 41 | 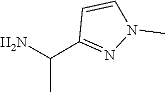 | 1-(1-methyl-1H-pyrazol-3- yl)ethan-1-amine hydrochloride | ¹H NMR (400 MHz, MeOH-d₄) δ ppm 7.61 (d, J = 2.0 Hz, 1H), 6.34 (d, J = 2.0 Hz, 1H), 4.48 (q, J = 6.8 Hz, 1H), 3.89 (s, 3H), 1.62 (d, J = 6.8 Hz, 3H) |
| 42 | 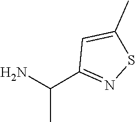 | 1-(5-methylisothiazol-3- yl)ethan-1-amine hydrochloride | ¹H NMR (400 MHz, DMSO-d₆) δ ppm 8.52 (br s, 2H), 7.26 (s, 1H), 4.59-4.46 (m, 1H), 2.58 (s, 3H), 1.51 (d, J = 6.8 Hz, 3H) |

### EXAMPLES

### Example 1

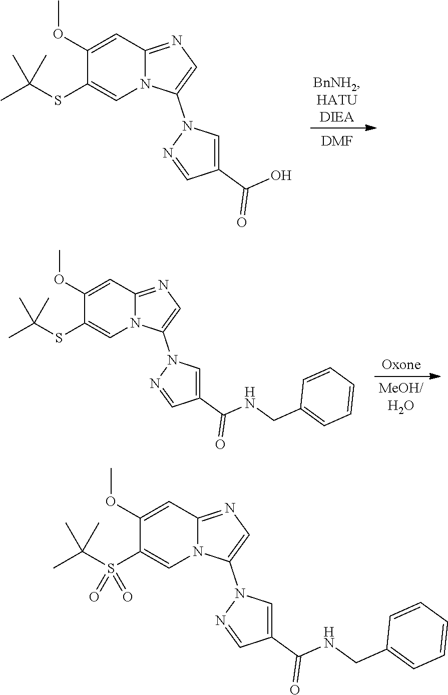

N-benzyl-1-(6-(tert-butylthio)-7-methoxyimidazo[1,2-a]pyridin-3-yl)-1H-pyrazole-4-carboxamide: To a solution of 1-(6-(tert-butylthio)-7-methoxyimidazo[1,2-a]pyridin-3-yl)-1H-pyrazole-4-carboxylic acid (100 mg, 259.81 μmol, 90% purity, 1 eq) and benzylamine (41.76 mg, 389.72 μmol, 42.48 μL, 1.5 eq) in DMF (2 mL) was added HATU (197.58 mg, 519.63 μmol, 2 eq) and DIEA (100.74 mg, 779.44 μmol, 135.76 μL, 3 eq). The mixture was stirred at 25° C. for 12 hours before it was diluted with H₂O (10 mL) and extracted with EtOAc (2×10 mL). The combined organic layers were washed with brine (10 mL), dried over Na₂SO₄, filtered and concentrated under reduced pressure to give a residue which was purified by column chromatography (SiO₂, 3-10% MeOH/DCM) to give the title compound (70 mg, 56% yield) as a white solid. [M+H]⁺=436.1. ¹H NMR (400 MHZ, DMSO-d₆) δ ppm 8.85 (t, J=6.0 Hz, 1H), 8.64 (s, 1H), 8.34 (s, 1H), 8.27 (s, 1H), 7.76 (s, 1H), 7.37-7.31 (m, 4H), 7.29-7.22 (m, 1H), 7.13 (s, 1H), 4.48 (d, J=5.6 Hz, 2H), 3.90 (s, 3H), 1.22 (s, 9H).

N-benzyl-1-(6-(tert-butylsulfonyl)-7-methoxyimidazo[1,2-a]pyridin-3-yl)-1H-pyrazole-4-carboxamide: To a solution of N-benzyl-1-(6-(tert-butylthio)-7-methoxyimidazo[1,2-a]pyridin-3-yl)-1H-pyrazole-4-carboxamide (70 mg, 144.65 μmol, 90% purity, 1 eq) in MeOH (4 mL) and H₂O (1 mL) was added Oxone® (533.55 mg, 867.89 μmol, 6 eq). The mixture was stirred at 25° C. for 12 hours before it was quenched with sat. Na₂SO₃ (5 mL) and extracted with EtOAc (2×10 mL). The combined organic layers were washed with brine (10 mL), dried over Na₂SO₄, filtered and concentrated under reduced pressure to give a residue which was purified by RP-HPLC to give the title compound (7.0 mg, 10% yield) as a white solid. [M+H]⁺=468.1. ¹H NMR (400 MHz, DMSO-d₆) δ ppm 8.89 (t, J=6.0 Hz, 1H), 8.74 (s, 1H), 8.68 (s, 1H), 8.38 (s, 1H), 7.92 (s, 1H), 7.39-7.21 (m, 6H), 4.48 (d, J=6.0 Hz, 2H), 3.94 (s, 3H), 1.29 (s, 9H).

The following compounds were prepared following procedures analogous to that described for Example 1.

| Ex. | | | |
|---|---|---|---|
| No. | Structure | Name | LCMS, ¹H NMR |
| 2 | 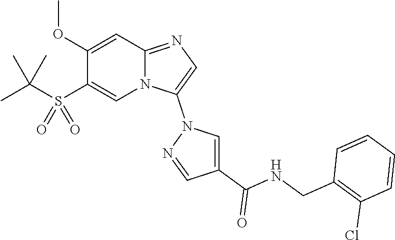 [SMILES: COc1cc2ncc(-n3cc(C(=O)NCc4ccccc4Cl)cn3)n2cc1S(=O)(=O)C(C)(C)C] | 1-(6-(tert- butylsulfonyl)-7- methoxyimidazo [1,2-a] pyridin- 3-yl)-N-(2- chlorobenzyl)- 1H-pyrazole- 4-carboxamide | [M + H]⁺ = 502.1 ¹H NMR (400 MHz, DMSO-d₆) δ ppm 8.92- 8.83 (m, 1H), 8.77-8.73 (m, 1H), 8.71 (s, 1H), 8.40 (s, 1H), 7.93 (s, 1H), 7.47 (dd, J = 7.2, 1.6 Hz, 1H), 7.43-7.39 (m, 1H), 7.37- 7.28 (m, 3H), 4.55 (d, J = 5.6 Hz, 2H), 3.95 (s, 3H), 1.30 (s, 9H) |
| 3 | 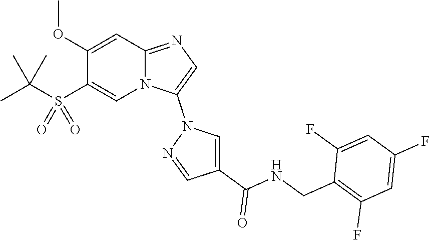 [SMILES: COc1cc2ncc(-n3cc(C(=O)NCc4c(F)cc(F)cc4F)cn3)n2cc1S(=O)(=O)C(C)(C)C] | 1-(6-(tert- butylsulfonyl)-7- methoxyimidazo [1,2-a] pyridin-3- yl)-N-(2,4,6- trifluorobenzyl)- 1H- pyrazole-4- carboxamide | [M + H]⁺ = 522.0 ¹H NMR (400 MHz, DMSO-d₆) δ ppm 8.70 (s, 2H), 8.64 (s, 1H), 8.32 (s, 1H), 7.90 (s, 1H), 7.30 (s, 1H), 7.20 (t, J = 8.8 Hz, 2H), 4.49-4.40 (m, 2H), 3.94 (s, 3H), 1.28 (s, 9H) |

### Example 4

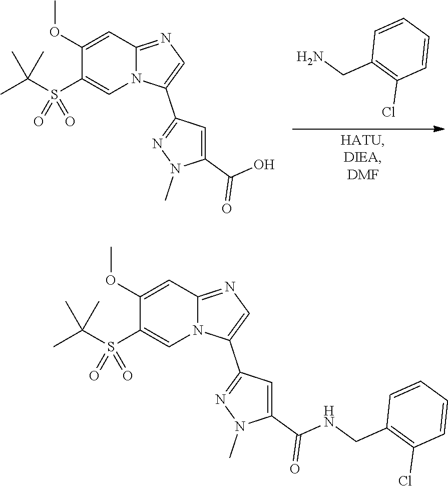 [SMILES: COc1cc2ncc(-c3cc(C(=O)O)n(C)n3)n2cc1S(=O)(=O)C(C)(C)C ; NCc1ccccc1Cl ; "HATU,DIEA,DMF" ; COc1cc2ncc(-c3cc(C(=O)NCc4ccccc4Cl)n(C)n3)n2cc1S(=O)(=O)C(C)(C)C]

3-(6-(tert-butylsulfonyl)-7-methoxyimidazo[1,2-a]pyridin-3-yl)-N-(2-chlorobenzyl)-1-methyl-1H-pyrazole-5-carboxamide: A mixture of 3-(6-(tert-butylsulfonyl)-7-methoxyimidazo[1,2-a]pyridin-3-yl)-1-methyl-1H-pyrazole-5-carboxylic acid (40 mg, 91.74 μmol, 1 eq), HATU (52.32 mg, 137.60 μmol, 1.5 eq) and DIEA (35.57 mg, 275.21 μmol, 47.94 μL, 3 eq) in DMF (1 mL) was degassed and purged with N₂ three times, and then the mixture was stirred at 25° C. for 5 minutes before (2-chlorophenyl) methanamine (15.59 mg, 110.08 μmol, 13.32 μL, 1.2 eq) was added. The resulting mixture was stirred at 25° C. for 2 hours under a N₂ atmosphere before it was filtered and concentrated under reduced pressure. The residue was purified by RP-HPLC give the title compound (4.12 mg, 9% yield) as a white solid. [M+H]⁺=516.3. ¹H NMR (400 MHZ, DMSO-d₆) δ ppm 9.87 (s, 1H), 9.23 (t, J=5.6 Hz, 1H), 7.99 (s, 1H), 7.48 (dd, J=7.2, 1.2 Hz, 1H), 7.45-7.40 (m, 1H), 7.38 (s, 1H), 7.36-7.30 (m, 2H), 7.29 (s, 1H), 4.55 (d, J=6.0 Hz, 2H), 4.14 (s, 3H), 3.94 (s, 3H), 1.33 (s, 9H).

The following compounds were prepared following procedures analogous to that described for Example 4. Detailed chiral analytical data are provided for examples obtained from racemic material by chiral SFC separation.

| Ex. | | | |
|---|---|---|---|
| No. | Structure | Name | LCMS, ¹H NMR |
| 5 | 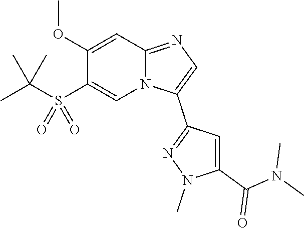 [SMILES: COc1cc2ncc(-c3cc(C(=O)N(C)C)n(C)n3)n2cc1S(=O)(=O)C(C)(C)C] | 3-(6-(tert- butylsulfonyl)-7- methoxyimidazo[1,2- a]pyridin-3-yl)-N,N,1- trimethyl-1H-pyrazole- 5-carboxamide | [M + H]⁺ = 420.0 ¹H NMR (400 MHz, DMSO- d₆) δ ppm 9.87 (s, 1H), 8.06 (s, 1H), 7.27 (s, 1H), 7.05 (s, 1H), 3.93 (d, J = 4.8 Hz, 6H), 3.13- 2.97 (m, 6H), 1.33 (s, 9H) |
| 6 | 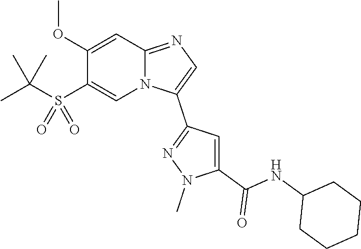 [SMILES: COc1cc2ncc(-c3cc(C(=O)NC4CCCCC4)n(C)n3)n2cc1S(=O)(=O)C(C)(C)C] | 3-(6-(tert- butylsulfonyl)-7- methoxyimidazo[1,2- a]pyridin-3-yl)-N- cyclohexyl-1-methyl- 1H-pyrazole-5- carboxamide | [M + H]⁺ = 474.2 ¹H NMR (400 MHz, DMSO- d₆) δ ppm 9.87 (s, 1H), 8.44 (d, J = 8.0 Hz, 1H), 7.98 (s, 1H), 7.28 (s, 1H), 7.27 (s, 1H), 4.11 (s, 3H), 3.94 (s, 3H), 3.79-3.70 (m, 1H), 1.86-1.72 (m, 4H), 1.62-1.59(m, 1H), 1.33 (s, 9H), 1.31-1.11 (m, 5H) |
| 7 | 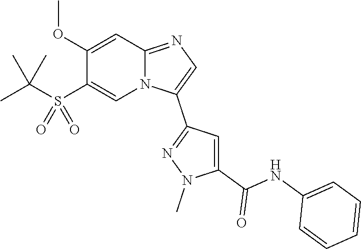 [SMILES: COc1cc2ncc(-c3cc(C(=O)Nc4ccccc4)n(C)n3)n2cc1S(=O)(=O)C(C)(C)C] | 3-(6-(tert- butylsulfonyl)-7- methoxyimidazo[1,2- a]pyridin-3-yl)-1- methyl-N-phenyl-1H- pyrazole-5- carboxamide | [M + H]⁺ = 468.2 ¹H NMR (400 MHz, DMSO- d₆) δ ppm 10.48 (s, 1H), 9.90 (s, 1H), 8.06 (s, 1H), 7.76 (d, J = 7.6 Hz, 2H), 7.48 (s, 1H), 7.39 (t, J = 8.4 Hz, 2H), 7.31 (s, 1H), 7.18-7.11 (m, 1H), 4.17 (s, 3H), 3.95 (s, 3H), 1.34 (s, 9H) |
| 8 | 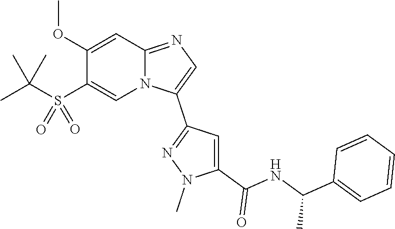 [SMILES: COc1cc2ncc(-c3cc(C(=O)N[C@@H](C)c4ccccc4)n(C)n3)n2cc1S(=O)(=O)C(C)(C)C] | (S)-3-(6-(tert- butylsulfonyl)-7- methoxyimidazo[1,2- a]pyridin-3-yl)-1- methyl-N-(1- phenylethyl)-1H- pyrazole-5- carboxamide | [M + H]⁺ = 496.1 ¹H NMR (400 MHz, DMSO- d₆) δ ppm 9.92 (s, 1H), 9.07 (d, J = 8.0 Hz, 1H), 8.16 (s, 1H), 7.43-7.39 (m, 3H), 7.37-7.32 (m, 3H), 7.28-7.22 (m, 1H), 5.16 (t, J = 7.2 Hz, 1H), 4.10 (s, 3H), 3.98 (s, 3H), 1.49 (d, J = 7.2 Hz, 3H), 1.33 (s, 9H) |
| 9 | 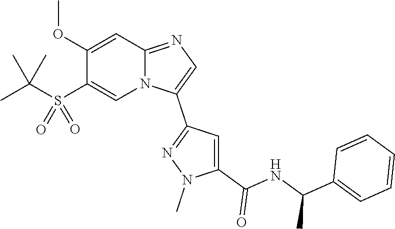 [SMILES: COc1cc2ncc(-c3cc(C(=O)N[C@H](C)c4ccccc4)n(C)n3)n2cc1S(=O)(=O)C(C)(C)C] | (R)-3-(6-(tert- butylsulfonyl)-7- methoxyimidazo[1,2- a]pyridin-3-yl)-1- methyl-N-(1- phenylethyl)-1H- pyrazole-5- carboxamide | [M + H]⁺ = 496.2 ¹H NMR (400 MHz, DMSO- d₆) δ ppm 9.89 (s, 1H), 9.04 (d, J = 8.0 Hz, 1H), 8.06 (s, 1H), 7.42-7.39 (m, 3H), 7.37-7.30 (m, 3H), 7.27-7.22 (m, 1H), 5.16 (t, J = 7.2 Hz, 1H), 4.10 (s, 3H), 3.95 (s, 3H), 1.49 (d, J = 7.2 Hz, 3H), 1.33 (s, 9H) |
| 10 | 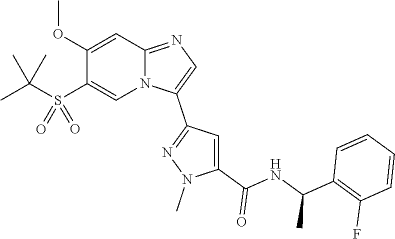 [SMILES: COc1cc2ncc(-c3cc(C(=O)N[C@H](C)c4ccccc4F)n(C)n3)n2cc1S(=O)(=O)C(C)(C)C] | (R)-3-(6-(tert- butylsulfonyl)-7- methoxyimidazo[1,2- a]pyridin-3-yl)-N-(1- (2-fluorophenyl)ethyl)- 1-methyl-1H-pyrazole- 5-carboxamide | [M + H]⁺ = 514.1 ¹H NMR (400 MHz, DMSO- d₆) δ ppm 9.90 (s, 1H), 9.12 (d, J = 8.0 Hz, 1H), 8.09 (s, 1H), 7.56-7.47 (m, 1H), 7.44 (s, 1H), 7.35-7.27 (m, 2H), 7.24- 7.14 (m, 2H), 5.38 (t, J = 7.2 Hz, 1H), 4.08 (s, 3H), 3.96 (s, 3H), 1.49 (d, J = 7.2 Hz, 3H), 1.33 (s, 9H) |
| 11 | 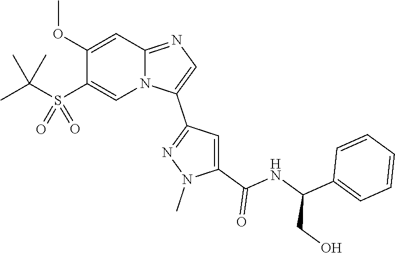 [SMILES: COc1cc2ncc(-c3cc(C(=O)N[C@H](CO)c4ccccc4)n(C)n3)n2cc1S(=O)(=O)C(C)(C)C] | (S)-3-(6-(tert- butylsulfonyl)-7- methoxyimidazo[1,2- a]pyridin-3-yl)-N-(2- hydroxy-1- phenylethyl)-1-methyl- 1H-pyrazole-5- carboxamide | [M + H]⁺ = 512.1 ¹H NMR (400 MHz, DMSO- d₆) δ ppm 9.88 (s, 1H), 8.92 (d, J = 8.0 Hz, 1H), 8.01 (s, 1H), 7.44 (s, 1H), 7.43-7.39 (m, 2H), 7.34 (t, J = 7.6 Hz, 2H), 7.29 (s, 1H), 7.28-7.23 (m, 1H), 5.14-4.92 (m, 2H), 4.09 (s, 3H), 3.94 (s, 3H), 3.73-3.64 (m, 2H), 1.33 (s, 9H) |
| 12 | 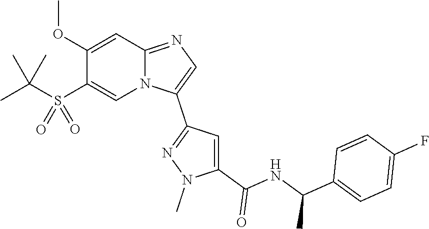 [SMILES: COc1cc2ncc(-c3cc(C(=O)N[C@H](C)c4ccc(F)cc4)n(C)n3)n2cc1S(=O)(=O)C(C)(C)C] | (R)-3-(6-(tert- butylsulfonyl)-7- methoxyimidazo[1,2- a]pyridin-3-yl)-N-(1- (4-fluorophenyl)ethyl)- 1-methyl-1H-pyrazole- 5-carboxamide | [M + H]⁺ = 514.1 ¹H NMR (400 MHz, DMSO- d₆) δ ppm 9.87 (s, 1H), 9.03 (d, J = 8.0 Hz, 1H), 8.00 (s, 1H), 7.47-7.42 (m, 2H), 7.38 (s, 1H), 7.28 (s, 1H), 7.17 (t, J = 8.8 Hz, 2H), 5.19-5.12 (m, 1H), 4.09 (s, 3H), 3.94 (s, 3H), 1.48 (d, J = 7.2 Hz, 3H), 1.32 (s, 9H) |
| 13 | 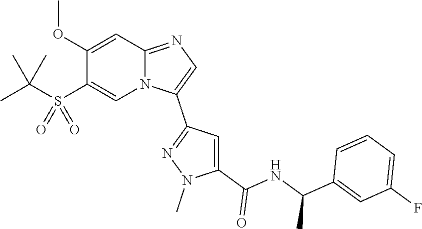 [SMILES: COc1cc2ncc(-c3cc(C(=O)N[C@H](C)c4cccc(F)c4)n(C)n3)n2cc1S(=O)(=O)C(C)(C)C] | (R)-3-(6-(tert- butylsulfonyl)-7- methoxyimidazo[1,2- a]pyridin-3-yl)-N-(1- (3-fluorophenyl)ethyl)- 1-methyl-1H-pyrazole- 5-carboxamide | [M + H]⁺ = 514.1 ¹H NMR (400 MHz, DMSO- d₆) δ ppm 9.88 (s, 1H), 9.07 (d, J = 8.0 Hz, 1H), 8.02 (s, 1H), 7.42-7.36 (m, 2H), 7.29 (s, 1H), 7.27-7.22 (m, 2H), 7.11- 7.05 (m, 1H), 5.16 (t, J = 7.6 Hz, 1H), 4.09 (s, 3H), 3.94 (s, 3H), 1.48 (d, J = 6.8 Hz, 3H), 1.32 (s, 9H) |
| 14 | 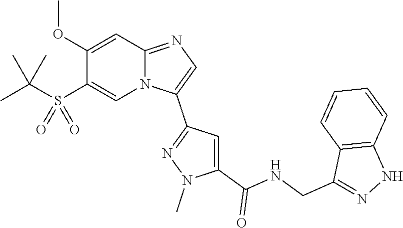 [SMILES: COc1cc2ncc(-c3cc(C(=O)NCc4n[nH]c5ccccc45)n(C)n3)n2cc1S(=O)(=O)C(C)(C)C] | N-((1H-indazol-3- yl)methyl)-3-(6-(tert- butylsulfonyl)-7- methoxyimidazo[1,2- a]pyridin-3-yl)-1- methyl-1H-pyrazole-5- carboxamide | [M + H]⁺ = 522.1 ¹H NMR (400 MHz, DMSO- d₆) δ ppm 12.89 (s, 1H), 9.84 (s, 1H), 9.26 (t, J = 5.6 Hz, 1H), 7.92 (s, 1H), 7.83 (d, J = 8.4 Hz, 1H), 7.50 (d, J = 8.4 Hz, 1H), 7.38-7.32 (m, 1H), 7.28 (d, J = 9.2 Hz, 2H), 7.10 (t, J = 7.2 Hz, 1H), 4.82 (d, J = 5.6 Hz, 2H), 4.17 (s, 3H), 3.93 (s, 3H), 1.32 (s, 9H) |
| 15 | 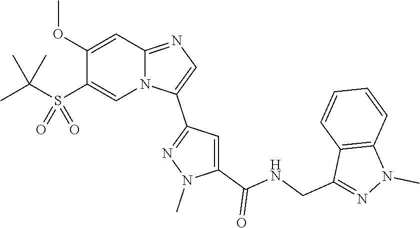 [SMILES: COc1cc2ncc(-c3cc(C(=O)NCc4nn(C)c5ccccc45)n(C)n3)n2cc1S(=O)(=O)C(C)(C)C] | 3-(6-(tert- butylsulfonyl)-7- methoxyimidazo[1,2- a]pyridin-3-yl)-1- methyl-N-((1-methyl- 1H-indazol-3- yl)methyl)-1H- pyrazole-5- carboxamide | [M + H]⁺ = 536.0 ¹H NMR (400 MHz, DMSO- d₆) δ ppm 9.84 (s, 1H), 9.28 (t, J = 6.0 Hz, 1H), 7.93 (s, 1H), 7.84 (d, J = 8.0 Hz, 1H), 7.61 (d, J = 8.4 Hz, 1H), 7.43-7.36 (m, 1H), 7.28 (d, J = 6.8 Hz, 2H), 7.17-7.08 (m, 1H), 4.79 (d, J = 5.6 Hz, 2H), 4.16 (s, 3H), 4.02 (s, 3H), 3.93 (s, 3H), 1.32 (s, 9H) |
| 16 | 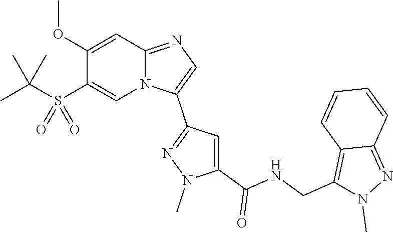 [SMILES: COc1cc2ncc(-c3cc(C(=O)NCc4c5ccccc5nn4C)n(C)n3)n2cc1S(=O)(=O)C(C)(C)C] | 3-(6-(tert- butylsulfonyl)-7- methoxyimidazo[1,2- a]pyridin-3-yl)-1- methyl-N-((2-methyl- 2H-indazol-3- yl)methyl)-1H- pyrazole-5- carboxamide | [M + H]⁺ = 536.0 ¹H NMR (400 MHz, DMSO- d₆) δ ppm 9.85 (s, 1H), 9.34 (t, J = 5.2 Hz, 1H), 7.96 (s, 1H), 7.82 (d, J = 8.4 Hz, 1H), 7.55 (d, J = 8.8 Hz, 1H), 7.27 (s, 2H), 7.22 (t, J = 7.6, 1H), 7.04 (t, J = 7.6 Hz, 1H), 4.92 (d, J = 5.2 Hz, 2H), 4.21 (s, 3H), 4.14 (s, 3H), 3.93 (s, 3H), 1.32 (s, 9H) |
| 17 | 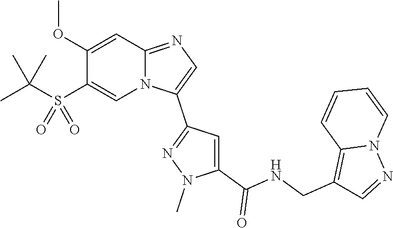 [SMILES: COc1cc2ncc(-c3cc(C(=O)NCc4cnn5ccccc45)n(C)n3)n2cc1S(=O)(=O)C(C)(C)C] | 3-(6-(tert- butylsulfonyl)-7- methoxyimidazo[1,2- a]pyridin-3-yl)-1- methyl-N- (pyrazolo[1,5- a]pyridin-3-ylmethyl)- 1H-pyrazole-5- carboxamide | [M + H]⁺ = 522.3 ¹H NMR (400 MHz, DMSO- d₆) δ ppm 9.85 (s, 1H), 9.11 (t, J = 5.5 Hz, 1H), 8.64 (d, J = 7.0 Hz, 1H), 8.02-7.97 (m, 1H), 7.94 (s, 1H), 7.80 (d, J = 9.0 Hz, 1H), 7.27-7.22 (m, 3H), 6.90-6.85 (m, 1H), 4.61 (d, J = 6.0 Hz, 2H), 4.15 (s, 3H), 3.93 (s, 3H), 1.32 (s, 9H) |
| 18 | 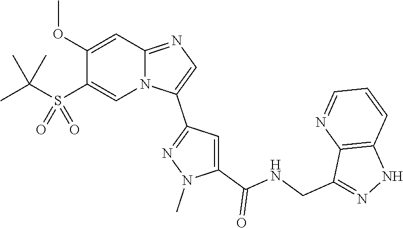 [SMILES: COc1cc2ncc(-c3cc(C(=O)NCc4n[nH]c5cccnc45)n(C)n3)n2cc1S(=O)(=O)C(C)(C)C] | N-((1H-pyrazolo[4,3- b]pyridin-3-yl)methyl)- 3-(6-(tert- butylsulfonyl)-7- methoxyimidazo[1,2- a]pyridin-3-yl)-1- methyl-1H-pyrazole-5- carboxamide | [M + H]⁺ = 523.0 ¹H NMR (400 MHz, DMSO- d₆) δ ppm 13.18 (s, 1H), 9.86 (s, 1H), 9.17 (t, J = 5.2 Hz, 1H), 8.53 (dd, J = 4.4, 1.2 Hz, 1H), 7.99 (dd, J = 7.6, 1.2 Hz, 1H), 7.93 (s, 1H), 7.38 (dd, J = 8.4, 4.0 Hz, 1H), 7.33 (s, 1H), 7.28 (s, 1H), 4.89 (d, J = 5.6 Hz, 2H), 4.16 (s, 3H), 3.93 (s, 3H), 1.33 (s, 9H) |
| 19 | 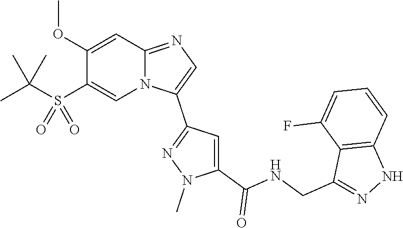 [SMILES: COc1cc2ncc(-c3cc(C(=O)NCc4n[nH]c5cccc(F)c45)n(C)n3)n2cc1S(=O)(=O)C(C)(C)C] | 3-(6-(tert- butylsulfonyl)-7- methoxyimidazo[1,2- a]pyridin-3-yl)-N-((4- fluoro-1H-indazol-3- yl)methyl)-1-methyl- 1H-pyrazole-5- carboxamide | [M + H]⁺ = 540.0 ¹H NMR (400 MHz, DMSO- d₆) δ ppm 13.21 (s, 1H), 9.86 (s, 1H), 9.14 (t, J = 5.2 Hz, 1H), 7.93 (s, 1H), 7.38-7.29 (m, 3H), 7.27 (s, 1H), 6.90- 6.80 (m, 1H), 4.85 (d, J = 5.2 Hz, 2H), 4.16 (s, 3H), 3.93 (s, 3H), 1.33 (s, 9H) |
| 20 | 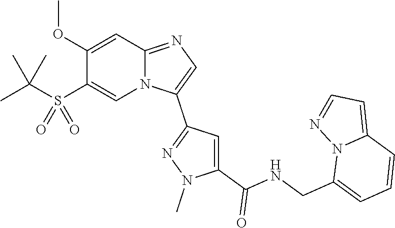 [SMILES: COc1cc2ncc(-c3cc(C(=O)NCc4cccc5ccnn45)n(C)n3)n2cc1S(=O)(=O)C(C)(C)C] | 3-(6-(tert- butylsulfonyl)-7- methoxyimidazo[1,2- a]pyridin-3-yl)-1- methyl-N- (pyrazolo[1,5- a]pyridin-7-ylmethyl)- 1H-pyrazole-5- carboxamide | [M + H]⁺ = 522.0 ¹H NMR (400 MHz, DMSO- d₆) δ ppm 9.88 (s, 1H), 9.42 (t, J = 5.2 Hz, 1H), 8.10 (d, J = 2.4 Hz, 1H), 8.02 (s, 1H), 7.70 (d, J = 8.4 Hz, 1H), 7.45 (s, 1H), 7.30 (s, 1H), 7.28-7.22 (m, 1H), 6.86 (d, J = 6.8 Hz, 1H), 6.73 (d, J = 2.4 Hz, 1H), 4.92 (d, J = 5.6 Hz, 2H), 4.16 (s, 3H), 3.95 (s, 3H), 1.33 (s, 9H) |
| 21 | 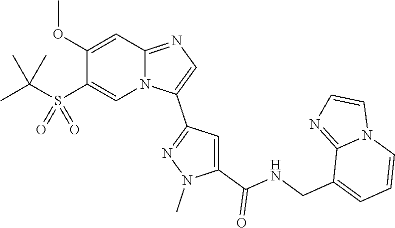 [SMILES: COc1cc2ncc(-c3cc(C(=O)NCc4cccn5ccnc45)n(C)n3)n2cc1S(=O)(=O)C(C)(C)C] | 3-(6-(tert- butylsulfonyl)-7- methoxyimidazo[1,2- a]pyridin-3-yl)-N- (imidazo[1,2-a]pyridin- 8-ylmethyl)-1-methyl- 1H-pyrazole-5- carboxamide | [M + H]⁺ = 522.0 ¹H NMR (400 MHz, DMSO- d₆) δ ppm 9.87 (s, 1H), 9.28 (t, J = 5.6 Hz, 1H), 8.49 (d, J = 6.4 Hz, 1H), 8.08-7.92 (m, 2H), 7.59 (d, J = 0.8 Hz, 1H), 7.40 (s, 1H), 7.29 (s, 1H), 7.13 (d, J = 6.8 Hz, 1H), 6.88 (t, J = 7.2 Hz, 1H), 4.79 (d, J = 5.6 Hz, 2H), 4.15 (s, 3H), 3.94 (s, 3H), 1.33 (s, 9H) |
| 22 | 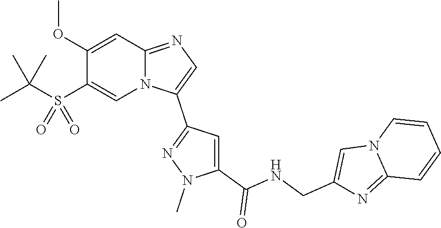 [SMILES: COc1cc2ncc(-c3cc(C(=O)NCc4cn5ccccc5n4)n(C)n3)n2cc1S(=O)(=O)C(C)(C)C] | 3-(6-(tert- butylsulfonyl)-7- methoxyimidazo[1,2- a]pyridin-3-yl)-N- (imidazo[1,2-a]pyridin- 2-ylmethyl)-1-methyl- 1H-pyrazole-5- carboxamide | [M + H]⁺ = 522.0 ¹H NMR (400 MHz, DMSO- d₆) δ ppm 9.86 (s, 1H), 9.20 (t, J = 5.6 Hz, 1H), 8.50 (d, J = 6.8 Hz, 1H), 7.96 (s, 1H), 7.85 (s, 1H), 7.50 (d, J = 9.2 Hz, 1H), 7.36 (s, 1H), 7.29 (s, 1H), 7.24-7.18 (m, 1H), 6.93-6.78 (m, 1H), 4.58 (d, J = 5.6 Hz, 2H), 4.16 (s, 3H), 3.94 (s, 3H), 1.33 (s, 9H) |
| 23 | 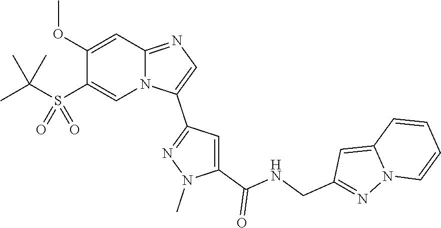 [SMILES: COc1cc2ncc(-c3cc(C(=O)NCc4cc5ccccn5n4)n(C)n3)n2cc1S(=O)(=O)C(C)(C)C] | 3-(6-(tert- butylsulfonyl)-7- methoxyimidazo[1,2- a]pyridin-3-yl)-1- methyl-N- (pyrazolo[1,5- a]pyridin-2-ylmethyl)- 1H-pyrazole-5- carboxamide | [M + H]⁺ = 522.2 ¹H NMR (400 MHz, DMSO- d₆) δ ppm 9.86 (s, 1H), 9.27 (t, J = 5.2 Hz, 1H), 8.62 (d, J = 7.2 Hz, 1H), 7.97 (s, 1H), 7.63 (d, J = 9.2 Hz, 1H), 7.35 (s, 1H), 7.29 (s, 1H), 7.26-7.11 (m, 1H), 6.84 (t, J = 7.2 Hz, 1H), 6.54 (s, 1H), 4.64 (d, J = 5.6 Hz, 2H), 4.16 (s, 3H), 3.94 (s, 3H), 1.33 (s, 9H) |
| 24 | 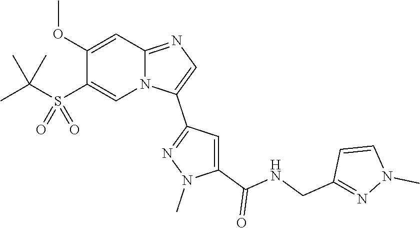 [SMILES: COc1cc2ncc(-c3cc(C(=O)NCc4ccn(C)n4)n(C)n3)n2cc1S(=O)(=O)C(C)(C)C] | 3-(6-(tert- butylsulfonyl)-7- methoxyimidazo[1,2- a]pyridin-3-yl)-1- methyl-N-((1-methyl- 1H-pyrazol-3- yl)methyl)-1H- pyrazole-5- carboxamide | [M + H]⁺ = 486.1 ¹H NMR (400 MHz, DMSO- d₆) δ ppm 9.87 (s, 1H), 9.07 (t, J = 6.0 Hz, 1H), 8.00 (s, 1H), 7.60 (d, J = 2.0 Hz, 1H), 7.31 (d, J = 6.0 Hz, 2H), 6.16 (d, J = 2.0 Hz, 1H), 4.40 (d, J = 6.0 Hz, 2H), 4.14 (s, 3H), 3.95 (s, 3H), 3.79 (s, 3H), 1.33 (s, 9H) |
| 25 | 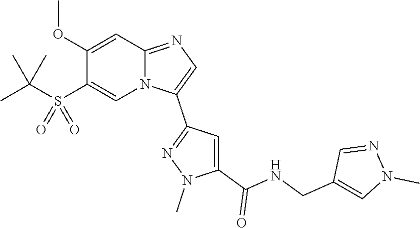 [SMILES: COc1cc2ncc(-c3cc(C(=O)NCc4cnn(C)c4)n(C)n3)n2cc1S(=O)(=O)C(C)(C)C] | 3-(6-(tert- butylsulfonyl)-7- methoxyimidazo[1,2- a]pyridin-3-yl)-1- methyl-N-((1-methyl- 1H-pyrazol-4- yl)methyl)-1H- pyrazole-5- carboxamide | [M + H]⁺ = 486.1 ¹H NMR (400 MHz, DMSO- d₆) δ ppm 9.85 (s, 1H), 8.99 (t, J = 5.6 Hz, 1H), 7.95 (s, 1H), 7.63 (s, 1H), 7.38 (s, 1H), 7.27 (d, J = 4.8 Hz, 2H), 4.28 (d, J = 5.6 Hz, 2H), 4.14 (s, 3H), 3.94 (s, 3H), 3.79 (s, 3H), 1.33 (s, 9H) |
| 26 | 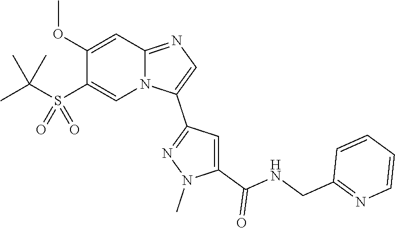 [SMILES: COc1cc2ncc(-c3cc(C(=O)NCc4ccccn4)n(C)n3)n2cc1S(=O)(=O)C(C)(C)C] | 3-(6-(tert- butylsulfonyl)-7- methoxyimidazo[1,2- a]pyridin-3-yl)-1- methyl-N-(pyridin-2- ylmethyl)-1H-pyrazole- 5-carboxamide | [M + H]⁺ = 483.2 ¹H NMR (400 MHz, DMSO- d₆) δ ppm 9.89 (s, 1H), 9.30 (t, J = 6.0 Hz, 1H), 8.55 (d, J = 4.8 Hz, 1H), 8.07 (s, 1H), 7.84-7.78 (m, 1H), 7.44-7.38 (m, 2H), 7.36-7.27 (m, 2H), 4.58 (d, J = 6.0 Hz, 2H), 4.15 (s, 3H), 3.96 (s, 3H), 1.33 (s, 9H) |
| 27 | 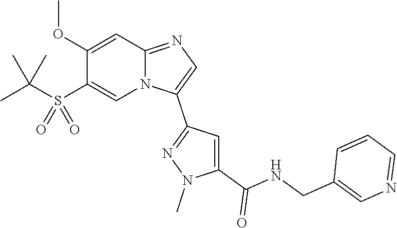 [SMILES: COc1cc2ncc(-c3cc(C(=O)NCc4cccnc4)n(C)n3)n2cc1S(=O)(=O)C(C)(C)C] | 3-(6-(tert- butylsulfonyl)-7- methoxyimidazo[1,2- a]pyridin-3-yl)-1- methyl-N-(pyridin-3- ylmethyl)-1H-pyrazole- 5-carboxamide | [M + H]⁺ = 483.1 ¹H NMR (400 MHz, DMSO- d₆) δ ppm 9.86 (s, 1H), 9.27 (t, J = 6.0 Hz, 1H), 8.59 (d, J = 1.2 Hz, 1H), 8.49 (dd, J = 1.2, 4.4 Hz, 1H), 7.99 (s, 1H), 7.82-7.73 (m, 1H), 7.44-7.37 (m, 1H), 7.32 (s, 1H), 7.29 (s, 1H), 4.50 (d, J = 5.6 Hz, 2H), 4.14 (s, 3H), 3.94 (s, 3H), 1.33 (s, 9H) |
| 28 | 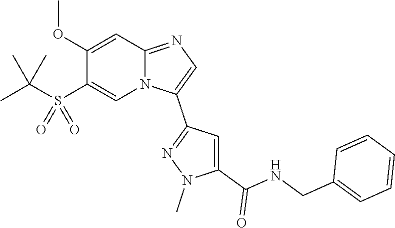 [SMILES: COc1cc2ncc(-c3cc(C(=O)NCc4ccccc4)n(C)n3)n2cc1S(=O)(=O)C(C)(C)C] | N-benzyl-3-(6-(tert- butylsulfonyl)-7- methoxyimidazo[1,2- a]pyridin-3-yl)-1- methyl-1H-pyrazole-5- carboxamide | [M + H]⁺ = 482.1 ¹H NMR (400 MHz, DMSO- d₆) δ ppm 9.86 (s, 1H), 9.23 (t, J = 5.2 Hz, 1H), 7.97 (s, 1H), 7.48-7.09 (m, 7H), 4.48 (d, J = 6.0 Hz, 2H), 4.15 (s, 3H), 3.94 (s, 3H), 1.33 (s, 9H) |
| 29 | 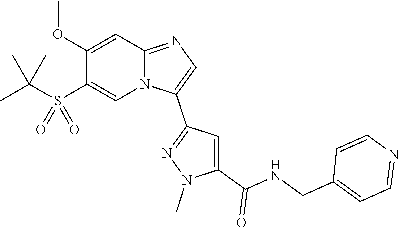 [SMILES: COc1cc2ncc(-c3cc(C(=O)NCc4ccncc4)n(C)n3)n2cc1S(=O)(=O)C(C)(C)C] | 3-(6-(tert- butylsulfonyl)-7- methoxyimidazo[1,2- a]pyridin-3-yl)-1- methyl-N-(pyridin-4- ylmethyl)-1H-pyrazole- 5-carboxamide | [M + H]⁺ = 483.1 ¹H NMR (400 MHz, DMSO- d₆) δ ppm 9.89 (s, 1H), 9.41 (t, J = 6.0 Hz, 1H), 8.67 (d, J = 5.2 Hz, 2H), 8.10 (s, 1H), 7.62 (s, 2H), 7.39 (s, 1H), 7.34 (s, 1H), 4.61 (d, J = 5.6 Hz, 2H), 4.14 (s, 3H), 3.97 (s, 3H), 1.34 (s, 9H) |
| 30 | 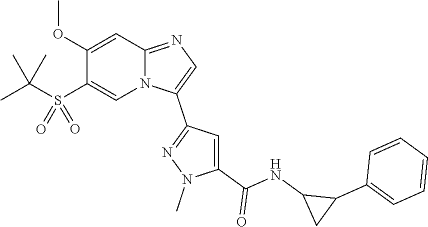 [SMILES: COc1cc2ncc(-c3cc(C(=O)NC4CC4c4ccccc4)n(C)n3)n2cc1S(=O)(=O)C(C)(C)C] | cis-(1S,2S) or (1R,2R) 3-(6-(tert- butylsulfonyl)-7- methoxyimidazo[1,2- a]pyridin-3-yl)-1- methyl-N-(2- phenylcyclopropyl)- 1H-pyrazole-5- carboxamide | [M + H]⁺ = 508.1 ¹H NMR (400 MHz, DMSO- d₆) δ ppm 9.79 (s, 1H), 8.62 (d, J = 4.0 Hz, 1H), 7.93 (s, 1H), 7.29-7.18 (m, 5H), 7.14-7.08 (m, 1H), 6.98 (s, 1H), 3.93 (s, 3H), 3.83 (s, 3H), 3.16-3.12 (m, 1H), 2.34-2.28 (m, 1H), 1.36-1.29 (m, 11H) SFC: Rₜ = 1.524 min, 99.7% ee; (S,S) Whelk-O 1 50 x 4.6 mm, 3.5 μm; 40% i- PrOH/MeCN (7:3 + 0.05% DEA) in CO₂, 3 mL/min; 35º C., 100 Bar, 220 nm |
| 31 | 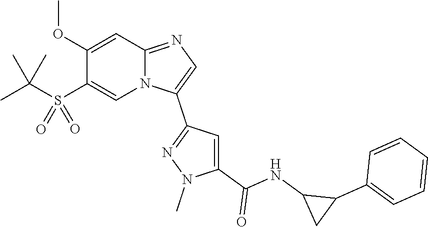 [SMILES: COc1cc2ncc(-c3cc(C(=O)NC4CC4c4ccccc4)n(C)n3)n2cc1S(=O)(=O)C(C)(C)C] | cis-(1R,2R) or (1S,2S) 3-(6-(tert- butylsulfonyl)-7- methoxyimidazo[1,2- a]pyridin-3-yl)-1- methyl-N-(2- phenylcyclopropyl)- 1H-pyrazole-5- carboxamide | [M + H]⁺ = 508.2 ¹H NMR (400 MHz, DMSO- d₆) δ ppm 9.79 (s, 1H), 8.62 (d, J = 4.4 Hz, 1H), 7.93 (s, 1H), 7.28-7.18 (m, 5H), 7.14-7.08 (m, 1H), 6.98 (s, 1H), 3.93 (s, 3H), 3.83 (s, 3H), 3.16-3.13 (m, 1H), 2.34-2.28 (m, 1H), 1.36-1.29 (m, 11H) SFC: Rₜ = 2.102 min, 98.1% ee; (S,S) Whelk-O 1 50 x 4.6 mm, 3.5 μm; 40% i- PrOH/MeCN (7:3 + 0.05% DEA) in CO₂, 3 mL/min; 35º C., 100 Bar, 220 nm |
| 32 | 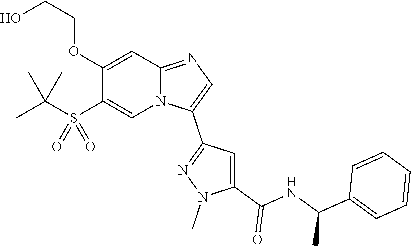 [SMILES: C[C@@H](NC(=O)c1cc(-c2cnc3cc(OCCO)c(S(=O)(=O)C(C)(C)C)cn23)nn1C)c1ccccc1] | (R)-3-(6-(tert- butylsulfonyl)-7-(2- hydroxyethoxy)imidazo [1,2-a]pyridin-3-yl)-1- methyl-N-(1- phenylethyl)-1H- pyrazole-5- carboxamide | [M + H]⁺ = 526.1 ¹H NMR (400 MHz, DMSO- d₆) δ ppm 9.88 (s, 1H), 9.04 (d, J = 7.6 Hz, 1H), 8.00 (s, 1H), 7.42-7.40 (m, 3H), 7.36-7.33 (m, 2H), 7.30 (s, 1H), 7.26- 7.25 (m, 1H), 5.19-5.13 (m, 1H), 4.82 (t, J = 5.2 Hz, 1H), 4.19 (t, J = 4.8 Hz, 2H), 4.10 (s, 3H), 3.80-3.76 (m, 2H), 1.49 (d, J = 6.8 Hz, 3H), 1.34 (s, 9H) |
| 33 | 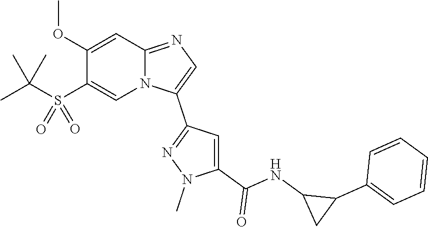 [SMILES: COc1cc2ncc(-c3cc(C(=O)NC4CC4c4ccccc4)n(C)n3)n2cc1S(=O)(=O)C(C)(C)C] | trans-(1R,2S) or (1S,2R) 3-(6-(tert- butylsulfonyl)-7- methoxyimidazo[1,2- a]pyridin-3-yl)-1- methyl-N-(2- phenylcyclopropyl)- 1H-pyrazole-5- carboxamide | [M + H]⁺ = 508.1 ¹H NMR (400 MHz, DMSO- d₆) δ ppm 9.87 (s, 1H), 8.92 (d, J = 4.0 Hz, 1H), 7.98 (s, 1H), 7.31-7.26 (m, 4H), 7.20-7.17 (m, 3H), 4.13 (s, 3H), 3.94 (s, 3H), 3.05-3.01 (m, 1H), 2.14- 2.09 (m, 1H), 1.34-1.33 (m, 11H) SFC: Rₜ = 1.884 min, 98.4% ee; CHIRALCEL OD-3 50 x 4.6 mm, 3 μm; 5-40% EtOH (0.05% DEA) in CO₂, 3 mL/min; 35° C., 100 Bar, 220 nm |
| 34 | 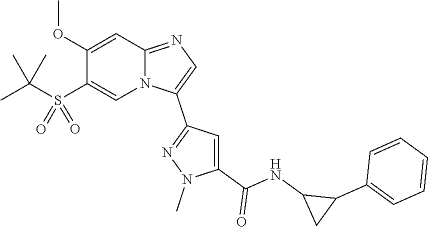 [SMILES: COc1cc2ncc(-c3cc(C(=O)NC4CC4c4ccccc4)n(C)n3)n2cc1S(=O)(=O)C(C)(C)C] | trans-(1S,2R) or (1R,2S) 3-(6-(tert- butylsulfonyl)-7- methoxyimidazo[1,2- a]pyridin-3-yl)-1- methyl-N-(2- phenylcyclopropyl)- 1H-pyrazole-5- carboxamide | [M + H]⁺ = 508.1 ¹H NMR (400 MHz, DMSO- d₆) δ ppm 9.87 (s, 1H), 8.92 (d, J = 4.0 Hz, 1H), 7.98 (s, 1H), 7.31-7.26 (m, 4H), 7.20-7.17 (m, 3H), 4.13 (s, 3H), 3.94 (s, 3H), 3.04-3.01 (m, 1H), 2.14- 2.09 (m, 1H), 1.37-1.33 (m, 11H) SFC: Rₜ = 2.093 min, 99.7% ee; CHIRALCEL OD-3 50 x 4.6 mm, 3 μm; 5-40% EtOH (0.05% DEA) in CO₂, 3 mL/min; 35° C., 100 Bar, 220 nm |
| 35 | 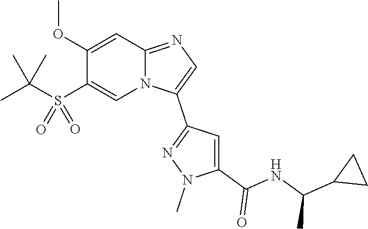 [SMILES: COc1cc2ncc(-c3cc(C(=O)N[C@H](C)C4CC4)n(C)n3)n2cc1S(=O)(=O)C(C)(C)C] | (R)-3-(6-(tert- butylsulfonyl)-7- methoxyimidazo[1,2- a]pyridin-3-yl)-N-(1- cyclopropylethyl)-1- methyl-1H-pyrazole-5- carboxamide | [M + H]⁺ = 460.1 ¹H NMR (400 MHz, DMSO- d₆) δ ppm 9.90 (s, 1H), 8.58 (d, J = 8.4 Hz, 1H), 8.07 (br s, 1H), 7.37-7.23 (m, 2H), 4.12 (s, 3H), 3.96 (s, 3H), 3.19 (d, J = 2.0 Hz, 1H), 1.33 (s, 9H), 1.23 (d, J = 6.8 Hz, 3H), 1.05- 0.93 (m, 1H), 0.52-0.36 (m, 2H), 0.35-0.19 (m, 2H) |
| 36 | 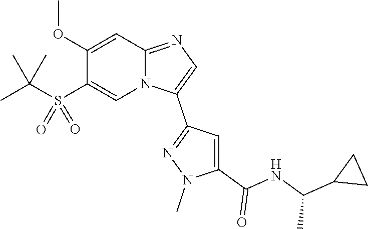 [SMILES: COc1cc2ncc(-c3cc(C(=O)N[C@@H](C)C4CC4)n(C)n3)n2cc1S(=O)(=O)C(C)(C)C] | (S)-3-(6-(tert- butylsulfonyl)-7- methoxyimidazo[1,2- a]pyridin-3-yl)-N-(1- cyclopropylethyl)-1- methyl-1H-pyrazole-5- carboxamide | [M + H]⁺ = 460.1 ¹H NMR (400 MHz, DMSO- d₆) δ ppm 9.88 (s, 1H), 8.57 (d, J = 8.4 Hz, 1H), 8.00 (s, 1H), 7.29 (d, J = 4.4 Hz, 2H), 4.11 (s, 3H), 3.94 (s, 3H), 3.45 (d, J = 7.6 Hz, 1H), 1.33 (s, 9H), 1.23 (d, J = 6.4 Hz, 3H), 1.03- 0.94 (m, 1H), 0.51-0.38 (m, 2H), 0.35-0.20 (m, 2H) |
| 37 | 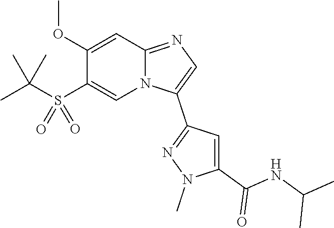 [SMILES: COc1cc2ncc(-c3cc(C(=O)NC(C)C)n(C)n3)n2cc1S(=O)(=O)C(C)(C)C] | 3-(6-(tert- butylsulfonyl)-7- methoxyimidazo[1,2- a]pyridin-3-yl)-N- isopropyl-1-methyl- 1H-pyrazole-5- carboxamide | [M + H]⁺ = 434.1 ¹H NMR (400 MHz, DMSO- d₆) δ ppm 9.90 (s, 1H), 8.46 (d, J = 7.6 Hz, 1H), 8.04 (s, 1H), 7.39-7.23 (m, 2H), 4.12 (s, 3H), 4.10-4.02 (m, 1H), 3.96 (s, 3H), 1.33 (s, 9H), 1.18 (d, J = 6.4 Hz, 6H) |
| 38 | 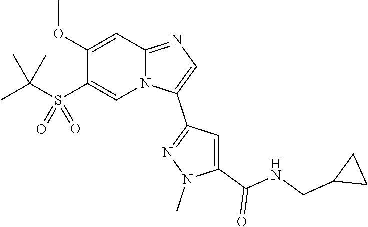 [SMILES: COc1cc2ncc(-c3cc(C(=O)NCC4CC4)n(C)n3)n2cc1S(=O)(=O)C(C)(C)C] | 3-(6-(tert- butylsulfonyl)-7- methoxyimidazo[1,2- a]pyridin-3-yl)-N- (cyclopropylmethyl)-1- methyl-1H-pyrazole-5- carboxamide | [M + H]⁺ = 446.1 ¹H NMR (400 MHz, DMSO- d₆) δ ppm 9.87 (s, 1H), 8.76 (s, 1H), 7.99 (s, 1H), 7.28 (d, J = 2.4 Hz, 2H), 4.13 (s, 3H), 3.94 (s, 3H), 3.14 (t, J = 6.4 Hz, 2H), 1.33 (s, 9H), 1.03 (s, 1H), 0.50-0.41 (m, 2H), 0.30-0.22 (m, 2H) |
| 39 | 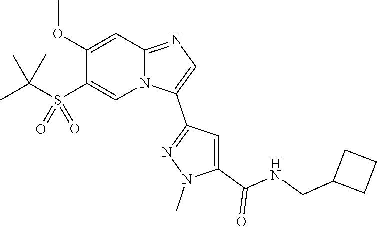 [SMILES: COc1cc2ncc(-c3cc(C(=O)NCC4CCC4)n(C)n3)n2cc1S(=O)(=O)C(C)(C)C] | 3-(6-(tert- butylsulfonyl)-7- methoxyimidazo[1,2- a]pyridin-3-yl)-N- (cyclobutylmethyl)-1- methyl-1H-pyrazole-5- carboxamide | [M + H]⁺ = 460.1 ¹H NMR (400 MHz, DMSO- d₆) δ ppm 9.87 (s, 1H), 8.64 (t, J = 5.6 Hz, 1H), 7.98 (s, 1H), 7.28 (s, 1H), 7.24 (s, 1H), 4.12 (s, 3H), 3.94 (s, 3H), 3.28 (d, J = 6.4 Hz, 2H), 2.58-2.52 (m, 1H), 2.05-1.96 (m, 2H), 1.88- 1.80 (m, 2H), 1.77-1.69 (m, 2H), 1.33 (s, 9H) |
| 40 | 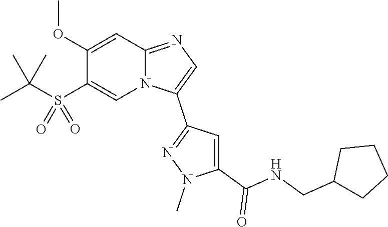 [SMILES: COc1cc2ncc(-c3cc(C(=O)NCC4CCCC4)n(C)n3)n2cc1S(=O)(=O)C(C)(C)C] | 3-(6-(tert- butylsulfonyl)-7- methoxyimidazo[1,2- a]pyridin-3-yl)-N- (cyclopentylmethyl)-1- methyl-1H-pyrazole-5- carboxamide | [M + H]⁺ = 474.0 ¹H NMR (400 MHz, DMSO- d₆) δ ppm 9.87 (s, 1H), 8.69 (t, J = 5.6 Hz, 1H), 7.28 (s, 1H), 7.26 (s, 1H), 4.11 (s, 3H), 3.94 (s, 3H), 3.18 (dd, J = 6.0, 7.2 Hz, 2H), 2.20-2.08 (m, 1H), 1.74-1.66 (m, 2H), 1.63-1.55 (m, 2H), 1.55-1.47 (m, 2H), 1.33 (s, 9H), 1.29-1.20 (m, 2H) |
| 41 | 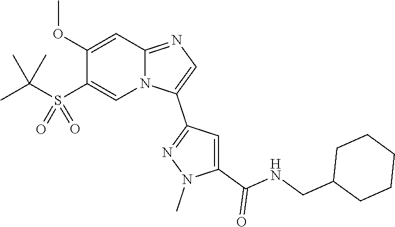 [SMILES: COc1cc2ncc(-c3cc(C(=O)NCC4CCCCC4)n(C)n3)n2cc1S(=O)(=O)C(C)(C)C] | 3-(6-(tert- butylsulfonyl)-7- methoxyimidazo[1,2- a]pyridin-3-yl)-N- (cyclohexylmethyl)-1- methyl-1H-pyrazole-5- carboxamide | [M + H]⁺ = 488.1 ¹H NMR (400 MHz, DMSO- d₆) δ ppm 9.95 (s, 1H), 8.68 (t, J = 5.6 Hz, 1H), 8.25 (s, 1H), 7.39 (s, 1H), 7.31 (s, 1H), 4.13 (s, 3H), 4.01 (s, 3H), 3.10 (t, J = 6.4 Hz, 2H), 1.76-1.66 (m, 4H), 1.64 (s, 1H), 1.58-1.50 (m, 1H), 1.34 (s, 9H), 1.25- 1.16 (m, 3H), 0.98-0.88 (m, 2H) |
| 42 | 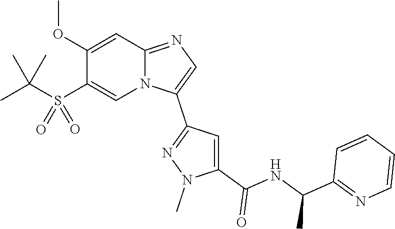 [SMILES: COc1cc2ncc(-c3cc(C(=O)N[C@H](C)c4ccccn4)n(C)n3)n2cc1S(=O)(=O)C(C)(C)C] | (R)-3-(6-(tert- butylsulfonyl)-7- methoxyimidazo[1,2- a]pyridin-3-yl)-1- methyl-N-(1-(pyridin- 2-yl)ethyl)-1H- pyrazole-5- carboxamide | [M + H]⁺ = 497.1 ¹H NMR (400 MHz, DMSO- d₆) δ ppm 9.88 (s, 1H), 9.04 (d, J = 7.6 Hz, 1H), 8.60-8.48 (m, 1H), 8.01 (s, 1H), 7.80-7.76 (m, 1H), 7.47-7.41 (m, 2H), 7.31-7.25 (m, 2H), 5.19 (t, J = 7.2 Hz, 1H), 4.10 (s, 3H), 3.94 (s, 3H), 1.52 (d, J = 7.2 Hz, 3H), 1.33 (s, 9H) |
| 43 | 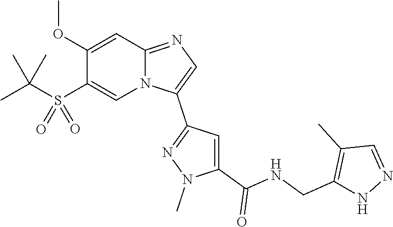 [SMILES: COc1cc2ncc(-c3cc(C(=O)NCc4[nH]ncc4C)n(C)n3)n2cc1S(=O)(=O)C(C)(C)C] | 3-(6-(tert- butylsulfonyl)-7- methoxyimidazo [1,2- a]pyridin-3-yl)-1- methyl-N-((4-methyl- 1H-pyrazol-5- yl)methyl)-1H- pyrazole-5- carboxamide | [M + H]⁺ = 486.0 ¹H NMR (400 MHz, DMSO- d₆) δ ppm 12.42 (s, 1H), 9.85 (s, 1H), 8.96 (d, J = 4.8 Hz, 1H), 7.94 (s, 1H), 7.39 (d, J = 2.4 Hz, 1H), 7.31 (s, 1H), 7.28 (s, 1H), 4.43 (d, J = 5.6 Hz, 2H), 4.14 (s, 3H), 3.93 (s, 3H), 2.01 (s, 3H), 1.32 (s, 9H) |
| 44 | 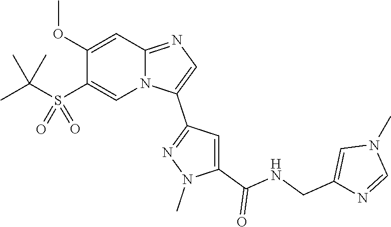 [SMILES: COc1cc2ncc(-c3cc(C(=O)NCc4cn(C)cn4)n(C)n3)n2cc1S(=O)(=O)C(C)(C)C] | 3-(6-(tert- butylsulfonyl)-7- methoxyimidazo[1,2- a]pyridin-3-yl)-1- methyl-N-((1-methyl- 1H-imidazol-4- yl)methyl)-1H- pyrazole-5- carboxamide | [M + H]⁺ = 486.1 ¹H NMR (400 MHz, DMSO- d₆) δ ppm 9.85 (s, 1H), 8.97 (d, J = 5.6 Hz, 1H), 7.94 (s, 1H), 7.50 (s, 1H), 7.32 (s, 1H), 7.28 (s, 1H), 7.00 (s, 1H), 4.31 (d, J = 5.6 Hz, 2H), 4.14 (s, 3H), 3.94 (s, 3H), 3.61 (s, 3H), 1.33 (s, 9H) |
| 45 | 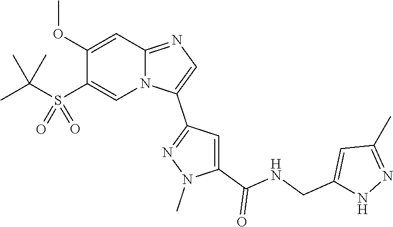 [SMILES: COc1cc2ncc(-c3cc(C(=O)NCc4cc(C)n[nH]4)n(C)n3)n2cc1S(=O)(=O)C(C)(C)C] | 3-(6-(tert- butylsulfonyl)-7- methoxyimidazo[1,2- a]pyridin-3-yl)-1- methyl-N-((3-methyl- 1H-pyrazol-5- yl)methyl)-1H- pyrazole-5- carboxamide | [M + H]⁺ = 486.1 ¹H NMR (400 MHz, DMSO- d₆) δ ppm 12.45-12.17 (m, 1H), 9.85 (s, 1H), 9.03 (t, J = 5.2 Hz, 1H), 7.95 (s, 1H), 7.29 (d, J = 9.6 Hz, 2H), 5.94 (s, 1H), 4.37 (d, J = 5.6 Hz, 2H), 4.14 (s, 3H), 3.94 (s, 3H), 2.18 (s, 3H), 1.33 (s, 9H) |
| 46 | 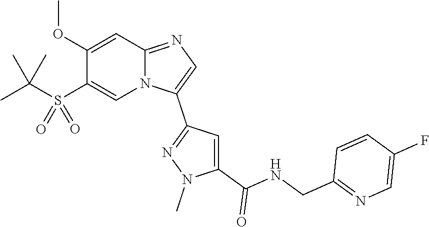 [SMILES: COc1cc2ncc(-c3cc(C(=O)NCc4ccc(F)cn4)n(C)n3)n2cc1S(=O)(=O)C(C)(C)C] | 3-(6-(tert- butylsulfonyl)-7- methoxyimidazo[1,2- a]pyridin-3-yl)-N-((5- fluoropyridin-2- yl)methyl)-1-methyl- 1H-pyrazole-5- carboxamide | [M + H]⁺ = 501.0 ¹H NMR (400 MHz, DMSO- d₆) δ ppm 9.86 (s, 1H), 9.30 (t, J = 6.0 Hz, 1H), 8.53 (d, J = 2.8 Hz, 1H), 7.98 (s, 1H), 7.75-7.72 (m, 1H), 7.48-7.44 (m, 1H), 7.36 (s, 1H), 7.29 (s, 1H), 4.56 (d, J = 5.6 Hz, 2H), 4.14 (s, 3H), 3.94 (s, 3H), 1.33 (s, 9H) |
| 47 | 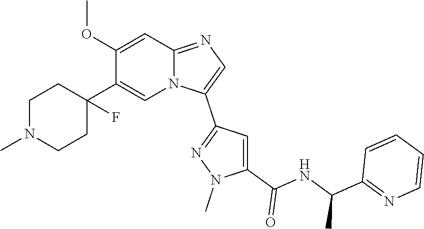 [SMILES: COc1cc2ncc(-c3cc(C(=O)N[C@H](C)c4ccccn4)n(C)n3)n2cc1C1(F)CCN(C)CC1] | (R)-3-(6-(4-fluoro-1- methylpiperidin-4-yl)- 7-methoxyimidazo[1,2- a]pyridin-3-yl)-1- methyl-N-(1-(pyridin- 2-yl)ethyl)-1H- pyrazole-5- carboxamide | [M + H]⁺ = 492.1 ¹H NMR (400 MHz, DMSO- d₆) δ ppm 9.36 (s, 1H), 9.00 (d, J = 7.6 Hz, 1H), 8.54 (d, J = 4.8 Hz, 1H), 7.85 (s, 1H), 7.78 (d, J = 7.6 Hz, 1H), 7.44 (d, J = 7.6 Hz, 1H), 7.41 (s, 1H), 7.30-7.25 (m, 1H), 7.15 (s, 1H), 5.18 (d, J = 7.6 Hz, 1H), 4.11 (s, 3H), 3.93 (s, 3H), 2.75-2.54 (m, 4H), 2.29-2.20 (m, 5H), 1.78 (t, J = 12.4 Hz, 2H), 1.52 (d, J = 7.2 Hz, 3H) |
| 48 | 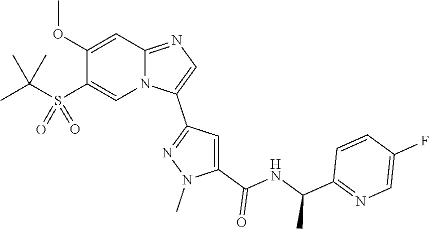 [SMILES: COc1cc2ncc(-c3cc(C(=O)N[C@H](C)c4ccc(F)cn4)n(C)n3)n2cc1S(=O)(=O)C(C)(C)C] | (R)-3-(6-(tert- butylsulfonyl)-7- methoxyimidazo[1,2- a]pyridin-3-yl)-N-(1- (5-fluoropyridin-2- yl)ethyl)-1-methyl-1H- pyrazole-5- carboxamide | [M + H]⁺ = 515.1 ¹H NMR (400 MHz, DMSO- d₆) δ ppm 9.87 (s, 1H), 9.06 (d, J = 7.6 Hz, 1H), 8.53 (d, J = 3.2 Hz, 1H), 8.00 (s, 1H), 7.71 (t, J = 3.2, 8.8 Hz, 1H), 7.52 (dd, J = 4.4, 8.8 Hz, 1H), 7.44 (s, 1H), 7.29 (s, 1H), 5.28-5.10 (m, 1H), 4.10 (s, 3H), 3.94 (s, 3H), 1.51 (d, J = 7.2 Hz, 3H), 1.33 (s, 9H) |
| 49 | 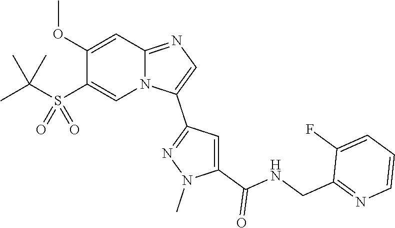 [SMILES: COc1cc2ncc(-c3cc(C(=O)NCc4ncccc4F)n(C)n3)n2cc1S(=O)(=O)C(C)(C)C] | 3-(6-(tert- butylsulfonyl)-7- methoxyimidazo[1,2- a]pyridin-3-yl)-N-((3- fluoropyridin-2- yl)methyl)-1-methyl- 1H-pyrazole-5- carboxamide | [M + H]⁺ = 501.0 ¹H NMR (400 MHz, DMSO- d₆) δ ppm 9.85 (s, 1H), 9.17 (t, J = 5.2 Hz, 1H), 8.40 (d, J = 4.8 Hz, 1H), 7.98 (s, 1H), 7.72 (t, J = 9.6 Hz, 1H), 7.44-7.41 (m, 1H), 7.35 (s, 1H), 7.29 (s, 1H), 4.65 ( d, J = 5.2 Hz, 2H), 4.13 (s, 3H), 3.94 (s, 3H), 1.33 (s, 9H) |
| 50 | 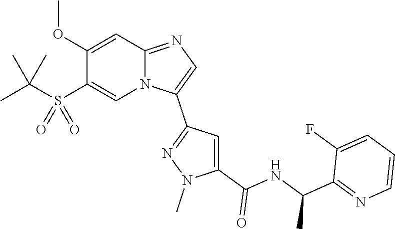 [SMILES: COc1cc2ncc(-c3cc(C(=O)N[C@H](C)c4ncccc4F)n(C)n3)n2cc1S(=O)(=O)C(C)(C)C] | (R)-3-(6-(tert- butylsulfonyl)-7- methoxyimidazo[1,2- a]pyridin-3-yl)-N-(1- (3-fluoropyridin-2- yl)ethyl)-1-methyl-1H- pyrazole-5- carboxamide | [M + H]⁺ = 515.0 ¹H NMR (400 MHz, DMSO- d₆) δ ppm 9.86 (s, 1H), 9.04 (d, J = 7.2 Hz, 1H), 8.42 (d, J = 4.4 Hz, 1H), 7.98 (s, 1H), 7.79-7.61 (m, 1H), 7.47-7.37 (m, 2H), 7.28 (s, 1H), 5.48- 5.44 (m, 1H), 4.08 (s, 3H), 3.94-3.89 (m, 3H), 1.51 (d, J = 6.8 Hz, 3H), 1.32 (s, 9H) SFC: Rₜ = 1.842 min, 99.6% ee; (S,S) Whelk-O 1 50 x 4.6 mm, 3.5 μm; 40% MeOH (0.05% DEA) in CO₂, 3 mL/min; 35° C., 100 Bar, 220 nm |
| 51 | 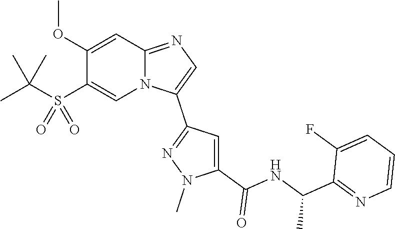 [SMILES: COc1cc2ncc(-c3cc(C(=O)N[C@@H](C)c4ncccc4F)n(C)n3)n2cc1S(=O)(=O)C(C)(C)C] | (S)-3-(6-(tert- butylsulfonyl)-7- methoxyimidazo[1,2- a]pyridin-3-yl)-N-(1- (3-fluoropyridin-2- yl)ethyl)-1-methyl-1H- pyrazole-5- carboxamide | [M + H]⁺ = 515.1 ¹H NMR (400 MHz, DMSO- d₆) δ ppm 9.86 (s, 1H), 9.04 (d, J = 7.6 Hz, 1H), 8.42 (d, J = 4.8 Hz, 1H), 7.98 (s, 1H), 7.76-7.67 (m, 1H), 7.47-7.37 (m, 2H), 7.28 (s, 1H), 5.52- 5.39 (m, 1H), 4.08 (s, 3H), 3.94 (s, 3H), 1.51 (d, J = 6.8 Hz, 3H), 1.32 (s, 9H) SFC: Rₜ = 2.189 min, 99.2% ee; (S,S) Whelk-O 1 50 x 4.6 mm, 3.5 μm; 40% MeOH (0.05% DEA) in CO₂, 3 mL/min; 35º C., 100 Bar, 220 nm |
| 52 | 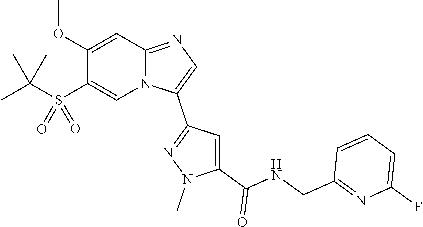 [SMILES: COc1cc2ncc(-c3cc(C(=O)NCc4cccc(F)n4)n(C)n3)n2cc1S(=O)(=O)C(C)(C)C] | 3-(6-(tert- butylsulfonyl)-7- methoxyimidazo[1,2- a]pyridin-3-yl)-N-((6- fluoropyridin-2- yl)methyl)-1-methyl- 1H-pyrazole-5- carboxamide | [M + H]⁺ = 501.1 ¹H NMR (400 MHz, DMSO- d₆) δ ppm 9.86 (s, 1H), 9.35 (t, J = 6.0 Hz, 1H), 8.02-7.94 (m, 2H), 7.38 (s, 1H), 7.35-7.32 (m, 1H), 7.29 (s, 1H), 7.09- 7.06(m, 1H), 4.51 (d, J = 5.6 Hz, 2H), 4.14 (s, 3H), 3.94 (s, 3H), 1.33 (s, 9H) |
| 53 | 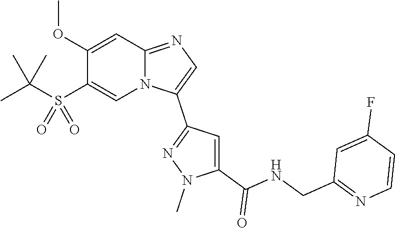 [SMILES: COc1cc2ncc(-c3cc(C(=O)NCc4cc(F)ccn4)n(C)n3)n2cc1S(=O)(=O)C(C)(C)C] | 3-(6-(tert- butylsulfonyl)-7- methoxyimidazo[1,2- a]pyridin-3-yl)-N-((4- fluoropyridin-2- yl)methyl)-1-methyl- 1H-pyrazole-5- carboxamide | [M + H]⁺ = 501.3 ¹H NMR (400 MHz, DMSO- d₆) δ ppm 9.86 (s, 1H), 9.31 (t, J = 6.0 Hz, 1H), 8.57 (dd, J = 8.8, 5.6 Hz, 1H), 8.00 (s, 1H), 7.38 (s, 1H), 7.30-7.21 (m, 3H), 4.59 (d, J = 6.0 Hz, 2H), 4.14 (s, 3H), 3.94 (s, 3H), 1.33 (s, 9H) |
| 54 | 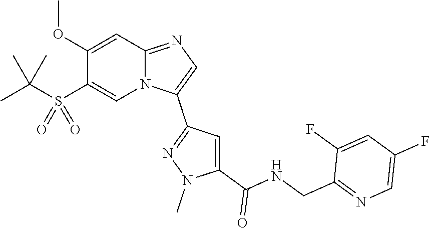 [SMILES: COc1cc2ncc(-c3cc(C(=O)NCc4ncc(F)cc4F)n(C)n3)n2cc1S(=O)(=O)C(C)(C)C] | 3-(6-(tert- butylsulfonyl)-7- methoxyimidazo[1,2- a]pyridin-3-yl)-N-((3,5- difluoropyridin-2- yl)methyl)-1-methyl- 1H-pyrazole-5- carboxamide | [M + H]⁺ = 519.3 ¹H NMR (400 MHz, DMSO- d₆) δ ppm 9.85 (s, 1H), 9.20 (t, J = 5.6 Hz, 1H), 8.50 (d, J = 2.4 Hz, 1H), 8.00-7.92 (m, 2H), 7.33 (s, 1H), 7.29 (s, 1H), 4.62 (d, J = 5.2 Hz, 2H), 4.12 (s, 3H), 3.94 (s, 3H), 1.33 (s, 9H) |
| 55 | 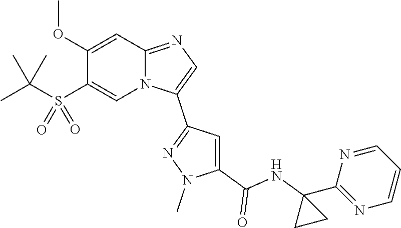 [SMILES: COc1cc2ncc(-c3cc(C(=O)NC4(c5ncccn5)CC4)n(C)n3)n2cc1S(=O)(=O)C(C)(C)C] | 3-(6-(tert- butylsulfonyl)-7- methoxyimidazo[1,2- a]pyridin-3-yl)-1- methyl-N-(1- (pyrimidin-2- yl)cyclopropyl)-1H- pyrazole-5- carboxamide | [M + H]⁺ = 510.1 ¹H NMR (400 MHz, DMSO- d₆) δ ppm 9.86 (s, 1H), 9.34 (s, 1H), 8.70 (d, J = 4.8 Hz, 2H), 7.99 (s, 1H), 7.38 (s, 1H), 7.31-7.28 (m, 2H), 4.12 (s, 3H), 3.95 (s, 3H), 1.65-1.60 (m, 2H), 1.39-1.35 (m, 2H), 1.33 (s, 9H) |
| 56 | 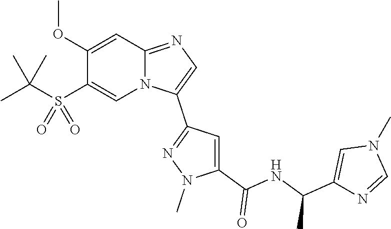 [SMILES: COc1cc2ncc(-c3cc(C(=O)N[C@H](C)c4cn(C)cn4)n(C)n3)n2cc1S(=O)(=O)C(C)(C)C] | (R)-3-(6-(tert- butylsulfonyl)-7- methoxyimidazo[1,2- a]pyridin-3-yl)-1- methyl-N-(1-(1-methyl- 1H-imidazol-4- yl)ethyl)-1H-pyrazole- 5-carboxamide | [M + H]⁺ = 500.0 ¹H NMR (400 MHz, DMSO- d₆) δ ppm 9.87 (s, 1H), 8.72 (d, J = 8.4 Hz, 1H), 7.95 (s, 1H), 7.50 (s, 1H), 7.37 (s, 1H), 7.28 (s, 1H), 6.99 (s, 1H), 5.15-5.02 (m, 1H), 4.13 (s, 3H), 3.94 (s, 3H), 3.61 (s, 3H), 1.45 (d, J = 7.2 Hz, 3H), 1.33 (s, 9H) SFC: Rₜ = 1.187 min, 100% ee; CHIRALPAK IC-3 50 x 4.6 mm, 3 μm; 50% i- PrOH/MeCN (7:3 + 0.05% DEA) in CO₂, 3 mL/min; 35º C., 100 Bar, 220 nm |
| 57 | 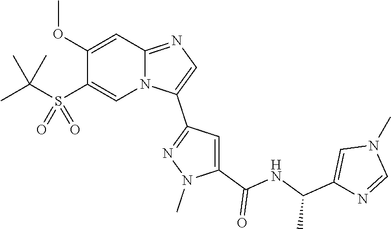 [SMILES: COc1cc2ncc(-c3cc(C(=O)N[C@@H](C)c4cn(C)cn4)n(C)n3)n2cc1S(=O)(=O)C(C)(C)C] | (S)-3-(6-(tert- butylsulfonyl)-7- methoxyimidazo[1,2- a]pyridin-3-yl)-1- methyl-N-(1-(1-methyl- 1H-imidazol-4- yl)ethyl)-1H-pyrazole- 5-carboxamide | [M + H]⁺ = 500.1 ¹H NMR (400 MHz, DMSO- d₆) δ ppm 9.87 (s, 1H), 8.72 (d, J = 8.4 Hz, 1H), 7.95 (s, 1H), 7.50 (s, 1H), 7.37 (s, 1H), 7.28 (s, 1H), 6.99 (s, 1H), 5.19- 5.01 (m, 1H), 4.13 (s, 3H), 3.94 (s, 3H), 3.61 (s, 3H), 1.45 (d, J = 6.8 Hz, 3H), 1.33 (s, 9H) SFC: Rₜ = 2.562 min, 99.4% ee; CHIRALPAK IC-3 50 x 4.6 mm, 3 μm; 50% i- PrOH/MeCN (7:3 + 0.05% DEA) in CO₂, 3 mL/min; 35º C., 100 Bar, 220 nm |
| 58 | 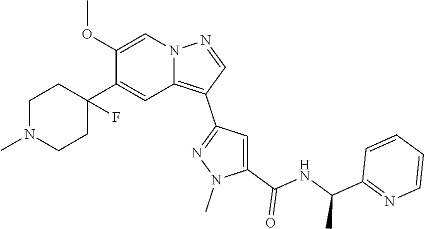 [SMILES: COc1cn2ncc(-c3cc(C(=O)N[C@H](C)c4ccccn4)n(C)n3)c2cc1C1(F)CCN(C)CC1] | (R)-3-(5-(4-fluoro-1- methylpiperidin-4-yl)- 6- methoxypyrazolo[1,5- a]pyridin-3-yl)-1- methyl-N-(1-(pyridin- 2-yl)ethyl)-1H- pyrazole-5- carboxamide | [M + H]⁺ = 492.1 ¹H NMR (400 MHz, DMSO- d₆) δ ppm 8.93 (d, J = 8.0 Hz, 1H), 8.54 (d, J = 4.8 Hz, 1H), 8.49 (s, 1H), 8.22 (s, 1H), 8.18 (s, 1H), 7.83-7.73 (m, 1H), 7.43 (d, J = 8.0 Hz, 1H), 7.32 (s, 1H), 7.27 (dd, J = 7.6, 4.8 Hz, 1H), 5.25-5.11 (m, 1H), 4.07 (s, 3H), 3.91 (s, 3H), 2.75-2.55 (m, 4H), 2.29-2.22 (m, 5H), 1.77-1.68 (m, 2H), 1.52 (d, J = 7.2 Hz, 3H) |
| 59 | 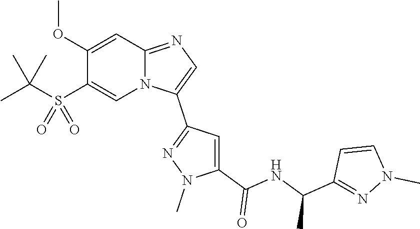 [SMILES: COc1cc2ncc(-c3cc(C(=O)N[C@H](C)c4ccn(C)n4)n(C)n3)n2cc1S(=O)(=O)C(C)(C)C] | (R)-3-(6-(tert- butylsulfonyl)-7- methoxyimidazo[1,2- a]pyridin-3-yl)-1- methyl-N-(1-(1-methyl- 1H-pyrazol-3-yl)ethyl)- 1H-pyrazole-5- carboxamide | [M + H]⁺ = 500.0 ¹H NMR (400 MHz, DMSO- d₆) δ ppm 9.87 (s, 1H), 8.86 (d, J = 8.4 Hz, 1H), 7.96 (s, 1H), 7.59 (d, J = 2.0 Hz, 1H), 7.36 (s, 1H), 7.28 (s, 1H), 6.18 (d, J = 2.0 Hz, 1H), 5.30-5.10 (m, 1H), 4.13 (s, 3H), 3.94 (s, 3H), 3.80 (s, 3H), 1.48 (d, J = 7.2 Hz, 3H), 1.33 (s, 9H) SFC: Rₜ = 2.394 min, 99.9% ee; CHIRALPAK IK-3 50 x 4.6 mm, 3 μm; 5-40% MeOH (0.05% DEA) in CO₂, 3 mL/min; 35° C., 100 Bar, 220 nm |
| 60 | 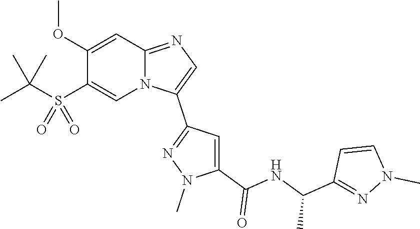 [SMILES: COc1cc2ncc(-c3cc(C(=O)N[C@@H](C)c4ccn(C)n4)n(C)n3)n2cc1S(=O)(=O)C(C)(C)C] | (S)-3-(6-(tert- butylsulfonyl)-7- methoxyimidazo[1,2- a]pyridin-3-yl)-1- methyl-N-(1-(1-methyl- 1H-pyrazol-3-yl)ethyl)- 1H-pyrazole-5- carboxamide | [M + H]⁺ = 500.1 ¹H NMR (400 MHz, DMSO- d₆) δ ppm 9.87 (s, 1H), 8.86 (d, J = 8.4 Hz, 1H), 7.96 (s, 1H), 7.59 (d, J = 2.0 Hz, 1H), 7.36 (s, 1H), 7.28 (s, 1H), 6.18 (d, J = 2.0 Hz, 1H), 5.29-5.13 (m, 1H), 4.13 (s, 3H), 3.94 (s, 3H), 3.80 (s, 3H), 1.48 (d, J = 6.8 Hz, 3H), 1.33 (s, 9H) SFC: Rₜ = 2.160 min, 99.4% ee; CHIRALPAK IK-3 50 x 4.6 mm, 3 μm; 5-40% MeOH (0.05% DEA) in CO₂, 3 mL/min; 35° C., 100 Bar, 220 nm |
| 61 | 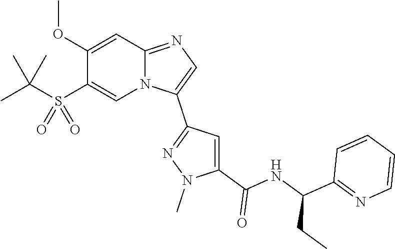 [SMILES: CC[C@@H](NC(=O)c1cc(-c2cnc3cc(OC)c(S(=O)(=O)C(C)(C)C)cn23)nn1C)c1ccccn1] | (R)-3-(6-(tert- butylsulfonyl)-7- methoxyimidazo[1,2- a]pyridin-3-yl)-1- methyl-N-(1-(pyridin- 2-yl)propyl)-1H- pyrazole-5- carboxamide | [M + H]⁺ = 511.1 ¹H NMR (400 MHz, DMSO- d₆) δ ppm 9.88 (s, 1H), 8.94 (d, J = 8.4 Hz, 1H), 8.56-8.52 (m, 1H), 8.01 (s, 1H), 7.82-7.74 (m, 1H), 7.48-7.42 (m, 2H), 7.30-7.26 (m, 2H), 5.04-4.95 (m, 1H), 4.10 (s, 3H), 3.94 (s, 3H), 2.01-1.93 (m, 1H), 1.89- 1.81 (m, 1H), 1.33 (s, 9H), 0.94 (t, J = 7.2 Hz, 3H) |
| 62 | 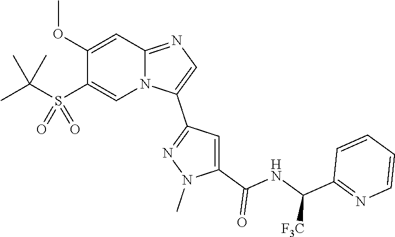 [SMILES: COc1cc2ncc(-c3cc(C(=O)N[C@@H](c4ccccn4)C(F)(F)F)n(C)n3)n2cc1S(=O)(=O)C(C)(C)C] | (S)-3-(6-(tert- butylsulfonyl)-7- methoxyimidazo[1,2- a]pyridin-3-yl)-1- methyl-N-(2,2,2- trifluoro-1-(pyridin-2- yl)ethyl)-1H-pyrazole- 5-carboxamide | [M + H]⁺ = 551.0 ¹H NMR (400 MHz, DMSO- d₆) δ ppm 9.87 (s, 1H), 9.64 (d, J = 9.2 Hz, 1H), 8.67 (d, J = 4.4 Hz, 1H), 8.04 (s, 1H), 7.97-7.92 (m, 1H), 7.80 (d, J = 8.0 Hz, 1H), 7.64 (s, 1H), 7.52-7.47 (m, 1H), 7.29 (s, 1H), 6.20-6.11 (m, 1H), 4.12 (s, 3H), 3.94 (s, 3H), 1.33 (s, 9H) |
| 63 | 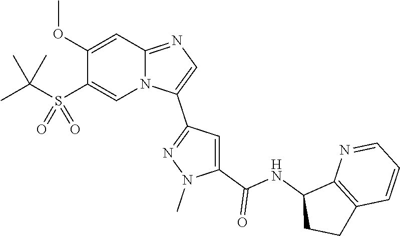 [SMILES: COc1cc2ncc(-c3cc(C(=O)N[C@@H]4CCc5cccnc54)n(C)n3)n2cc1S(=O)(=O)C(C)(C)C] | (R)-3-(6-(tert- butylsulfonyl)-7- methoxyimidazo[1,2- a]pyridin-3-yl)-N-(6,7- dihydro-5H- cyclopenta[b]pyridin-7- yl)-1-methyl-1H- pyrazole-5- carboxamide | [M + H]⁺ = 509.0 ¹H NMR (400 MHz, DMSO- d₆) δ ppm 9.88 (s, 1H), 8.98 (d, J = 8.4 Hz, 1H), 8.40 (d, J = 4.4 Hz, 1H), 7.94 (s, 1H), 7.70 (d, J = 7.6 Hz, 1H), 7.32-7.21 (m, 3H), 5.49 (q, J = 8.4 Hz, 1H), 4.18 (s, 3H), 3.94 (s, 3H), 3.03-2.86 (m, 2H), 2.58-2.53 (m, 1H), 2.06-1.94 (m, 1H), 1.33 (s, 9H) SFC: Rₜ = 1.598 min, 100% ee; CHIRALCEL OD-3 50 x 4.6 mm, 3 μm; 5-40% EtOH (0.05% DEA) in CO₂, 3 mL/min; 35° C., 100 Bar, 220 nm |
| 64 | 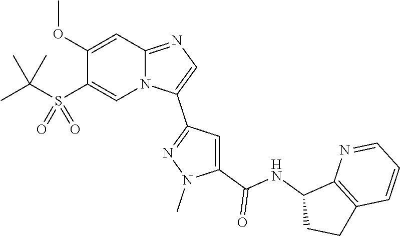 [SMILES: COc1cc2ncc(-c3cc(C(=O)N[C@H]4CCc5cccnc54)n(C)n3)n2cc1S(=O)(=O)C(C)(C)C] | (S)-3-(6-(tert- butylsulfonyl)-7- methoxyimidazo[1,2- a]pyridin-3-yl)-N-(6,7- dihydro-5H- cyclopenta[b]pyridin-7- yl)-1-methyl-1H- pyrazole-5- carboxamide | [M + H]⁺ = 509.2 ¹H NMR (400 MHz, DMSO- d₆) δ ppm 9.88 (s, 1H), 8.98 (d, J = 8.4 Hz, 1H), 8.40 (d, J = 4.8 Hz, 1H), 7.94 (s, 1H), 7.70 (d, J = 7.6 Hz, 1H), 7.35-7.19 (m, 3H), 5.49 (q, J = 8.4 Hz, 1H), 4.18 (s, 3H), 3.94 (s, 3H), 3.04-2.85 (m, 2H), 2.54 (d, J = 4.0 Hz, 1H), 1.99 (q, J = 8.8, 12.4 Hz, 1H), 1.33 (s, 9H) SFC: Rₜ = 1.797 min, 98.8% ee; CHIRALCEL OD-3 50 x 4.6 mm, 3 μm; 5-40% EtOH (0.05% DEA) in CO₂, 3 mL/min; 35° C., 100 Bar, 220 nm |
| 65 | 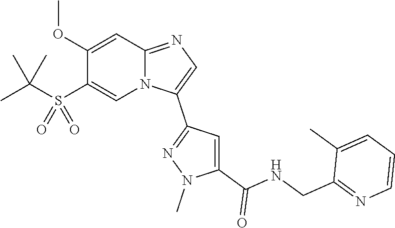 [SMILES: COc1cc2ncc(-c3cc(C(=O)NCc4ncccc4C)n(C)n3)n2cc1S(=O)(=O)C(C)(C)C] | 3-(6-(tert- butylsulfonyl)-7- methoxyimidazo[1,2- a]pyridin-3-yl)-1- methyl-N-((3- methylpyridin-2- yl)methyl)-1H- pyrazole-5- carboxamide | [M + H]⁺ = 497.0 ¹H NMR (400 MHz, DMSO- d₆) δ ppm 9.86 (s, 1H), 9.01 (t, J = 5.2 Hz, 1H), 8.37 (d, J = 3.6 Hz, 1H), 7.99 (s, 1H), 7.60 (d, J = 7.2 Hz, 1H), 7.38 (s, 1H), 7.29 (s, 1H), 7.26-7.21 (m, 1H), 4.59 (d, J = 5.6 Hz, 2H), 4.14 (s, 3H), 3.94 (s, 3H), 2.35 (s, 3H), 1.33 (s, 9H) |
| 66 | 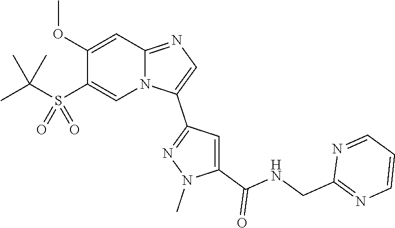 [SMILES: COc1cc2ncc(-c3cc(C(=O)NCc4ncccn4)n(C)n3)n2cc1S(=O)(=O)C(C)(C)C] | 3-(6-(tert- butylsulfonyl)-7- methoxyimidazo[1,2- a]pyridin-3-yl)-1- methyl-N-(pyrimidin-2- ylmethyl)-1H-pyrazole- 5-carboxamide | [M + H]⁺ = 484.0 ¹H NMR (400 MHz, DMSO- d₆) δ ppm 9.86 (s, 1H), 9.26 (t, J = 6.4 Hz, 1H), 8.79 (d, J = 4.8 Hz, 2H), 8.00 (s, 1H), 7.42 (t, J = 4.8 Hz, 1H), 7.38 (s, 1H), 7.30 (s, 1H), 4.66 (d, J = 6.0 Hz, 2H), 4.13 (s, 3H), 3.94 (s, 3H), 1.33 (s, 9H) |
| 67 | 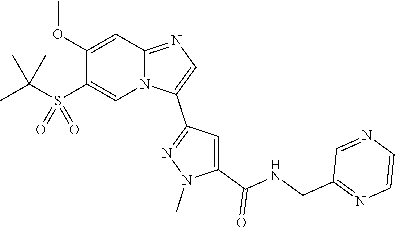 [SMILES: COc1cc2ncc(-c3cc(C(=O)NCc4cnccn4)n(C)n3)n2cc1S(=O)(=O)C(C)(C)C] | 3-(6-(tert- butylsulfonyl)-7- methoxyimidazo[1,2- a]pyridin-3-yl)-1- methyl-N-(pyrazin-2- ylmethyl)-1H-pyrazole- 5-carboxamide | [M + H]⁺ = 484.0 ¹H NMR (400 MHz, DMSO- d₆) δ ppm 9.86 (s, 1H), 9.36 (t, J = 5.6 Hz, 1H), 8.69 (s, 1H), 8.62 (s, 1H), 8.57 (d, J = 2.4 Hz, 1H), 7.99 (s, 1H), 7.36 (s, 1H), 7.29 (s, 1H), 4.63 (d, J = 5.6 Hz, 2H), 4.13 (s, 3H), 3.94 (s, 3H), 1.33 (s, 9H) |
| 68 | 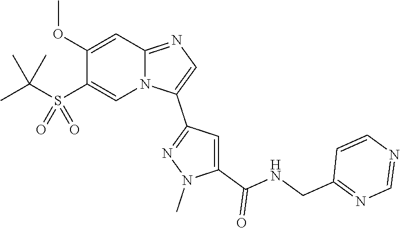 [SMILES: COc1cc2ncc(-c3cc(C(=O)NCc4ccncn4)n(C)n3)n2cc1S(=O)(=O)C(C)(C)C] | 3-(6-(tert- butylsulfonyl)-7- methoxyimidazo[1,2- a]pyridin-3-yl)-1- methyl-N-(pyrimidin-4- ylmethyl)-1H-pyrazole- 5-carboxamide | [M + H]⁺ = 484.1 ¹H NMR (400 MHz, DMSO- d₆) δ ppm 9.86 (s, 1H), 9.36 (t, J = 6.0 Hz, 1H), 9.14 (d, J = 1.2 Hz, 1H), 8.76 (d, J = 5.2 Hz, 1H), 8.00 (s, 1H), 7.50 (dd, J = 0.8, 5.2 Hz, 1H), 7.39 (s, 1H), 7.30 (s, 1H), 4.56 (d, J = 6.0 Hz, 2H), 4.14 (s, 3H), 3.94 (s, 3H), 1.33 (s, 9H) |
| 69 | 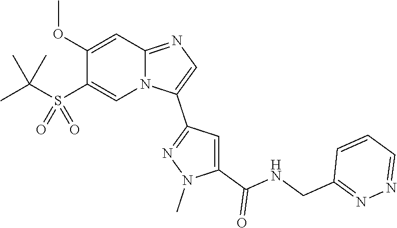 [SMILES: COc1cc2ncc(-c3cc(C(=O)NCc4cccnn4)n(C)n3)n2cc1S(=O)(=O)C(C)(C)C] | 3-(6-(tert- butylsulfonyl)-7- methoxyimidazo[1,2- a]pyridin-3-yl)-1- methyl-N-(pyridazin-3- ylmethyl)-1H-pyrazole- 5-carboxamide | [M + H]⁺ = 484.1 ¹H NMR (400 MHz, DMSO- d₆) δ ppm 9.86 (s, 1H), 9.41 (t, J = 6.0 Hz, 1H), 9.19-9.10 (m, 1H), 7.98 (s, 1H), 7.72-7.64 (m, 2H), 7.37 (s, 1H), 7.29 (s, 1H), 4.77 (d, J = 5.6 Hz, 2H), 4.14 (s, 3H), 3.94 (s, 3H), 1.33 (s, 9H) |
| 70 | 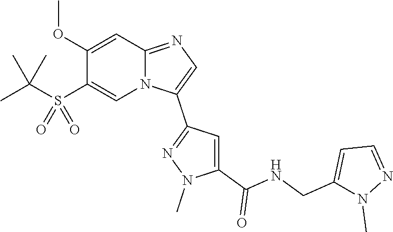 [SMILES: COc1cc2ncc(-c3cc(C(=O)NCc4ccnn4C)n(C)n3)n2cc1S(=O)(=O)C(C)(C)C] | 3-(6-(tert- butylsulfonyl)-7- methoxyimidazo[1,2- a]pyridin-3-yl)-1- methyl-N-((1-methyl- 1H-pyrazol-5- yl)methyl)-1H- pyrazole-5- carboxamide | [M + H]⁺ = 486.0 ¹H NMR (400 MHz, DMSO- d₆) δ ppm 9.86 (s, 1H), 9.18 (t, J = 6.0 Hz, 1H), 7.98 (s, 1H), 7.33 (s, 1H), 7.31 (s, 1H), 7.29 (s, 1H), 6.21 (s, 1H), 4.52 (d, J = 5.6 Hz, 2H), 4.14 (s, 3H), 3.94 (s, 3H), 3.83 (s, 3H), 1.32 (s, 9H) |
| 71 | 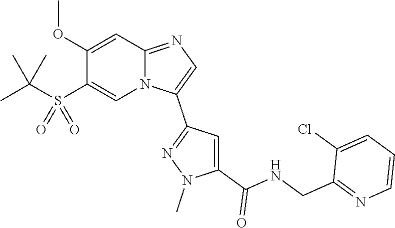 [SMILES: COc1cc2ncc(-c3cc(C(=O)NCc4ncccc4Cl)n(C)n3)n2cc1S(=O)(=O)C(C)(C)C] | 3-(6-(tert- butylsulfonyl)-7- methoxyimidazo[1,2- a]pyridin-3-yl)-N-((3- chloropyridin-2- yl)methyl)-1-methyl- 1H-pyrazole-5- carboxamide | [M + H]⁺ = 517.0 ¹H NMR (400 MHz, DMSO- d₆) δ ppm 9.86 (s, 1H), 9.13 (t, J = 5.6 Hz, 1H), 8.52 (d, J = 4.8 Hz, 1H), 8.00 (s, 1H), 7.96 (d, J = 8.0 Hz, 1H), 7.44-7.37 (m, 2H), 7.29 (s, 1H), 4.69 (d, J = 5.6 Hz, 2H), 4.13 (s, 3H), 3.94 (s, 3H), 1.33 (s, 9H) |
| 72 | 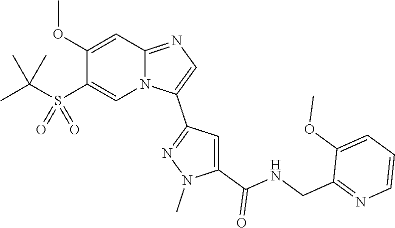 [SMILES: COc1cc2ncc(-c3cc(C(=O)NCc4ncccc4OC)n(C)n3)n2cc1S(=O)(=O)C(C)(C)C] | 3-(6-(tert- butylsulfonyl)-7- methoxyimidazo[1,2- a]pyridin-3-yl)-N-((3- methoxypyridin-2- yl)methyl)-1-methyl- 1H-pyrazole-5- carboxamide | [M + H]⁺ = 513.0 ¹H NMR (400 MHz, DMSO- d₆) δ ppm 9.87 (s, 1H), 8.90 (t, J = 4.8 Hz, 1H), 8.11 (d, J = 3.6 Hz, 1H), 7.99 (s, 1H), 7.44 (d, J = 8.4 Hz, 1H), 7.37 (s, 1H), 7.35-7.30 (m, 1H), 7.29 (s, 1H), 4.57 (d, J = 5.2 Hz, 2H), 4.14 (s, 3H), 3.94 (s, 3H), 3.87 (s, 3H), 1.33 (s, 9H) |
| 73 | 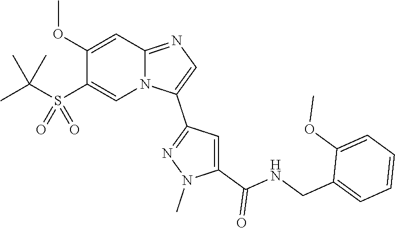 [SMILES: COc1ccccc1CNC(=O)c1cc(-c2cnc3cc(OC)c(S(=O)(=O)C(C)(C)C)cn23)nn1C] | 3-(6-(tert- butylsulfonyl)-7- methoxyimidazo[1,2- a]pyridin-3-yl)-N-(2- methoxybenzyl)-1- methyl-1H-pyrazole-5- carboxamide | [M + H]⁺ = 512.2 ¹H NMR (400 MHz, DMSO- d₆) δ ppm 9.87 (s, 1H), 9.05 (t, J = 6.0 Hz, 1H), 7.98 (s, 1H), 7.37 (s, 1H), 7.31-7.21 (m, 3H), 7.01 (d, J = 8.0 Hz, 1H), 6.96-6.89 (m, 1H), 4.44 (d, J = 6.0 Hz, 2H), 4.14 (s, 3H), 3.94 (s, 3H), 3.83 (s, 3H), 1.33 (s, 9H) |
| 74 | 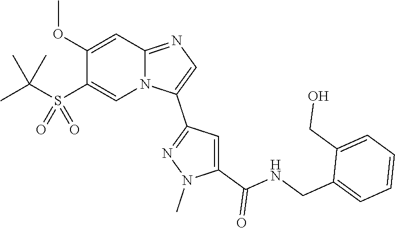 [SMILES: COc1cc2ncc(-c3cc(C(=O)NCc4ccccc4CO)n(C)n3)n2cc1S(=O)(=O)C(C)(C)C] | 3-(6-(tert- butylsulfonyl)-7- methoxyimidazo[1,2- a]pyridin-3-yl)-N-(2- (hydroxymethyl) benzyl)-1-methyl-1H- pyrazole-5- carboxamide | [M + H]⁺ = 512.1 ¹H NMR (400 MHz, DMSO- d₆) δ ppm 9.86 (s, 1H), 9.09 (t, J = 5.6 Hz, 1H), 7.97 (s, 1H), 7.44-7.39 (m, 1H), 7.35-7.24 (m, 5H), 5.22 (t, J = 4.8 Hz, 1H), 4.63 (d, J = 4.4 Hz, 2H), 4.52 (d, J = 5.6 Hz, 2H), 4.14 (s, 3H), 3.93 (s, 3H), 1.32 (s, 9H) |
| 75 | 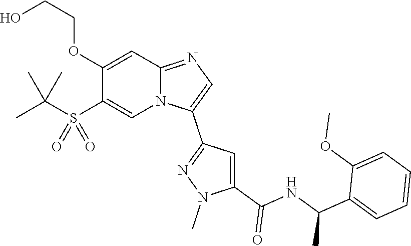 [SMILES: COc1ccccc1[C@@H](C)NC(=O)c1cc(-c2cnc3cc(OCCO)c(S(=O)(=O)C(C)(C)C)cn23)nn1C] | (R)-3-(6-(tert- butylsulfonyl)-7-(2- hydroxyethoxy)imidazo [1,2-a]pyridin-3-yl)-N- (1-(2- methoxyphenyl)ethyl)- 1-methyl-1H-pyrazole- 5-carboxamide | [M + H]⁺ = 556.3 ¹H NMR (400 MHz, DMSO- d₆) δ ppm 9.88 (s, 1H), 8.96 (d, J = 8.0 Hz, 1H), 8.02 (s, 1H), 7.44 (s, 1H), 7.37 (dd, J = 6.0, 1.6 Hz, 1H), 7.30 (s, 1H), 7.25-7.20 (m, 1H), 6.99 (d, J = 7.6 Hz, 1H), 6.93 (t, J = 7.2 Hz, 1H), 5.43 (t, J = 7.2 Hz, 1H), 4.83 (t, J = 5.2 Hz, 1H), 4.19 (t, J = 4.8 Hz, 2H), 4.08 (s, 3H), 3.84 (s, 3H), 3.80-3.75 (m, 2H), 1.40 (d, J = 7.6 Hz, 3H), 1.34 (s, 9H) |
| 76 | 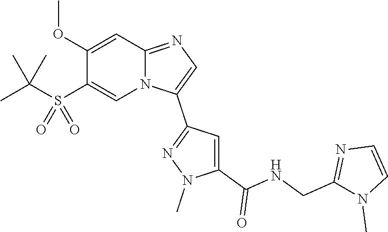 [SMILES: COc1cc2ncc(-c3cc(C(=O)NCc4nccn4C)n(C)n3)n2cc1S(=O)(=O)C(C)(C)C] | 3-(6-(tert- butylsulfonyl)-7- methoxyimidazo[1,2- a]pyridin-3-yl)-1- methyl-N-((1-methyl- 1H-imidazol-2- yl)methyl)-1H- pyrazole-5- carboxamide | [M + H]⁺ = 486.1 ¹H NMR (400 MHz, DMSO- d₆) δ ppm 9.85 (s, 1H), 9.15 (t, J = 5.6 Hz, 1H), 7.95 (s, 1H), 7.35 (s, 1H), 7.29 (s, 1H), 7.11 (d, J = 0.8 Hz, 1H), 6.81 (d, J = 1.2 Hz, 1H), 4.52 (d, J = 5.6 Hz, 2H), 4.14 (s, 3H), 3.93 (s, 3H), 3.66 (s, 3H), 1.32 (s, 9H) |
| 77 | 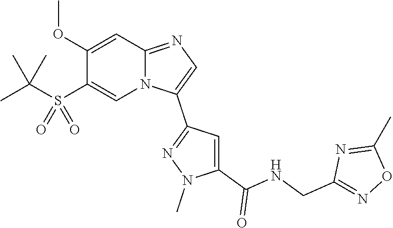 [SMILES: COc1cc2ncc(-c3cc(C(=O)NCc4noc(C)n4)n(C)n3)n2cc1S(=O)(=O)C(C)(C)C] | 3-(6-(tert- butylsulfonyl)-7- methoxyimidazo[1,2- a]pyridin-3-yl)-1- methyl-N-((5-methyl- 1,2,4-oxadiazol-3- yl)methyl)-1H- pyrazole-5- carboxamide | [M + H]⁺ = 488.1 ¹H NMR (400 MHz, DMSO- d₆) δ ppm 9.85 (s, 1H), 9.37- 9.29 (m, 1H), 7.99 (s, 1H), 7.33 (s, 1H), 7.29 (s, 1H), 4.55 (d, J = 5.6 Hz, 2H), 4.13 (s, 3H), 3.94 (s, 3H), 2.59 (s, 3H), 1.33 (s, 9H) |
| 78 | 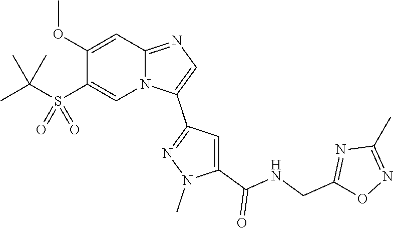 [SMILES: COc1cc2ncc(-c3cc(C(=O)NCc4nc(C)no4)n(C)n3)n2cc1S(=O)(=O)C(C)(C)C] | 3-(6-(tert- butylsulfonyl)-7- methoxyimidazo[1,2- a]pyridin-3-yl)-1- methyl-N-((3-methyl- 1,2,4-oxadiazol-5- yl)methyl)-1H- pyrazole-5- carboxamide | [M + H]⁺ = 488.2 ¹H NMR (400 MHz, DMSO- d₆) δ ppm 9.84 (s, 1H), 9.52 (t, J = 5.6 Hz, 1H), 8.01 (s, 1H), 7.36 (s, 1H), 7.30 (s, 1H), 4.73 (d, J = 5.6 Hz, 2H), 4.13 (s, 3H), 3.94 (s, 3H), 2.34 (s, 3H), 1.33 (s, 9H) |
| 79 | 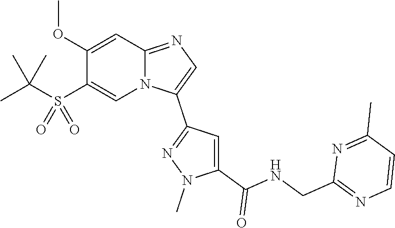 [SMILES: COc1cc2ncc(-c3cc(C(=O)NCc4nccc(C)n4)n(C)n3)n2cc1S(=O)(=O)C(C)(C)C] | 3-(6-(tert- butylsulfonyl)-7- methoxyimidazo[1,2- a]pyridin-3-yl)-1- methyl-N-((4- methylpyrimidin-2- yl)methyl)-1H- pyrazole-5- carboxamide | [M + H]⁺ = 498.3 ¹H NMR (400 MHz, DMSO- d₆) δ ppm 9.86 (s, 1H), 9.21 (t, J = 6.0 Hz, 1H), 8.62 (d, J = 5.2 Hz, 1H), 8.00 (s, 1H), 7.37 (s, 1H), 7.30-7.27 (m, 2H), 4.60 (d, J = 6.0 Hz, 2H), 4.13 (s, 3H), 3.94 (s, 3H), 2.46 (s, 3H), 1.33 (s, 9H) |
| 80 | 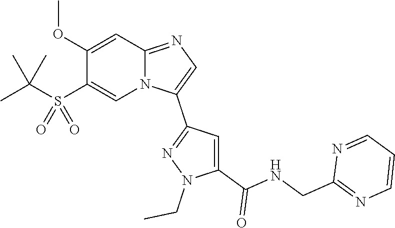 [SMILES: CCn1nc(-c2cnc3cc(OC)c(S(=O)(=O)C(C)(C)C)cn23)cc1C(=O)NCc1ncccn1] | 3-(6-(tert- butylsulfonyl)-7- methoxyimidazo[1,2- a]pyridin-3-yl)-1-ethyl- N-(pyrimidin-2- ylmethyl)-1H-pyrazole- 5-carboxamide | [M + H]⁺ = 498.1 ¹H NMR (400 MHz, DMSO- d₆) δ ppm 9.95 (s, 1H), 9.25 (t, J = 6.0 Hz, 1H), 8.79 (d, J = 5.2 Hz, 2H), 8.01 (s, 1H), 7.42 (t, J = 4.8 Hz, 1H), 7.34 (s, 1H), 7.29 (s, 1H), 4.66 (d, J = 6.0 Hz, 2H), 4.56 (q, J = 7.2 Hz, 2H), 3.94 (s, 3H), 1.42 (t, J = 7.2 Hz, 3H), 1.33 (s, 9H) |
| 81 | 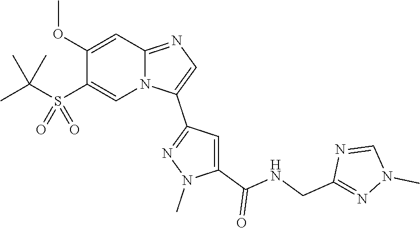 [SMILES: COc1cc2ncc(-c3cc(C(=O)NCc4ncn(C)n4)n(C)n3)n2cc1S(=O)(=O)C(C)(C)C] | 3-(6-(tert- butylsulfonyl)-7- methoxyimidazo[1,2- a]pyridin-3-yl)-1- methyl-N-((1-methyl- 1H-1,2,4-triazol-3- yl)methyl)-1H- pyrazole-5- carboxamide | [M + H]⁺ = 487.1 ¹H NMR (400 MHz, DMSO- d₆) δ ppm 9.85 (s, 1H), 9.16 (t, J = 5.6 Hz, 1H), 8.39 (s, 1H), 7.96 (s, 1H), 7.34 (s, 1H), 7.29 (s, 1H), 4.47 (d, J = 5.6 Hz, 2H), 4.14 (s, 3H), 3.94 (s, 3H), 3.83 (s, 3H), 1.33 (s, 9H) |
| 82 | 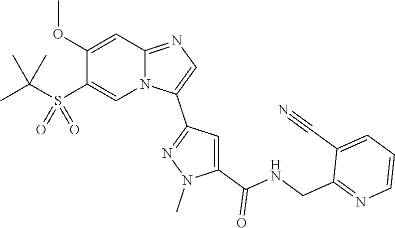 [SMILES: COc1cc2ncc(-c3cc(C(=O)NCc4ncccc4C#N)n(C)n3)n2cc1S(=O)(=O)C(C)(C)C] | 3-(6-(tert- butylsulfonyl)-7- methoxyimidazo[1,2- a]pyridin-3-yl)-N-((3- cyanopyridin-2- yl)methyl)-1-methyl- 1H-pyrazole-5- carboxamide | [M + H]⁺ = 508.0 |
| 83 | 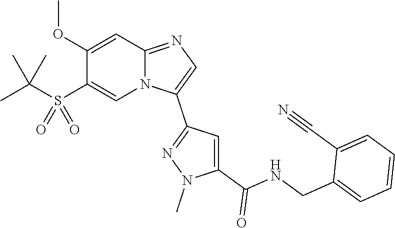 [SMILES: COc1cc2ncc(-c3cc(C(=O)NCc4ccccc4C#N)n(C)n3)n2cc1S(=O)(=O)C(C)(C)C] | 3-(6-(tert- butylsulfonyl)-7- methoxyimidazo[1,2- a]pyridin-3-yl)-N-(2- cyanobenzyl)-1- methyl-1H-pyrazole-5- carboxamide | [M + H]⁺ = 507.1 ¹H NMR (400 MHz, DMSO- d₆) δ ppm 9.86 (s, 1H), 9.38 (s, 1H), 7.98 (s, 1H), 7.86 (d, J = 7.2 Hz, 1H), 7.74-7.69 (m, 1H), 7.60-7.56 (m, 1H), 7.52- 7.47 (m, 1H), 7.35 (s, 1H), 7.29 (s, 1H), 4.65 (d, J = 5.6 Hz, 2H), 4.14 (s, 3H), 3.94 (s, 3H), 1.33 (s, 9H) |
| 84 | 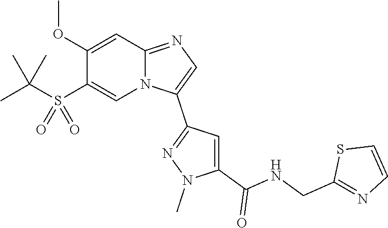 [SMILES: COc1cc2ncc(-c3cc(C(=O)NCc4nccs4)n(C)n3)n2cc1S(=O)(=O)C(C)(C)C] | 3-(6-(tert- butylsulfonyl)-7- methoxyimidazo[1,2- a]pyridin-3-yl)-1- methyl-N-(thiazol-2- ylmethyl)-1H-pyrazole- 5-carboxamide | [M + H]⁺ = 489.0 ¹H NMR (400 MHz, DMSO- d₆) δ ppm 9.85 (s, 1H), 9.58 (t, J = 5.6 Hz, 1H), 8.00 (s, 1H), 7.76 (d, J = 3.6 Hz, 1H), 7.67 (d, J = 3.2 Hz, 1H), 7.34 (s, 1H), 7.29 (s, 1H), 4.76 (d, J = 6.0 Hz, 2H), 4.15 (s, 3H), 3.94 (s, 3H), 1.33 (s, 9H) |
| 85 | 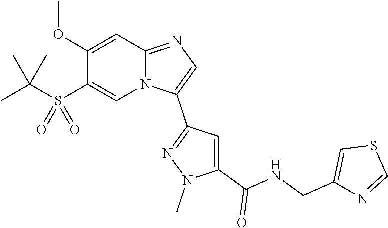 [SMILES: COc1cc2ncc(-c3cc(C(=O)NCc4cscn4)n(C)n3)n2cc1S(=O)(=O)C(C)(C)C] | 3-(6-(tert- butylsulfonyl)-7- methoxyimidazo[1,2- a]pyridin-3-yl)-1- methyl-N-(thiazol-4- ylmethyl)-1H-pyrazole- 5-carboxamide | [M + H]⁺ = 489.0 ¹H NMR (400 MHz, DMSO- d₆) δ ppm 9.86 (s, 1H), 9.25 (t, J = 6.0 Hz, 1H), 9.08 (d, J = 2.0 Hz, 1H), 7.97 (s, 1H), 7.54 (s, 1H), 7.35 (s, 1H), 7.29 (s, 1H), 4.61 (d, J = 6.0 Hz, 2H), 4.15 (s, 3H), 3.94 (s, 3H), 1.33 (s, 9H) |
| 86 | 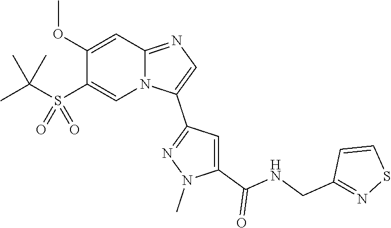 [SMILES: COc1cc2ncc(-c3cc(C(=O)NCc4ccsn4)n(C)n3)n2cc1S(=O)(=O)C(C)(C)C] | 3-(6-(tert- butylsulfonyl)-7- methoxyimidazo[1,2- a]pyridin-3-yl)-N- (isothiazol-3-ylmethyl)- 1-methyl-1H-pyrazole- 5-carboxamide | [M + H]⁺ = 489.1 ¹H NMR (400 MHz, DMSO- d₆) δ ppm 9.86 (s, 1H), 9.45- 9.24 (m, 1H), 9.04 (d, J = 4.8 Hz, 1H), 7.98 (s, 1H), 7.34 (t, J = 2.8 Hz, 2H), 7.29 (s, 1H), 4.62 (d, J = 6.0 Hz, 2H), 4.15 (s, 3H), 3.94 (s, 3H), 1.33 (s, 9H) |
| 87 | 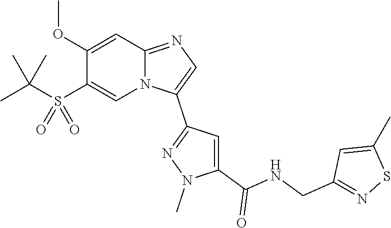 [SMILES: COc1cc2ncc(-c3cc(C(=O)NCc4cc(C)sn4)n(C)n3)n2cc1S(=O)(=O)C(C)(C)C] | 3-(6-(tert- butylsulfonyl)-7- methoxyimidazo[1,2- a]pyridin-3-yl)-1- methyl-N-((5- methylisothiazol-3- yl)methyl)-1H- pyrazole-5- carboxamide | [M + H]⁺ = 503.0 ¹H NMR (400 MHz, DMSO- d₆) δ ppm 9.85 (s, 1H), 9.29 (t, J = 5.6 Hz, 1H), 7.98 (s, 1H), 7.34 (s, 1H), 7.29 (s, 1H), 7.08 (d, J = 0.8 Hz, 1H), 4.51 (d, J = 6.0 Hz, 2H), 4.15 (s, 3H), 3.94 (s, 3H), 2.54 (d, J = 0.8 Hz, 3H), 1.33 (s, 9H) |
| 88 | 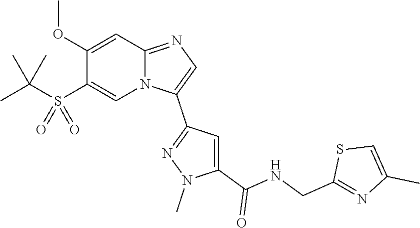 [SMILES: COc1cc2ncc(-c3cc(C(=O)NCc4nc(C)cs4)n(C)n3)n2cc1S(=O)(=O)C(C)(C)C] | 3-(6-(tert- butylsulfonyl)-7- methoxyimidazo[1,2- a]pyridin-3-yl)-1- methyl-N-((4- methylthiazol-2- yl)methyl)-1H- pyrazole-5- carboxamide | [M + H]⁺ = 503.2 ¹H NMR (500 MHz, DMSO- d₆) δ ppm 9.85 (s, 1H), 9.54 (t, J = 6.0 Hz, 1H), 7.99 (s, 1H), 7.34 (s, 1H), 7.29 (s, 1H), 7.18 (d, J = 0.8 Hz, 1H), 4.70 (d, J = 6.0 Hz, 2H), 4.15 (s, 3H), 3.94 (s, 3H), 2.35 (d, J = 0.8 Hz, 3H), 1.33 (s, 9H) |
| 89 | 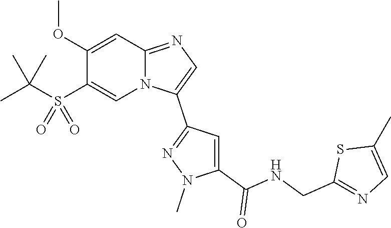 [SMILES: COc1cc2ncc(-c3cc(C(=O)NCc4ncc(C)s4)n(C)n3)n2cc1S(=O)(=O)C(C)(C)C] | 3-(6-(tert- butylsulfonyl)-7- methoxyimidazo[1,2- a]pyridin-3-yl)-1- methyl-N-((5- methylthiazol-2- yl)methyl)-1H- pyrazole-5- carboxamide | [M + H]⁺ = 503.3 ¹H NMR (400 MHz, DMSO- d₆) δ ppm 9.85 (s, 1H), 9.53 (t, J = 5.6 Hz, 1H), 7.99 (s, 1H), 7.40 (d, J = 1.2 Hz, 1H), 7.33 (s, 1H), 7.29 (s, 1H), 4.66 (d, J = 6.0 Hz, 2H), 4.15 (s, 3H), 3.94 (s, 3H), 2.40 (s, 3H), 1.33 (s, 9H) |
| 90 | 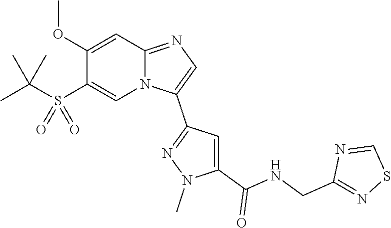 [SMILES: COc1cc2ncc(-c3cc(C(=O)NCc4ncsn4)n(C)n3)n2cc1S(=O)(=O)C(C)(C)C] | N-((1,2,4-thiadiazol-3- yl)methyl)-3-(6-(tert- butylsulfonyl)-7- methoxyimidazo[1,2- a]pyridin-3-yl)-1- methyl-1H-pyrazole-5- carboxamide | [M + H]⁺ = 490.3 ¹H NMR (400 MHz, DMSO- d₆) δ ppm 10.27 (s, 1H), 9.86- 9.84 (m, 1H), 9.41 (t, J = 6.0 Hz, 1H), 7.99 (s, 1H), 7.35 (s, 1H), 7.29 (s, 1H), 4.80-4.76 (m, 2H), 4.13 (s, 3H), 3.94 (s, 3H), 1.33 (s, 9H) |
| 91 | 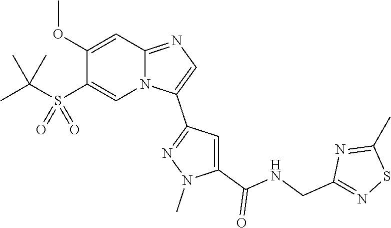 [SMILES: COc1cc2ncc(-c3cc(C(=O)NCc4nsc(C)n4)n(C)n3)n2cc1S(=O)(=O)C(C)(C)C] | 3-(6-(tert- butylsulfonyl)-7- methoxyimidazo[1,2- a]pyridin-3-yl)-1- methyl-N-((5-methyl- 1,2,4-thiadiazol-3- yl)methyl)-1H- pyrazole-5- carboxamide | [M + H]⁺ = 504.2 ¹H NMR (400 MHz, DMSO- d₆) δ ppm 9.85 (s, 1H), 9.37 (t, J = 5.6 Hz, 1H), 7.99 (s, 1H), 7.36 (s, 1H), 7.29 (s, 1H), 4.68 (d, J = 6.0 Hz, 2H), 4.14 (s, 3H), 3.94 (s, 3H), 2.79 (s, 3H), 1.33 (s, 9H) |
| 92 | 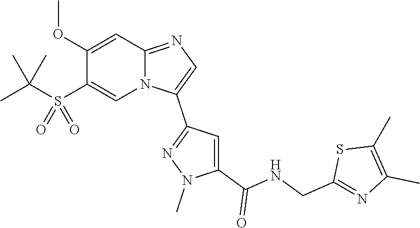 [SMILES: COc1cc2ncc(-c3cc(C(=O)NCc4nc(C)c(C)s4)n(C)n3)n2cc1S(=O)(=O)C(C)(C)C] | 3-(6-(tert- butylsulfonyl)-7- methoxyimidazo[1,2- a]pyridin-3-yl)-N-((4,5- dimethylthiazol-2- yl)methyl)-1-methyl- 1H-pyrazole-5- carboxamide | [M + H]⁺ = 517.0 ¹H NMR (400 MHz, DMSO- d₆) δ ppm 9.85 (s, 1H), 9.49 (t, J = 6.0 Hz, 1H), 7.99 (s, 1H), 7.32 (s, 1H), 7.29 (s, 1H), 4.61 (d, J = 6.0 Hz, 2H), 4.14 (s, 3H), 3.94 (s, 3H), 2.29 (s, 3H), 2.23 (s, 3H), 1.33 (s, 9H) |
| 93 | 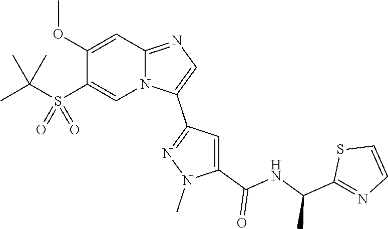 [SMILES: COc1cc2ncc(-c3cc(C(=O)N[C@H](C)c4nccs4)n(C)n3)n2cc1S(=O)(=O)C(C)(C)C] | (R)-3-(6-(tert- butylsulfonyl)-7- methoxyimidazo[1,2- a]pyridin-3-yl)-1- methyl-N-(1-(thiazol-2- yl)ethyl)-1H-pyrazole- 5-carboxamide | [M + H]⁺ = 503.1 ¹H NMR (400 MHz, DMSO- d₆) δ ppm 9.88 (s, 1H), 9.38 (d, J = 8.0 Hz, 1H), 8.01 (s, 1H), 7.76 (d, J = 3.2 Hz, 1H), 7.65 (d, J = 3.2 Hz, 1H), 7.40 (s, 1H), 7.29 (s, 1H), 5.56-5.40 (m, 1H), 4.14 (s, 3H), 3.94 (s, 3H), 1.63 (d, J = 7.2 Hz, 3H), 1.33 (s, 9H) |
| 94 | 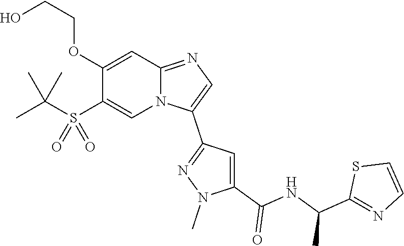 [SMILES: C[C@@H](NC(=O)c1cc(-c2cnc3cc(OCCO)c(S(=O)(=O)C(C)(C)C)cn23)nn1C)c1nccs1] | (R)-3-(6-(tert- butylsulfonyl)-7-(2- hydroxyethoxy)imidazo [1,2-a]pyridin-3-yl)-1- methyl-N-(1-(thiazol-2- yl)ethyl)-1H-pyrazole- 5-carboxamide | [M + H]⁺ = 533.0 ¹H NMR (400 MHz, DMSO- d₆) δ ppm 9.88 (s, 1H), 9.37 (d, J = 7.6 Hz, 1H), 8.00 (s, 1H), 7.76 (d, J = 3.2 Hz, 1H), 7.65 (d, J = 3.2 Hz, 1H), 7.40 (s, 1H), 7.30 (s, 1H), 5.44 (t, J = 7.2 Hz, 1H), 4.82 (s, 1H), 4.19 (t, J = 4.8 Hz, 2H), 4.14 (s, 3H), 3.79 (s, 2H), 1.64 (d, J = 6.8 Hz, 3H), 1.35 (s, 9H) |
| 95 | 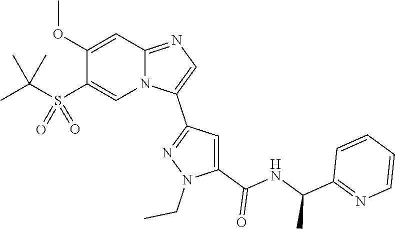 [SMILES: CCn1nc(-c2cnc3cc(OC)c(S(=O)(=O)C(C)(C)C)cn23)cc1C(=O)N[C@H](C)c1ccccn1] | (R)-3-(6-(tert- butylsulfonyl)-7- methoxyimidazo[1,2- a]pyridin-3-yl)-1-ethyl- N-(1-(pyridin-2- yl)ethyl)-1H-pyrazole- 5-carboxamide | [M + H]⁺ = 511.4 ¹H NMR (400 MHz, DMSO- d₆) δ ppm 9.96 (s, 1H), 9.04 (d, J = 7.6 Hz, 1H), 8.58-8.49 (m, 1H), 8.01 (s, 1H), 7.82-7.72 (m, 1H), 7.46-7.40 (m, 2H), 7.31-7.25 (m, 2H), 5.24-5.12 (m, 1H), 4.56-4.46 (m, 2H), 3.94 (s, 3H), 1.52 (d, J = 6.8 Hz, 3H), 1.41 (t, J = 7.6 Hz, 3H), 1.33 (s, 9H) |
| 96 | 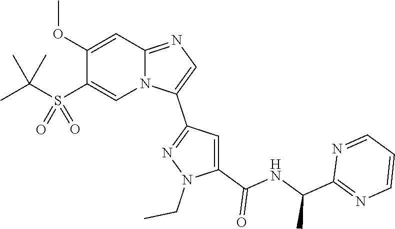 [SMILES: CCn1nc(-c2cnc3cc(OC)c(S(=O)(=O)C(C)(C)C)cn23)cc1C(=O)N[C@H](C)c1ncccn1] | (R)-3-(6-(tert- butylsulfonyl)-7- methoxyimidazo[1,2- a]pyridin-3-yl)-1-ethyl- N-(1-(pyrimidin-2- yl)ethyl)-1H-pyrazole- 5-carboxamide | [M + H]⁺ = 512.2 ¹H NMR (400 MHz, DMSO- d₆) δ ppm 9.96 (s, 1H), 9.03 (d, J = 7.6 Hz, 1H), 8.80 (d, J = 4.8 Hz, 2H), 8.00 (s, 1H), 7.46-7.34 (m, 2H), 7.28 (s, 1H), 5.25-5.13 (m, 1H), 4.51 (q, J = 7.2 Hz, 2H), 3.94 (s, 3H), 1.55 (d, J = 7.2 Hz, 3H), 1.40 (t, J = 7.2 Hz, 3H), 1.33 (s, 9H) |
| 97 | 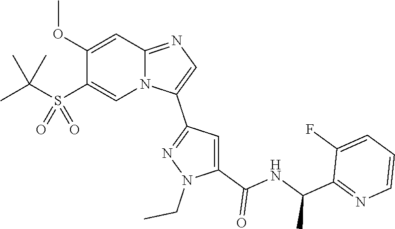 [SMILES: CCn1nc(-c2cnc3cc(OC)c(S(=O)(=O)C(C)(C)C)cn23)cc1C(=O)N[C@H](C)c1ncccc1F] | (R)-3-(6-(tert- butylsulfonyl)-7- methoxyimidazo[1,2- a]pyridin-3-yl)-1-ethyl- N-(1-(3-fluoropyridin- 2-yl)ethyl)-1H- pyrazole-5- carboxamide | [M + H]⁺ = 529.1 ¹H NMR (400 MHz, DMSO- d₆) δ ppm 9.94 (s, 1H), 9.04 (d, J = 7.2 Hz, 1H), 8.47-8.32 (m, 1H), 7.99 (s, 1H), 7.76-7.67 (m, 1H), 7.46-7.36 (m, 2H), 7.28 (s, 1H), 5.54-5.34 (m, 1H), 4.55-4.45 (m, 2H), 3.94 (s, 3H), 1.51 (d, J = 6.8 Hz, 3H), 1.38 (t, J = 7.2 Hz, 3H), 1.32 (s, 9H) |
| 98 | 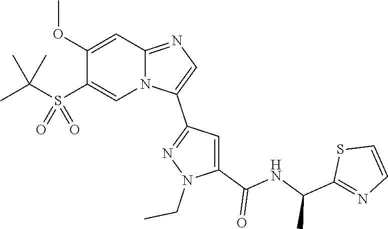 [SMILES: CCn1nc(-c2cnc3cc(OC)c(S(=O)(=O)C(C)(C)C)cn23)cc1C(=O)N[C@H](C)c1nccs1] | (R)-3-(6-(tert- butylsulfonyl)-7- methoxyimidazo[1,2- a]pyridin-3-yl)-1-ethyl- N-(1-(thiazol-2- yl)ethyl)-1H-pyrazole- 5-carboxamide | [M + H]⁺ = 517.1 ¹H NMR (400 MHz, DMSO- d₆) δ ppm 9.96 (s, 1H), 9.38 (d, J = 8.0 Hz, 1H), 8.01 (s, 1H), 7.76 (d, J = 3.2 Hz, 1H), 7.65 (d, J = 3.2 Hz, 1H), 7.37 (s, 1H), 7.29 (s, 1H), 5.44 (t, J = 7.6 Hz, 1H), 4.59-4.53 (m, 2H), 3.94 (s, 3H), 1.64 (d, J = 7.2 Hz, 3H), 1.44 (t, J = 7.2 Hz, 3H), 1.33 (s, 9H) |
| 99 | 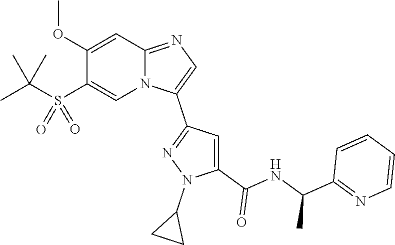 [SMILES: COc1cc2ncc(-c3cc(C(=O)N[C@H](C)c4ccccn4)n(C4CC4)n3)n2cc1S(=O)(=O)C(C)(C)C] | (R)-3-(6-(tert- butylsulfonyl)-7- methoxyimidazo[1,2- a]pyridin-3-yl)-1- cyclopropyl-N-(1- (pyridin-2-yl)ethyl)- 1H-pyrazole-5- carboxamide | [M + H]⁺ = 523.1 ¹H NMR (400 MHz, DMSO- d₆) δ ppm 9.92 (s, 1H), 9.07 (br d, J = 7.6 Hz, 1H), 8.54 (br d, J = 4.0 Hz, 1H), 8.01 (s, 1H), 7.83-7.74 (m, 1H), 7.45 (d, J = 7.6 Hz, 1H), 7.41 (s, 1H), 7.31-7.24 (m, 2H), 5.20 (q, J = 7.2 Hz, 1H), 4.61-4.53 (m, 1H), 3.94 (s, 3H), 1.52 (d, J = 7.2 Hz, 3H), 1.33 (s, 9H), 1.22 (br d, J = 3.6 Hz, 2H), 1.04-0.98 (m, 2H) |
| 100 | 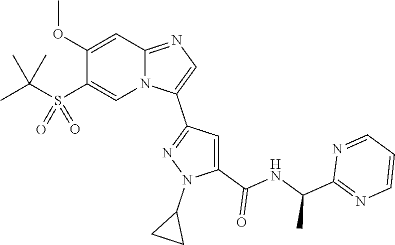 [SMILES: COc1cc2ncc(-c3cc(C(=O)N[C@H](C)c4ncccn4)n(C4CC4)n3)n2cc1S(=O)(=O)C(C)(C)C] | (R)-3-(6-(tert- butylsulfonyl)-7- methoxyimidazo[1,2- a]pyridin-3-yl)-1- cyclopropyl-N-(1- (pyrimidin-2-yl)ethyl)- 1H-pyrazole-5- carboxamide | [M + H]⁺ = 524.1 ¹H NMR (400 MHz, DMSO- d₆) δ ppm 9.92 (s, 1H), 9.04 (d, J = 7.6 Hz, 1H), 8.81 (d, J = 4.8 Hz, 2H), 8.01 (s, 1H), 7.45-7.37 (m, 2H), 7.27 (s, 1H), 5.24-5.18 (m, 1H), 4.60- 4.54 (m, 1H), 3.94 (s, 3H), 1.56 (d, J = 7.2 Hz, 3H), 1.33 (s, 9H), 1.23 (d, J = 3.6 Hz, 2H), 1.01 (t, J = 6.8 Hz, 2H) |
| 101 | 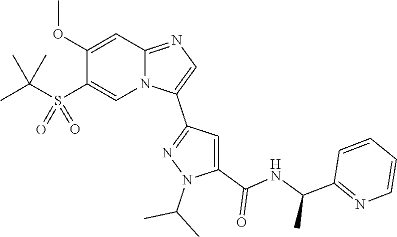 [SMILES: COc1cc2ncc(-c3cc(C(=O)N[C@H](C)c4ccccn4)n(C(C)C)n3)n2cc1S(=O)(=O)C(C)(C)C] | (R)-3-(6-(tert- butylsulfonyl)-7- methoxyimidazo[1,2- a]pyridin-3-yl)-1- isopropyl-N-(1- (pyridin-2-yl)ethyl)- 1H-pyrazole-5- carboxamide | [M + H]⁺ = 525.1 ¹H NMR (400 MHz, DMSO- d₆) δ ppm 10.09 (s, 1H), 9.05 (d, J = 7.6 Hz, 1H), 8.54 (d, J = 4.2 Hz, 1H), 8.02 (s, 1H), 7.85-7.74 (m, 1H), 7.43 (d, J = 8.0 Hz, 1H), 7.38 (s, 1H), 7.31-7.25 (m, 2H), 5.46-5.37 (m, 1H), 5.23-5.13 (m, 1H), 3.94 (s, 3H), 1.51 (d, J = 7.2 Hz, 3H), 1.48-1.43 (m, 6H), 1.33 (s, 9H) |
| 102 | 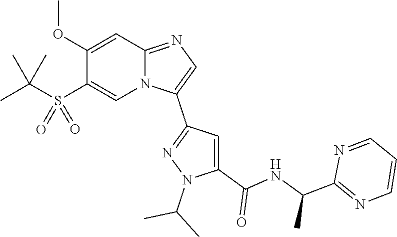 [SMILES: COc1cc2ncc(-c3cc(C(=O)N[C@H](C)c4ncccn4)n(C(C)C)n3)n2cc1S(=O)(=O)C(C)(C)C] | (R)-3-(6-(tert- butylsulfonyl)-7- methoxyimidazo[1,2- a]pyridin-3-yl)-1- isopropyl-N-(1- (pyrimidin-2-yl)ethyl)- 1H-pyrazole-5- carboxamide | [M + H]⁺ = 526.1 ¹H NMR (400 MHz, DMSO- d₆) δ ppm 10.09 (s, 1H), 9.03 (d, J = 7.6Hz, 1H), 8.80 (d, J = 4.8 Hz, 2H), 8.01 (s, 1H), 7.41 (t, J = 4.8 Hz, 1H), 7.36 (s, 1H), 7.28 (s, 1H), 5.47-5.35 (m, 1H), 5.24-5.13 (m, 1H), 3.94 (s, 3H), 1.55 (d, J = 7.2 Hz, 3H), 1.48-1.42 (m, 6H), 1.33 (s, 9H) |
| 103 | 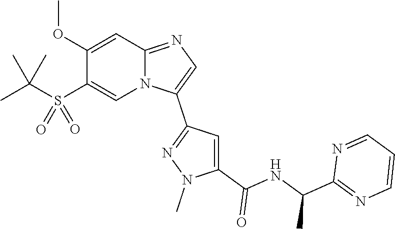 [SMILES: COc1cc2ncc(-c3cc(C(=O)N[C@H](C)c4ncccn4)n(C)n3)n2cc1S(=O)(=O)C(C)(C)C] | (R)-3-(6-(tert- butylsulfonyl)-7- methoxyimidazo[1,2- a]pyridin-3-yl)-1- methyl-N-(1- (pyrimidin-2-yl)ethyl)- 1H-pyrazole-5- carboxamide | [M + H]⁺ = 498.0 ¹H NMR (400 MHz, DMSO- d₆) δ ppm 9.87 (s, 1H), 9.03 (d, J = 7.2 Hz, 1H), 8.80 (d, J = 4.8 Hz, 2H), 8.00 (s, 1H), 7.46 (s, 1H), 7.41 (t, J = 4.8 Hz, 1H), 7.29 (s, 1H), 5.25-5.15 (m, 1H), 4.10 (s, 3H), 3.94 (s, 3H), 1.55 (d, J = 6.8 Hz, 3H), 1.33 (s, 9H) |
| 104 | 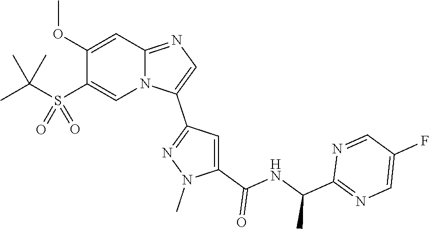 [SMILES: COc1cc2ncc(-c3cc(C(=O)N[C@H](C)c4ncc(F)cn4)n(C)n3)n2cc1S(=O)(=O)C(C)(C)C] | (R)-3-(6-(tert- butylsulfonyl)-7- methoxyimidazo[1,2- a]pyridin-3-yl)-N-(1- (5-fluoropyrimidin-2- yl)ethyl)-1-methyl-1H- pyrazole-5- carboxamide | [M + H]⁺ = 516.3 ¹H NMR (400 MHz, DMSO- d₆) δ ppm 9.87 (s, 1H), 9.09 (d, J = 7.6 Hz, 1H), 8.89 (s, 2H), 8.00 (s, 1H), 7.45 (s, 1H), 7.29 (s, 1H), 5.23 (q, J = 7.2 Hz, 1H), 4.09 (s, 3H), 3.94 (s, 3H), 1.55 (d, J = 7.2 Hz, 3H), 1.33 (s, 9H) |
| 105 | 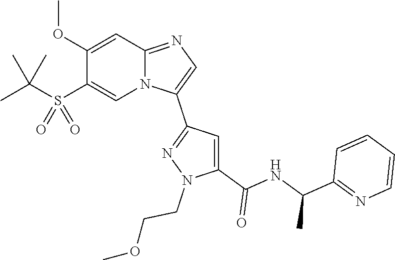 [SMILES: COCCn1nc(-c2cnc3cc(OC)c(S(=O)(=O)C(C)(C)C)cn23)cc1C(=O)N[C@H](C)c1ccccn1] | (R)-3-(6-(tert- butylsulfonyl)-7- methoxyimidazo[1,2- a]pyridin-3-yl)-1-(2- methoxyethyl)-N-(1- (pyridin-2-yl)ethyl)- 1H-pyrazole-5- carboxamide | [M + H]⁺ = 541.1 ¹H NMR (400 MHz, DMSO- d₆) δ ppm 9.93 (s, 1H), 9.07 (d, J = 7.6 Hz, 1H), 8.60-8.48 (m, 1H), 8.03 (s, 1H), 7.83-7.72 (m, 1H), 7.49-7.36 (m, 2H), 7.33-7.22 (m, 2H), 5.25-5.12 (m, 1H), 4.70 (t, J = 5.2 Hz, 2H), 3.94 (s, 3H), 3.77 (t, J = 5.6 Hz, 2H), 3.20 (s, 3H), 1.52 (d, J = 7.2 Hz, 3H), 1.32 (s, 9H) |
| 106 | 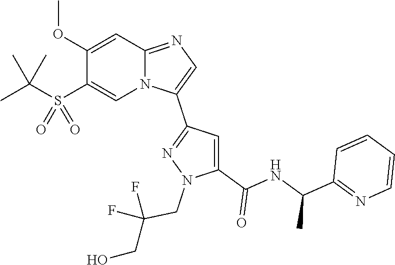 [SMILES: COc1cc2ncc(-c3cc(C(=O)N[C@H](C)c4ccccn4)n(CC(F)(F)CO)n3)n2cc1S(=O)(=O)C(C)(C)C] | (R)-3-(6-(tert- butylsulfonyl)-7- methoxyimidazo[1,2- a]pyridin-3-yl)-1-(2,2- difluoro-3- hydroxypropyl)-N-(1- (pyridin-2-yl)ethyl)- 1H-pyrazole-5- carboxamide | [M + H]⁺ = 577.1 ¹H NMR (400 MHz, DMSO- d₆) δ ppm 9.87 (s, 1H), 9.16 (d, J = 8.0 Hz, 1H), 8.54 (d, J = 4.0 Hz, 1H), 8.08 (s, 1H), 7.87-7.69 (m, 1H), 7.49 (s, 1H), 7.42 (d, J = 7.6 Hz, 1H), 7.30 (s, 1H), 7.30-7.24 (m, 1H), 5.59 (s, 1H), 5.26-5.04 (m, 3H), 3.94 (s, 3H), 3.67 (t, J = 13.6 Hz, 2H), 1.51 (d, J = 7.2 Hz, 3H), 1.32 (s, 9H) |
| 107 | 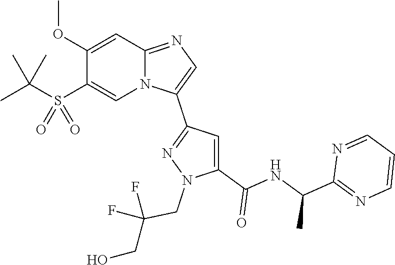 [SMILES: COc1cc2ncc(-c3cc(C(=O)N[C@H](C)c4ncccn4)n(CC(F)(F)CO)n3)n2cc1S(=O)(=O)C(C)(C)C] | (R)-3-(6-(tert- butylsulfonyl)-7- methoxyimidazo[1,2- a]pyridin-3-yl)-1-(2,2- difluoro-3- hydroxypropyl)-N-(1- (pyrimidin-2-yl)ethyl)- 1H-pyrazole-5- carboxamide | [M + H]⁺ = 578.0 ¹H NMR (400 MHz, DMSO- d₆) δ ppm 9.87 (s, 1H), 9.14 (d, J = 7.2 Hz, 1H), 8.80 (d, J = 4.8 Hz, 2H), 8.07 (s, 1H), 7.51 (s, 1H), 7.41 (t, J = 4.8 Hz, 1H), 7.30 (s, 1H), 5.74-5.38 (m, 1H), 5.29-5.15 (m, 2H), 5.07 (q, J = 14.0 Hz, 1H), 3.94 (s, 3H), 3.67 (t, J = 14.4 Hz, 2H), 1.55 (d, J = 7.2 Hz, 3H), 1.32 (s, 9H) |
| 108 | 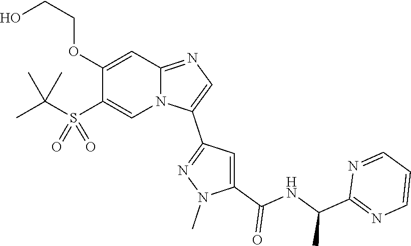 [SMILES: C[C@@H](NC(=O)c1cc(-c2cnc3cc(OCCO)c(S(=O)(=O)C(C)(C)C)cn23)nn1C)c1ncccn1] | (R)-3-(6-(tert- butylsulfonyl)-7-(2- hydroxyethoxy)imidazo [1,2-a]pyridin-3-yl)-1- methyl-N-(1- (pyrimidin-2-yl)ethyl)- 1H-pyrazole-5- carboxamide | [M + H]⁺ = 528.0 ¹H NMR (400 MHz, DMSO- d₆) δ ppm 9.89 (s, 1H), 9.04 (d, J = 7.2 Hz, 1H), 8.80 (d, J = 4.8 Hz, 2H), 8.02 (s, 1H), 7.46 (s, 1H), 7.41 (t, J = 4.8 Hz, 1H), 7.33-7.29 (m, 1H), 5.20 (t, J = 7.2 Hz, 1H), 4.90-4.75 (m, 1H), 4.20 (t, J = 4.8 Hz, 2H), 4.10 (s, 3H), 3.78 (t, J = 4.8 Hz, 2H), 1.55 (d, J = 7.2 Hz, 3H), 1.35 (s, 9H) |
| 109 | 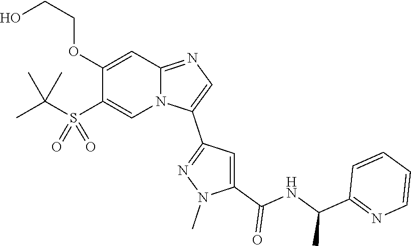 [SMILES: C[C@@H](NC(=O)c1cc(-c2cnc3cc(OCCO)c(S(=O)(=O)C(C)(C)C)cn23)nn1C)c1ccccn1] | (R)-3-(6-(tert- butylsulfonyl)-7-(2- hydroxyethoxy)imidazo [1,2-a]pyridin-3-yl)-1- methyl-N-(1-(pyridin- 2-yl)ethyl)-1H- pyrazole-5- carboxamide | [M + H]⁺ = 527.1 ¹H NMR (400 MHz, DMSO- d₆) δ ppm 9.90 (s, 1H), 9.07 (d, J = 7.6 Hz, 1H), 8.55 (d, J = 4.0 Hz, 1H), 8.07 (s, 1H), 7.80 (dd, J = 7.6, 1.6 Hz, 1H), 7.52- 7.41 (m, 2H), 7.34 (s, 1H), 7.30 (dd, J = 6.8, 5.2 Hz, 1H), 5.19 (t, J = 7.2 Hz, 1H), 5.07- 4.63 (m, 1H), 4.21 (t, J = 4.8 Hz, 2H), 4.11 (s, 3H), 3.79 (t, J = 4.8 Hz, 2H), 1.52 (d, J = 7.2 Hz, 3H), 1.35 (s, 9H) |
| 110 | 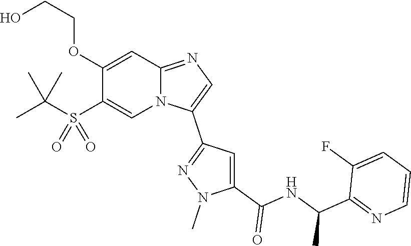 [SMILES: C[C@@H](NC(=O)c1cc(-c2cnc3cc(OCCO)c(S(=O)(=O)C(C)(C)C)cn23)nn1C)c1ncccc1F] | (R)-3-(6-(tert- butylsulfonyl)-7-(2- hydroxyethoxy)imidazo [1,2-a]pyridin-3-yl)-N- (1-(3-fluoropyridin-2- yl)ethyl)-1-methyl-1H- pyrazole-5- carboxamide | [M + H]⁺ = 545.1 ¹H NMR (400 MHz, DMSO- d₆) δ ppm 9.86 (s, 1H), 9.04 (d, J = 7.6 Hz, 1H), 8.46-8.38 (m, 1H), 7.98 (s, 1H), 7.75-7.65 (m, 1H), 7.45-7.38 (m, 2H), 7.29 (s, 1H), 5.50-5.40 (m, 1H), 4.86-4.80 (m, 1H), 4.19 (t, J = 5.2 Hz, 2H), 4.08 (s, 3H), 3.81-3.75 (m, 2H), 1.51 (d, J = 6.8 Hz, 3H), 1.34 (s, 9H) |
| 111 | 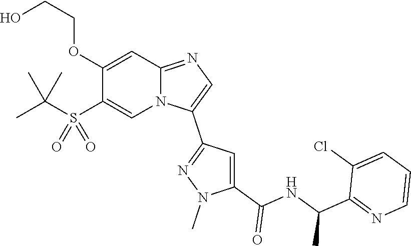 [SMILES: C[C@@H](NC(=O)c1cc(-c2cnc3cc(OCCO)c(S(=O)(=O)C(C)(C)C)cn23)nn1C)c1ncccc1Cl] | (R)-3-(6-(tert- butylsulfonyl)-7-(2- hydroxyethoxy)imidazo [1,2-a]pyridin-3-yl)-N- (1-(3-chloropyridin-2- yl)ethyl)-1-methyl-1H- pyrazole-5- carboxamide | [M + H]⁺ = 561.2 ¹H NMR (400 MHz, DMSO- d₆) δ ppm 9.87 (s, 1H), 9.04 (d, J = 7.2 Hz, 1H), 8.53 (dd, J = 4.8, 1.2 Hz, 1H), 7.98 (s, 1H), 7.94 (dd, J = 7.8, 1.2 Hz, 1H), 7.46 (s, 1H), 7.37 (dd, J = 8.4, 4.2 Hz, 1H), 7.30 (s, 1H), 5.54 (q, J = 6.8 Hz, 1H), 5.06-4.54 (m, 1H), 4.19 (t, J = 4.8 Hz, 2H), 4.08 (s, 3H), 3.78 (t, J = 4.8 Hz, 2H), 1.49 (d, J = 6.8 Hz, 3H), 1.34 (s, 9H) SFC: Rₜ = 1.583 min, 100% ee; CHIRALPAK AD-3 50 x 4.6 mm, 3 μm; 5-40% EtOH |
| | | | (0.05% DEA) in CO₂, 4 |
| | | | mL/min; 35° C., 100 Bar, 220 |
| | | | nm |
| 112 | 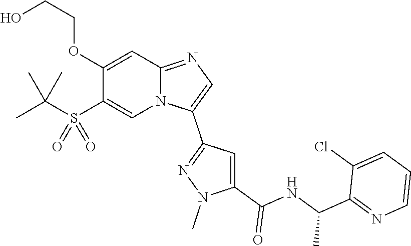 [SMILES: C[C@H](NC(=O)c1cc(-c2cnc3cc(OCCO)c(S(=O)(=O)C(C)(C)C)cn23)nn1C)c1ncccc1Cl] | (S)-3-(6-(tert- butylsulfonyl)-7-(2- hydroxyethoxy)imidazo [1,2-a]pyridin-3-yl)-N- (1-(3-chloropyridin-2- yl)ethyl)-1-methyl-1H- pyrazole-5- carboxamide | [M + H]⁺ = 561.2 ¹H NMR (400 MHz, DMSO- d₆) δ ppm 9.86 (s, 1H), 9.04 (d, J = 7.2 Hz, 1H), 8.53 (dd, J = 4.4, 1.2 Hz, 1H), 7.98 (s, 1H), 7.94 (dd, J = 8.0, 1.2 Hz, 1H), 7.46 (s, 1H), 7.38 (dd, J = 8.4, 4.8 Hz, 1H), 7.30 (s, 1H), 5.66-5.45 (m, 1H), 5.01-4.67 (m, 1H), 4.19 (t, J = 4.2 Hz, 2H), 4.08 (s, 3H), 3.78 (t, J = 5.2 Hz, 2H), 1.49 (d, J = 6.8 Hz, 3H), 1.34 (s, 9H) SFC: Rₜ = 1.880 min, 99.4% ee; CHIRALPAK AD-3 50 x 4.6 mm, 3 μm; 5-40% EtOH |
| | | | (0.05% DEA) in CO₂, 4 |
| | | | mL/min; 35° C., 100 Bar, 220 |
| | | | nm |
| 113 | 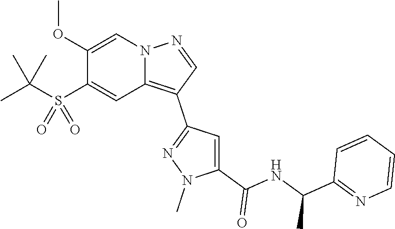 [SMILES: COc1cn2ncc(-c3cc(C(=O)N[C@H](C)c4ccccn4)n(C)n3)c2cc1S(=O)(=O)C(C)(C)C] | (R)-3-(5-(tert- butylsulfonyl)-6- methoxypyrazolo[1,5- a]pyridin-3-yl)-1- methyl-N-(1-(pyridin- 2-yl)ethyl)-1H- pyrazole-5- carboxamide | [M + H]⁺ = 497.1 ¹H NMR (400 MHz, DMSO- d₆) δ ppm 8.99 (d, J = 7.6 Hz, 1H), 8.72-8.67 (m, 2H), 8.54 (d, J = 4.8 Hz, 1H), 8.42 (s, 1H), 7.81-7.74 (m, 1H), 7.44 (d, J = 7.6 Hz, 1H), 7.37 (s, 1H), 7.28 (dd, J = 7.2, 5.2 Hz, 1H), 5.22-5.14 (m, 1H), 4.09 (s, 3H), 3.92 (s, 3H), 1.52 (d, J = 6.8 Hz, 3H), 1.32 (s, 9H) |
| 114 | 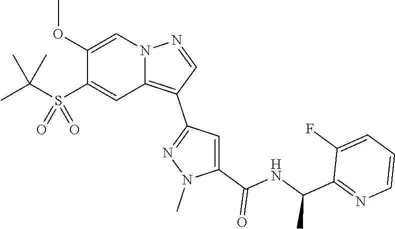 [SMILES: COc1cn2ncc(-c3cc(C(=O)N[C@H](C)c4ncccc4F)n(C)n3)c2cc1S(=O)(=O)C(C)(C)C] | (R)-3-(5-(tert- butylsulfonyl)-6- methoxypyrazolo[1,5- a]pyridin-3-yl)-N-(1- (3-fluoropyridin-2- yl)ethyl)-1-methyl-1H- pyrazole-5- carboxamide | [M + H]⁺ = 515.0 ¹H NMR (400 MHz, DMSO- d₆) δ ppm 8.98 (d, J = 7.6 Hz, 1H), 8.71 (s, 1H), 8.66 (s, 1H), 8.46-8.32 (m, 2H), 7.78-7.63 (m, 1H), 7.45-7.36 (m, 1H), 7.36 (s, 1H), 5.56-5.33 (m, 1H), 4.06 (s, 3H), 3.92 (s, 3H), 1.51 (d, J = 7.2 Hz, 3H), 1.32 (s, 9H) |
| 115 | 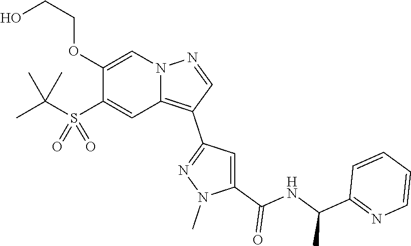 [SMILES: C[C@@H](NC(=O)c1cc(-c2cnn3cc(OCCO)c(S(=O)(=O)C(C)(C)C)cc23)nn1C)c1ccccn1] | (R)-3-(5-(tert- butylsulfonyl)-6-(2- hydroxyethoxy)pyrazolo [1,5-a]pyridin-3-yl)-1- methyl-N-(1-(pyridin- 2-yl)ethyl)-1H- pyrazole-5- carboxamide | [M + H]⁺ = 527.2 ¹H NMR (400 MHz, DMSO- d₆) δ ppm 8.99 (br d, J = 7.6 Hz, 1H), 8.75 (s, 1H), 8.68 (s, 1H), 8.54 (br d, J = 4.4 Hz, 1H), 8.42 (s, 1H), 7.81-7.75 (m, 1H), 7.44 (d, J = 7.6 Hz, 1H), 7.37 (s, 1H), 7.30-7.26 1H), 4.87-4.80 (m, 1H), 4.20- 4.14 (m, 2H), 4.09 (s, 3H), 3.77 (q, J = 5.2 Hz, 2H), 1.52 (d, J = 7.2 Hz, 3H), 1.34 (s, |
| 116 | 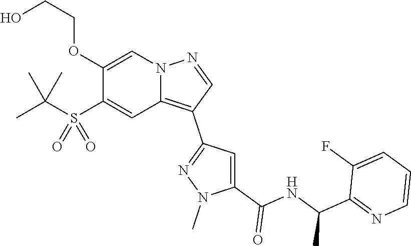 [SMILES: C[C@@H](NC(=O)c1cc(-c2cnn3cc(OCCO)c(S(=O)(=O)C(C)(C)C)cc23)nn1C)c1ncccc1F] | (R)-3-(5-(tert- butylsulfonyl)-6-(2- hydroxyethoxy)pyrazolo [1,5-a]pyridin-3-yl)- N-(1-(3-fluoropyridin- 2-yl)ethyl)-1-methyl- 1H-pyrazole-5- carboxamide | [M + H]⁺ = 545.1 ¹H NMR (400 MHz, DMSO- d₆) δ ppm 8.98 (d, J = 7.2 Hz, 1H), 8.75 (s, 1H), 8.66 (s, 1H), 8.43-8.40 (m, 1H), 8.40 (s, 1H), 7.75-7.68 (m, 1H), 7.45- 7.39 (m, 1H), 7.36 (s, 1H), 5.49-5.42 (m, 1H), 4.86-4.81 (m, 1H), 4.16 (t, J = 5.2 Hz, 2H), 4.06 (s, 3H), 3.79-3.74 (m, 2H), 1.51 (d, J = 7.2 Hz, 3H), 1.34 (s, 9H) |
| 117 | 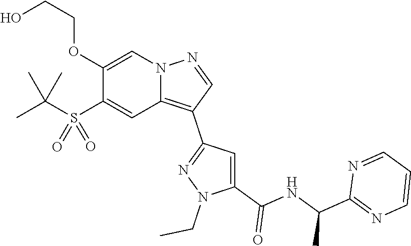 [SMILES: CCn1nc(-c2cnn3cc(OCCO)c(S(=O)(=O)C(C)(C)C)cc23)cc1C(=O)N[C@H](C)c1ncccn1] | (R)-3-(5-(tert- butylsulfonyl)-6-(2- hydroxyethoxy)pyrazolo [1,5-a]pyridin-3-yl)-1- ethyl-N-(1-(pyrimidin- 2-yl)ethyl)-1H- pyrazole-5- carboxamide | [M + H]⁺ = 542.1 ¹H NMR (400 MHz, DMSO- d₆) δ ppm 8.98 (d, J = 7.6 Hz, 1H), 8.80 (d, J = 5.2 Hz, 2H), 8.75 (s, 1H), 8.71 (s, 1H), 8.41 (s, 1H), 7.41 (t, J = 4.8 Hz, 1H), 7.33 (s, 1H), 5.25-5.10 (m, 1H), 4.83 (t, J = 5.2 Hz, 1H), 4.49 (q, J = 7.2 Hz, 2H), 4.16 (t, J = 4.8 Hz, 2H), 3.77 (q, J = 4.8 Hz, 2H), 1.55 (d, J = 7.2 Hz, 3H), 1.43-1.30 (m, 12H) |
| 118 | 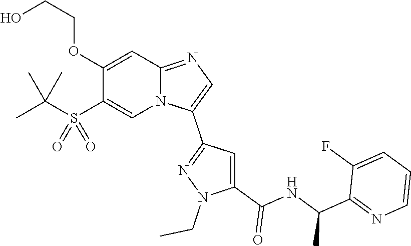 [SMILES: CCn1nc(-c2cnc3cc(OCCO)c(S(=O)(=O)C(C)(C)C)cn23)cc1C(=O)N[C@H](C)c1ncccc1F] | (R)-3-(6-(tert- butylsulfonyl)-7-(2- hydroxyethoxy)imidazo [1,2-a]pyridin-3-yl)-1- ethyl-N-(1-(3- fluoropyridin-2- yl)ethyl)-1H-pyrazole- 5-carboxamide | [M + H]⁺ = 559.2 ¹H NMR (400 MHz, DMSO- d₆) δ ppm 9.95 (s, 1H), 9.06 (d, J = 7.2 Hz, 1H), 8.41 (d, J = 4.8 Hz, 1H), 7.99 (s, 1H), 7.76-7.68 (m, 1H), 7.45-7.38 (m, 2H), 7.29 (s, 1H), 5.50- 5.40 (m, 1H), 4.82 (t, J = 5.2 Hz, 1H), 4.50 (q, J = 7.2 Hz, 2H), 4.19 (t, J = 5.2 Hz, 2H), 3.78 (q, J = 4.8 Hz, 2H), 1.51 (d, J = 7.2 Hz, 3H), 1.38 (t, J = 7.2 Hz, 3H), 1.34 (s, 9H) |
| 119 | 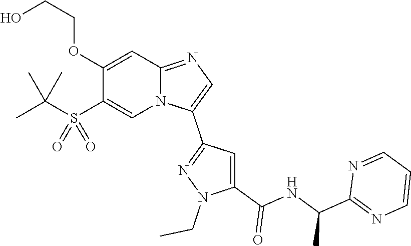 [SMILES: CCn1nc(-c2cnc3cc(OCCO)c(S(=O)(=O)C(C)(C)C)cn23)cc1C(=O)N[C@H](C)c1ncccn1] | (R)-3-(6-(tert- butylsulfonyl)-7-(2- hydroxyethoxy)imidazo [1,2-a]pyridin-3-yl)-1- ethyl-N-(1-(pyrimidin- 2-yl)ethyl)-1H- pyrazole-5- carboxamide | [M + H]⁺ = 542.1 ¹H NMR (400 MHz, DMSO- d₆) δ ppm 9.96 (s, 1H), 9.04 (d, J = 7.2 Hz, 1H), 8.80 (d, J = 4.8 Hz, 2H), 8.00 (s, 1H), 7.47-7.32 (m, 2H), 7.30 (s, 1H), 5.29-5.11 (m, 1H), 4.81 (t, J = 5.2 Hz, 1H), 4.58-4.41 (m, 2H), 4.19 (t, J = 4.8 Hz, 2H), 3.78 (d, J = 4.8 Hz, 2H), 1.55 (d, J = 7.2 Hz, 3H), 1.40 (t, J = 7.2 Hz, 3H), 1.34 (s, 9H) |
| 120 | 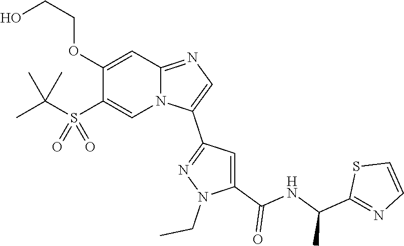 [SMILES: CCn1nc(-c2cnc3cc(OCCO)c(S(=O)(=O)C(C)(C)C)cn23)cc1C(=O)N[C@H](C)c1nccs1] | (R)-3-(6-(tert- butylsulfonyl)-7-(2- hydroxyethoxy)imidazo [1,2-a]pyridin-3-yl)-1- ethyl-N-(1-(thiazol-2- yl)ethyl)-1H-pyrazole- 5-carboxamide | [M + H]⁺ = 547.0 ¹H NMR (400 MHz, DMSO- d₆) δ ppm 9.96 (s, 1H), 9.38 (d, J = 8.0 Hz, 1H), 8.00 (s, 1H), 7.76 (d, J = 3.2 Hz, 1H), 7.65 (d, J = 3.2 Hz, 1H), 7.36 (s, 1H), 7.30 (s, 1H), 5.44 (t, J = 7.6 Hz, 1H), 4.81 (t, J = 4.8 Hz, 1H), 4.63-4.50 (m, 2H), 4.19 (t, J = 5.2 Hz, 2H), 3.84- 3.73 (m, 2H), 1.63 (d, J = 7.2 Hz, 3H), 1.43 (t, J = 7.2 Hz, 3H), 1.35 (s, 9H) |
| 121 | 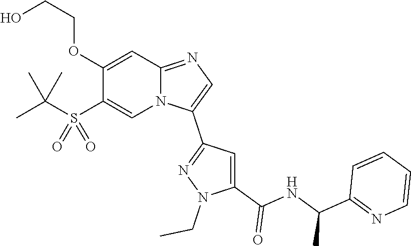 [SMILES: CCn1nc(-c2cnc3cc(OCCO)c(S(=O)(=O)C(C)(C)C)cn23)cc1C(=O)N[C@H](C)c1ccccn1] | (R)-3-(6-(tert- butylsulfonyl)-7-(2- hydroxyethoxy)imidazo [1,2-a]pyridin-3-yl)-1- ethyl-N-(1-(pyridin-2- yl)ethyl)-1H-pyrazole- 5-carboxamide | [M + H]⁺ = 541.1 ¹H NMR (400 MHz, DMSO- d₆) δ ppm 9.96 (s, 1H), 9.04 (d, J = 8.0 Hz, 1H), 8.54 (d, J = 4.8 Hz, 1H), 8.00 (s, 1H), 7.84-7.73 (m, 1H), 7.48-7.38 (m, 2H), 7.33-7.24 (m, 2H), 5.18 (t, J = 7.2 Hz, 1H), 4.80 (t, J = 4.8 Hz, 1H), 4.52 (q, J = 7.2 Hz, 2H), 4.20 (t, J = 4.8 Hz, 2H), 3.78 (q, J = 4.8 Hz, 2H), 1.52 (d, J = 7.2 Hz, 3H), 1.41 (t, J = 7.2 Hz, 3H), 1.34 (s, 9H) |
| 122 | 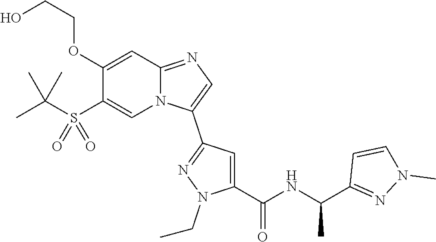 [SMILES: CCn1nc(-c2cnc3cc(OCCO)c(S(=O)(=O)C(C)(C)C)cn23)cc1C(=O)N[C@H](C)c1ccn(C)n1] | (R)-3-(6-(tert- butylsulfonyl)-7-(2- hydroxyethoxy)imidazo [1,2-a]pyridin-3-yl)-1- ethyl-N-(1-(1-methyl- 1H-pyrazol-3-yl)ethyl)- 1H-pyrazole-5- carboxamide | [M + H]⁺ = 544.1 ¹H NMR (400 MHz, DMSO- d₆) δ ppm 9.96 (s, 1H), 8.87 (d, J = 8.4 Hz, 1H), 7.96 (s, 1H), 7.59 (d, J = 2.0 Hz, 1H), 7.32 (s, 1H), 7.29 (s, 1H), 6.18 (d, J = 2.0 Hz, 1H), 5.25-5.13 (m, 1H), 4.81 (t, J = 4.4 Hz, 1H), 4.55 (q, J = 7.2 Hz, 2H), 4.19 (t, J = 4.8 Hz, 2H), 3.82-3.75 (m, 5H), 1.47 (d, J = 7.2 Hz, 3H), 1.43 (t, J = 7.6 Hz, 3H), 1.34 (s, 9H) SFC: Rₜ = 0.616 min, 100% ee; CHIRALPAK AD-3 50 x |
| | | | 4.6 mm, 3 μm; 40% i- |
| | | | PrOH/MeCN (7:3 + 0.05% |
| | | | DEA) in CO₂, 3 mL/min; 35º |
| | | | C., 100 Bar, 220 nm |
| 123 | 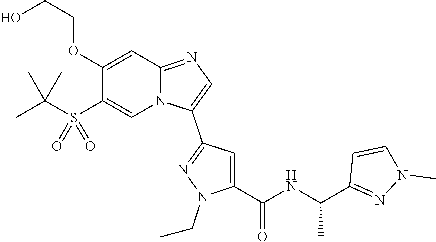 [SMILES: CCn1nc(-c2cnc3cc(OCCO)c(S(=O)(=O)C(C)(C)C)cn23)cc1C(=O)N[C@@H](C)c1ccn(C)n1] | (S)-3-(6-(tert- butylsulfonyl)-7-(2- hydroxyethoxy)imidazo [1,2-a]pyridin-3-yl)-1- ethyl-N-(1-(1-methyl- 1H-pyrazol-3-yl)ethyl)- 1H-pyrazole-5- carboxamide | [M + H]⁺ = 544.2 ¹H NMR (400 MHz, DMSO- d₆) δ ppm 9.96 (s, 1H), 8.87 (d, J = 8.4 Hz, 1H), 7.96 (s, 1H), 7.59 (d, J = 1.9 Hz, 1H), 7.32 (s, 1H), 7.29 (s, 1H), 6.18 (d, J = 2.0 Hz, 1H), 5.24-5.14 (m, 1H), 4.81 (t, J = 5.2 Hz, 1H), 4.55 (q, J = 7.2 Hz, 2H), 4.19 (t, J = 4.8 Hz, 2H), 3.82-3.75 (m, 5H), 1.47 (d, J = 7.8 Hz, 3H), 1.43 (t, J = 7.2 Hz, 3H), 1.34 (s, 9H) SFC: Rₜ = 1.413 min, 99.2% ee; CHIRALPAK AD-3 50 x 4.6 mm, 3 μm; 40% i- |
| | | | PrOH/MeCN (7:3 + 0.05% |
| | | | DEA) in CO₂, 3 mL/min; 35º |
| | | | C., 100 Bar, 220 nm |
| 124 | 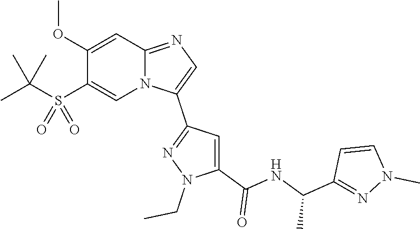 [SMILES: CCn1nc(-c2cnc3cc(OC)c(S(=O)(=O)C(C)(C)C)cn23)cc1C(=O)N[C@@H](C)c1ccn(C)n1] | (S)-3-(6-(tert- butylsulfonyl)-7- methoxyimidazo[1,2- a]pyridin-3-yl)-1-ethyl- N-(1-(1-methyl-1H- pyrazol-3-yl)ethyl)-1H- pyrazole-5- carboxamide | [M + H]⁺ = 514.1 ¹H NMR (400 MHz, DMSO- d₆) δ ppm 9.96 (s, 1H), 8.87 (d, J = 8.4 Hz, 1H), 7.97 (s, 1H), 7.59 (d, J = 2.0 Hz, 1H), 7.32 (s, 1H), 7.28 (s, 1H), 6.18 (d, J = 2.0 Hz, 1H), 5.26-5.10 (m, 1H), 4.55 (q, J = 7.2 Hz, 2H), 3.94 (s, 3H), 3.79 (s, 3H), 1.47 (d, J = 7.2 Hz, 3H), 1.43 (t, J = 7.2 Hz, 3H), 1.32 (s, 9H) SFC: Rₜ = 1.388 min, 100% ee; CHIRALCEL OD-3 50 x 4.6 mm, 3 μm; 5-40% MeOH |
| | | | (0.05% DEA) in CO₂, 3 |
| | | | mL/min; 35° C., 100 Bar, 220 |
| | | | nm |
| 125 | 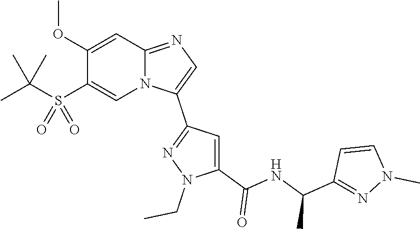 [SMILES: CCn1nc(-c2cnc3cc(OC)c(S(=O)(=O)C(C)(C)C)cn23)cc1C(=O)N[C@H](C)c1ccn(C)n1] | (R)-3-(6-(tert- butylsulfonyl)-7- methoxyimidazo[1,2- a]pyridin-3-yl)-1-ethyl- N-(1-(1-methyl-1H- pyrazol-3-yl)ethyl)-1H- pyrazole-5- carboxamide | [M + H]⁺ = 514.1 ¹H NMR (400 MHz, DMSO- d₆) δ ppm 9.96 (s, 1H), 8.87 (d, J = 8.8 Hz, 1H), 7.97 (s, 1H), 7.59 (d, J = 2.0 Hz, 1H), 7.32 (s, 1H), 7.28 (s, 1H), 6.18 (d, J = 2.0 Hz, 1H), 5.23-5.15 (m, 1H), 4.55 (q, J = 7.2 Hz, 2H), 3.94 (s, 3H), 3.79 (s, 3H), 1.47 (d, J = 6.8 Hz, 3H), 1.43 (t, J = 7.2 Hz, 3H), 1.32 (s, 9H) SFC: Rₜ = 1.513 min, 97.8% ee; CHIRALCEL OD-3 50 x 4.6 mm, 3 μm; 5-40% MeOH |
| | | | (0.05% DEA) in CO₂, 3 |
| | | | mL/min; 35° C., 100 Bar, 220 |
| | | | nm |
| 126 | 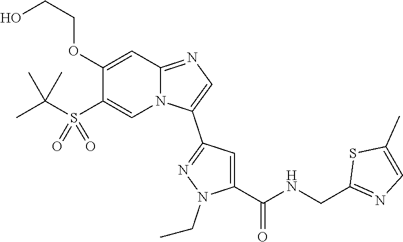 [SMILES: CCn1nc(-c2cnc3cc(OCCO)c(S(=O)(=O)C(C)(C)C)cn23)cc1C(=O)NCc1ncc(C)s1] | 3-(6-(tert- butylsulfonyl)-7-(2- hydroxyethoxy)imidazo [1,2-a]pyridin-3-yl)-1- ethyl-N-((5- methylthiazol-2- yl)methyl)-1H- pyrazole-5- carboxamide | [M + H]⁺ = 547.2 ¹H NMR (400 MHz, DMSO- d₆) δ ppm 9.93 (s, 1H), 9.54 (t, J = 6.0 Hz, 1H), 7.99 (s, 1H), 7.40 (d, J = 1.2 Hz, 1H), 7.30 (s, 2H), 4.82 (t, J = 5.2 Hz, 1H), 4.66 (d, J = 6.0 Hz, 2H), 4.58 (q, J = 7.2 Hz, 2H), 4.19 (t, J = 4.8 Hz, 2H), 3.78 (q, J = 4.8 Hz, 2H), 2.40 (d, J = 0.8 Hz, 3H), 1.44 (t, J = 7.2 Hz, 3H), 1.34 (s, 9H) |
| 127 | 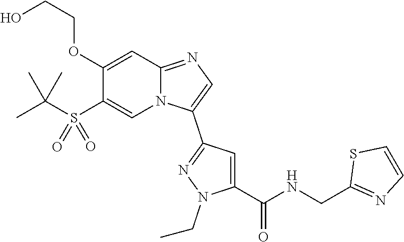 [SMILES: CCn1nc(-c2cnc3cc(OCCO)c(S(=O)(=O)C(C)(C)C)cn23)cc1C(=O)NCc1nccs1] | 3-(6-(tert- butylsulfonyl)-7-(2- hydroxyethoxy)imidazo [1,2-a]pyridin-3-yl)-1- ethyl-N-(thiazol-2- ylmethyl)-1H-pyrazole- 5-carboxamide | [M + H]⁺ = 533.0 ¹H NMR (400 MHz, DMSO- d₆) δ ppm 9.94 (s, 1H), 9.58 (s, 1H), 7.99 (s, 1H), 7.76 (d, J = 3.2 Hz, 1H), 7.66 (d, J = 3.2 Hz, 1H), 7.30 (d, J = 4.8 Hz, 2H), 4.81 (s, 1H), 4.76 (s, 2H), 4.58 (q, J = 7.2 Hz, 2H), 4.19 (t, J = 4.8 Hz, 2H), 3.78 (s, 2H), 1.44 (t, J = 7.2 Hz, 3H), 1.35 (s, 9H) |
| 128 | 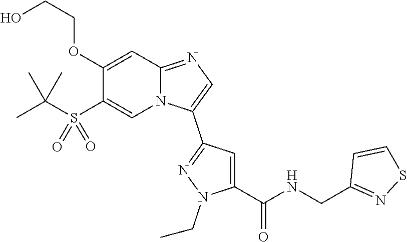 [SMILES: CCn1nc(-c2cnc3cc(OCCO)c(S(=O)(=O)C(C)(C)C)cn23)cc1C(=O)NCc1ccsn1] | 3-(6-(tert- butylsulfonyl)-7-(2- hydroxyethoxy)imidazo [1,2-a]pyridin-3-yl)-1- ethyl-N-(isothiazol-3- ylmethyl)-1H-pyrazole- 5-carboxamide | [M + H]⁺ = 533.3 ¹H NMR (400 MHz, DMSO- d₆) δ ppm 9.94 (s, 1H), 9.35 (t, J = 6.0 Hz, 1H), 9.04 (d, J = 4.4 Hz, 1H), 8.00-7.95 (m, 1H), 7.35-7.28 (m, 3H), 4.83 (t, J = 5.6 Hz, 1H), 4.63-4.54 (m, 4H), 4.21-4.17 (m, 2H), 3.78 (q, J = 4.8 Hz, 2H), 1.43 (t, J = 7.2 Hz, 3H), 1.34 (s, 9H) |
| 129 | 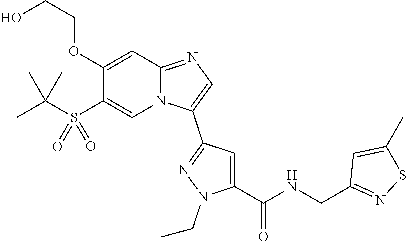 [SMILES: CCn1nc(-c2cnc3cc(OCCO)c(S(=O)(=O)C(C)(C)C)cn23)cc1C(=O)NCc1cc(C)sn1] | 3-(6-(tert- butylsulfonyl)-7-(2- hydroxyethoxy)imidazo [1,2-a]pyridin-3-yl)-1- ethyl-N-((5- methylisothiazol-3- yl)methyl)-1H- pyrazole-5- carboxamide | [M + H]⁺ = 547.0 ¹H NMR (400 MHz, DMSO- d₆) δ ppm 9.94 (s, 1H), 9.29 (t, J = 6.0 Hz, 1H), 7.97 (s, 1H), 7.30 (d, J = 4.8 Hz, 2H), 7.07 (s, 1H), 4.81 (t, J = 5.2 Hz, 1H), 4.58 (q, J = 7.2 Hz, 2H), 4.51 (d, J = 6.0 Hz, 2H), 4.19 (t, J = 4.8 Hz, 2H), 3.78 (q, J = 5.2 Hz, 2H), 2.55 (s, 3H), 1.44 (t, J = 7.2 Hz, 3H), 1.34 (s, 9H) |
| 130 | 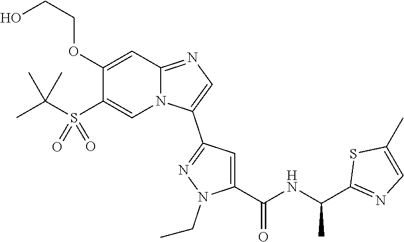 [SMILES: CCn1nc(-c2cnc3cc(OCCO)c(S(=O)(=O)C(C)(C)C)cn23)cc1C(=O)N[C@H](C)c1ncc(C)s1] | (R)-3-(6-(tert- butylsulfonyl)-7-(2- hydroxyethoxy)imidazo [1,2-a]pyridin-3-yl)-1- ethyl-N-(1-(5- methylthiazol-2- yl)ethyl)-1H-pyrazole- 5-carboxamide | [M + H]⁺ = 561.2 ¹H NMR (400 MHz, DMSO- d₆) δ ppm 9.96 (s, 1H), 9.33 (d, J = 8.0 Hz, 1H), 8.00 (s, 1H), 7.40 (d, J = 1.2 Hz, 1H), 7.35 (s, 1H), 7.30 (s, 1H), 5.34 (t, J = 7.2 Hz, 1H), 4.83 (t, J = 5.2 Hz, 1H), 4.55 (q, J = 7.2 Hz, 2H), 4.19 (t, J = 5.2 Hz, 2H), 3.78 (q, J = 4.8 Hz, 2H), 2.40 (d, J = 0.4 Hz, 3H), 1.59 (d, J = 7.2 Hz, 3H), 1.43 (t, J = 7.2 Hz, 3H), 1.34 (s, 9H) SFC: Rₜ = 2.556 min, 98.8% ee; (S,S) Whelk-O 1 100 x 4.6 |
| | | | mm, 5.0 μm; 50% EtOH |
| | | | (0.05% DEA) in CO₂, 2.5 |
| | | | mL/min; 40° C., 100 Bar, 220 |
| | | | nm |
| 131 | 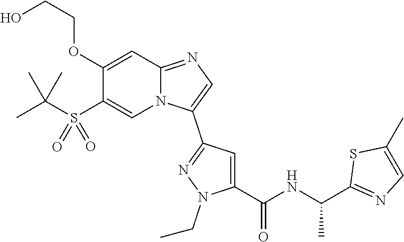 [SMILES: CCn1nc(-c2cnc3cc(OCCO)c(S(=O)(=O)C(C)(C)C)cn23)cc1C(=O)N[C@@H](C)c1ncc(C)s1] | (S)-3-(6-(tert- butylsulfonyl)-7-(2- hydroxyethoxy)imidazo [1,2-a]pyridin-3-yl)-1- ethyl-N-(1-(5- methylthiazol-2- yl)ethyl)-1H-pyrazole- 5-carboxamide | [M + H]⁺ = 561.2 ¹H NMR (500 MHz, DMSO- d₆) δ ppm 9.96 (s, 1H), 9.32 (d, J = 6.0 Hz, 1H), 8.00 (s, 1H), 7.40 (d, J = 1.2 Hz, 1H), 7.35 (s, 1H), 7.30 (s, 1H), 5.37-5.31 (m, 1H), 4.82 (t, J = 5.2 Hz, 1H), 4.59-4.53 (m, 2H), 4.19 (t, J = 4.0 Hz, 2H), 3.78 (q, J = 4.0 Hz, 2H), 2.40 (d, J = 0.9 Hz, 3H), 1.59 (d, J = 5.6 Hz, 3H), 1.44 (t, J = 7.2 Hz, 3H), 1.34 (s, 9H) SFC: Rₜ = 3.261 min, 100% ee; (S,S) Whelk-O 1 100 x 4.6 mm, 5.0 μm; 50% EtOH |
| | | | (0.05% DEA) in CO₂, 2.5 |
| | | | mL/min; 40° C., 100 Bar, 220 |
| | | | nm |
| 132 | 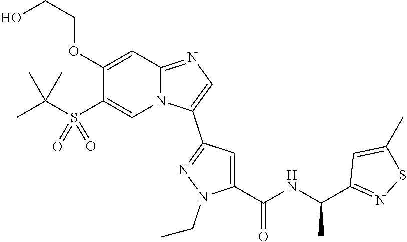 [SMILES: CCn1nc(-c2cnc3cc(OCCO)c(S(=O)(=O)C(C)(C)C)cn23)cc1C(=O)N[C@H](C)c1cc(C)sn1] | (R)-3-(6-(tert- butylsulfonyl)-7-(2- hydroxyethoxy)imidazo [1,2-a]pyridin-3-yl)-1- ethyl-N-(1-(5- methylisothiazol-3- yl)ethyl)-1H-pyrazole- 5-carboxamide | [M + H]⁺ = 561.1 ¹H NMR (400 MHz, DMSO- d₆) δ ppm 9.96 (s, 1H), 9.08 (d, J = 8.0 Hz, 1H), 7.98 (s, 1H), 7.36 (s, 1H), 7.29 (s, 1H), 7.11 (d, J = 0.8 Hz, 1H), 5.24 (q, J = 7.2 Hz, 1H), 4.82 (t, J = 5.2 Hz, 1H), 4.55 (q, J = 7.2 Hz, 2H), 4.19 (t, J = 4.8 Hz, 2H), 3.78 (q, J = 5.2 Hz, 2H), 2.54 (s, 3H), 1.52 (d, J = 7.2 Hz, 3H), 1.43 (t, J = 7.2 Hz, 3H), 1.34 (s, 9H) SFC: Rₜ = 0.785 min, 100% ee; (S,S) Whelk-O 1 50 x 4.6 |
| | | | mm, 3.5 μm; 50% i- |
| | | | PrOH/MeCN (7:3 + 0.05% |
| | | | DEA) in CO₂, 3 mL/min; 35º |
| | | | C., 100 Bar, 220 nm |
| 133 | 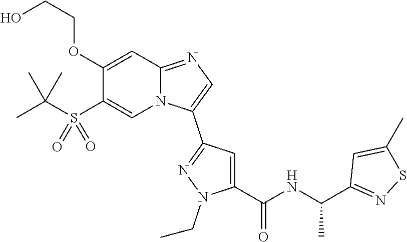 [SMILES: CCn1nc(-c2cnc3cc(OCCO)c(S(=O)(=O)C(C)(C)C)cn23)cc1C(=O)N[C@@H](C)c1cc(C)sn1] | (S)-3-(6-(tert- butylsulfonyl)-7-(2- hydroxyethoxy)imidazo [1,2-a]pyridin-3-yl)-1- ethyl-N-(1-(5- methylisothiazol-3- yl)ethyl)-1H-pyrazole- 5-carboxamide | [M + H]⁺ = 561.1 ¹H NMR (400 MHz, DMSO- d₆) δ ppm 9.96 (s, 1H), 9.08 (d, J = 8.4 Hz, 1H), 7.98 (s, 1H), 7.35 (s, 1H), 7.29 (s, 1H), 7.11 (d, J = 0.8 Hz, 1H), 5.29-5.18 (m, 1H), 4.81 (t, J = 5.2 Hz, 1H), 4.55 (q, J = 7.2 Hz, 2H), 4.19 (t, J = 5.2 Hz, 2H), 3.78 (q, J = 5.2 Hz, 2H), 2.54 (d, J = 0.8 Hz, 3H), 1.52 (d, J = 7.2 Hz, 3H), 1.43 (t, J = 7.2 Hz, 3H), 1.34 (s, 9H) SFC: Rₜ = 0.972 min, 99.0% ee; (S,S) Whelk-O 1 50 x 4.6 |
| | | | mm, 3.5 μm; 50% i- |
| | | | PrOH/MeCN (7:3 + 0.05% |
| | | | DEA) in CO₂, 3 mL/min; 35º |
| | | | C., 100 Bar, 220 nm |
| 134 | 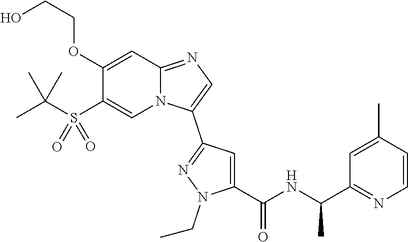 [SMILES: CCn1nc(-c2cnc3cc(OCCO)c(S(=O)(=O)C(C)(C)C)cn23)cc1C(=O)N[C@H](C)c1cc(C)ccn1] | (R)-3-(6-(tert- butylsulfonyl)-7-(2- hydroxyethoxy)imidazo [1,2-a]pyridin-3-yl)-1- ethyl-N-(1-(4- methylpyridin-2- yl)ethyl)-1H-pyrazole- 5-carboxamide | [M + H]⁺ = 555.3 ¹H NMR (400 MHz, DMSO- d₆) δ ppm 9.96 (s, 1H), 9.01 (d, J = 8.0 Hz, 1H), 8.39 (d, J = 4.8 Hz, 1H), 8.00 (s, 1H), 7.41 (s, 1H), 7.30 (s, 1H), 7.25 (s, 1H), 7.11 (d, J = 4.0 Hz, 1H), 5.14 (q, J = 7.2 Hz, 1H), 4.82 (t, J = 4.8 Hz, 1H), 4.53 (q, J = 7.2 Hz, 2H), 4.19 (t, J = 5.2 Hz, 2H), 3.78 (q, J = 4.8 Hz, 2H), 2.31 (s, 3H), 1.49 (d, J = 7.2 Hz, 3H), 1.41 (t, J = 7.2 Hz, 3H), 1.34 (s, 9H) SFC: Rₜ = 2.535 min, 100% |
| | | | ee; (S,S) Whelk-O 1 100 x 4.6 |
| | | | mm, 5.0 μm; 50% EtOH |
| | | | (0.05% DEA) in CO₂, 2.5 |
| | | | mL/min; 40° C., 100 Bar, 220 |
| | | | nm |
| 135 | 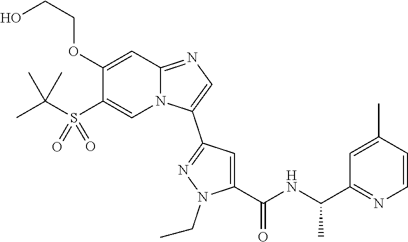 [SMILES: CCn1nc(-c2cnc3cc(OCCO)c(S(=O)(=O)C(C)(C)C)cn23)cc1C(=O)N[C@@H](C)c1cc(C)ccn1] | (S)-3-(6-(tert- butylsulfonyl)-7-(2- hydroxyethoxy)imidazo [1,2-a]pyridin-3-yl)-1- ethyl-N-(1-(4- methylpyridin-2- yl)ethyl)-1H-pyrazole- 5-carboxamide | [M + H]⁺ = 555.3 ¹H NMR (400 MHz, DMSO- d₆) δ ppm 9.96 (s, 1H), 9.01 (d, J = 8.0 Hz, 1H), 8.39 (d, J = 5.2 Hz, 1H), 8.01 (s, 1H), 7.41 (s, 1H), 7.30 (s, 1H), 7.25 (s, 1H), 7.11 (d, J = 4.8 Hz, 1H), 5.14 (q, J = 7.2 Hz, 1H), 4.83 (t, J = 5.2 Hz, 1H), 4.52 (q, J = 7.2 Hz, 2H), 4.19 (t, J = 5.2 Hz, 2H), 3.78 (q, J = 5.2 Hz, 2H), 2.31 (s, 3H), 1.49 (d, J = 7.2 Hz, 3H), 1.40 (t, J = 7.2 Hz, 3H), 1.34 (s, 9H) SFC: Rₜ = 3.713 min, 98.6% |
| | | | ee; (S,S) Whelk-O 1 100 x 4.6 |
| | | | mm, 5.0 μm; 50% EtOH |
| | | | (0.05% DEA) in CO₂, 2.5 |
| | | | mL/min; 40° C., 100 Bar, 220 |
| | | | nm |
| 136 | 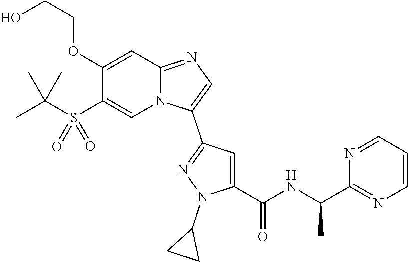 [SMILES: C[C@@H](NC(=O)c1cc(-c2cnc3cc(OCCO)c(S(=O)(=O)C(C)(C)C)cn23)nn1C1CC1)c1ncccn1] | (R)-3-(6-(tert- butylsulfonyl)-7-(2- hydroxyethoxy)imidazo [1,2-a]pyridin-3-yl)-1- cyclopropyl-N-(1- (pyrimidin-2-yl)ethyl)- 1H-pyrazole-5- carboxamide | [M + H]⁺ = 554.1 ¹H NMR (400 MHz, DMSO- d₆) δ ppm 9.92 (s, 1H), 9.06 (d, J = 7.2 Hz, 1H), 8.81 (d, J = 4.8 Hz, 2H), 8.00 (s, 1H), 7.45-7.38 (m, 2H), 7.29 (s, 1H), 5.21 (q, J = 7.2 Hz, 1H), 4.82 (t, J = 5.2 Hz, 1H), 4.59- 4.55 (m, 1H), 4.19 (t, J = 5.2 Hz, 2H), 3.78 (q, J = 4.8 Hz, 2H), 1.56 (d, J = 7.2 Hz, 3H), 1.35 (s, 9H), 1.25-1.20 (m, 2H), 1.04-0.97 (m, 2H) |
| 137 | 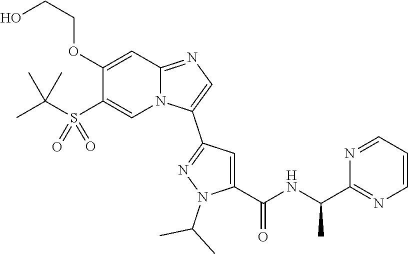 [SMILES: CC(C)n1nc(-c2cnc3cc(OCCO)c(S(=O)(=O)C(C)(C)C)cn23)cc1C(=O)N[C@H](C)c1ncccn1] | (R)-3-(6-(tert- butylsulfonyl)-7-(2- hydroxyethoxy)imidazo [1,2-a]pyridin-3-yl)-1- isopropyl-N-(1- (pyrimidin-2-yl)ethyl)- 1H-pyrazole-5- carboxamide | [M + H]⁺ = 556.1 ¹H NMR (400 MHz, DMSO- d₆) 8 ppm 10.09 (s, 1H), 9.04 (d, J = 7.6 Hz, 1H), 8.80 (d, J = 4.8 Hz, 2H), 8.01 (s, 1H), 7.41 (t, J = 4.8 Hz, 1H), 7.36 (s, 1H), 7.29 (s, 1H), 5.47-5.34 (m, 1H), 5.25-5.13 (m, 1H), 4.82 (s, 1H), 4.19 (t, J = 4.8 Hz, 2H), 3.78 (t, J = 4.4 Hz, 2H), 1.55 (d, J = 7.2 Hz, 3H), 1.48-1.42 (m, 6H), 1.35 (s, 9H) |
| 138 | 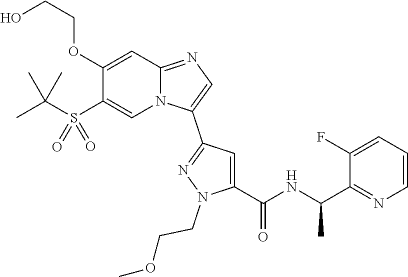 [SMILES: COCCn1nc(-c2cnc3cc(OCCO)c(S(=O)(=O)C(C)(C)C)cn23)cc1C(=O)N[C@H](C)c1ncccc1F] | (R)-3-(6-(tert- butylsulfonyl)-7-(2- hydroxyethoxy)imidazo [1,2-a]pyridin-3-yl)-N- (1-(3-fluoropyridin-2- yl)ethyl)-1-(2- methoxyethyl)-1H- pyrazole-5- carboxamide | [M + H]⁺ = 589.1 ¹H NMR (400 MHz, DMSO- d₆) δ ppm 9.92 (s, 1H), 9.07 (d, J = 7.6 Hz, 1H), 8.46-8.37 (m, 1H), 8.00 (s, 1H), 7.74-7.68 (m, 1H), 7.45-7.38 (m, 2H), 7.30 (s, 1H), 5.45 (t, J = 6.8 Hz, 1H), 4.82 (t, J = 5.2 Hz, 1H), 4.68 (t, J = 5.6 Hz, 2H), 4.19 (t, J = 4.8 Hz, 2H), 3.81- 3.72 (m, 4H), 3.19 (s, 3H), 1.51 (d, J = 6.8 Hz, 3H), 1.34 (s, 9H) |
| 139 | 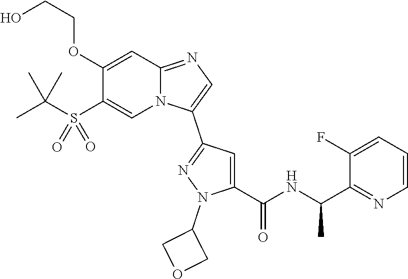 [SMILES: C[C@@H](NC(=O)c1cc(-c2cnc3cc(OCCO)c(S(=O)(=O)C(C)(C)C)cn23)nn1C1COC1)c1ncccc1F] | (R)-3-(6-(tert- butylsulfonyl)-7-(2- hydroxyethoxy)imidazo [1,2-a]pyridin-3-yl)-N- (1-(3-fluoropyridin-2- yl)ethyl)-1-(oxetan-3- yl)-1H-pyrazole-5- carboxamide | [M + H]⁺ = 587.1 ¹H NMR (400 MHz, DMSO- d₆) δ ppm 10.05 (s, 1H), 9.13 (d, J = 7.2 Hz, 1H), 8.41 (d, J = 4.4 Hz, 1H), 8.04 (s, 1H), 7.77-7.65 (m, 1H), 7.53 (s, 1H), 7.44-7.41 (m, 1H), 7.32 (s, 1H), 6.06 (q, J = 6.8 Hz, 1H), 5.43 (q, J = 6.8 Hz, 1H), 5.00 (q, J = 6.4 Hz, 2H), 4.94- 4.86 (m, 2H), 4.82 (s, 1H), 4.20 (t, J = 4.8 Hz, 2H), 3.78 (d, J = 3.6 Hz, 2H), 1.50 (d, J = 7.2 Hz, 3H), 1.35 (s, 9H) |
| 140 | 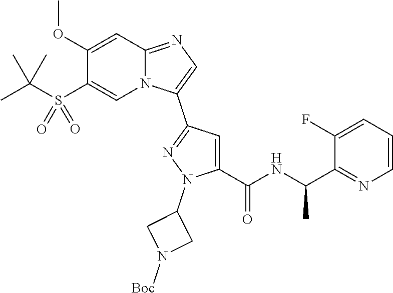 [SMILES: COc1cc2ncc(-c3cc(C(=O)N[C@H](C)c4ncccc4F)n(C4CN(C(=O)OC(C)(C)C)C4)n3)n2cc1S(=O)(=O)C(C)(C)C] | tert-butyl (R)-3-(3-(6- (tert-butylsulfonyl)-7- methoxyimidazo[1,2- a]pyridin-3-yl)-5-((1- (3-fluoropyridin-2- yl)ethyl)carbamoyl)- 1H-pyrazol-1- yl)azetidine-1- carboxylate | [M + H]⁺ = 656.1 ¹H NMR (400 MHz, DMSO- d₆) δ ppm 9.97 (s, 1H), 9.15 (d, J = 7.6 Hz, 1H), 8.45-8.38 (m, 1H), 8.05 (s, 1H), 7.76-7.67 (m, 1H), 7.51 (s, 1H), 7.46- 7.38 (m, 1H), 7.30 (s, 1H), 5.87-5.75 (m, 1H), 5.50-5.38 (m, 1H), 4.44-4.13 (m, 4H), 3.94 (s, 3H), 1.51 (d, J = 7.2 Hz, 3H), 1.40 (s, 9H), 1.33 (s, 9H) |
| 141 | 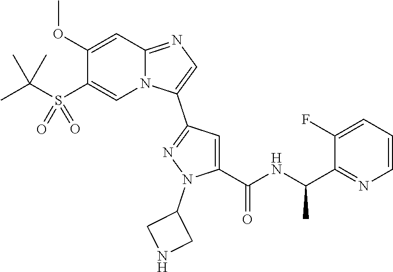 [SMILES: COc1cc2ncc(-c3cc(C(=O)N[C@H](C)c4ncccc4F)n(C4CNC4)n3)n2cc1S(=O)(=O)C(C)(C)C] | (R)-1-(azetidin-3-yl)-3- (6-(tert-butylsulfonyl)- 7-methoxyimidazo[1,2- a]pyridin-3-yl)-N-(1- (3-fluoropyridin-2- yl)ethyl)-1H-pyrazole- 5-carboxamide | [M + H]⁺ = 556.0 ¹H NMR (400 MHz, DMSO- d₆) δ ppm 10.07 (s, 1H), 9.08 (d, J = 7.2 Hz, 1H), 8.41 (d, J = 4.0 Hz, 1H), 8.03 (s, 1H), 7.71 (t, J = 8.8 Hz, 1H), 7.46- 7.39 (m, 2H), 7.30 (s, 1H), 5.80-5.72 (m, 1H), 5.48-5.39 (m, 1H), 4.05-3.97 (m, 2H), 3.95 (s, 3H), 3.78-3.63 (m, 3H), 1.50 (d, J = 6.8 Hz, 3H), 1.34 (s. 9H) |

### Example 142

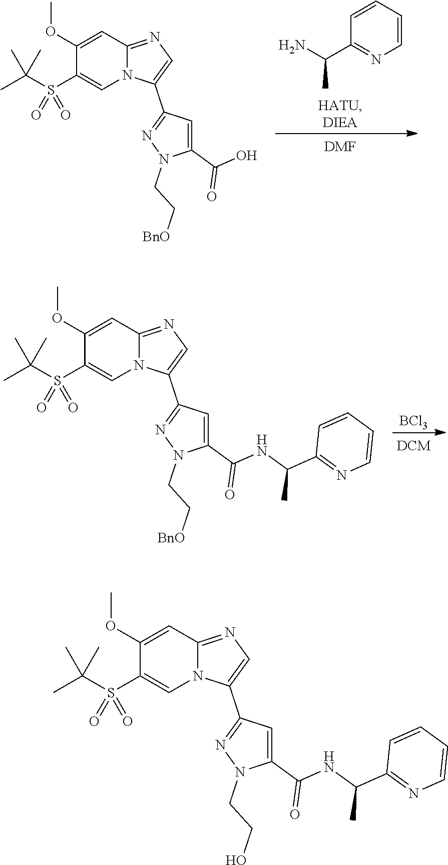

(R)-1-(2-(benzyloxy)ethyl)-3-(6-(tert-butylsulfonyl)-7-methoxyimidazo[1,2-a]pyridin-3-yl)-N-(1-(pyridin-2-yl)ethyl)-1H-pyrazole-5-carboxamide: To a solution of 1-(2-(benzyloxy)ethyl)-3-(6-(tert-butylsulfonyl)-7-methoxyimidazo[1,2-a]pyridin-3-yl)-1H-pyrazole-5-carboxylic acid (40 mg, 77.14 μmol, 1 eq) in DMF (2 mL) was added HATU (58.67 mg, 154.29 μmol, 2.0 eq), (R)-1-(pyridin-2-yl) ethan-1-amine (18.85 mg, 154.29 μmol, 2 eq) and DIEA (29.91 mg, 231.43 μmol, 40.31 μL, 3.0 eq). The mixture was stirred at 25° C. for 30 min before it was filtered and concentrated. The residue was purified by column chromatography (SiO₂, 0-10% MeOH/DCM) to give the title compound (30 mg, crude) as yellow oil. [M+H]⁺=617.1.

(R)-3-(6-(tert-butylsulfonyl)-7-methoxyimidazo[1,2-a]pyridin-3-yl)-1-(2-hydroxyethyl)-N-(1-(pyridin-2-yl)ethyl)-1H-pyrazole-5-carboxamide: To a solution of (R)-1-(2-(benzyloxy)ethyl)-3-(6-(tert-butylsulfonyl)-7-methoxyimidazo[1,2-a]pyridin-3-yl)-N-(1-(pyridin-2-yl)ethyl)-1H-pyrazole-5-carboxamide (25 mg, 40.54 μmol, 1 eq) in DCM (0.5 mL) was added BCl₃ (0.5 mL). The mixture was stirred at 25° C. for 2 hours before it was quenched with saturated aqueous NaHCO₃ (5 mL) and extracted with EtOAc (3×5 mL). The combined organic extracts were washed with brine (5 mL), dried over Na₂SO₄, filtered and concentrated under reduced pressure. The residue was purified by RP-HPLC to give the title compound (2.7 mg, 13% yield) as a white solid. [M+H]⁺=527.1. ¹H NMR (400 MHZ, DMSO-d₆) δ ppm 9.93 (s, 1H), 9.06 (d, J=7.6 Hz, 1H), 8.54 (d, J=4.4 Hz, 1H), 8.02 (s, 1H), 7.82-7.73 (m, 1H), 7.48-7.42 (m, 1H), 7.39 (8, 1H), 7.32-7.24 (m, 2H), 5.18 (t, J=7.2 Hz, 1H), 4.94 (t, J=5.6 Hz, 1H), 4.58 (t, J=5.6 Hz, 2H), 3.94 (s, 3H), 3.86-3.75 (m, 2H), 1.52 (d, J=7.2 Hz, 3H), 1.33 (8, 9H).

The following compounds were prepared following procedures analogous to that described for Example 142. Example 146 was prepared from the corresponding THP-protected intermediate by treatment with TFA in DCM rather than BCl₃.

| Ex. | | | |
|---|---|---|---|
| No. | Structure | Name | LCMS, ¹H NMR |
| 143 | 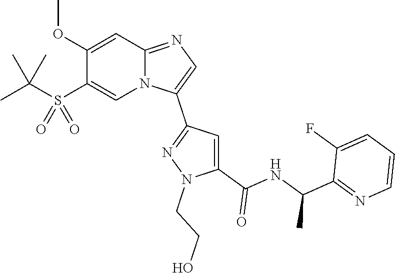 [SMILES: COc1cc2ncc(-c3cc(C(=O)N[C@H](C)c4ncccc4F)n(CCO)n3)n2cc1S(=O)(=O)C(C)(C)C] | (R)-3-(6-(tert- butylsulfonyl)-7- methoxyimidazo[1,2- a]pyridin-3-yl)-N-(1- (3-fluoropyridin-2- yl)ethyl)-1-(2- hydroxyethyl)-1H- pyrazole-5- carboxamide | [M + H]⁺ = 545.1 ¹H NMR (400 MHz, DMSO- d₆) δ ppm 9.91 (s, 1H), 9.05 (d, J = 7.6 Hz, 1H), 8.41 (d, J = 4.4 Hz, 1H), 8.00 (s, 1H), 7.71 (t, J = 8.8 Hz, 1H), 7.42 (td, J = 8.0, 4.0 Hz, 1H), 7.38 (s, 1H), 7.28 (s, 1H), 5.51-5.39 (m, 1H), 4.93 (t, J = 5.2 Hz, 1H), 4.62-4.52 (m, 2H), 3.94 (s, 3H), 3.78 (q, J = 5.6 Hz, 2H), 1.51 (d, J = 7.2 Hz, 3H), 1.32 (s, 9H) |
| 144 | 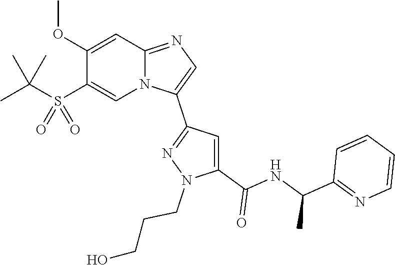 [SMILES: COc1cc2ncc(-c3cc(C(=O)N[C@H](C)c4ccccn4)n(CCCO)n3)n2cc1S(=O)(=O)C(C)(C)C] | (R)-3-(6-(tert- butylsulfonyl)-7- methoxyimidazo[1,2- a]pyridin-3-yl)-1-(3- hydroxypropyl)-N-(1- (pyridin-2-yl)ethyl)- 1H-pyrazole-5- carboxamide | [M + H]⁺ = 541.2 ¹H NMR (400 MHz, DMSO- d₆) δ ppm 9.92 (s, 1H), 9.07 (d, J = 7.6 Hz, 1H), 8.54 (d, J = 4.4 Hz, 1H), 8.02 (s, 1H), 7.81- 7.75 (m, 1H), 7.45-7.40 (m, 2H), 7.30-7.26 (m, 2H), 5.22- 5.14 (m, 1H), 4.57-4.50 (m, 3H), 3.94 (s, 3H), 3.41 (q, J = 6.4 Hz, 2H), 2.02-1.95 (m, 2H), 1.51 (d, J = 7.2 Hz, 3H), 1.32 (s, 9H) |
| 145 | 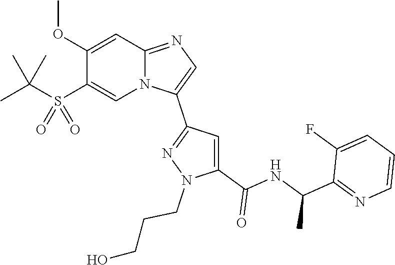 [SMILES: COc1cc2ncc(-c3cc(C(=O)N[C@H](C)c4ncccc4F)n(CCCO)n3)n2cc1S(=O)(=O)C(C)(C)C] | (R)-3-(6-(tert- butylsulfonyl)-7- methoxyimidazo[1,2- a]pyridin-3-yl)-N-(1- (3-fluoropyridin-2- yl)ethyl)-1-(3- hydroxypropyl)-1H- pyrazole-5- carboxamide | [M + H]⁺ = 559.0 ¹H NMR (400 MHz, DMSO- d₆) δ ppm 9.90 (s, 1H), 9.05 (d, J = 7.2 Hz, 1H), 8.41 (d, J = 4.8 Hz, 1H), 7.99 (s, 1H), 7.74- 7.68 (m, 1H), 7.44-7.38 (m, 2H), 7.28 (s, 1H), 5.49-5.41 (m, 1H), 4.56-4.47 (m, 3H), 3.94 (s, 3H), 3.43-3.38 (m, 2H), 1.96 (t, J = 6.8 Hz, 2H), 1.51 (d, J = 7.2 Hz, 3H), 1.32 (s, 9H) |
| 146 | 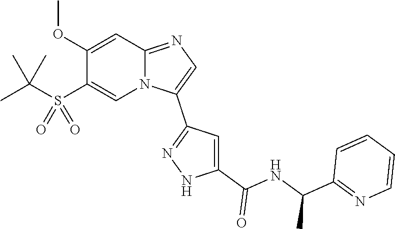 [SMILES: COc1cc2ncc(-c3cc(C(=O)N[C@H](C)c4ccccn4)[nH]n3)n2cc1S(=O)(=O)C(C)(C)C] | (R)-3-(6-(tert- butylsulfonyl)-7- methoxyimidazo[1,2- a]pyridin-3-yl)-N-(1- (pyridin-2-yl)ethyl)- 1H-pyrazole-5- carboxamide | [M + H]⁺ = 483.1 ¹H NMR (400 MHz, DMSO- d₆) δ ppm 14.20-13.38 (m, 1H), 9.99-9.80 (m, 1H), 9.04-8.86 (m, 1H), 8.54 (d, J = 4.4 Hz, 1H), 7.99 (s, 1H), 7.81-7.75 (m, 1H), 7.43 (d, J = 7.6 Hz, 2H), 7.30-7.26 (m, 2H), 5.23-5.16 (m, 1H), 3.94 (s, 3H), 1.52 (d, J = 7.2 Hz, 3H), 1.32 (s, 9H) |

### Example 147

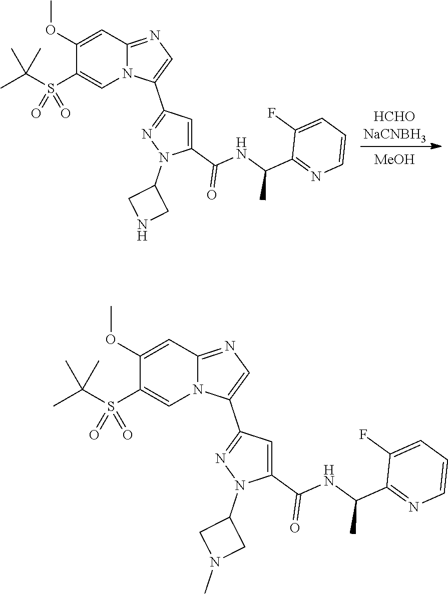

(R)-3-(6-(tert-butylsulfonyl)-7-methoxyimidazo[1,2-a]pyridin-3-yl)-N-(1-(3-fluoropyridin-2-yl)ethyl)-1-(1-methylazetidin-3-yl)-1H-pyrazole-5-carboxamide: To a solution of (R)-1-(azetidin-3-yl)-3-(6-(tert-butylsulfonyl)-7-methoxyimidazo[1,2-a]pyridin-3-yl)-N-(1-(3-fluoropyridin-2-yl)ethyl)-1H-pyrazole-5-carboxamide (20 mg, 36.00 μmol, 1 eq) in MeOH (2 mL) was added formalin (29.21 mg, 359.96 μmol, 26.80 μL, 37% purity, 10 eq). The mixture was stirred at 20° C. for 1 hour before NaBH₃CN (4.52 mg, 71.99 μmol, 2 eq) was added. The resulting mixture was stirred at 20° C. for 12 hours, and then it was concentrated under reduced pressure. The residue was purified by RP-HPLC to yield the title compound (2.4 mg, 24% yield) as a white solid. [M+H]⁺=570.1. ¹H NMR (400 MHZ, DMSO-d₆) δ ppm 10.14 (s, 1H), 9.09 (d, J=7.2 Hz, 1H), 8.41 (d, J=4.4 Hz, 1H), 8.05 (s, 1H), 7.79-7.68 (m, 1H), 7.45-7.39 (m, 2H), 7.30 (s, 1H), 5.66 (q, J=6.8 Hz, 1H), 5.44 (q, J=7.2 Hz, 1H), 3.95 (s, 3H), 3.75-3.65 (m, 2H), 3.47 (q, J=7.2 Hz, 2H), 2.37 (s, 3H), 1.50 (d, J=7.2 Hz, 3H), 1.35 (s, 9H).

### Example 148

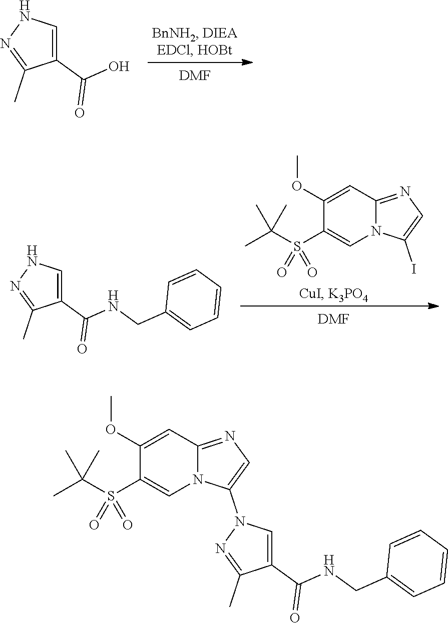

N-benzyl-3-methyl-1H-pyrazole-4-carboxamide: A mixture of 3-methyl-1H-pyrazole-4-carboxylic acid (100 mg, 792.94 μmol, 1 eq), EDCI (304.01 mg, 1.59 mmol, 2 eq), HOBt (214.29 mg, 1.59 mmol, 2 eq) and DIEA (204.96 mg, 1.59 mmol, 276.23 μL, 2 eq) in DMF (1 mL) was cooled to 0° C., and then a solution of benzylamine (169.93 mg, 1.59 mmol, 172.87 μL, 2 eq) in DMF (2 mL) was added dropwise. The resulting mixture was stirred at 25° C. for 12 hours before it was partitioned between EtOAc (20 mL) and water (10 mL). The organic phase was separated, washed with brine (3×10 mL), dried over Na₂SO₄, filtered and concentrated under reduced pressure. The residue was purified by column chromatography (SiO₂, 0-60% EtOAc/petroleum ether) to give the title compound (140 mg, 74% yield) as a white solid. 1H NMR (400 MHZ, CDCl₃) δ ppm 7.73 (s, 1H), 7.43-7.26 (m, 5H), 6.06 (s, 1H), 4.60 (d, J=5.6 Hz, 2H), 2.58 (s, 3H).

N-benzyl-1-(6-(tert-butylsulfonyl)-7-methoxyimidazo[1,2-a]pyridin-3-yl)-3-methyl-1H-pyrazole-4-carboxamide: A mixture of N-benzyl-3-methyl-1H-pyrazole-4-carboxamide (98.28 mg, 410.93 μmol, 1 eq), 6-(tert-butylsulfonyl)-3-iodo-7-methoxyimidazo[1,2-a]pyridine (180 mg, 410.93 μmol, 1 eq), Cul (39.13 mg, 205.47 μmol, 0.5 eq), trans-N,N′-dimethylcyclohexane-1,2-diamine (46.76 mg, 328.74 μmol, 0.8 eq) and K₃PO₄ (174.46 mg, 821.86 μmol, 2 eq) in DMF (0.5 mL) was degassed and purged with N₂ three times. The mixture was stirred at 80° C. for 12 hours under a N₂ atmosphere before it was partitioned between EtOAc (20 mL) and water (10 mL). The organic phase was separated, washed with brine (3×10 mL), dried over Na₂SO₄, filtered and concentrated under reduced pressure. The residue was purified by column chromatography (SiO₂, 0-7% MeOH/DCM) and RP-HPLC to give the title compound (2.62 mg, 1.3% yield) as a white solid. [M+H]⁺=482.1. ¹H NMR (400 MHz, DMSO-d₆) δ ppm 8.73 (s, 1H), 8.66 (s, 1H), 8.62 (s, 1H), 7.83 (s, 1H), 7.37-7.31 (m, 4H), 7.29 (s, 1H), 7.27-7.22 (m, 1H), 4.45 (d, J=6.0 (Hz, 2H), 3.94 (s, 2H), 2.47 (s, 3H), 1.30 (s, 9H).

### Example 149

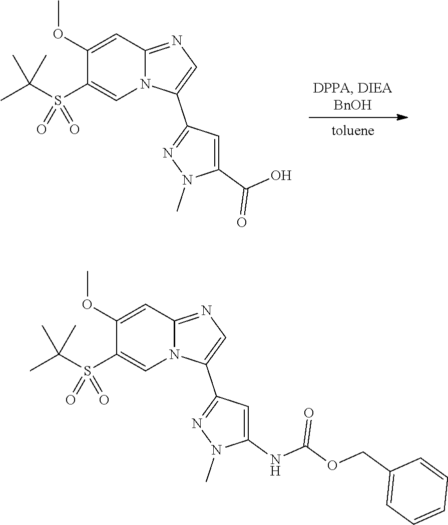 [SMILES: COc1cc2ncc(-c3cc(C(=O)O)n(C)n3)n2cc1S(=O)(=O)C(C)(C)C ; "DPPA, DIEABnOH" ; "toluene" ; COc1cc2ncc(-c3cc(NC(=O)OCc4ccccc4)n(C)n3)n2cc1S(=O)(=O)C(C)(C)C]

A mixture of 3-(6-(tert-butylsulfonyl)-7-methoxyimidazo[1,2-a]pyridin-3-yl)-1-methyl-1H-pyrazole-5-carboxylic acid (20 mg, 50.96 μmol, 1 eq), DPPA (16.83 mg, 61.16 μmol, 13.20 μL, 1.2 eq) and DIEA (9.88 mg, 76.45 μmol, 13.32 μL, 1.5 eq) in toluene (1 mL) and benzyl alcohol (1 mL) was degassed and purged with N₂ three times, and then the mixture was stirred at 90° C. for 3 hours under a N₂ atmosphere (gas evolution). The reaction mixture was filtered and concentrated under reduced pressure to give a residue, which was purified by column chromatography (SiO₂, 75% EtOAc/petroleum ether). The resulting material was purified by RP-HPLC to provide the title compound (3.07 mg, 12% yield) as a white solid. [M+H]⁺=498.1. ¹H NMR (400 MHZ, DMSO-d₆) δ ppm 10.03-9.97 (m, 1H), 9.89 (s, 1H), 8.01 (s, 1H), 7.46-7.34 (m, 5H), 7.26-7.23 (m, 1H), 6.66 (s, 1H), 5.20 (s, 2H), 3.93 (s, 3H), 3.75 (s, 3H), 1.32 (s, 9H).

The following compounds were prepared following procedures analogous to that described for Example 149.

| Ex. | | | |
|---|---|---|---|
| No. | Structure | Name | LCMS, ¹H NMR |
| 150 | 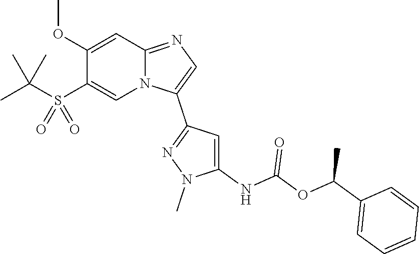 [SMILES: COc1cc2ncc(-c3cc(NC(=O)O[C@@H](C)c4ccccc4)n(C)n3)n2cc1S(=O)(=O)C(C)(C)C] | (S)-1-phenylethyl (3- (6-(tert- butylsulfonyl)-7- methoxyimidazo[1,2- a]pyridin-3-yl)-1- methyl-1H-pyrazol- 5-yl)carbamate | [M + H]⁺ = 512.2 ¹H NMR (400 MHz, DMSO- d₆) δ ppm 10.00-9.94 (m, 1H), 9.88 (s, 1H), 7.99 (s, 1H), 7.45-7.37 (m, 4H), 7.35-7.32 (m, 1H), 7.25 (s, 1H), 6.63 (s, 1H), 5.86-5.80 (m, 1H), 3.92 (s, 3H), 3.74 (s, 3H), 1.57 (d, J = 6.4 Hz, 3H), 1.31 (s, 9H) |
| 151 | 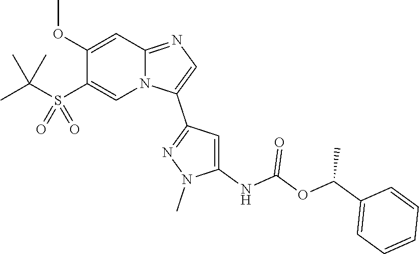 [SMILES: COc1cc2ncc(-c3cc(NC(=O)O[C@H](C)c4ccccc4)n(C)n3)n2cc1S(=O)(=O)C(C)(C)C] | (R)-1-phenylethyl (3- (6-(tert- butylsulfonyl)-7- methoxyimidazo[1,2- a]pyridin-3-yl)-1- methyl-1H-pyrazol- 5-yl)carbamate | [M + H]⁺ = 512.2 ¹H NMR (400 MHz, DMSO- d₆) δ ppm 10.01-9.97 (m, 1H), 9.88 (s, 1H), 8.49-8.45 (m, 0.15H), 7.99 (s, 1H), 7.45-7.36 (m, 4H), 7.33 (d, J = 7.2 Hz, 1H), 7.25 (s, 1H), 6.63 (s, 1H), 5.86-5.78 (m, 1H), 3.92 (s, 3H), 3.74 (s, 3H), 1.57 (s, J = 6.8 Hz, 3H), 1.31 (s, 9H) |

### Example 152

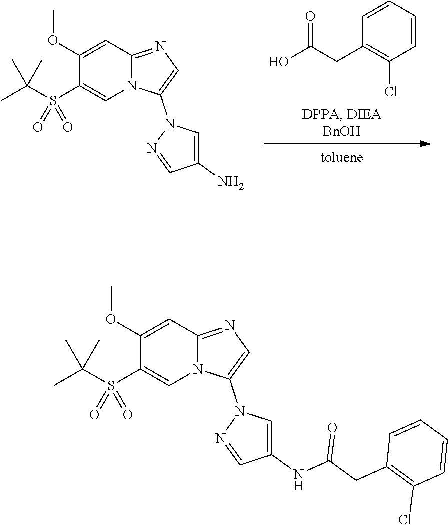 [SMILES: COc1cc2ncc(-n3cc(N)cn3)n2cc1S(=O)(=O)C(C)(C)C ; O=C(O)Cc1ccccc1Cl ; "DPPA, DIEABnOH" ; "toluene" ; COc1cc2ncc(-n3cc(NC(=O)Cc4ccccc4Cl)cn3)n2cc1S(=O)(=O)C(C)(C)C]

N-(1-(6-(tert-butylsulfonyl)-7-methoxyimidazo[1,2-a]pyridin-3-yl)-1H-pyrazol-4-yl)-2-(2-chlorophenyl) acetamide: To a stirred solution of 2-(2-chlorophenyl) acetic acid (13.18 mg, 77.27 μmol, 1.2 eq) and HATU (48.97 mg, 128.79 μmol, 2 eq) in DMF (4 mL) was added DIEA (24.97 mg, 193.18 μmol, 33.65 μL, 3 eq) and 1-(6-(tert-butylsulfonyl)-7-methoxyimidazo[1,2-a]pyridin-3-yl)-1H-pyrazol-4-amine (25 mg, 64.39 μmol, 90% purity, 1 eq). The reaction mixture was stirred at 25° C. for 1 hour under N₂ before it was diluted with water (15 mL) and extracted with EtOAc (3×15 mL). The combined organic layers were washed with brine (10 mL), dried over Na₂SO₄, filtered and concentrated under reduced pressure to give a residue, which was purified by RP-HPLC to give the title compound (5.1 mg, 16% yield) as a white solid. [M+H]⁺=501.9. ¹H NMR (400 MHZ, DMSO-d₆) δ ppm 10.55 (s, 1H), 8.74 (s, 1H), 8.34 (s, 1H), 7.96 (s, 1H), 7.84 (s, 1H), 7.49-7.39 (m, 2H), 7.35-7.29 (m, 2H), 7.28 (s, 1H), 3.93 (s, 3H), 3.83 (s, 2H), 1.29 (s, 9H).

The following compounds were prepared following procedures analogous to that described for Example 152.

| Ex. No. | Structure | Name | LCMS, ¹H NMR |
|---|---|---|---|
| 153 | 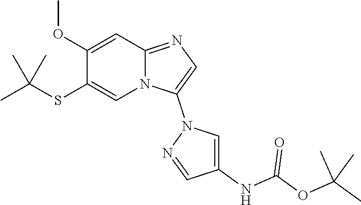 [SMILES: COc1cc2ncc(-n3cc(NC(=O)OC(C)(C)C)cn3)n2cc1SC(C)(C)C] | tert-butyl (1-(6-(tert- butylthio)-7- methoxyimidazo[1,2- a]pyridin-3-yl)-1H- pyrazol-4-yl)carbamate | [M + H]⁺ = 418.0 ¹H NMR (400 MHz, DMSO-d₆) δ ppm 9.47 (s, 1H), 8.25 (s, 1H), 8.03 (s, 1H), 7.77 (s, 1H), 7.68 (s, 1H), 7.10 (s, 1H), 3.89 (s, 3H), 1.47 (s, 9H), 1.22 (s, 9H) |
| 154 | 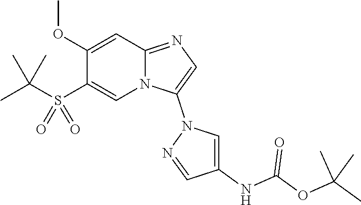 [SMILES: COc1cc2ncc(-n3cc(NC(=O)OC(C)(C)C)cn3)n2cc1S(=O)(=O)C(C)(C)C] | tert-butyl (1-(6-(tert- butylsulfonyl)-7- methoxyimidazo[1,2- a]pyridin-3-yl)-1H- pyrazol-4-yl)carbamate | [M + H]⁺ = 450.1 ¹H NMR (400 MHz, DMSO-d₆) δ ppm 9.51 (s, 1H), 8.73 (s, 1H), 8.10 (s, 1H), 7.84 (s, 1H), 7.78 (s, 1H), 7.27 (s, 1H), 3.93 (s, 3H), 1.47 (s, 9H), 1.29 (s, 9H) |
| 155 | 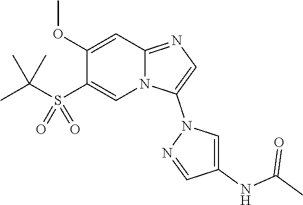 [SMILES: COc1cc2ncc(-n3cc(NC(C)=O)cn3)n2cc1S(=O)(=O)C(C)(C)C] | N-(1-(6-(tert-butylsulfonyl)- 7-methoxyimidazo[1,2- a]pyridin-3-yl)-1H- pyrazol-4-yl)acetamide | [M + H]⁺ = 392.1 ¹H NMR (400 MHz, DMSO-d₆) δ ppm 10.27 (s, 1H), 8.71 (s, 1H), 8.33 (s, 1H), 7.90 (s, 1H), 7.85 (s, 1H), 7.28 (s, 1H), 3.93 (s, 3H), 2.04 (s, 3H), 1.29 (s, 9H) |
| 156 | 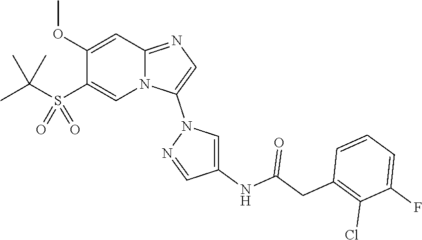 [SMILES: COc1cc2ncc(-n3cc(NC(=O)Cc4cccc(F)c4Cl)cn3)n2cc1S(=O)(=O)C(C)(C)C] | N-(1-(6-(tert-butylsulfonyl)- 7-methoxyimidazo[1,2- a]pyridin-3-yl)-1H- pyrazol-4-yl)-2-(2-chloro-3- fluorophenyl)acetamide | [M + H]⁺ = 520.1 ¹H NMR (400 MHz, DMSO-d₆) δ ppm 10.60 (s, 1H), 8.77 (s, 1H), 8.36 (s, 1H), 7.97 (s, 1H), 7.92 (s, 1H), 7.41-7.33 (m, 2H), 7.32-7.28 (m, 2H), 3.95 (s, 3H), 3.89 (s, 2H), 1.29 (s, 9H) |
| 157 | 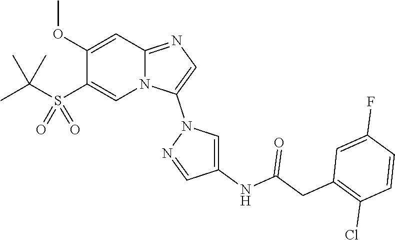 [SMILES: COc1cc2ncc(-n3cc(NC(=O)Cc4cc(F)ccc4Cl)cn3)n2cc1S(=O)(=O)C(C)(C)C] | N-(1-(6-(tert-butylsulfonyl)- 7-methoxyimidazo[1,2- a]pyridin-3-yl)-1H- pyrazol-4-yl)-2-(2- chloro-5-fluorophenyl) acetamide | [M + H]⁺ = 520.1 ¹H NMR (400 MHz, DMSO-d₆) δ ppm 10.56 (s, 1H), 8.75 (s, 1H), 8.35 (s, 1H), 7.96 (s, 1H), 7.88 (s, 1H), 7.50 (dd, J = 8.8, 5.2 Hz, 1H), 7.35 (dd, J = 9.5, 3.1 Hz, 1H), 7.29 (s, 1H), 7.19 (dt, J = 8.5, 3.0 Hz, 1H), 3.94 (s, 3H), 3.85 (s, 2H), 1.29 (s, 9H) |
| 158 | 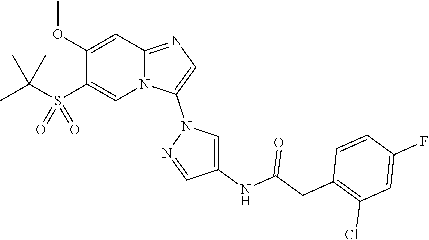 [SMILES: COc1cc2ncc(-n3cc(NC(=O)Cc4ccc(F)cc4Cl)cn3)n2cc1S(=O)(=O)C(C)(C)C] | N-(1-(6-(tert- butylsulfonyl)-7- methoxyimidazo[1,2- a]pyridin-3-yl)-1H- pyrazol-4-yl)-2-(2- chloro-4-fluorophenyl) acetamide | [M + H]⁺ = 520.1 ¹H NMR (500 MHz, DMSO-d₆) δ ppm 10.55 (s, 1H), 8.77-8.73 (m, 1H), 8.34 (s, 1H), 7.96 (s, 1H), 7.87 (s, 1H), 7.49 (dd, J = 8.6, 6.3 Hz, 1H), 7.44 (dd, J = 8.9, 2.7 Hz, 1H), 7.29 (s, 1H), 7.22 (dd, J = 8.5, 2.7 Hz, 1H), 3.94 (s, 3H), 3.82 (s, 2H), 1.29 (s, 9H) |
| 159 | 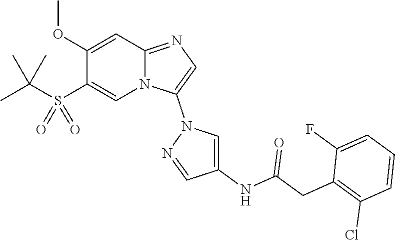 [SMILES: COc1cc2ncc(-n3cc(NC(=O)Cc4c(F)cccc4Cl)cn3)n2cc1S(=O)(=O)C(C)(C)C] | N-(1-(6-(tert-butylsulfonyl)- 7-methoxyimidazo[1,2-a] pyridin-3-yl)-1H- pyrazol-4-yl)-2-(2- chloro-6- fluorophenyl)acetamide | [M + H]⁺ = 520.1 ¹H NMR (500 MHz, DMSO-d₆) δ ppm 10.65 (s, 1H), 8.75 (s, 1H), 8.33 (s, 1H), 7.96 (s, 1H), 7.87 (s, 1H), 7.41-7.33 (m, 2H), 7.29 (s, 1H), 7.27-7.22 (m, 1H), 3.94 (s, 3H), 3.88 (s, 2H), 1.29 (s, 9H) |
| 160 | 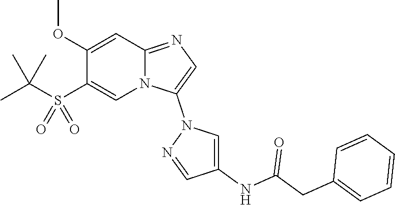 [SMILES: COc1cc2ncc(-n3cc(NC(=O)Cc4ccccc4)cn3)n2cc1S(=O)(=O)C(C)(C)C] | N-(1-(6-(tert- butylsulfonyl)-7- methoxyimidazo[1,2-a] pyridin-3-yl)-1H- pyrazol-4-yl)-2- phenylacetamide | [M + H]⁺ = 468.1 ¹H NMR (500 MHz, DMSO-d₆) δ ppm 10.52 (s, 1H), 8.76 (s, 1H), 8.37 (s, 1H), 7.95 (s, 1H), 7.92 (s, 1H), 7.33 (d, J = 4.4 Hz, 4H), 7.30 (s, 1H), 7.25 (dd, J = 8.6, 4.3 Hz, 1H), 3.95 (s, 3H), 3.64 (s, 2H), 1.29 (s, 9H) |
| 161 | 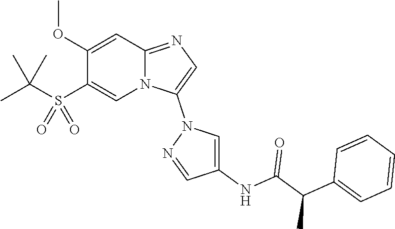 [SMILES: COc1cc2ncc(-n3cc(NC(=O)[C@H](C)c4ccccc4)cn3)n2cc1S(=O)(=O)C(C)(C)C] | (R)-N-(1-(6-(tert- butylsulfonyl)-7- methoxyimidazo[1,2- a]pyridin-3-yl)-1H- pyrazol-4-yl)-2- phenylpropanamide | [M + H]⁺ = 482.1 ¹H NMR (500 MHz, DMSO-d₆) δ ppm 10.42 (s, 1H), 8.75 (s, 1H), 8.37 (s, 1H), 7.93 (s, 1H), 7.88 (s, 1H), 7.40-7.36 (m, 2H), 7.35-7.31 (m, 2H), 7.30-7.21 (m, 2H), 3.94 (s, 3H), 3.81 (q, J = 7.0 Hz, 1H), 1.43 (d, J = 7.0 Hz, 3H), 1.28 (s, 9H) |
| 162 | 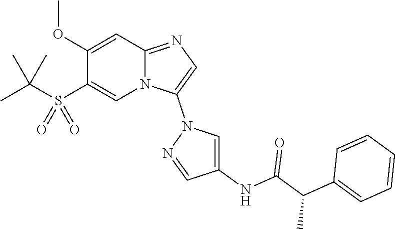 [SMILES: COc1cc2ncc(-n3cc(NC(=O)[C@@H](C)c4ccccc4)cn3)n2cc1S(=O)(=O)C(C)(C)C] | (S)-N-(1-(6-(tert- butylsulfonyl)-7- methoxyimidazo[1,2- a]pyridin-3-yl)-1H- pyrazol-4-yl)-2- phenylpropanamide | [M + H]⁺ = 482.2 ¹H NMR (400 MHz, DMSO-d₆) δ ppm 10.41 (s, 1H), 8.75 (s, 1H), 8.37 (s, 1H), 7.93 (s, 1H), 7.90 (s, 1H), 7.39-7.31 (m, 4H), 7.30-7.21 (m, 2H), 3.94 (s, 3H), 3.81 (q, J = 7.0 Hz, 1H), 1.43 (d, J = 7.0 Hz, 3H), 1.30-1.27 (m, 1H), 1.28 (s, 8H) |
| 163 | 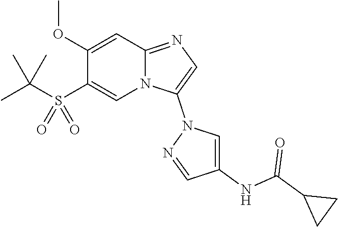 [SMILES: COc1cc2ncc(-n3cc(NC(=O)C4CC4)cn3)n2cc1S(=O)(=O)C(C)(C)C] | N-(1-(6-(tert- butylsulfonyl)-7- methoxyimidazo[1,2- a]pyridin-3-yl)-1H- pyrazol-4- yl)cyclopropanecarboxamide | [M + H]⁺ = 418.1 ¹H NMR (500 MHz, DMSO-d₆) δ ppm 10.51 (s, 1H), 8.73 (s, 1H), 8.32 (s, 1H), 7.92 (s, 1H), 7.84 (s, 1H), 7.28 (s, 1H), 3.93 (s, 3H), 1.74 (quin, J = 6.1 Hz, 1H), 1.29 (s, 9H), 0.82-0.78 (m, 4H) |
| 164 | 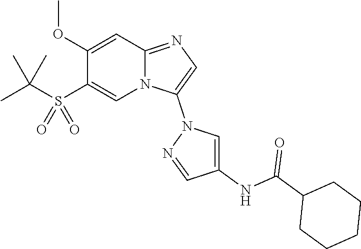 [SMILES: COc1cc2ncc(-n3cc(NC(=O)C4CCCCC4)cn3)n2cc1S(=O)(=O)C(C)(C)C] | N-(1-(6-(tert- butylsulfonyl)-7- methoxyimidazo[1,2- a]pyridin-3-yl)-1H- pyrazol-4- yl)cyclohexanecarboxamide | [M + H]⁺ = 460.2 ¹H NMR (500 MHz, DMSO-d₆) δ ppm 10.15 (s, 1H), 8.72 (s, 1H), 8.34 (s, 1H), 7.91 (s, 1H), 7.84 (s, 1H), 7.28 (s, 1H), 3.93 (s, 3H), 2.30 (tt, J = 11.6, 3.3 Hz, 1H), 1.84-1.71 (m, 4H), 1.65 (d, J = 12.5 Hz, 1H), 1.47-1.36 (m, 2H), 1.29 (s, 10H), 1.26-1.14 (m, 2H) |
| 165 | 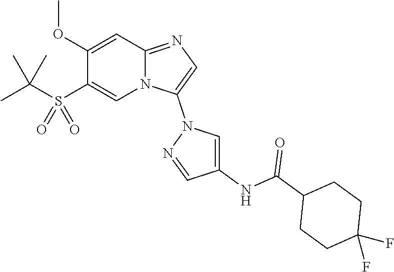 [SMILES: COc1cc2ncc(-n3cc(NC(=O)C4CCC(F)(F)CC4)cn3)n2cc1S(=O)(=O)C(C)(C)C] | N-(1-(6-(tert- butylsulfonyl)-7- methoxyimidazo[1,2- a]pyridin-3-yl)-1H- pyrazol-4-yl)-4,4- difluorocyclohexane- 1-carboxamide | [M + H]⁺ = 496.2 ¹H NMR (400 MHz, DMSO-d₆) δ ppm 10.32 (s, 1H), 8.73 (s, 1H), 8.37 (s, 1H), 7.93 (s, 1H), 7.87 (d, J = 3.6 Hz, 1H), 7.29 (s, 1H), 3.94 (s, 3H), 2.48-2.44 (m, 1H), 2.10 (d, J = 9.5 Hz, 2H), 1.96-1.80 (m, 4H), 1.77-1.63 (m, 2H), 1.29 (s, 9H) |
| 166 | 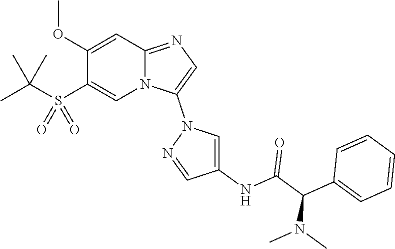 [SMILES: COc1cc2ncc(-n3cc(NC(=O)[C@@H](c4ccccc4)N(C)C)cn3)n2cc1S(=O)(=O)C(C)(C)C] | (R)-N-(1-(6-(tert- butylsulfonyl)-7- methoxyimidazo[1,2- a]pyridin-3-yl)-1H- pyrazol-4-yl)-2- (dimethylamino)-2- phenylacetamide | [M + H]⁺ = 511.2 ¹H NMR (500 MHz, DMSO-d₆) δ ppm 10.58 (s, 1H), 8.73 (s, 1H), 8.35 (s, 1H), 8.13 (s, 1H), 8.03 (s, 1H), 7.86-7.80 (m, 1H), 7.49 (d, J = 7.2 Hz, 2H), 7.39-7.34 (m, 2H), 7.34-7.29 (m, 1H), 7.27 (s, 1H), 3.92 (s, 3H), 3.84 (s, 1H), 2.19 (s, 6H), 1.28 (s, 9H) |
| 167 | 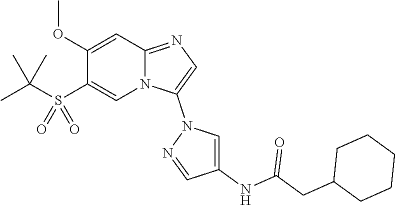 [SMILES: COc1cc2ncc(-n3cc(NC(=O)CC4CCCCC4)cn3)n2cc1S(=O)(=O)C(C)(C)C] | N-(1-(6-(tert- butylsulfonyl)-7- methoxyimidazo[1,2- a]pyridin-3-yl)-1H- pyrazol-4-yl)-2- cyclohexylacetamide | [M + H]⁺ = 474.2 ¹H NMR (500 MHz, DMSO-d₆) δ ppm 10.20 (s, 1H), 8.74 (s, 1H), 8.35 (s, 1H), 7.91 (s, 1H), 7.85 (s, 1H), 7.28 (s, 1H), 3.93 (s, 3H), 2.17 (d, J = 7.2 Hz, 2H), 1.83-1.56 (m, 6H), 1.29 (s, 9H), 1.25-1.08 (m, 3H), 1.03-0.91 (m, 2H) |
| 168 | 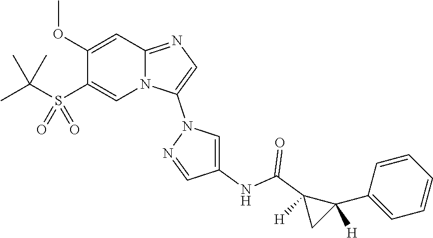 [SMILES: COc1cc2ncc(-n3cc(NC(=O)[C@@H]4C[C@H]4c4ccccc4)cn3)n2cc1S(=O)(=O)C(C)(C)C] | (1R,2R)-N-(1-(6- (tert-butylsulfonyl)-7- methoxyimidazo[1,2-a] pyridin-3-yl)-1H- pyrazol-4-yl)-2- phenylcyclopropane- 1-carboxamide | [M + H]⁺ = 494.2 ¹H NMR (400 MHz, DMSO-d₆) δ ppm 10.59 (s, 1H), 8.74 (d, J = 3.6 Hz, 1H), 8.36 (s, 1H), 7.93 (s, 1H), 7.89 (d, J = 8.0 Hz, 1H), 7.32-7.27 (m, 3H), 7.22-7.17 (m, 3H), 3.94 (s, 3H), 2.40-2.34 (m, 1H), 2.06-2.00 (m, 1H), 1.53-1.47 (m, 1H), 1.41- 1.35 (m, 1H), 1.29 (s, 9H) |
| 169 | 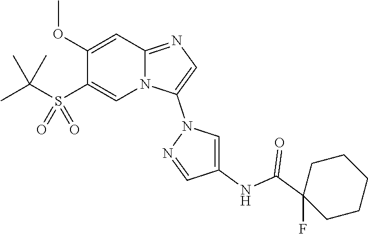 [SMILES: COc1cc2ncc(-n3cc(NC(=O)C4(F)CCCCC4)cn3)n2cc1S(=O)(=O)C(C)(C)C] | N-(1-(6-(tert- butylsulfonyl)-7- methoxyimidazo[1,2- a]pyridin-3-yl)-1H- pyrazol-4-yl)-1- fluorocyclohexane- 1-carboxamide | [M + H]⁺ = 478.2 ¹H NMR (500 MHz, DMSO-d₆) δ ppm 10.51 (d, J = 2.7 Hz, 1H), 8.70 (s, 1H), 8.40 (s, 1H), 8.05 (s, 1H), 7.86 (s, 1H), 7.28 (s, 1H), 3.94 (s, 3H), 1.99-1.78 (m, 4H), 1.67 (d, J = 9.9 Hz, 3H), 1.60-1.47 (m, 2H), 1.29 (s, 10H) |
| 170 | 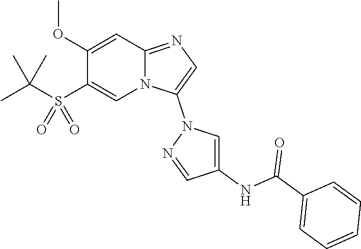 [SMILES: COc1cc2ncc(-n3cc(NC(=O)c4ccccc4)cn3)n2cc1S(=O)(=O)C(C)(C)C] | N-(1-(6-(tert- butylsulfonyl)-7- methoxyimidazo[1,2- a]pyridin-3-yl)-1H- pyrazol-4- yl)benzamide | [M + H]⁺ = 454.1 ¹H NMR (400 MHz, DMSO-d₆) δ ppm 10.76 (s, 1H), 8.76 (s, 1H), 8.54 (s, 1H), 8.11 (s, 1H), 8.02-7.97 (m, 2H), 7.91 (s, 1H), 7.64-7.53 (m, 3H), 7.30 (s, 1H), 3.95 (s, 3H), 1.30 (s, 9H) |
| 171 | 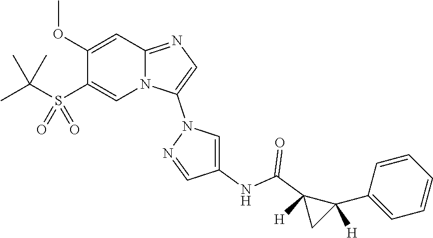 [SMILES: COc1cc2ncc(-n3cc(NC(=O)[C@H]4C[C@H]4c4ccccc4)cn3)n2cc1S(=O)(=O)C(C)(C)C] | (1S,2R)-N-(1-(6- (tert-butylsulfonyl)-7- methoxyimidazo[1,2-a] pyridin-3-yl)-1H- pyrazol-4-yl)-2- phenylcyclopropane-1- carboxamide | [M + H]⁺ = 494.2 ¹H NMR (400 MHz, DMSO-d₆) δ ppm 10.47 (s, 1H), 8.72 (s, 1H), 8.16 (s, 1H), 7.86-7.72 (m, 2H), 7.26-7.18 (m, 5H), 7.15-7.09 (m, 1H), 3.92 (s, 3H), 2.58-2.53 (m, 1H), 2.21 (dt, J = 8.4, 5.8 Hz, 1H), 1.65-1.57 (m, 1H), 1.33 (dt, J = 8.3, 4.4 Hz, 1H), 1.27 (s, 9H) |
| 172 | 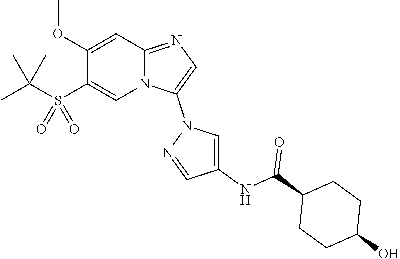 [SMILES: COc1cc2ncc(-n3cc(NC(=O)[C@H]4CC[C@@H](O)CC4)cn3)n2cc1S(=O)(=O)C(C)(C)C] | (1s,4s)-N-(1-(6- (tert-butylsulfonyl)- 7- methoxyimidazo[1,2-a] pyridin-3-yl)-1H- pyrazol-4-yl)-4- hydroxycyclohexane-1- carboxamide | [M + H]⁺ = 476.0 ¹H NMR (400 MHz, DMSO-d₆) δ ppm 10.11 (s, 1H), 8.73 (s, 1H), 8.35 (s, 1H), 7.91 (s, 1H), 7.85 (s, 1H), 7.28 (s, 1H), 4.35 (s, 1H), 3.93 (s, 3H), 3.80 (s, 1H), 2.39-2.29 (m, 1H), 1.92-1.82 (m, 2H), 1.71-1.63 (m, 2H), 1.53-1.43 (m, 4H), 1.29 (s, 9H) |
| 173 | 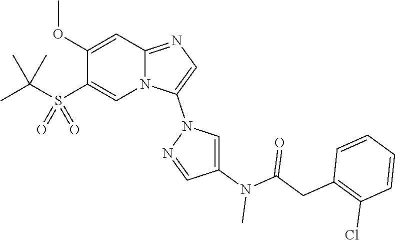 [SMILES: COc1cc2ncc(-n3cc(N(C)C(=O)Cc4ccccc4Cl)cn3)n2cc1S(=O)(=O)C(C)(C)C] | N-(1-(6-(tert- butylsulfonyl)-7- methoxyimidazo[1,2-a] pyridin-3-yl)-1H- pyrazol-4-yl)-2-(2- chlorophenyl)-N- methylacetamide | [M + H]⁺ = 516.1 ¹H NMR (400 MHz, DMSO-d₆) δ ppm 8.80 (s, 1H), 8.43 (s, 1H), 8.21-7.95 (m,1H), 7.82 (s, 1H), 7.45-7.22 (m, 5H), 3.96 (s, 3H), 3.95-3.61 (m, 2H), 3.48-3.15 (m, 3H), 1.31 (s, 9H) |
| 174 | 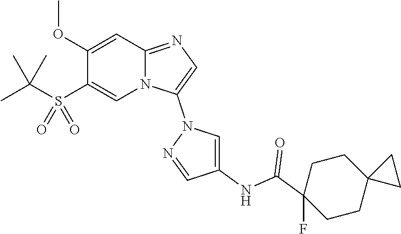 [SMILES: COc1cc2ncc(-n3cc(NC(=O)C4(F)CCC5(CC5)CC4)cn3)n2cc1S(=O)(=O)C(C)(C)C] | N-(1-(6-(tert- butylsulfonyl)-7- methoxyimidazo[1,2-a] pyridin-3-yl)-1H- pyrazol-4-yl)-6- fluorospiro[2.5]octane-6- carboxamide | [M + H]⁺ = 504.1 ¹H NMR (400 MHz, DMSO-d₆) δ ppm 10.56 (d, J = 2.8 Hz, 1H), 8.70 (s, 1H), 8.41 (s, 1H), 8.06 (s, 1H), 7.86 (s, 1H), 7.29 (s, 1H), 3.94 (s, 3H), 2.07-1.86 (m, 6H), 1.29 (s, 9H), 0.95-0.87 (m, 2H), 0.38-0.27 (m, 4H) |
| 175 | 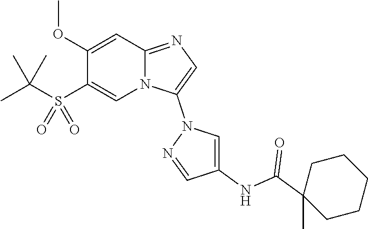 [SMILES: COc1cc2ncc(-n3cc(NC(=O)C4(C)CCCCC4)cn3)n2cc1S(=O)(=O)C(C)(C)C] | N-(1-(6-(tert- butylsulfonyl)-7- methoxyimidazo[1,2- a]pyridin-3-yl)-1H- pyrazol-4-yl)-1- methylcyclohexane- 1-carboxamide | [M + H]⁺ = 474.1 ¹H NMR (400 MHz, DMSO-d₆) δ ppm 9.74 (s, 1H), 8.76 (s, 1H), 8.39 (s, 1H), 7.99 (s, 1H), 7.85 (s, 1H), 7.28 (s, 1H), 3.94 (s, 3H), 2.10-2.02 (m, 2H), 1.56-1.43 (m, 4H), 1.37- 1.31 (m, 4H), 1.29 (s, 9H), 1.16 (s, 3H) |
| 176 | 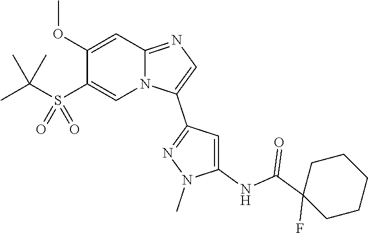 [SMILES: COc1cc2ncc(-c3cc(NC(=O)C4(F)CCCCC4)n(C)n3)n2cc1S(=O)(=O)C(C)(C)C] | N-(3-(6-(tert- butylsulfonyl)-7- methoxyimidazo[1,2- a]pyridin-3-yl)-1- methyl-1H-pyrazol- 5-yl)-1- fluorocyclohexane- 1-carboxamide | [M + H]⁺ = 492.1 ¹H NMR (400 MHz, DMSO-d₆) δ ppm 10.22 (s, 1H), 9.89 (s, 1H), 8.02 (s, 1H), 7.27 (s, 1H), 6.70 (s, 1H), 3.93 (s, 3H), 3.72 (s, 3H), 1.98-1.91 (m, 3H), 1.88-1.79 (m, 1H), 1.73-1.46 (m, 6H), 1.32 (s, 9H) |

### Example 177

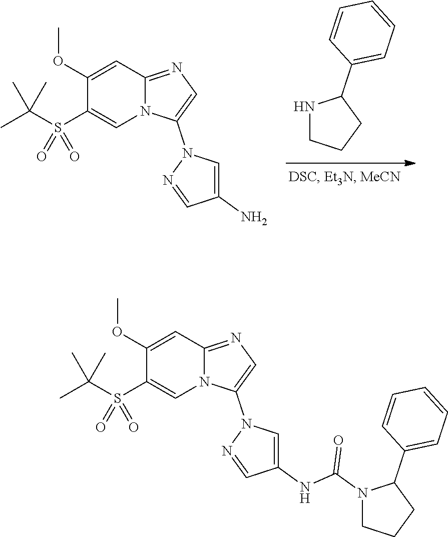

N-(1-(6-(tert-butylsulfonyl)-7-methoxyimidazo[1,2-a]pyridin-3-yl)-1H-pyrazol-4-yl)-2-phenylpyrrolidine-1-carboxamide: solution of 1-(6-(tert-butylsulfonyl)-7-methoxyimidazo[1,2-a]pyridin-3-yl)-1H-pyrazol-4-amine (30 mg, 85.86 μmol, 1 eq) in MeCN (1.5 mL) was added Et₃N (26.06 mg, 257.58 μmol, 35.85 μL, 3 eq) and N,N′-disuccinimidyl carbonate (26.39 mg, 103.03 μmol, 1.2 eq). The mixture was stirred at 20° C. for 0.3 hour, and then 2-phenylpyrrolidine was added (18.96 mg, 128.79 μmol, 1.5 eq). The resulting mixture was stirred at 20° C. for 12 hours before it was filtered and concentrated under reduced pressure to give a residue, which was purified by RP-HPLC to give the title compound (7.42 mg, 22% yield) as a white solid. [M+H]⁺=523.1. ¹H NMR (400 MHZ, DMSO-d₆) δ ppm 8.75 (s, 1H), 8.63 (s, 1H), 8.11 (s, 1H), 7.87 (s, 1H), 7.77 (s, 1H), 7.33-7.28 (m, 2H), 7.26 (s, 1H), 7.22-7.17 (m, 3H), 5.07 (d, J=7.6 Hz, 1H), 3.92 (s, 3H), 3.75-3.69 (s, 1H), 3.59-3.52 (m, 1H), 2.19-2.16 (m, 1H), 1.94-1.90 (m, 1H), 1.87-1.82 (m, 1H), 1.78-1.73 (m, 1H), 1.28 (s, 9H).

Examples 178 and 179 were obtained from racemic 177 by chiral SFC separation.

| Ex. | | | |
|---|---|---|---|
| No. | Structure | Name | LCMS, ¹H NMR |
| 178 | 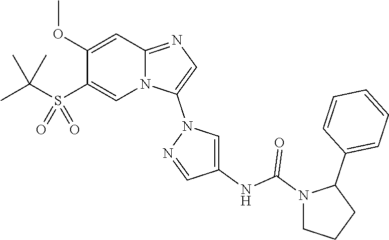 [SMILES: COc1cc2ncc(-n3cc(NC(=O)N4CCCC4c4ccccc4)cn3)n2cc1S(=O)(=O)C(C)(C)C] | (R)- or (S)-N-(1-(6- (tert-butylsulfonyl)-7- methoxyimidazo[1,2-a]pyridin- 3-yl)-1H- pyrazol-4-yl)-2-phenylpyrrolidine- 1-carboxamide | [M + H]⁺ = 523.1 ¹H NMR (400 MHz, DMSO-d₆) δ ppm 8.75 (s, 1H), 8.64 (s, 1H), 8.11 (s, 1H), 7.87 (s, 1H), 7.77 (s, 1H), 7.33-7.28 (m, 2H), 7.27-7.25 (s, 1H), 7.22-7.18 (m, 3H), 5.07 (dd, J = 7.6, 2.0 Hz, 1H), 3.92 (s, 3H), 3.76-3.69 (m, 1H), 3.59-3.51 (m, 1H), 2.33-2.25 (m, 1H), 1.95-1.90 (m, 1H), 1.88-1.80 (m, 1H), 1.78-1.73 (m, 1H), 1.28 (s, 9H). SFC: Rₜ = 0.833 min, 100% ee; CHIRALPAK AD-3 50 × 4.6 mm, 3 μm; 40% EtOH (0.05% DEA) in CO₂, 3 mL/min; 35° C., 100 Bar, 220 nm |
| 179 | 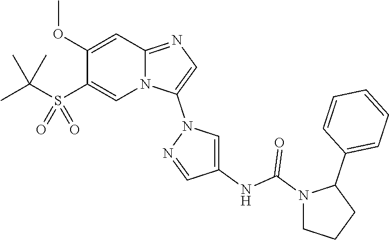 [SMILES: COc1cc2ncc(-n3cc(NC(=O)N4CCCC4c4ccccc4)cn3)n2cc1S(=O)(=O)C(C)(C)C] | (S)- or (R)-N-(1-(6-(tert- butylsulfonyl)-7-methoxyimidazo [1,2-a]pyridin-3-yl)-1H- pyrazol-4-yl)-2-phenylpyrrolidine- 1-carboxamide | [M + H]⁺ = 523.2 ¹H NMR (400 MHz, DMSO-d₆) δ ppm 8.75 (s, 1H), 8.64 (s, 1H), 8.11 (s, 1H), 7.87 (s, 1H), 7.77 (s, 1H), 7.33-7.28 (m, 2H), 7.26 (s, 1H), 7.22- 7.18 (m, 3H), 5.09-5.03 (m, 1H), 3.92 (s, 3H), 3.76-3.69 (m, 1H), 3.58-3.51 (m, 1H), 2.33-2.26 (m, 1H), 1.95-1.90 (m, 1H), 1.88-1.80 (m, 1H), 1.79-1.73 (m, 1H), 1.28 (s, 9H). SFC: Rₜ = 1.535 min, 99.1% ee; CHIRALPAK AD-3 50 × 4.6 mm, 3 μm; 40% EtOH (0.05% DEA) in CO₂, 3 mL/min; 35° C., 100 Bar, 220 nm |

### Example 180

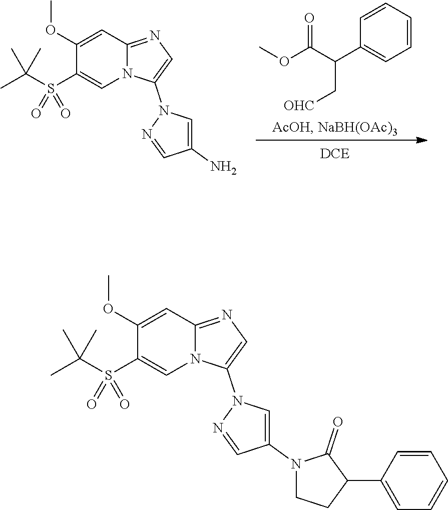

1-(1-(6-(tert-butylsulfonyl)-7-methoxyimidazo[1,2-a]pyridin-3-yl)-1H-pyrazol-4-yl)-3-phenylpyrrolidin-2-one: To a solution of methyl 4-oxo-2-phenylbutanoate (110.02 mg, 515.16 μmol, 1.0 eq) and 1-(6-(tert-butylsulfonyl)-7-methoxyimidazo[1,2-a]pyridin-3-yl)-1H-pyrazol-4-amine (200 mg, 515.16 μmol, 1 eq) in DCE (2 mL) was added AcOH (92.81 mg, 1.55 mmol, 88.47 μL, 3 eq). The mixture was stirred at 25° C. for 1 hour under a N₂ atmosphere, and then NaBH(OAc)₃ (338.47 mg, 1.60 mmol, 3.1 eq) was added. The mixture was stirred at 25° C. for 12 hours before it was concentrated under reduced pressure to give a residue, which was purified by RP-HPLC to give the title compound (40 mg, 16% yield) as an off-white solid. [M+H]⁺=493.9.

¹H NMR (400 MHZ, DMSO-d₆) δ ppm 8.75 (s, 1H), 8.51 (s, 1H), 8.26 (s, 1H), 7.88 (s, 1H), 7.38-7.24 (m, 6H), 3.95 (s, 1H), 3.94-3.93 (m, 3H), 3.91 (s, 1H), 3.88-3.80 (m, 1H), 2.70-2.62 (m, 1H), 2.35-2.23 (m, 1H), 1.29 (s, 9H).

The following compounds were prepared following procedures analogous to that described for Example 180. Examples 182 and 183 were obtained from racemic 180 by chiral SFC separation.

| Ex. No. | Structure | Name | LCMS, ¹H NMR |
|---|---|---|---|
| 181 | 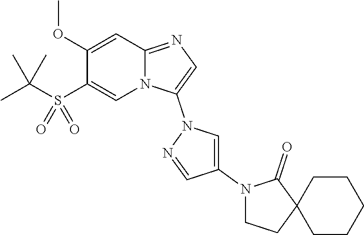 [SMILES: COc1cc2ncc(-n3cc(N4CCC5(CCCCC5)C4=O)cn3)n2cc1S(=O)(=O)C(C)(C)C] | 2-(1-(6-(tert-butylsulfonyl)-7- methoxyimidazo[1,2-a] pyridin-3-yl)-1H-pyrazol-4- yl)-2-azaspiro[4.5]decan-1-one | [M + H]⁺ = 485.9 ¹H NMR (400 MHz, DMSO-d₆) δ ppm 8.74 (s, 1H), 8.47 (s, 1H), 8.20 (s, 1H), 7.86 (s, 1H), 7.29 (s, 1H), 3.94 (s, 3H), 3.72 (t, J = 6.8 Hz, 2H), 2.09 (t, J = 6.8 Hz, 2H), 1.72-1.30 (m, 10H), 1.29 (s, 9H). |
| 182 | 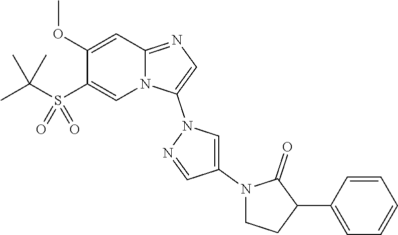 [SMILES: COc1cc2ncc(-n3cc(N4CCC(c5ccccc5)C4=O)cn3)n2cc1S(=O)(=O)C(C)(C)C] | (R)- or (S)-1-(1-(6-(tert- butylsulfonyl)-7- methoxyimidazo[1,2-a]pyridin- 3-yl)-1H-pyrazol-4-yl)-3- phenylpyrrolidin-2-one | [M + H]⁺ = 493.9 ¹H NMR (400 MHz, DMSO-d₆) δ ppm 8.75 (s, 1H), 8.51 (s, 1H), 8.26 (s, 1H), 7.88 (s, 1H), 7.38-7.24 (m, 6H), 3.95 (s, 1H), 3.95-3.92 (m, 3H), 3.91 (s, 1H), 3.85 (s, 1H), 2.69-2.63 (m, 1H), 2.31-2.21 (m, 1H), 1.29 (s, 9H). SFC: Rₜ = 0.809 min, 100% ee; CHIRALPAK AD-3 50 × 4.6 mm, 3 μm; 40% MeOH/MeCN (0.05% DEA) in CO₂, 3 mL/min; 35° C., 100 Bar, 220 nm |
| 183 | 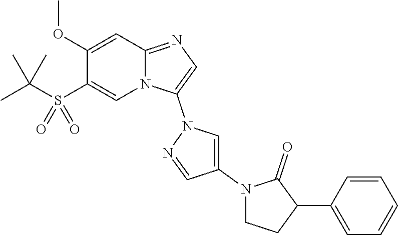 [SMILES: COc1cc2ncc(-n3cc(N4CCC(c5ccccc5)C4=O)cn3)n2cc1S(=O)(=O)C(C)(C)C] | (S)- or (R)-1-(1-(6-(tert- butylsulfonyl)-7-methoxy- imidazo[1,2-a]pyridin-3-yl)-1H- pyrazol-4-yl)-3- phenylpyrrolidin-2-one | [M + H]⁺ = 493.9 ¹H NMR (400 MHz, DMSO-d₆) δ ppm 8.75 (s, 1H), 8.51 (s, 1H), 8.26 (s, 1H), 7.88 (s, 1H), 7.38-7.25 (m, 6H), 3.97-3.95 (m, 1H), 3.94 (s, 3H), 3.93- 3.86 (m, 1H), 3.85-3.82 (m, 1H), 2.72-2.60 (m, 1H), 2.32- 2.21 (m, 1H), 1.29 (s, 9H) SFC: Rₜ = 1.695 min, 99.9% ee; CHIRALPAK AD-3 50 × 4.6 mm, 3 μm; 40% MeOH/MeCN (0.05% DEA) in CO₂, 3 mL/min; 35° C., 100 Bar, 220 nm |

### Example 184

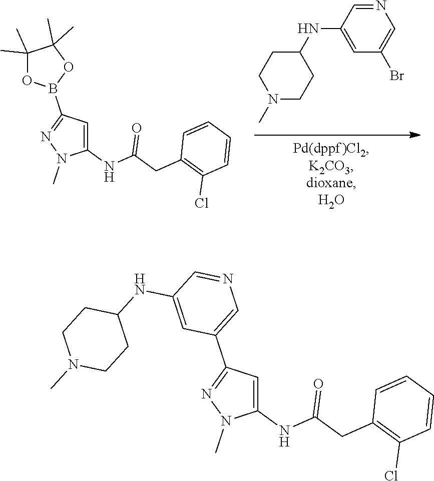

2-(2-chlorophenyl)-N-(1-methyl-3-(5-((1-methylpiperidin-4-yl)amino)pyridin-3-yl)-1H-pyrazol-5-yl) acetamide: A mixture of 2-(2-chlorophenyl)-N-(1-methyl-3-(4,4,5,5-tetramethyl-1,3,2-dioxaborolan-2-yl)-1H-pyrazol-5-yl) acetamide (50 mg, 133.10 μmol, 1 eq), 5-bromo-N-(1-methylpiperidin-4-yl) pyridin-3-amine (47.95 mg, 159.72 μmol, 1.2 eq), Pd (dppf) Cl₂ (9.74 mg, 13.31 μmol, 0.1 eq), and K₂CO₃ (55.19 mg, 399.30 μmol, 3 eq) in dioxane (2 mL) and H₂O (0.5 mL) was degassed and purged with N₂ three times, and then the mixture was stirred at 100° C. for 2 hours under a N₂ atmosphere. The reaction mixture was filtered and concentrated under reduced pressure to give a residue, which was purified by RP-HPLC to give the title compound (3.96 mg, 7% yield) as a white solid. [M+H]⁺=439.1. ¹H NMR (400 MHZ, DMSO-d₆) δ ppm 10.37 (s, 1H), 8.26 (s, 1.4H), 8.11 (s, 1H), 7.89 (s, 1H), 7.54-7.41 (m, 2H), 7.37-7.28 (m, 2H), 7.22 (s, 1H), 6.64 (s, 1H), 5.83 (d, J=7.2 Hz, 1H), 3.91 (s, 2H), 3.73 (s, 3H), 3.48-3.29 (m, 1H), 2.91-2.80 (m, 2H), 2.39-2.15 (m, 5H), 1.99-1.88 (m, 2H), 1.57-1.39 (m, 2H).

The following compounds were prepared following procedures analogous to that described for Example 184.

| Ex. | | | |
|---|---|---|---|
| No. | Structure | Name | LCMS, ¹H NMR |
| 185 | 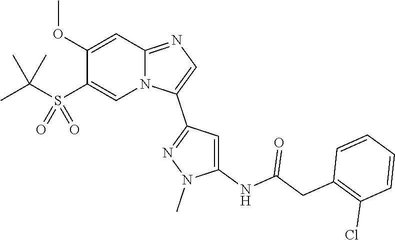 [SMILES: COc1cc2ncc(-c3cc(NC(=O)Cc4ccccc4Cl)n(C)n3)n2cc1S(=O)(=O)C(C)(C)C] | N-(3-(6-(tert- butylsulfonyl)-7- methoxyimidazo[1,2- a]pyridin-3-yl)-1- methyl-1H-pyrazol-5- yl)-2-(2- chlorophenyl)acetamide | [M + H]⁺ = 516.0 ¹H NMR (400 MHz, DMSO- d₆) δ ppm 10.41 (s, 1H), 9.91 (s, 1H), 8.00 (s, 1H), 7.49-7.43 (m, 2H), 7.36-7.30 (m, 2H), 7.25 (s, 1H), 6.74 (s, 1H), 3.96-3.90 (m, 5H), 3.79 (s, 3H), 1.32 (s, 9H) |
| 186 | 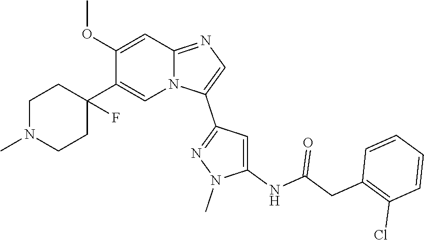 [SMILES: COc1cc2ncc(-c3cc(NC(=O)Cc4ccccc4Cl)n(C)n3)n2cc1C1(F)CCN(C)CC1] | 2-(2-chlorophenyl)-N- (3-(6-(4-fluoro-1- methylpiperidin-4-yl)-7- methoxyimidazo[1,2-a] pyridin-3-yl)-1-methyl-1H- pyrazol-5-yl)acetamide | [M + H]⁺ = 511.0 ¹H NMR (400 MHz, DMSO- d₆) δ ppm 10.40 (s, 1H), 9.38 (s, 1H), 8.38 (s, 1H), 7.82 (s, 1H), 7.54-7.42 (m, 2H), 7.38- 7.28 (m, 2H), 7.11 (s, 1H), 6.65 (s, 1H), 3.92 (s, 2H), 3.90 (s, 3H), 3.78 (s, 3H), 2.75-2.68 (m, 2H), 2.67-2.60 (m, 2H), 2.29-2.24 (m, 2H), 2.22 (s, 3H), 1.80-1.75 (m, 2H) |
| 187 | 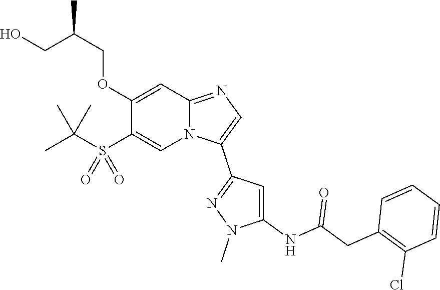 [SMILES: C[C@H](CO)COc1cc2ncc(-c3cc(NC(=O)Cc4ccccc4Cl)n(C)n3)n2cc1S(=O)(=O)C(C)(C)C] | (R)-N-(3-(6-(tert- butylsulfonyl)-7-(3- hydroxy-2- methylpropoxy)imidazo [1,2-a]pyridin-3-yl)-1- methyl-1H-pyrazol-5-yl)-2- (2-chlorophenyl)acetamide | [M + H]⁺ = 574.3 ¹H NMR (400 MHz, DMSO- d₆) δ ppm 10.44 (s, 1H), 9.91 (s, 1H), 7.99 (s, 1H), 7.51-7.41 (m, 2H), 7.38-7.29 (m, 2H), 7.20 (s, 1H), 6.73 (s, 1H), 4.57 (s, 1H), 4.12-3.98 (m, 2H), 3.94 (s, 2H), 3.79 (s, 3H), 3.52-3.41 (m, 2H), 2.09-1.98 (m, 1H), 1.33 (s, 9H), 1.01 (d, J = 6.8 Hz, 3H) SFC: Rₜ = 2.256 min, 94.4% ee; CHIRALPAK IK-3 50 × 4.6 mm, 3 μm; 30% MeOH (0.05% DEA) in CO₂, 3 mL/min; 35° C., 100 Bar, 220 nm |
| 188 | 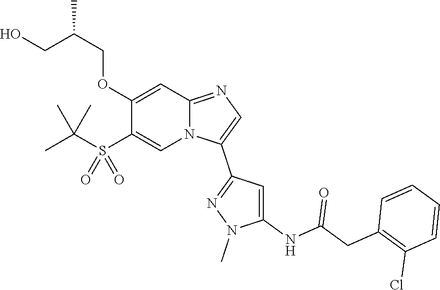 [SMILES: C[C@@H](CO)COc1cc2ncc(-c3cc(NC(=O)Cc4ccccc4Cl)n(C)n3)n2cc1S(=O)(=O)C(C)(C)C] | (S)-N-(3-(6-(tert- butylsulfonyl)-7-(3- hydroxy-2- methylpropoxy)imidazo [1,2-a]pyridin-3-yl)-1- methyl-1H-pyrazol-5- yl)-2-(2-chlorophenyl) acetamide | [M + H]⁺ = 574.3 ¹H NMR (400 MHz, DMSO- d₆) δ ppm 10.43 (s, 1H), 9.91 (s, 1H), 8.00 (s, 1H), 7.51-7.43 (m, 2H), 7.37-7.30 (m, 2H), 7.21 (s, 1H), 6.74 (s, 1H), 4.58 (t, J = 5.2 Hz, 1H), 4.13-4.08 (m, 1H), 4.04-3.99 (m, 1H), 3.94 (s, 2H), 3.80 (s, 3H), 3.53-3.44 (m, 2H), 2.09-2.00 (m, 1H), 1.34 (s, 9H), 1.02 (d, J = 6.8 Hz, 3H) SFC: Rₜ = 2.000 min, 99.2% ee; CHIRALPAK IK-3 50 × 4.6 mm, 3 μm; 30% MeOH (0.05% DEA) in CO₂, 3 mL/min; 35° C., 100 Bar, 220 nm |
| 189 | 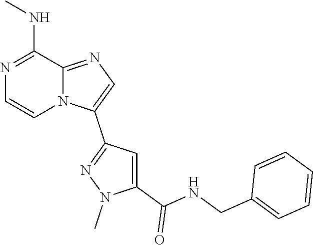 [SMILES: CNc1nccn2c(-c3cc(C(=O)NCc4ccccc4)n(C)n3)cnc12] | N-benzyl-1-methyl-3- (8-(methylamino) imidazo[1,2-a]pyrazin-3-yl)- 1H-pyrazole-5-carboxamide | [M + H]⁺ = 362.2 ¹H NMR (400 MHz, DMSO- d₆) δ ppm 9.18 (t, J = 6.0 Hz, 1H), 8.35 (d, J = 4.8 Hz, 1H), 7.83 (s, 1H), 7.52-7.49 (m, 1H), 7.43 (d, J = 4.4 Hz, 1H), 7.38-7.31 (m, 5H), 7.30-7.22 (m, 1H), 4.48 (d, J = 6.0 Hz, 2H), 4.17 (s, 3H), 2.96 (d, J = 4.8 Hz, 3H) |
| 190 | 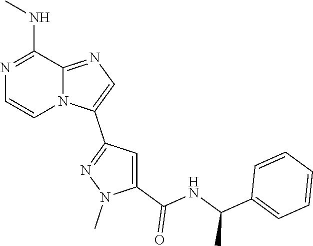 [SMILES: CNc1nccn2c(-c3cc(C(=O)N[C@H](C)c4ccccc4)n(C)n3)cnc12] | (R)-1-methyl-3-(8- (methylamino)imidazo [1,2-a]pyrazin-3-yl)-N- (1-phenylethyl)-1H- pyrazole-5-carboxamide | [M + H]⁺ = 376.3 ¹H NMR (400 MHz, DMSO- d₆) δ ppm 8.97 (d, J = 8.0 Hz, 1H), 8.36 (d, J = 4.8 Hz, 1H), 7.86 (s, 1H), 7.52-7.47 (m, 1H), 7.45-7.39 (m, 4H), 7.38- 7.32 (m, 2H), 7.29-7.21 (m, 1H), 5.24-5.09 (m, 1H), 4.12 (s, 3H), 2.96 (d, J = 5.2 Hz, 3H), 1.49 (d, J = 6.8 Hz, 3H) |
| 191 | 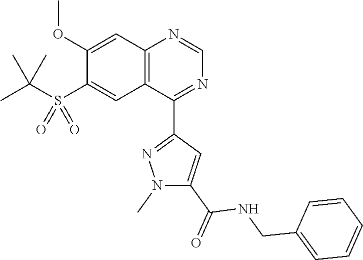 [SMILES: COc1cc2ncnc(-c3cc(C(=O)NCc4ccccc4)n(C)n3)c2cc1S(=O)(=O)C(C)(C)C] | N-benzyl-3-(6-(tert- butylsulfonyl)-7- methoxyquinazolin-4- yl)-1-methyl-1H- pyrazole-5-carboxamide | [M + H]⁺ = 494.1 ¹H NMR (400 MHz, DMSO- d₆) δ ppm 10.06 (s, 1H), 9.38- 9.26 (m, 2H), 7.93 (s, 1H), 7.61 (s, 1H), 7.35 (d, J = 4.4 Hz, 4H), 7.32-7.20 (m, 1H), 4.49 (d, J = 6.0 Hz, 2H), 4.26 (s, 3H), 4.07 (s, 3H), 1.33 (s, 9H) |
| 192 | 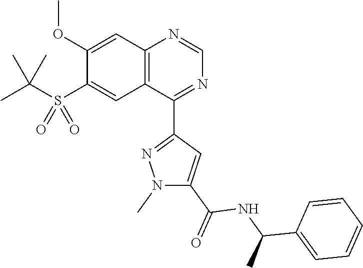 [SMILES: COc1cc2ncnc(-c3cc(C(=O)N[C@H](C)c4ccccc4)n(C)n3)c2cc1S(=O)(=O)C(C)(C)C] | (R)-3-(6-(tert- butylsulfonyl)-7- methoxyquinazolin-4- yl)-1-methyl-N-(1- phenylethyl)-1H- pyrazole-5-carboxamide | [M + H]⁺ = 508.1 ¹H NMR (400 MHz, DMSO- d₆) δ ppm 10.07 (s, 1H), 9.34 (s, 1H), 9.14 (d, J = 84 Hz, 1H), 8.04 (s, 1H), 7.16 (s, 1H), 7.45-7.39 (m, 2H), 7.35 (t, J = 7.2 Hz, 2H), 7.28-7.21 (m, 1H), 5.26-5.11 (m, 1H), 4.21 (s, 3H), 4.07 (s, 3H), 1.50 (d, J = 7.2 Hz, 3H), 1.32 (s, 9H) |

### Example 193

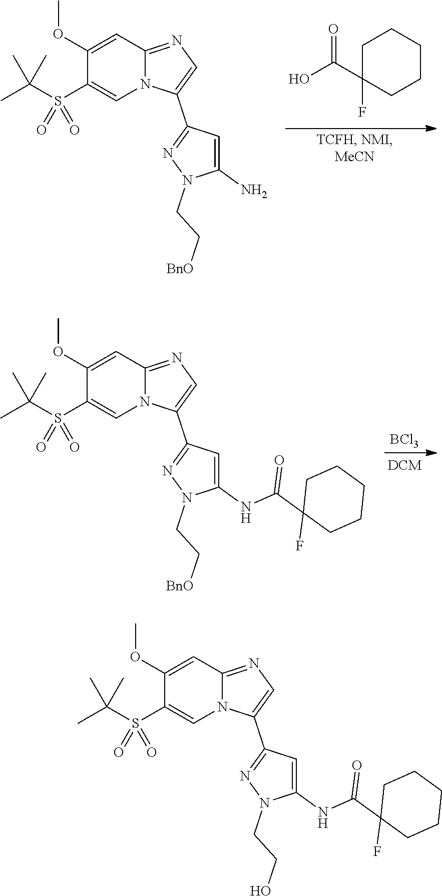

N-(1-(2-(benzyloxy)ethyl)-3-(6-(tert-butylsulfonyl)-7-methoxyimidazo[1,2-a]pyridin-3-yl)-1H-pyrazol-5-yl)-1-fluorocyclohexane-1-carboxamide: To a solution of 1-(2-(benzyloxy)ethyl)-3-(6-(tert-butylsulfonyl)-7-methoxyimidazo[1,2-a]pyridin-3-yl)-1H-pyrazol-5-amine (50 mg, 93.06 μmol, 1 eq) and 1-fluorocyclohexane-1-carboxylic acid (27.20 mg, 186.11 μmol, 2 eq) in MeCN (3 mL) was added TCFH (52.22 mg, 186.11 μmol, 2 eq) and NMI (22.92 mg, 279.17 μmol, 22.25 μL, 3 eq). The mixture was stirred at 25° C. for 4 hours before it was concentrated under reduced pressure to give a residue, which was purified by column chromatography (SiO₂, 0-100% EtOAc/petroleum ether) to give the title compound (40 mg, 63% yield) as colorless oil. [M+H]⁺=612.4.

N-(3-(6-(tert-butylsulfonyl)-7-methoxyimidazo[1,2-a]pyridin-3-yl)-1-(2-hydroxyethyl)-1H-pyrazol-5-yl)-1-fluorocyclohexane-1-carboxamide: To a solution of N-(1-(2-(benzyloxy)ethyl)-3-(6-(tert-butylsulfonyl)-7-methoxyimidazo[1,2-a]pyridin-3-yl)-1H-pyrazol-5-yl)-1-fluorocyclohexane-1-carboxamide (30 mg, 44.14 μmol, 1 eq) in DCM (30 mL) was added BCl₃ (0.3 mL) at 0° C. The mixture was stirred at 25° C. for 0.5 hour before it was quenched with saturated NaHCO₃ (20 mL) at 0° C., and then extracted with DCM (3×20 mL). The combined organic layers were washed with brine (20 mL), dried over Na₂SO₄, filtered and concentrated under reduced pressure to give a residue, which was purified by RP-HPLC to give the title compound (2.09 mg, 9% yield) as a white solid. [M+H]⁺=522.0. ¹H NMR (400 MHZ, DMSO-d₆) δ ppm 10.63-10.21 (m, 1H), 9.93 (s, 1H), 8.04 (s, 1H), 7.27 (s, 1H), 6.80 (s, 1H), 5.82-5.34 (m, 1H), 4.16 (t, J=5.6 Hz, 2H), 3.93 (s, 3H), 3.85-3.77 (m, 2H), 1.96-1.89 (m, 3H), 1.87-1.78 (m, 1H), 1.71-1.65 (m, 3H), 1.59-1.49 (m, 2H), 1.36 (s, 1H), 1.32 (s, 9H).

The following compound was prepared following procedures analogous to that described for Example 193.

| Ex. | | | |
|---|---|---|---|
| No. | Structure | Name | LCMS, ¹H NMR |
| 194 | 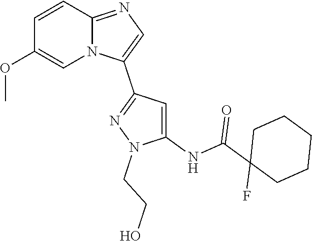 [SMILES: COc1ccc2ncc(-c3cc(NC(=O)C4(F)CCCCC4)n(CCO)n3)n2c1] | 1-fluoro-N-(1-(2- hydroxyethyl)-3-(5- methoxypyrazolo[1,5- a]pyrimidin-3-yl)-1H- pyrazol-5- yl)cyclohexane-1- carboxamide | [M + H]⁺ = 402.9 ¹H NMR (400 MHz, DMSO- d₆) δ ppm 10.28 (br s, 1H), 8.90 (d, J = 7.6 Hz, 1H), 8.32 (s, 1H), 6.89 (s, 1H), 6.57 (d, J = 7.6 Hz, 1H), 5.56 (br s, 1H), 4.13 (t, J = 5.6 Hz, 2H), 4.01 (s, 3H), 3.74 (t, J = 5.2 Hz, 2H), 1.95-1.91 (m, 3H), 1.91-1.88 (m, 1H), 1.69-1.60 (m, 3H), 1.57-1.48 (m, 2H), 1.37-1.28 (m, 1H) |

### Biological Data

### RIPK2 Binding Competition Assay

The ability of selected compounds of Formulas (I) and (II) to inhibit the binding of an Alexa647-labelled ATP-competitive kinase inhibitor to a GST-RIPK2 fusion protein was quantified employing the TR-FRET-based RIPK2 binding competition assay as described in the following paragraphs.

Recombinant fusion protein of N-terminal Glutathione-S-Transferase (GST) and a fragment of human RIPK2 (amino acids 1-310 of accession number 043353), expressed in baculovirus infected Sf9 cells, purified via glutathione affinity chromatography, was used as GST-RIPK2 fusion protein. Tracer 199 from Life Technologies (catalogue no. PR9115B) was used as Alexa647-labelled ATP-competitive kinase inhibitor.

For the assay, 50 nL of a 100-fold concentrated solution of each test compound in DMSO was pipetted into either a black low volume 384-well microtiter plate or a black 1536-well microtiter plate (both Greiner Bio-One, Frickenhausen, Germany), 3 μL solution of Tracer 199 (16.7 nM=>final concentration in 5 μL assay volume is 10 nM) in aqueous assay buffer (25 mM Tris/HCl pH 7.5, 10 mM magnesium chloride (MgCl₂), 5 mM β-glycerophosphate, 2.5 mM dithiothreitol (DTT), 0.5 mM ethylene glycol-bis(2-aminoethylether)-N,N,N′,N′-tetraacetic acid (EGTA), 0.5 mM sodium ortho-vanadate, 0.01% (w/v) bovine serum albumin (BSA), 0.005% (w/v) Pluronic F-127 (Sigma)) were added. Then the binding competition was started by the addition of 2 μL of a solution of the GST-RIPK2 fusion protein (2.5 nM=>final conc. in the 5 μL assay volume is 1 nM) and of Anti-GST-Tb (1.25 nM=>final conc. in the 5 μL assay volume is 0.5 nM), a Lumi4®—Tb cryptate-conjugated anti-GST antibody from PerkinElmer (catalogue no 61GSTTAH), in assay buffer.

The resulting mixture was incubated 45 min at 22° C. to allow the formation of a complex between the Tracer 199, the fusion protein and Anti-GST-Tb. Subsequently, the amount of this complex was evaluated by measurement of the resonance energy transfer from the Tb-cryptate to the Tracer 199. Therefore, the fluorescence emissions at 620 nm and 665 nm after excitation at 337 nm were measured in a TR-FRET reader, e.g. a Pherastar FS(BMG Labtechnologies, Offenburg, Germany) or a Viewlux (PerkinElmer). The ratio of the emissions at 665 nm and at 620 nm was taken as the measure of the amount of the complex. The data were normalized (assay reaction without inhibitor=0% activity, all other assay components but no GST-RIPK2 fusion protein=−100% activity). Typically the test compounds were tested on the same microtiter plate in 11 different concentrations in the range of 20 μM to 0.07 nM (20 μM, 5.7 μM, 1.6 μM, 0.47 μM, 0.13 μM, 38 nM, 11 nM, 3.1 nM, 0.9 nM, 0.25 nM and 0.07 nM, is the dilution series prepared separately before the assay on the level of the 100-fold concentrated solutions in DMSO by serial dilutions; exact concentrations may vary depending on pipettors used) in duplicate values for each concentration and IC₅₀ values were calculated using Genedata Screener™ software.

Table A1 below lists IC₅₀ values of selected compounds of Formula (I) measured in the RIPK2 binding competition assay. For the RIPK2 competition assay IC₅₀ values, “A” denotes an IC₅₀ of <200 nM; “B” denotes an IC₅₀ of 200 nM≤B≤500; “C” denotes an IC₅₀ of 500 nM<C≤1,000; and “D” denotes an IC₅₀ of 1,000 nM<D.

TABLE A1

| Ex. No. | RIPK2 IC₅₀ |
|---|---|
| 1 | B |
| 2 | B |
| 3 | D |
| 4 | A |
| 5 | A |
| 6 | D |
| 152 | A |
| 153 | B |
| 154 | C |
| 155 | D |
| 156 | C |
| 157 | C |
| 158 | C |
| 159 | A |
| 160 | B |
| 161 | B |
| 162 | C |
| 163 | D |
| 164 | D |
| 165 | D |
| 166 | D |
| 167 | D |
| 168 | D |
| 169 | B |
| 170 | D |
| 171 | A |
| 172 | D |
| 173 | D |
| 174 | C |
| 175 | D |
| 176 | A |
| 177 | D |
| 178 | D |
| 179 | C |
| 180 | D |
| 181 | D |
| 182 | D |
| 183 | D |
| 184 | A |
| 185 | A |
| 186 | A |
| 193 | A |
| 194 | D |

### RIPK2-XIAP Binding Competition PPI Assay

The ability of selected compounds of Formula (I) to inhibit the binding of an His-XIAP (BIR2)-avi fusion protein to a GST-RIPK2 fusion protein was quantified employing the TR-FRET-based competition assay as described in the following paragraphs.

Recombinant fusion protein of N-terminal Glutathione-S-Transferase (GST) and a fragment of human RIPK2 (amino acids 1-310 of accession number 043353), expressed in baculovirus infected Sf9 cells, purified via glutathione affinity chromatography, was used as GST-RIPK2 fusion protein. Recombinant fusion protein of N-terminal 6xHistidine (His), a fragment of human XIAP (amino acids 152-231 of accession number P98170), and C-terminal avi tag, expressed in baculovirus infected Sf9 cells, purified via Nickel NTA affinity chromatography, was used as the His-XIAP (BIR2)-avi fusion protein.

For the assay, a working solution of 1 nM GST-RIPK2, 25 nM His-XIAP (BIR2)-avi, 6 nM Anti-His-XL665 nM (Revvity Catalogue No. 61HISXLB), and 0.5 nM Anti-GST-Tb(Revvity Catalogue No. 61GSTTLB) in 25 mM Tris-HCl 7.5, 0.5 mM ethylene glycol-bis(2-aminoethylether)-N,N,N′,N′-tetraacetic acid (EGTA), 10 mM magnesium chloride (MgCl₂), 2.5 mM dithiothreitol (DTT), 0.01% (w/v) bovine serum albumin (BSA), 0.005% (w/v) Pluronic F-127 (Sigma), 0.5 mM sodium ortho-vanadate, 5 mM-Glycerophosphate disodium salt hydrate (BGP) was prepared. 50 nL of a 100-fold concentrated solution of each test compound in DMSO was dispensed via acoustic dispensing into a white low volume 384-well microtiter plate (Perkin Elmer Catalogue No. 6008280). Subsequently, 5 μL of the assay working solution were added.

The resulting mixture was incubated 120 min at 23° C. Subsequently, the amount of complex formed by the GST-RIPK2, XIAP (BIR2)-avi, Anti-His-XL665, and Anti-GST-Tb was evaluated by measurement of the resonance energy transfer from the Tb-cryptate to the XL665. Therefore, the fluorescence emissions at 520 nm and 665 nm after excitation at 337 nm were measured in a TR-FRET reader, e.g. a Pherastar FS(BMG Labtechnologies, Offenburg, Germany) or an Envision (PerkinElmer). The ratio of the emissions at 665 nm and at 520 nm was taken as the measure of the amount of the complex. The data were normalized (assay reaction without inhibitor=0% activity, 50 nM of GSK583=−100% activity). Typically the test compounds were tested on the same microtiter plate in 11 different concentrations in the range of 10 μM to 0.04 nM (10 μM, 2.86μ, 0.82μ, 0.23μ, 67 nM, 19 nM, 5.4 nM, 1.6 nM, 0.4 nM, 0.1 nM and 0.04 nM, is the dilution series prepared separately before the assay on the level of the 100-fold concentrated solutions in DMSO by serial dilutions; exact concentrations may vary depending on pipettors used) in duplicate values for each concentration and IC₅₀ values were calculated using Collaborative Drug Discovery software.

### THP-1 Dual Cell SEAP and Viability Assays

THP1-Dual™ cells (InvivoGen Cat #thpd-nfis) allow simultaneous assessment of the NF-κB pathway, by monitoring the activity of secreted embryonic alkaline phosphatase (SEAP) as well as the IRF pathway, by assessing the activity of a secreted luciferase (Lucia). Cells were cultured according to manufacturers' recommendations. For the assay, cell concentration was adjusted to 7.14×10⁵ cells/mL in assay media (RPMI 1640, 2 mM L-Glutamine, 25 mM HEPES, 10% fetal bovine serum (heat-inactivated for 30 min at 56° C.), Pen-Strep (100 U/mL)) and 35 μL of cell suspension (˜25,000 cells) per well were added to a flat bottom 384-well plate (white opaque). Plate was centrifuged at 300 g for 2 min, compounds were added in serial dilution series according to the plate layout (volumes ˜500 nL-0.0152 nL) and plate was incubated at 37° C. in 5% CO₂ for 30 min. Using assay media, a L18-MDP stock solution, that results in a final concentration of 10 ng/mL L18-MDP in each well when adding 15 μL to each well, was prepared. 15 μL of L18-MDP stock solution was added to each well, while 15 μL of assay media was added to the negative control wells. Plates were subsequently incubated at 37° C. in 5% CO₂ overnight (20-24 h).

For the SEAP assay: without disturbing the cells at the bottom of the well, 15 μL supernatant was transferred to a clear 384-well flat bottom plate and 35 μL QUANTI-Blue Solution per well was added. Following a 2 h incubation at 37° C., the optical density (OD) was measured at 620-655 nm using a microplate reader (PheraStar FS microplate reader with protocol for OD 620-655 nm).

For the viability assay: the cells remaining in the plate were used to assess cell viability. For this, CellTiter-Glo buffer and lyophilized CellTiter-Glo substrate was allowed to equilibrate to room temperature and lyophilized CellTiter-Glo substrate was reconstituted according to the manufacturers' recommendations. The equal volume (here 35 μL) of CTG was added to each well, contents were mixed on an orbital shaker for 2 min to induce lysis followed by a 10 min incubation at room temperature. Luminescence signal was subsequently measured in a microplate reader (PheraStar).

### Analysis

Values of media-only wells were subtracted and % inhibition for each compound concentration relative to the DMSO/L18-MDP-treated controls was calculated. Inhibition values+/−SD were fitted by non-linear regression using Prism software (GraphPad PRISM Software) to calculate IC₅₀ values.

Table A below lists potency values of selected compounds of Formula (I) measured in the RIPK2 (KD)/XIAP (BIR2) binding competition PPI assay and the THP-1 Dual cell SEAP and viability assays. “n.t.” denotes not tested.

For the RIPK2 (KD)/XIAP (BIR2) competition PPI assay IC₅₀ values, “A” denotes an IC₅₀ of <10 nM; “B” denotes an IC₅₀ of 10 nM≤B≤100; “C” denotes an IC₅₀ of 100 nM<C≤1000; and “D” denotes an IC₅₀ of 1000 nM<D.

For the RIPK2 (KD)/XIAP (BIR2) competition assay Aₘₐₓ values, “A” denotes an Aₘₐₓ of ≥80%; “B” denotes an Aₘₐₓ of 80%>B≥55; “C” denotes an Aₘₐₓ of 55%>C>30; and “D” denotes an Aₘₐₓ of ≤30%.

For the THP assay IC₅₀ values, “A” denotes an IC₅₀ of <100 nM; “B” denotes an IC₅₀ of 100 nM≤B≤500; “C” denotes an IC₅₀ of 500 nM<C≤2,500; and “D” denotes an IC₅₀ of 2,500 nM<D.

TABLE A
Results from Selected Biological Assays

| Ex. No. | PPI IC₅₀ | PPI Aₘₐₓ | IFNγ THP1 IC₅₀ |
|---|---|---|---|
| 1 | C | A | D |
| 2 | C | A | D |
| 3 | D | C | D |
| 4 | B | A | C |
| 5 | D | D | D |
| 6 | D | A | D |
| 7 | D | A | D |
| 8 | D | A | D |
| 9 | B | A | A |
| 10 | B | A | B |
| 11 | D | D | D |
| 12 | C | A | C |
| 13 | C | A | D |
| 14 | A | A | B |
| 15 | C | B | D |
| 16 | D | B | D |
| 17 | D | B | D |
| 18 | B | A | C |
| 19 | A | A | B |
| 20 | C | A | D |
| 21 | C | A | D |
| 22 | D | D | D |
| 23 | D | D | C |
| 24 | C | A | C |
| 25 | D | D | D |
| 26 | C | A | C |
| 27 | D | B | D |
| 28 | C | A | C |
| 29 | D | D | D |
| 30 | D | C | D |
| 31 | D | D | D |
| 32 | A | A | A |
| 33 | C | A | D |
| 34 | D | A | D |
| 35 | D | C | D |
| 36 | D | C | D |
| 37 | D | C | D |
| 38 | D | C | D |
| 39 | C | A | D |
| 40 | C | A | D |
| 41 | C | A | D |
| 42 | B | A | B |
| 43 | C | A | D |
| 44 | C | A | D |
| 45 | B | A | D |
| 46 | C | A | D |
| 47 | B | A | C |
| 48 | B | A | C |
| 49 | B | A | C |
| 50 | B | A | B |
| 51 | D | B | D |
| 52 | C | A | D |
| 53 | C | A | D |
| 54 | C | A | C |
| 55 | D | C | D |
| 56 | C | A | D |
| 57 | D | A | D |
| 58 | C | A | C |
| 59 | C | A | C |
| 60 | D | A | D |
| 61 | C | A | D |
| 62 | C | A | C |
| 63 | C | A | D |
| 64 | B | A | C |
| 65 | C | A | D |
| 66 | C | A | C |
| 67 | D | B | D |
| 68 | D | D | D |
| 69 | D | D | D |
| 70 | D | C | D |
| 71 | B | A | C |
| 72 | C | A | D |
| 73 | B | A | C |
| 74 | B | A | C |
| 75 | B | A | C |
| 76 | D | D | D |
| 77 | C | A | D |
| 78 | D | B | D |
| 79 | C | A | D |
| 80 | B | A | B |
| 81 | C | A | D |
| 82 | B | A | C |
| 83 | C | A | D |
| 84 | B | A | C |
| 85 | B | A | C |
| 86 | B | A | B |
| 87 | B | A | C |
| 88 | C | A | C |
| 89 | B | A | B |
| 90 | B | B | C |
| 91 | C | B | D |
| 92 | C | A | D |
| 93 | B | A | B |
| 94 | B | A | B |
| 95 | A | A | A |
| 96 | A | A | A |
| 97 | A | A | A |
| 98 | A | A | A |
| 99 | A | A | A |
| 100 | A | A | A |
| 101 | A | A | A |
| 102 | A | A | A |
| 103 | B | A | B |
| 104 | D | A | D |
| 105 | B | A | C |
| 106 | B | A | B |
| 107 | B | A | C |
| 108 | B | A | B |
| 109 | B | A | B |
| 110 | B | A | A |
| 111 | B | A | B |
| 112 | D | B | D |
| 113 | C | A | C |
| 114 | B | A | B |
| 115 | B | A | C |
| 116 | B | A | B |
| 117 | B | A | A |
| 118 | A | A | A |
| 119 | A | A | A |
| 120 | A | A | A |
| 121 | A | A | A |
| 122 | B | A | A |
| 123 | C | A | D |
| 124 | C | A | D |
| 125 | B | A | A |
| 126 | A | A | A |
| 127 | A | A | A |
| 128 | A | A | A |
| 129 | A | A | A |
| 130 | A | A | A |
| 131 | B | A | C |
| 132 | A | A | A |
| 133 | C | A | C |
| 134 | B | A | B |
| 135 | D | A | D |
| 136 | A | A | A |
| 137 | A | A | A |
| 138 | B | A | B |
| 139 | B | A | B |
| 140 | B | A | C |
| 141 | C | A | D |
| 142 | B | A | C |
| 143 | B | A | B |
| 144 | B | A | B |
| 145 | B | A | A |
| 146 | B | A | B |
| 147 | C | A | C |
| 148 | C | A | D |
| 149 | B | A | B |
| 150 | C | A | C |
| 151 | A | A | A |
| 152 | C | A | D |
| 153 | D | A | D |
| 154 | D | A | D |
| 155 | D | D | D |
| 156 | D | A | D |
| 157 | D | A | D |
| 158 | D | A | D |
| 159 | C | A | C |
| 160 | C | A | D |
| 161 | C | A | D |
| 162 | D | A | D |
| 163 | D | D | D |
| 164 | D | A | D |
| 165 | D | B | D |
| 166 | D | A | D |
| 167 | D | A | D |
| 168 | D | A | D |
| 169 | D | A | D |
| 170 | D | B | D |
| 171 | C | A | C |
| 172 | D | C | D |
| 173 | D | A | D |
| 174 | D | A | D |
| 175 | D | B | D |
| 176 | C | A | C |
| 177 | D | A | D |
| 178 | D | C | D |
| 179 | D | A | D |
| 180 | D | A | D |
| 181 | D | C | D |
| 182 | D | C | D |
| 183 | D | A | D |
| 184 | C | A | D |
| 185 | B | A | C |
| 186 | B | A | C |
| 187 | A | A | B |
| 188 | A | A | B |
| 189 | D | B | D |
| 190 | D | C | D |
| 191 | C | A | D |
| 192 | B | A | C |
| 193 | C | A | D |
| 194 | D | D | D |

What is claimed is:

1. A compound of Formula (I): 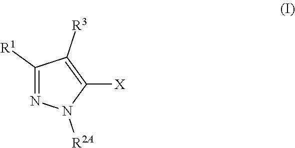
or a pharmaceutically acceptable salt of either of the foregoing, wherein:
R¹ is a 5-10 membered heteroaryl optionally substituted with 1-3 substituents independently selected from C1-C6 alkyl, C1-C6 thioalkyl, C1-C6 alkoxy optionally substituted with hydroxyl, —S(O₂) C1-C6 alkyl, NR^{1A}R^{1B}, 4-10 membered heterocyclyl optionally substituted with 1-3 substituents independently selected from halogen and C1-C6 alkyl, —(NH)—C1-C6 alkyl, and —(NH)-4-10 membered heterocyclyl optionally substituted with 1-3 substituents independently selected from halogen and C1-C6 alkyl;
R^{1A} and R^{1B} are independently selected from hydrogen, C1-C6 alkyl, and C1-C6 haloalkyl;
R^{2A} is selected from hydrogen, halogen, C1-C6 alkyl, C1-C6 haloalkyl, C1-C6 alkoxyalkyl, C1-C6 hydroxyalkyl optionally substituted with 1 or 2 halogens, C3-C6 cycloalkyl, 4-10 membered heterocyclyl optionally substituted with 1 or 2 substituents independently selected from halogen, C1-C6 alkyl, and C(═O) OC1-C6 alkyl;
R³ is hydrogen or halogen;
X is selected from 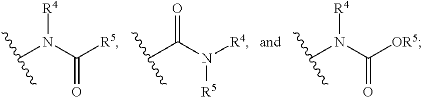
R⁴ is hydrogen or C1-C6 alkyl;
R⁵ is selected from:
(i) C1-C6 alkyl optionally substituted with 1-4 substituents independently selected from:
(a) C3-C6 cycloalkyl optionally substituted with 1-3 substituents independently from halogen or phenyl,
(b) phenyl optionally substituted with 1-3 substituents independently selected from halogen, C1-C6 alkoxy, C1-C6 hydroxyalkyl, and cyano,
(c) —NR^{A}R^{B},
(d) hydroxyl,
(e) halogen,
(f) 4-10 membered heterocyclyl optionally substituted with 1-3 substituents independently selected from benzyl, C1-C6 alkyl, and phenyl optionally substituted with C1-C6 alkyl, and
(g) 5-10 membered heteroaryl optionally substituted with 1-3 substituents independently selected from halogen, cyano, C1-C6 alkyl, and C1-C6 alkoxy;
(ii) C3-C10 cycloalkyl optionally substituted with 1-3 substituents independently selected from halogen, hydroxyl, C1-C6 alkyl, phenyl optionally substituted with 1-2 independently selected halogen and 5-10 membered heteroaryl;
(iii) 4-10 membered heterocyclyl optionally substituted with 1-3 substituents independently selected from benzyl, C1-C6 alkyl, C3-C10 cycloalkyl, and phenyl optionally substituted with C1-C6 alkyl;
(iv) phenyl optionally substituted with 1-3 substituents independently selected from halogen, nitro, and C1-C6 haloalkyl;
(v) 5-10 membered heteroaryl optionally substituted with 1-3 substituents independently selected from halogen and C1-C6 alkyl;
(vi) C1-C6 alkoxy; or
(vii) C3-C6 cycloalkoxy;
or R⁴ and R⁵ with the atom to which they are attached together form a 5-12 membered heterocyclyl or 9-10 membered heteroaryl each optionally substituted with phenyl; and
R^{A} and R^{B} are independently selected from hydrogen and C1-C6 alkyl.

2. The compound of claim 1, wherein Formula (I) is (I-a): 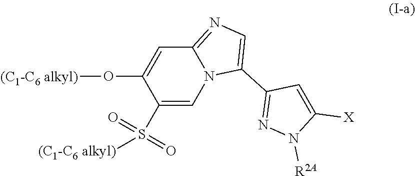
or a pharmaceutically acceptable salt thereof.

3. The compound of claim 1, wherein Formula (I) is (I-b): 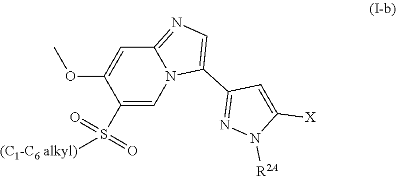
or a pharmaceutically acceptable salt thereof.

4. The compound of claim 1, wherein Formula (I) is (I-c): 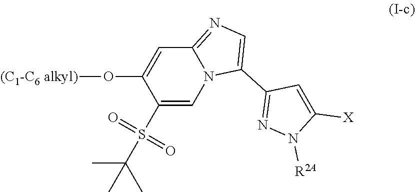
or a pharmaceutically acceptable salt thereof.

5. The compound of claim 1, wherein Formula (I) is (I-d): 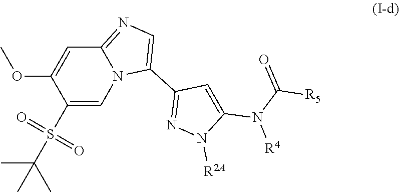
or a pharmaceutically acceptable salt thereof.

6. The compound of claim 1, wherein Formula (I) is (I-e): 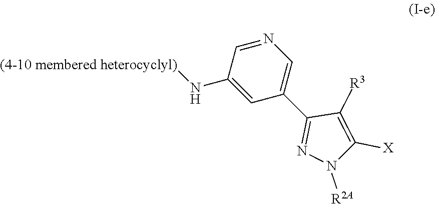
or a pharmaceutically acceptable salt thereof.

7. The compound of claim 1, wherein Formula (I) is (I-f): 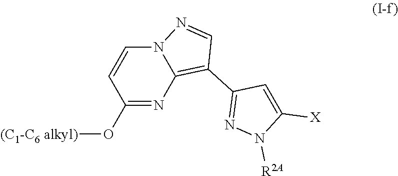
or a pharmaceutically acceptable salt thereof.

8. The compound of claim 1, wherein Formula (I) is (I-g): 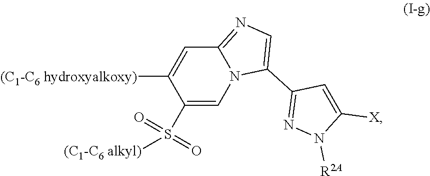
or a pharmaceutically acceptable salt thereof.

9. The compound of claim 1, wherein Formula (I) is (I-h): 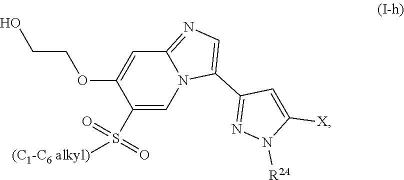
or a pharmaceutically acceptable salt thereof.

10. The compound of claim 1, wherein Formula (I) is (I-i): 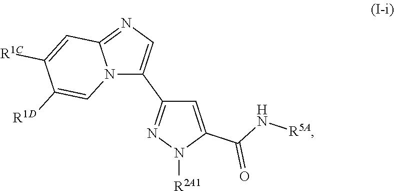
or a pharmaceutically acceptable salt thereof; wherein:
R^{1C} is C1-C6 alkoxy optionally substituted with hydroxyl;
R^{1D} is —S(O₂)—C1-C6 alkyl;
R^{2A1} is C1-C3 alkyl or C3-C4 cycloalkyl; and
R^{5A} is phenyl optionally substituted with halogen or C1-C6 alkyl; or 5-6 membered heteroaryl optionally substituted with halogen or C1-C6 alkyl.

11. The compound of claim 1, wherein Formula (I) is (I-j): 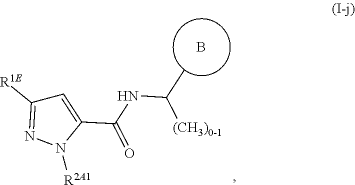
or a pharmaceutically acceptable salt thereof, wherein:
R^{1E} is a 5-10 membered heteroaryl substituted with 2 substituents independently selected from C1-C6 alkyl, C1-C6 thioalkyl, C1-C6 alkoxy optionally substituted with hydroxyl, and —S(O₂) C1-C6 alkyl;
R^{2A1} is C1-C3 alkyl or C3-C4 cycloalkyl; and
Ring B is (i) phenyl optionally substituted with 1-3 substituents independently selected from halogen, C1-C6 alkoxy, C1-C6 hydroxyalkyl, and cyano; or (ii) 5-6 membered heteroaryl optionally substituted with 1-3 substituents independently selected from halogen, cyano, C1-C6 alkyl, and C1-C6 alkoxy.

12. A compound selected from the group consisting of the compounds in Examples 1-194, or a pharmaceutically acceptable salt of any of the foregoing.

13. A pharmaceutical composition comprising a compound of any one of claims 1-12, or a pharmaceutically acceptable salt thereof, and one or more pharmaceutically acceptable excipients.

14. A method of treating a RIPK2-associated disease or disorder in a subject in need thereof, comprising administering to the subject an effective amount of a compound of any one of claims 1-12, or a pharmaceutically acceptable salt thereof, or the pharmaceutical composition of claim 13.

15. A method of treating inflammatory bowel disease in a subject in need thereof, comprising administering to the subject an effective amount of the compound of any one of claims 1-12, or a pharmaceutically acceptable salt thereof, or the pharmaceutical composition of claim 13.

16. A method of treating inflammatory bowel disease in a subject in need thereof, comprising (a) determining that the subject is suffering from inflammatory bowel disease; and (b) administering to the subject an effective amount of the compound of the compound of any one of claims 1-12, or a pharmaceutically acceptable salt thereof, or the pharmaceutical composition of claim 13.

17. A method of treating inflammatory bowel disease in a subject previously identified or diagnosed as having inflammatory bowel disease, the method comprising administering to the subject an effective amount of the compound of the compound of any one of claims 1-12, or a pharmaceutically acceptable salt thereof, or the pharmaceutical composition of claim 13.

18. A method of treating Crohn's disease in a subject in need thereof, comprising administering to the subject an effective amount of the compound of the compound of any one of claims 1-12, or a pharmaceutically acceptable salt thereof, or the pharmaceutical composition of claim 13.

19. A method of treating Crohn's disease in a subject in need thereof, comprising (a) determining that the subject is suffering from Crohn's disease; and (b) administering to the subject an effective amount of the compound of the compound of any one of claims 1-12, or a pharmaceutically acceptable salt thereof, or the pharmaceutical composition of claim 13.

20. A method of treating Crohn's disease in a subject previously identified or diagnosed as having Crohn's disease, the method comprising administering to the subject an effective amount of the compound of the compound of any one of claims 1-12, or a pharmaceutically acceptable salt thereof, or the pharmaceutical composition of claim 13.
